(19) United States
(12) Patent Application Publication
(10) Pub. No.: US 2026/0130109 A1
(43) Pub. Date: May 7, 2026

(54) COMPOUND FOR ORGANIC ELECTRONIC ELEMENT, ORGANIC ELECTRONIC ELEMENT USING THE SAME, AND AN ELECTRONIC DEVICE THEREOF

(71) Applicant: DUK SAN NEOLUX CO., LTD., Cheonan-si (KR)

(72) Inventors: Ji Su AN, Cheonan-si (KR); Se Hoon LEE, Cheonan-si (KR); Hyun Ju SONG, Cheonan-si (KR); Hyun Ji OH, Cheonan-si (KR)

(73) Assignee: DUK SAN NEOLUX CO., LTD., Cheonan-si (KR)

(21) Appl. No.: 19/427,568

(22) Filed: Dec. 19, 2025

Related U.S. Application Data
(63) Continuation-in-part of application No. 19/231,353, filed on Jun. 6, 2025.
Application No. 19/231,353 is a continuation-in-part of application No. 18/180,625, filed on Mar. 8, 2023.
Application No. 18/180,625 is a continuation-in-part of application No. 17/212,886, filed on Mar. 25, 2021, now Pat. No. 11,678,577.
Application No. 17/212,886 is a continuation of application No. 17/096,790, filed on Nov. 12, 2020, now Pat. No. 11,063,226.

(30) Foreign Application Priority Data
Oct. 26, 2020 (KR) 10-2020-0139441

(51) Int. Cl. H10K 85/60; C07D 251/24; C09K 11/06; H10K 50/11; H10K 50/15; H10K 50/16; H10K 101/00; H10K 101/10

(52) U.S. Cl. CPC H10K 85/636; C07D 251/24; C09K 11/06; H10K 85/626; H10K 85/631; H10K 85/653; H10K 85/654; H10K 85/6572; H10K 85/6574; H10K 85/6576; C07B 2200/05; C09K 2211/1018; H10K 50/11; H10K 50/15; H10K 50/16; H10K 2101/10; H10K 2101/90

(57) ABSTRACT

Provided are a compound of Formula 1, a composition comprising the same, an organic electronic element using the same, and an electronic device thereof, wherein the compound can improve the luminous efficiency, stability, and lifespan of the element.

### BACKGROUND

### Technical Field

The present invention relates to compounds for organic electronic elements, organic electronic elements using the same, and an electronic device thereof.

### Background Art

In general, organic light emitting phenomenon refers to a phenomenon that converts electric energy into light energy by using an organic material. An organic electronic element using an organic light emitting phenomenon usually has a structure including an anode, a cathode, and an organic material layer interposed therebetween. Here, in order to increase the efficiency and stability of the organic electronic element, the organic material layer is often composed of a multi-layered structure composed of different materials, and for example, may include a hole injection layer, a hole transport layer, an emitting layer, an electron transport layer, an electron injection layer etc.

A material used as an organic material layer in an organic electronic element may be classified into a light emitting material and a charge transport material, such as a hole injection material, a hole transport material, an electron transport material, an electron injection material etc. depending on its function. And the light emitting material can be classified into a high molecular weight type and a low molecular weight type according to the molecular weight, and it can be classified into a fluorescent material derived from a singlet excited state of an electron and a phosphorescent material derived from a triplet excited state of an electron depending on the light emission mechanism. Further, the light emitting material may be divided into blue, green, and red light emitting materials and yellow and orange light emitting materials necessary for realizing a better natural color according to the emission color.

However, when only one material is used as a light emitting material, due to intermolecular interaction, the maximum emission wavelength shifts to a longer wavelength, and there are problems in that the color purity is lowered or the device efficiency is reduced due to the emission attenuation effect, therefore in order to increase color purity and increase luminescence efficiency through energy transfer, a host/dopant system may be used as a light emitting material. The principle is that when a small amount of a dopant having a smaller energy band gap than that of the host forming the emitting layer is mixed in the emitting layer, excitons generated in the emitting layer are transported to the dopant to emit light with high efficiency. Here, since the wavelength of the host moves to the wavelength band of the dopant, light having a desired wavelength can be obtained according to the type of dopant used.

Currently, the portable display market is a large-area display, and the size thereof is increasing, and thus, more power consumption than the power consumption required for the existing portable display is required. Therefore, power consumption has become a very important factor for a portable display having a limited power supply such as a battery, and the problem of efficiency and lifetime must also be solved.

Efficiency, lifespan, and driving voltage are interrelated. As efficiency increases, the driving voltage decreases relatively. As the driving voltage decreases, the crystallization of organic materials due to Joule heating generated during operation decreases, which results in a tendency for the lifespan to increase. However, the efficiency cannot be maximized simply by improving the organic material layer. This is because, when the energy level and T1 value between each organic material layer, and the intrinsic properties (mobility, interfacial properties, etc.) of materials are optimally combined, long lifetime and high efficiency can be achieved at the same time.

Therefore, while delaying the penetration and diffusion of metal oxide from the anode electrode (ITO) into the organic layer, which is one of the causes of shortening the lifetime of the organic electronic element, it should have stable characteristics against Joule heating generated during device driving, and OLED devices are mainly formed by a deposition method, and it is necessary to develop a material that can withstand a long time during deposition, that is, a material with strong heat resistance.

That is, in order to fully exhibit the excellent characteristics of an organic electronic element, it should be preceded that the material constituting the organic material layer in the device, such as a hole injection material, a hole transport material, a light emitting material, an electron transport material, an electron injection material, etc., is supported by a stable and efficient material. But the development of a stable and efficient organic material layer material for an organic electronic element has not yet been sufficiently made. Therefore, the development of new materials is continuously required, and in particular, the development of a host material for the emitting layer is urgently needed.

### BRIEF DESCRIPTION OF THE INVENTION

### Summary

In order to solve the problems of the above-mentioned background technology, the present invention has discovered a compound with a novel structure, and also discovered that when the compound is applied to an organic electronic element, the luminescence efficiency, stability, and lifetime of the element can be greatly improved.

Accordingly, the purpose of the present invention is to provide a novel compound, an organic electronic element using the same, and an electronic device thereof.

### Technical Solution

The present invention provides a compound represented by Formula 1.

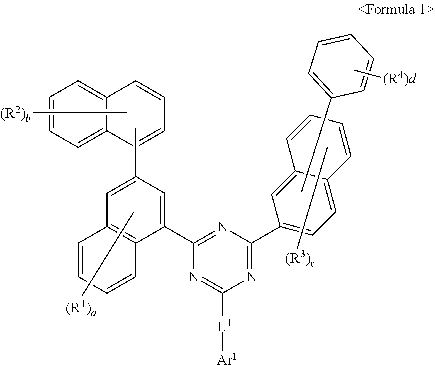

In another aspect, the present invention provides a compound for an organic electronic element comprising a first compound represented by Formula 1; and a second compound represented by Formula 4 or Formula 5;

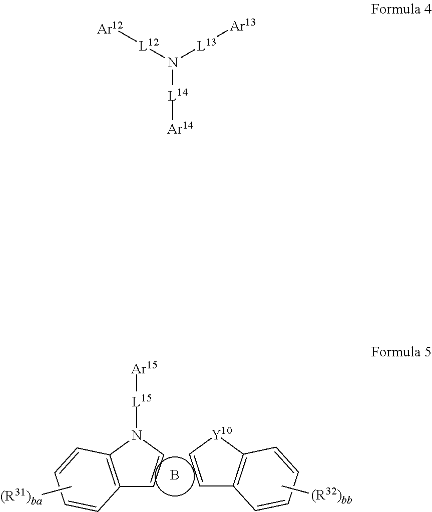

In another aspect, the present invention provides an organic electronic element comprising a compound represented by the Formula 1 or a compound for the organic electronic element and an electronic device thereof.

### Effects of the Invention

By using the compound according to the present invention, high luminescence efficiency, low driving voltage and high heat resistance of the element can be achieved, and color purity and lifetime of the element can be greatly improved.

### BRIEF DESCRIPTION OF THE DRAWINGS

FIG. 1 are exemplary views of an organic electroluminescent device according to the present invention.

FIG. 2 is a Formula according to one aspect of the present invention.

### DETAILED DESCRIPTION OF THE INVENTION

Hereinafter, some embodiments of the present invention will be described in detail. Further, in the following description of the present invention, a detailed description of known functions and configurations incorporated herein will be omitted when it may make the subject matter of the present invention rather unclear.

In addition, terms, such as first, second, A, B, (a), (b) or the like may be used herein when describing components of the present invention. Each of these terminologies is not used to define an essence, order or sequence of a corresponding component but used merely to distinguish the corresponding component from other component(s). It should be noted that if a component is described as being “connected”, “coupled”, or “connected” to another component, the component may be directly connected or connected to the other component, but another component may be “connected coupled” or “connected” between each component.

As used in the specification and the accompanying claims, unless otherwise stated, the following is the meaning of the term as follows.

Unless otherwise stated, the term “halo” or “halogen”, as used herein, includes fluorine (F), bromine (Br), chlorine (Cl), or iodine (I).

Unless otherwise stated, the term “alkyl” or “alkyl group”, as used herein, has a single bond of 1 to 60 carbon atoms, 1 to 30 carbon atoms, 1 to 25 carbon atoms, 1 to 18 carbon atoms, or 1 to 12 carbon atoms, and means saturated aliphatic functional radicals including a linear alkyl group, a branched chain alkyl group, a cycloalkyl group (alicyclic), an cycloalkyl group substituted with a alkyl or an alkyl group substituted with a cycloalkyl.

Unless otherwise stated, the term “alkenyl” or “alkynyl”, as used herein, has double or triple bonds of 2 to 60 carbon atoms, 2 to 30 carbon atoms, 2 to 25 carbon atoms, 2 to 18 carbon atoms, 2 to 12 carbon atoms, but is not limited thereto, and includes a linear or a branched chain group.

Unless otherwise stated, the term “cycloalkyl”, as used herein, means alkyl forming a ring having 3 to 60 carbon atoms, 3 to 30 carbon atoms, 3 to 25 carbon atoms, 3 to 18 carbon atoms, 3 to 12 carbon atoms, but is not limited thereto.

Unless otherwise stated, the term “alkoxyl group”, “alkoxy group” or “alkyloxy group”, as used herein, means an alkyl group bonded to oxygen radical, but is not limited thereto, and has 1 to 60 carbon atoms, 1 to 30 carbon atoms, 1 to 25 carbon atoms, 1 to 18 carbon atoms, or 1 to 12 carbon atoms.

Unless otherwise stated, the term “aryloxyl group” or “aryloxy group”, as used herein, means an aryl group bonded to oxygen radical, but is not limited thereto, and has 6 to 60 carbon atoms, 6 to 30 carbon atoms, 6 to 25 carbon atoms, 6 to 18 carbon atoms, or 6 to 12 carbon atoms.

Unless otherwise specified, the terms “aryl group” and “arylene group” used in the present invention have 6 to 60 carbon atoms, 6 to 30 carbon atoms, 6 to 25 carbon atoms, 6 to 18 carbon atoms, or 6 to 12 carbon atoms, respectively, but are not limited thereto. In the present invention, an aryl group or arylene group refers to an aromatic group of a single ring or multiple rings, and comprises an aromatic ring formed by the bonding or reaction of adjacent substituents. For example, the aryl group may be phenyl, biphenyl, naphthyl, a fluorene group or a spirofluorene group.

The prefix “aryl” or “ar” means a radical substituted with an aryl group. For example, an arylalkyl may be an alkyl substituted with an aryl, and an arylalkenyl may be an alkenyl substituted with aryl, and a radical substituted with an aryl has a number of carbon atoms as defined herein.

Also, when prefixes are named subsequently, it means that substituents are listed in the order described first. For example, an arylalkoxy means an alkoxy substituted with an aryl, an alkoxylcarbonyl means a carbonyl substituted with an alkoxyl, and an arylcarbonylalkenyl also means an alkenyl substituted with an arylcarbonyl, wherein the arylcarbonyl may be a carbonyl substituted with an aryl.

Unless otherwise stated, the term “heterocyclic group”, as used herein, contains one or more heteroatoms, and has 2 to 60 carbon atoms, 2 to 30 carbon atoms, 2 to 25 carbon atoms, 2 to 18 carbon atoms, 2 to 12 carbon atoms, and comprises any one of a single ring or multiple ring, and may include heteroaliphatic ring and heteroaromatic ring. Also, the heterocyclic group may also be formed in conjunction with an adjacent group.

Unless otherwise stated, the term “heteroatom”, as used herein, represents at least one of N, O, S, P, or Si.

Additionally, “heterocyclic group” means a single ring, ring aggregate, fused multiple ring system, spiro compound, etc. containing a heteroatom. Also, compounds containing heteroatom groups such as SO2, P═O, etc. instead of carbon forming a ring, such as the compounds below, can also be included in the heterocyclic group. For example, a “heterocyclic group” includes the following compound.

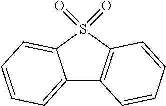 [SMILES: O=S1(=O)c2ccccc2-c2ccccc21]

The term “aliphatic ring group” used in the present invention refers to cyclic hydrocarbons excluding aromatic hydrocarbons, and includes single rings, ring aggregates, fused multiple ring systems, spiro compounds, etc., and means a ring having 3 to 60 carbon atoms, 3 to 30 carbon atoms, 3 to 25 carbon atoms, 3 to 18 carbon atoms, 3 to 12 carbon atoms, but is not limited thereto. For example, even when benzene, an aromatic ring, and cyclohexane, a non-aromatic ring, are fused, it is an aliphatic ring.

Unless otherwise stated, the term “fluorenyl group”, “fluorenylene group” or “fluorentriyl group” as used herein, means a monovalent, divalent or trivalent functional group, in which R, R′ and R″ are all hydrogen in the following structures, and the term “substituted fluorenyl group”, “substituted fluorenylene group” or “substituted fluorentriyl group” means that at least one of the substituents R, R′ and R″ is a substituent other than hydrogen, and include those in which R and R′ are bonded to each other to form a spiro compound together with the carbon to which they are bonded. In this specification, fluorenyl group, fluorenylene group, and fluorenetriyl group may all be referred to as fluorene groups, regardless of valence.

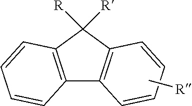

The term “spiro compound” used in the present invention has a ‘spiro union’, and a spiro union means a connection formed by 2 rings sharing only one atom. At this time, the atom shared between the 2 rings is called a ‘spiro atom’, and depending on the number of spiro atoms contained in a compound, they are called ‘monospiro-’, ‘dispiro-’, and ‘trispiro-’ compounds, respectively.

Unless otherwise stated, the term “aliphatic” as used herein means an aliphatic hydrocarbon having 1 to 60 carbon atoms, 1 to 30 carbon atoms, 1 to 25 carbon atoms, 1 to 18 carbon atoms or 1 to 12 carbon atoms, and “aliphatic ring” means an aliphatic hydrocarbon ring having 3 to 60 carbon atoms, 3 to 30 carbon atoms, 3 to 25 carbon atoms, 3 to 18 carbon atoms or 3 to 12 carbon atoms.

Unless otherwise stated, the term “ring”, as used herein, means an aliphatic ring having 3 to 60 carbon atoms, 3 to 30 carbon atoms, 3 to 25 carbon atoms, 3 to 18 carbon atoms or 3 to 12 carbon atoms; or an aromatic ring having 6 to 60 carbon atoms, 6 to 30 carbon atoms, 6 to 25 carbon atoms, 6 to 18 carbon atoms, or 6 to 12 carbon atoms; or a heterocyclic having 2 to 60 carbon atoms, 2 to 30 carbon atoms, 2 to 25 carbon atoms, 2 to 18 carbon atoms, 2 to 12 carbon atoms, or a fused ring formed by the combination thereof, and includes a saturated or unsaturated ring.

Other hetero compounds or hetero radicals other than the above-mentioned hetero compounds include, but are not limited thereto, one or more heteroatoms.

Also, unless expressly stated, as used herein, “substituted” in the term “substituted or unsubstituted” means substituted with one or more substituents selected from the group consisting of deuterium, halogen, an amino group, a nitrile group, a nitro group, a C1-C20 alkyl group, a C1-C20 alkoxyl group, a C1-C20 alkylamine group, a C1-C20 alkylthiopen group, a C6-C20 arylthiopen group, a C2-C20 alkenyl group, a C2-C20 alkynyl group, a C3-C20 cycloalkyl group, a C6-C20 aryl group, a C6-C20 aryl group substituted by deuterium, a C8-C20 arylalkenyl group, a silane group, a boron group, a germanium group, and a C2-C20 heterocyclic group, but is not limited to these substituents.

In this specification, the ‘group name’ corresponding to the aryl group, arylene group, heterocyclic group, etc., as examples of each symbol and its substituent, may be written as the ‘name of the group reflecting the valence’, but is written as the ‘parent compound name’. For example, in the case of ‘phenanthrene’, a type of aryl group, the name of the group may be written by distinguishing the valence, such as the monovalent ‘group’ is ‘phenanthryl’ and the divalent group is ‘phenanthrylene’, but may be written as ‘phenanthrene’, which is the name of the parent compound, regardless of the valence. Similarly, in the case of pyrimidine, it can be written as ‘pyrimidine’ regardless of the valence, or it can be written as the ‘name of the group’ of the valence, such as pyrimidineyl group in the case of monovalent group, pyrimidineylene in the case of divalent group, etc. Additionally, in this specification, when describing compound names or substituent names, numbers or alphabets indicating positions may be omitted. For example, pyrido[4,3-d]pyrimidine to pyridopyrimidine, benzofuro[2,3-d]pyrimidine to benzofuropyrimidine, 9,9-dimethyl-9H-fluorene can be described as dimethylfluorene, etc. Therefore, both benzo[g]quinoxaline and benzo[f]quinoxaline can be described as benzoquinoxaline.

Also, unless there is an explicit explanation, the formula used in the present invention is the same as the definition of the substituent by the exponent definition of the following formula.

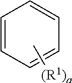

Here, when a is an integer of 0, the substituent R¹ is absent, when a is an integer of 1, the sole substituent R¹ is linked to any one of the carbon constituting the benzene ring, when a is an integer of 2 or 3, each is combined as follows, where R¹ may be the same or different from each other, when a is an integer of 4 to 6, it is bonded to the carbon of the benzene ring in a similar manner, while the indication of the hydrogen bonded to the carbon forming the benzene ring is omitted.

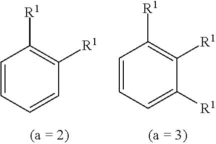

Unless otherwise expressly stated, the terms “ortho”, “meta”, and “para” used in the present invention refer to the substitution positions of all substituents, and the ortho position refers to a compound in which the position of the substituent is immediately adjacent, for example, when benzene is used, it means 1 or 2 position, and the meta position is the next substitution position of the neighbor substitution position, when benzene as an example stands for 1 or 3 position, and the para position is the next substitution position of the meta position, which means 1 and 4 position when benzene is taken as an example. A more detailed example of the substitution position is as follows, and it can be confirmed that the ortho-, and meta-position are substituted by non-linear type and para-positions are substituted by linear type.

### [Example of Ortho-Position]

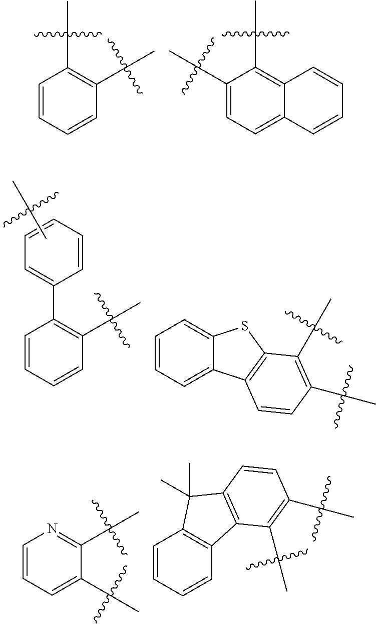

### [Example of Meta-Position]

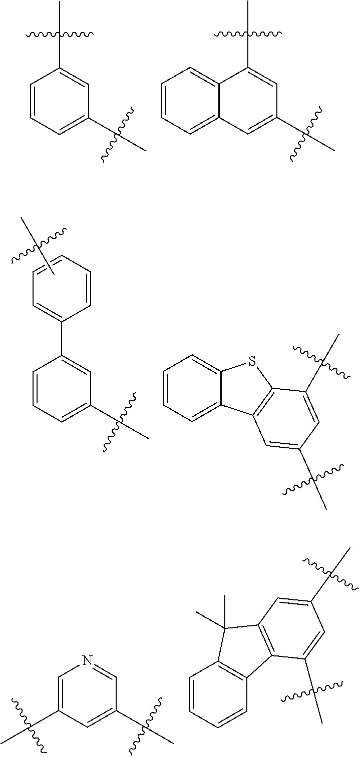

### [Example of Para-Position]

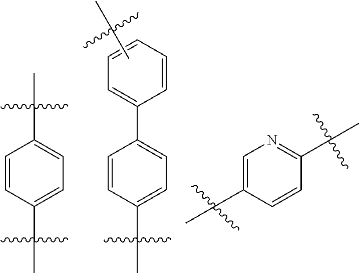

The term “composition” as used in the present invention is intended to be broadly interpreted to include not only compounds but also solutions, dispersions, liquid and solid mixtures (mixtures, admixtures). The composition of the present invention may contain the compound of the present invention alone, or may contain 2 or more different compounds in combination, or may contain the compound in combination with 2 or more other compounds. In other words, the composition may comprise a compound corresponding to Formula 1 alone, may comprise a mixture of 2 or more compounds of Formula 1, or may comprise a mixture of a compound of Formula 1 and a compound not corresponding to the present invention. Wherein, the compound not corresponding to the present invention may be a single compound or 2 or more compounds. Here, when the compound is contained in a combination of 2 or more other compounds, the other compounds may be already known compounds of each organic material layer or compounds to be developed in the future. Here, the compound contained in the organic material layer may be composed of only the same type of compound, but may also be a mixture of 2 or more types of heterogeneous compounds represented by Formula 1.

Hereinafter, a compound according to one aspect of the present invention, a composition for an organic electronic element, and an organic electronic element including the same will be described.

The present invention provides a compound represented by Formula 1.

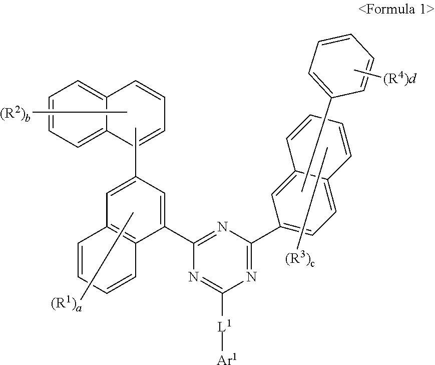

wherein:
R¹, R² and R³ are deuterium,
R⁴ is independently the same or different from each other and is independently selected from the group consisting of deuterium; a C₆-C₆₀ aryl group; a fluorenyl group; a C₃-C₆₀ aliphatic ring; a C₁-C₅₀ alkyl group; a C₂-C₂₀ alkenyl group; a C₂-C₂₀ alkynyl group; a C₁-C₃₀ alkoxy group; and a C₆-C₃₀ an aryloxy group; or a plurality of adjacent groups thereof may be bonded to each other to form an aromatic ring,

Wherein when R⁴ is an aryl group, preferably an C₆-C₃₀ aryl group, more preferably an C₆-C₂₅ aryl group, an C₆-C₁₈ aryl group or an C₆-C₁₂ aryl group, such as phenyl, biphenyl, terphenyl, naphthalene, phenanthrene, chryshen, etc.

Wherein when R⁴ is an aliphatic ring group, preferably a C₃-C₃₀ aliphatic ring group, more preferably a C₃-C₂₅ aliphatic ring group, a C₃-C₁₈ aliphatic ring group, and a C₃-C₁₂ aliphatic ring group, and specifically, cyclobutane, cyclopentane, cyclohexane, bicycloheptane, adamantyl, etc.

Wherein when R⁴ is an alkyl group, preferably a C₁-C₃₀ alkyl group, more preferably a C₁-C₂₅ alkyl group, a C₁-C₁₈ alkyl group or a C₁-C₁₂ alkyl group, such as a methyl group, ethyl group, propyl group, isopropyl group, butyl group, t-butyl group, pentyl group, etc.

Wherein when R⁴ is an aryloxy group, preferably an C₆-C₂₅ aryloxy group, more preferably an C₆-C₁₈ aryloxy group, or an C₆-C₁₂ aryloxy group.

L¹ is a single bond; or a C₆-C₁₂ arylene group;

Wherein when L¹ is an arylene group, it may be, for example, phenylene, biphenylene, or naphthylene.

Ar¹ is a C₆-C₁₂ aryl group; and may be, for example, phenyl, biphenyl, or naphthyl.

a and c are independently integers from 0 to 6, b is an integer from 0 to 7, and d is an integer from 0 to 5,
wherein the aryl group, arylene group, fluorenyl group, aliphatic ring group, alkyl group, alkenyl group, alkynyl group, alkoxyl group and aryloxy group may each be further substituted with one or more substituents selected from the group consisting of deuterium; a C₁-C₂₀ alkyl group; a C₁-C₂₀ alkyl group substituted with deuterium; a C₆-C₁₂ aryl group; and a C₆-C₁₂ aryl group substituted with deuterium;

Also, a compound represented by Formula 1 is represented by either Formula 1-1 or Formula 1-2.

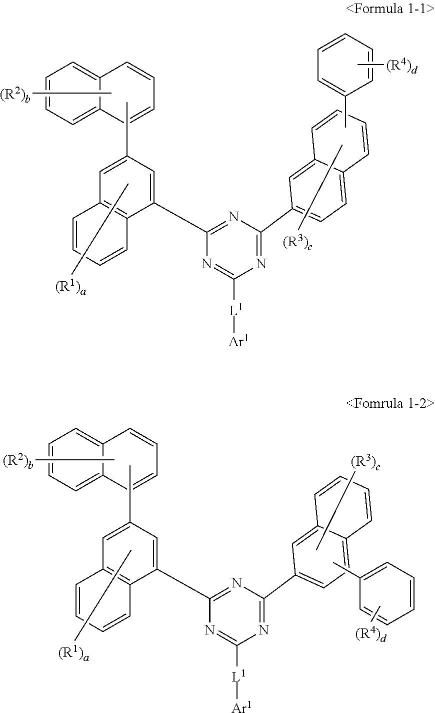

wherein R¹, R², R³, R⁴, L¹, Ar¹, a, b, c and d are the same as defined in Formula 1.

Also, the compound represented by Formula 1 is represented by any one of the following Formulas A-1 to A-7.

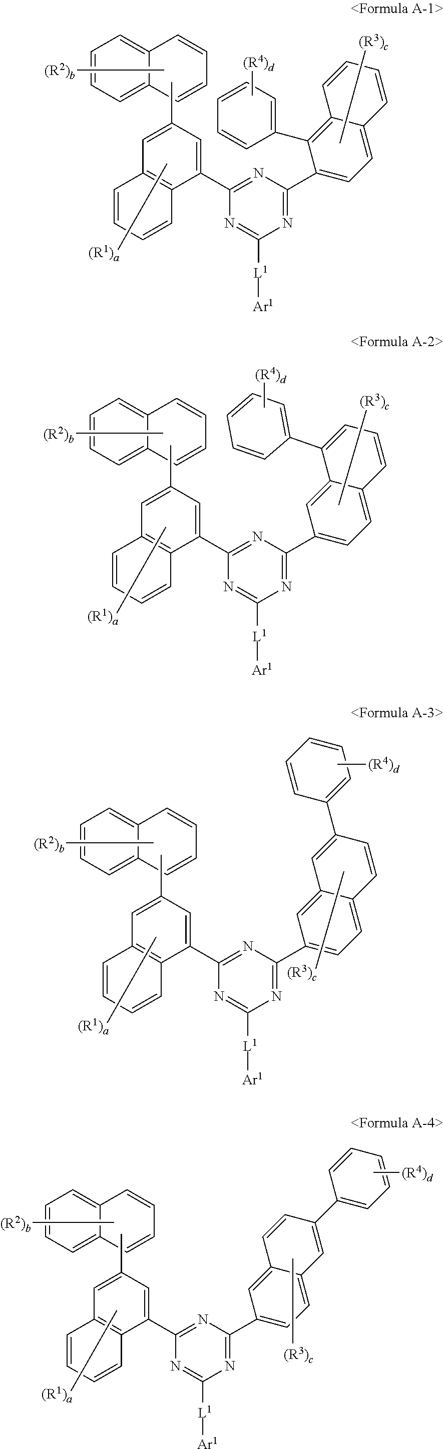

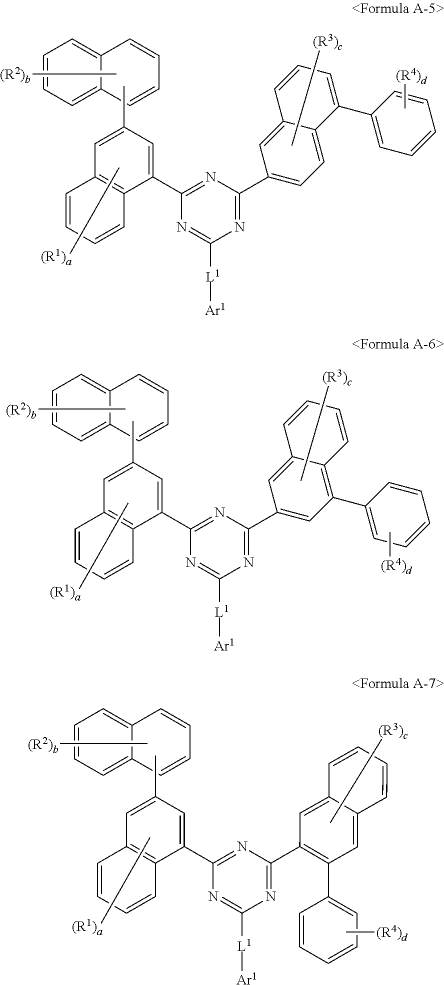

wherein R¹, R², R³, R⁴, L¹, Ar¹, a, b, c and d are the same as defined in Formula 1.

Also, the compound represented by Formula 1 is represented by any one of the following Formulas 1-3 to 1-6.

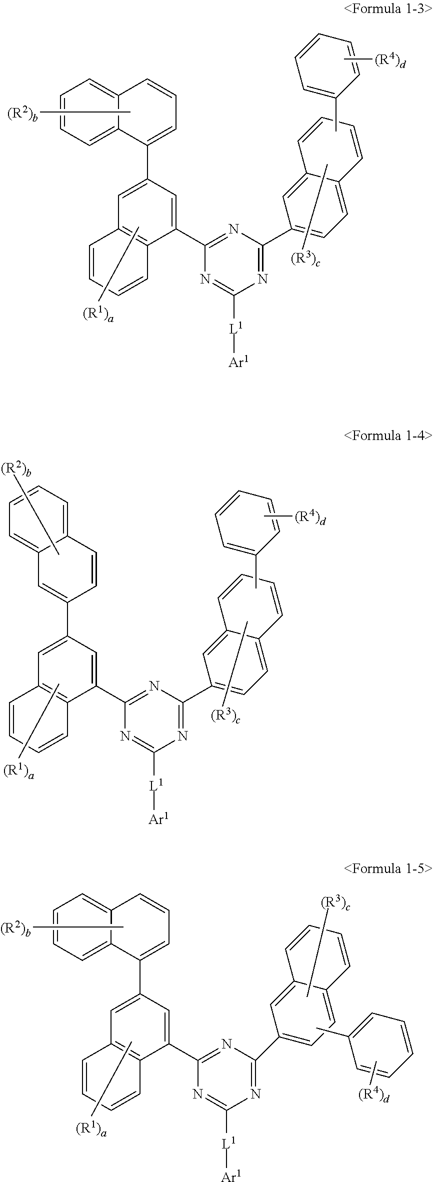

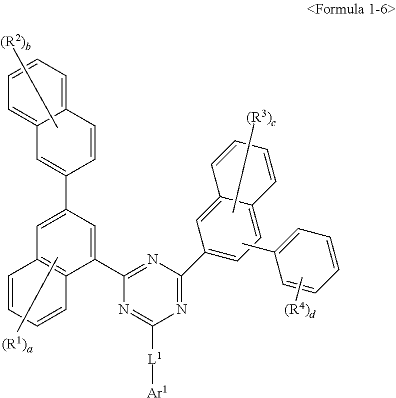

wherein: R¹, R², R³, R⁴, L¹, Ar¹, a, b, c and d are the same as defined in Formula 1.

Also, the compound represented by Formula 1 is represented by Formula 2-1 or Formula 2-2.

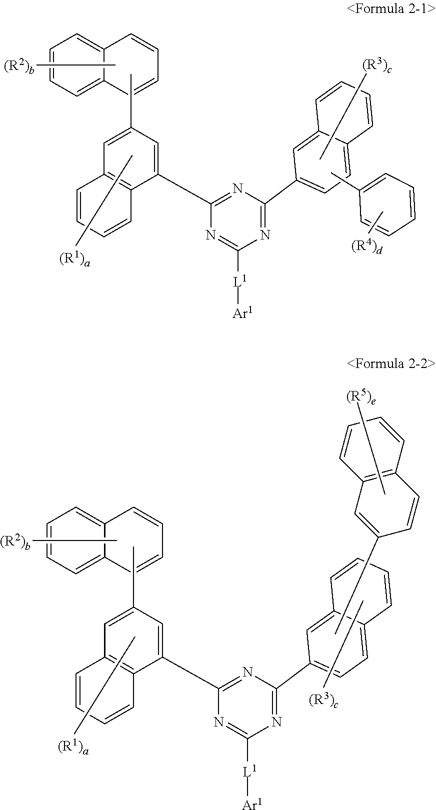

wherein: R¹, R², R³, R⁴, L¹, Ar¹, a, b, c and d are the same as defined in Formula 1.

Also, Ar1 of Formula 1 is represented by any one of Formulas Ar-1 to Ar-3.

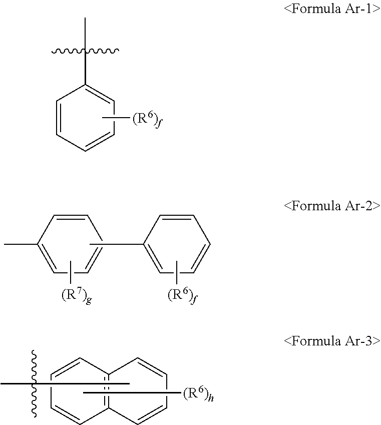

wherein:
R⁶ and R⁷ are independently the same or different from each other and are independently selected from the group consisting of deuterium; a C₁-C₂₀ alkyl group; a C₁-C₂₀ alkyl group substituted with deuterium; a C₆-C₁₂ aryl group; and a C₆-C₁₂ aryl group substituted with deuterium; or a plurality of adjacent groups thereof may be bonded to each other to form a ring,
f is an integer from 0 to 5; g is an integer from 0 to 4; h is an integer from 0 to 7;
[symbol] indicates the position to be bonded.
L¹ of Formula 1 is represented by any one of Formulas L-1 to L-4.

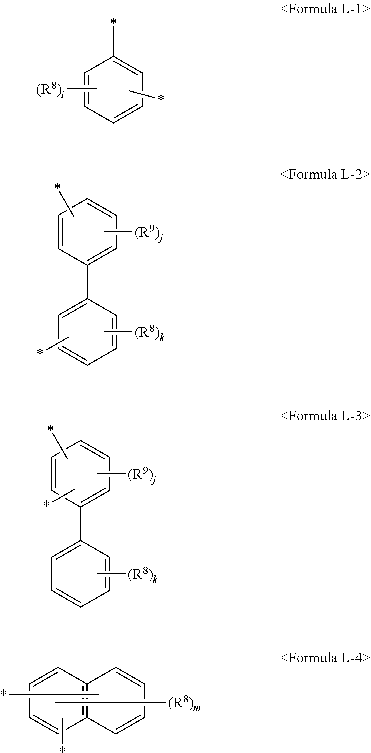

wherein:
R⁸ and R⁹ are independently the same or different from each other and are independently selected from the group consisting of deuterium; a C₁-C₂₀ alkyl group; a C₁-C₂₀ alkyl group substituted with deuterium; a C₆-C₁₂ aryl group; and a C₆-C₁₂ aryl group substituted with deuterium; or a plurality of adjacent groups thereof may be bonded to each other to form a ring,
i and j are independently an integer from 0 to 4; k is an integer from 0 to 5; l is an integer from 0 to 3; m is an integer from 0 to 6;
* indicates the position to be bonded.

Specifically, the compound of Formula 1 may be any one of the following compounds P1-1 to P1-189 and P2-1 to P2-44, but is not limited thereto.

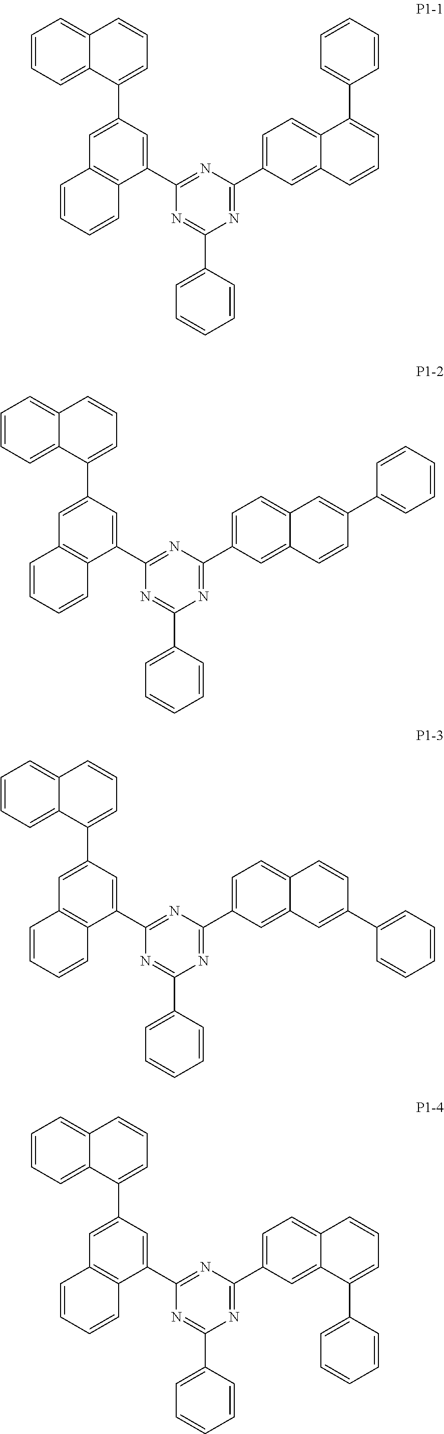 [SMILES: c1ccc(-c2nc(-c3ccc4c(-c5ccccc5)cccc4c3)nc(-c3cc(-c4cccc5ccccc45)cc4ccccc34)n2)cc1 ; c1ccc(-c2ccc3cc(-c4nc(-c5ccccc5)nc(-c5cc(-c6cccc7ccccc67)cc6ccccc56)n4)ccc3c2)cc1 ; c1ccc(-c2ccc3ccc(-c4nc(-c5ccccc5)nc(-c5cc(-c6cccc7ccccc67)cc6ccccc56)n4)cc3c2)cc1 ; c1ccc(-c2nc(-c3ccc4cccc(-c5ccccc5)c4c3)nc(-c3cc(-c4cccc5ccccc45)cc4ccccc34)n2)cc1]

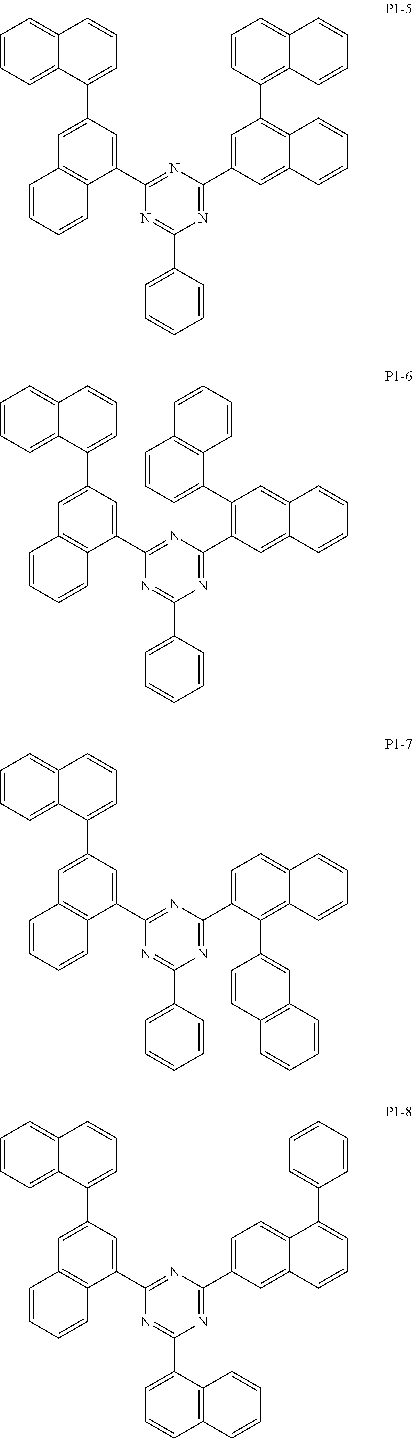 [SMILES: c1ccc(-c2nc(-c3cc(-c4cccc5ccccc45)c4ccccc4c3)nc(-c3cc(-c4cccc5ccccc45)cc4ccccc34)n2)cc1 ; c1ccc(-c2nc(-c3cc4ccccc4cc3-c3cccc4ccccc34)nc(-c3cc(-c4cccc5ccccc45)cc4ccccc34)n2)cc1 ; c1ccc(-c2nc(-c3ccc4ccccc4c3-c3ccc4ccccc4c3)nc(-c3cc(-c4cccc5ccccc45)cc4ccccc34)n2)cc1 ; c1ccc(-c2cccc3cc(-c4nc(-c5cccc6ccccc56)nc(-c5cc(-c6cccc7ccccc67)cc6ccccc56)n4)ccc23)cc1]

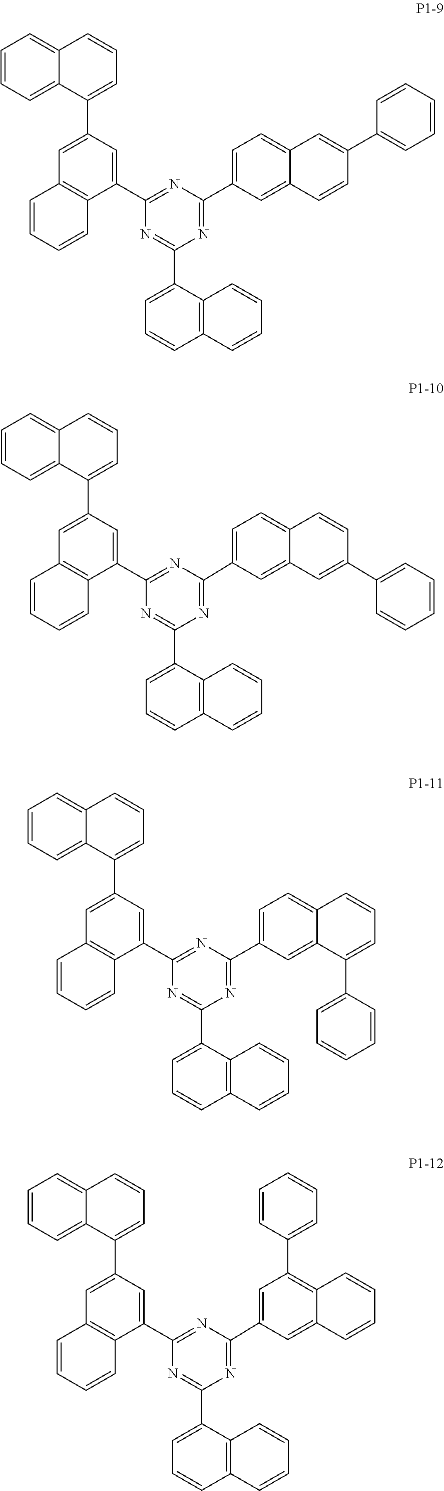 [SMILES: c1ccc(-c2ccc3cc(-c4nc(-c5cccc6ccccc56)nc(-c5cc(-c6cccc7ccccc67)cc6ccccc56)n4)ccc3c2)cc1 ; c1ccc(-c2ccc3ccc(-c4nc(-c5cccc6ccccc56)nc(-c5cc(-c6cccc7ccccc67)cc6ccccc56)n4)cc3c2)cc1 ; c1ccc(-c2cccc3ccc(-c4nc(-c5cccc6ccccc56)nc(-c5cc(-c6cccc7ccccc67)cc6ccccc56)n4)cc23)cc1 ; c1ccc(-c2cc(-c3nc(-c4cccc5ccccc45)nc(-c4cc(-c5cccc6ccccc56)cc5ccccc45)n3)cc3ccccc23)cc1]

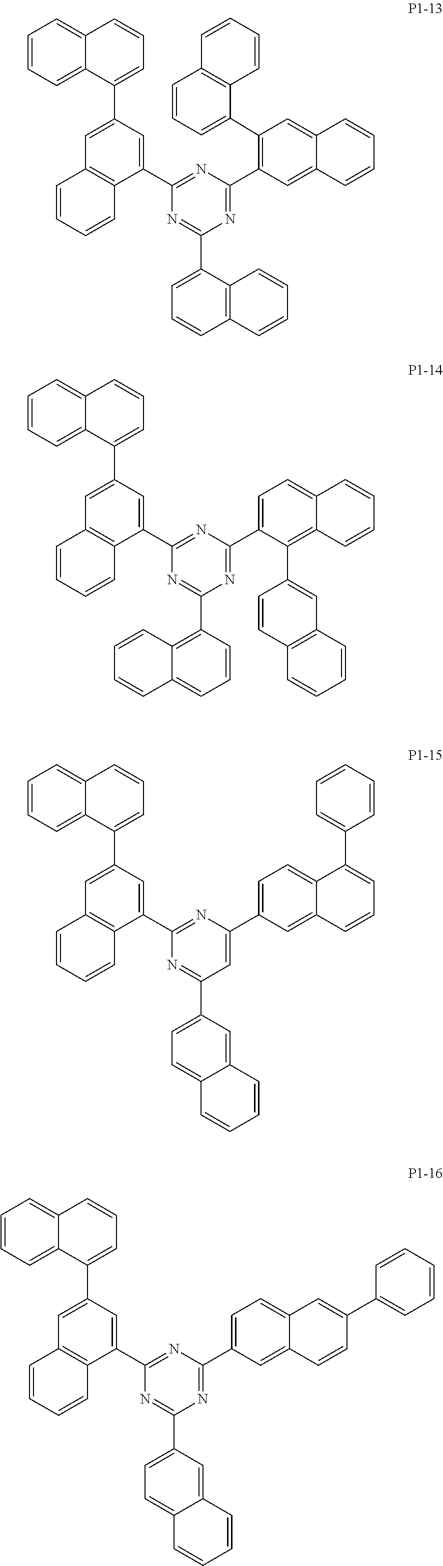 [SMILES: c1ccc2cc(-c3cccc4ccccc34)c(-c3nc(-c4cccc5ccccc45)nc(-c4cc(-c5cccc6ccccc56)cc5ccccc45)n3)cc2c1 ; c1ccc2cc(-c3c(-c4nc(-c5cccc6ccccc56)nc(-c5cc(-c6cccc7ccccc67)cc6ccccc56)n4)ccc4ccccc34)ccc2c1 ; c1ccc(-c2cccc3cc(-c4cc(-c5ccc6ccccc6c5)nc(-c5cc(-c6cccc7ccccc67)cc6ccccc56)n4)ccc23)cc1 ; c1ccc(-c2ccc3cc(-c4nc(-c5ccc6ccccc6c5)nc(-c5cc(-c6cccc7ccccc67)cc6ccccc56)n4)ccc3c2)cc1]

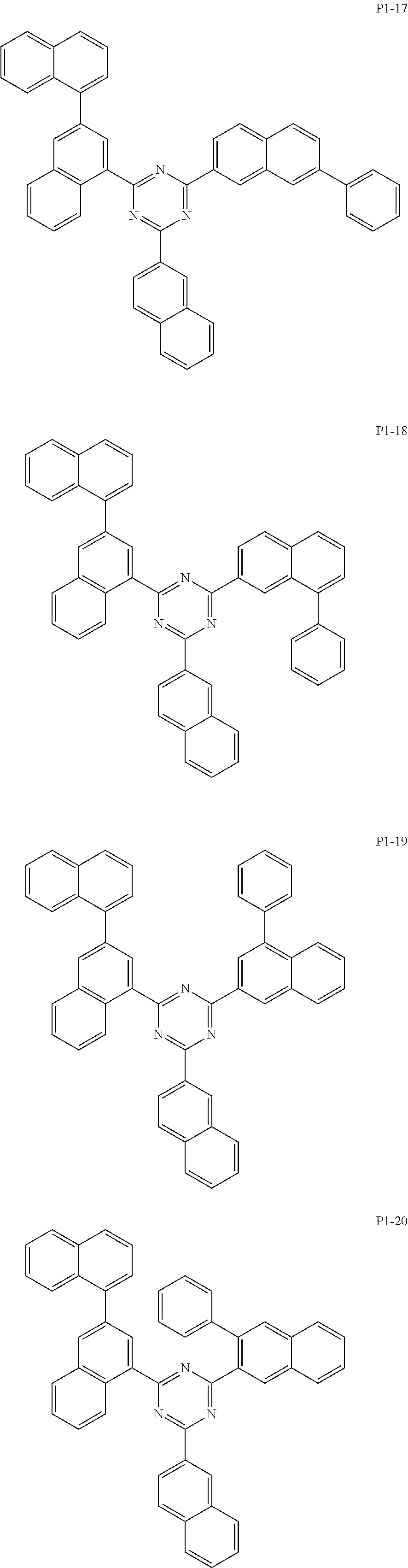 [SMILES: c1ccc(-c2ccc3ccc(-c4nc(-c5ccc6ccccc6c5)nc(-c5cc(-c6cccc7ccccc67)cc6ccccc56)n4)cc3c2)cc1 ; c1ccc(-c2cccc3ccc(-c4nc(-c5ccc6ccccc6c5)nc(-c5cc(-c6cccc7ccccc67)cc6ccccc56)n4)cc23)cc1 ; c1ccc(-c2cc(-c3nc(-c4ccc5ccccc5c4)nc(-c4cc(-c5cccc6ccccc56)cc5ccccc45)n3)cc3ccccc23)cc1 ; c1ccc(-c2cc3ccccc3cc2-c2nc(-c3ccc4ccccc4c3)nc(-c3cc(-c4cccc5ccccc45)cc4ccccc34)n2)cc1]

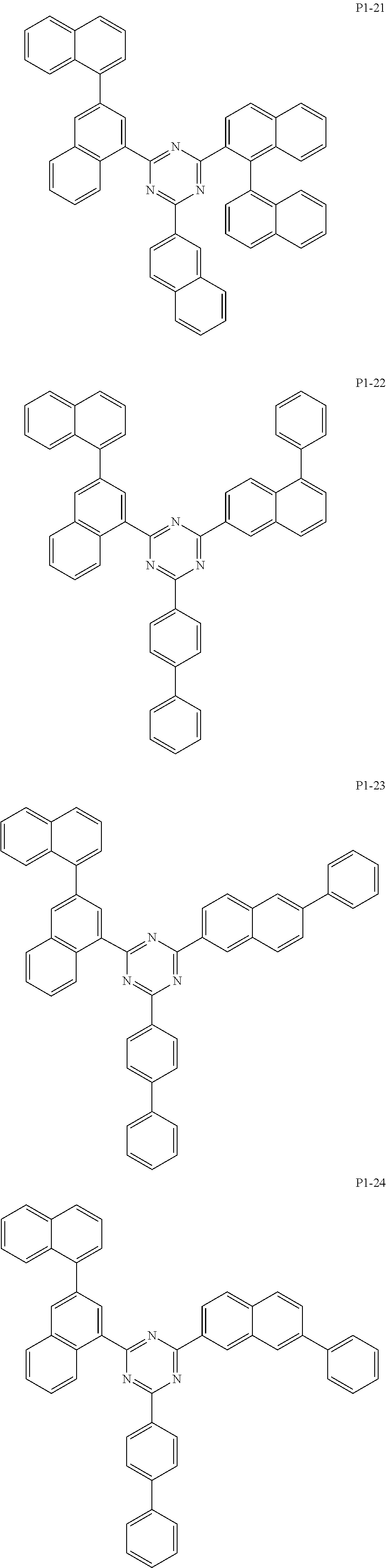 [SMILES: c1ccc2cc(-c3nc(-c4ccc5ccccc5c4-c4cccc5ccccc45)nc(-c4cc(-c5cccc6ccccc56)cc5ccccc45)n3)ccc2c1 ; c1ccc(-c2ccc(-c3nc(-c4ccc5c(-c6ccccc6)cccc5c4)nc(-c4cc(-c5cccc6ccccc56)cc5ccccc45)n3)cc2)cc1 ; c1ccc(-c2ccc(-c3nc(-c4ccc5cc(-c6ccccc6)ccc5c4)nc(-c4cc(-c5cccc6ccccc56)cc5ccccc45)n3)cc2)cc1 ; c1ccc(-c2ccc(-c3nc(-c4ccc5ccc(-c6ccccc6)cc5c4)nc(-c4cc(-c5cccc6ccccc56)cc5ccccc45)n3)cc2)cc1]

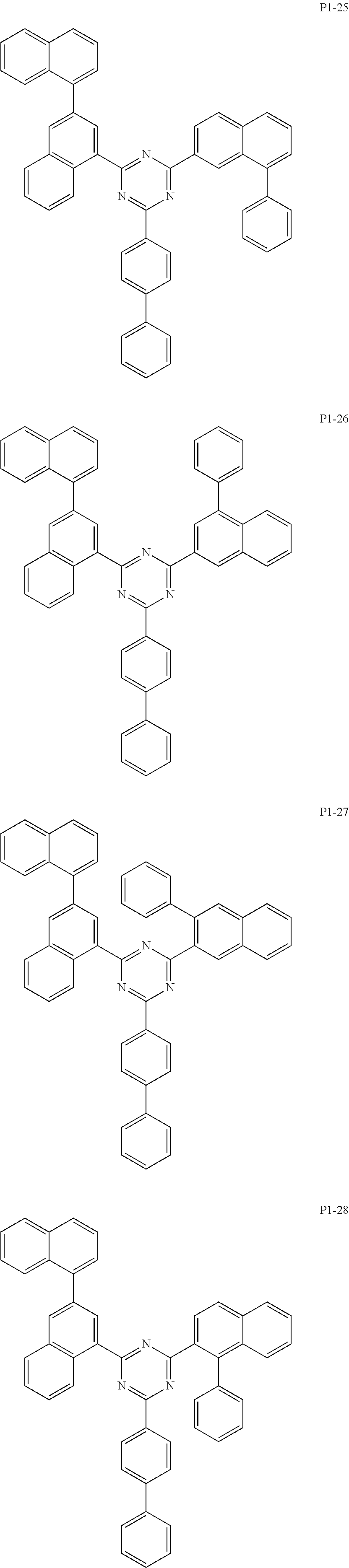 [SMILES: c1ccc(-c2ccc(-c3nc(-c4ccc5cccc(-c6ccccc6)c5c4)nc(-c4cc(-c5cccc6ccccc56)cc5ccccc45)n3)cc2)cc1 ; c1ccc(-c2ccc(-c3nc(-c4cc(-c5ccccc5)c5ccccc5c4)nc(-c4cc(-c5cccc6ccccc56)cc5ccccc45)n3)cc2)cc1 ; c1ccc(-c2ccc(-c3nc(-c4cc5ccccc5cc4-c4ccccc4)nc(-c4cc(-c5cccc6ccccc56)cc5ccccc45)n3)cc2)cc1 ; c1ccc(-c2ccc(-c3nc(-c4ccc5ccccc5c4-c4ccccc4)nc(-c4cc(-c5cccc6ccccc56)cc5ccccc45)n3)cc2)cc1]

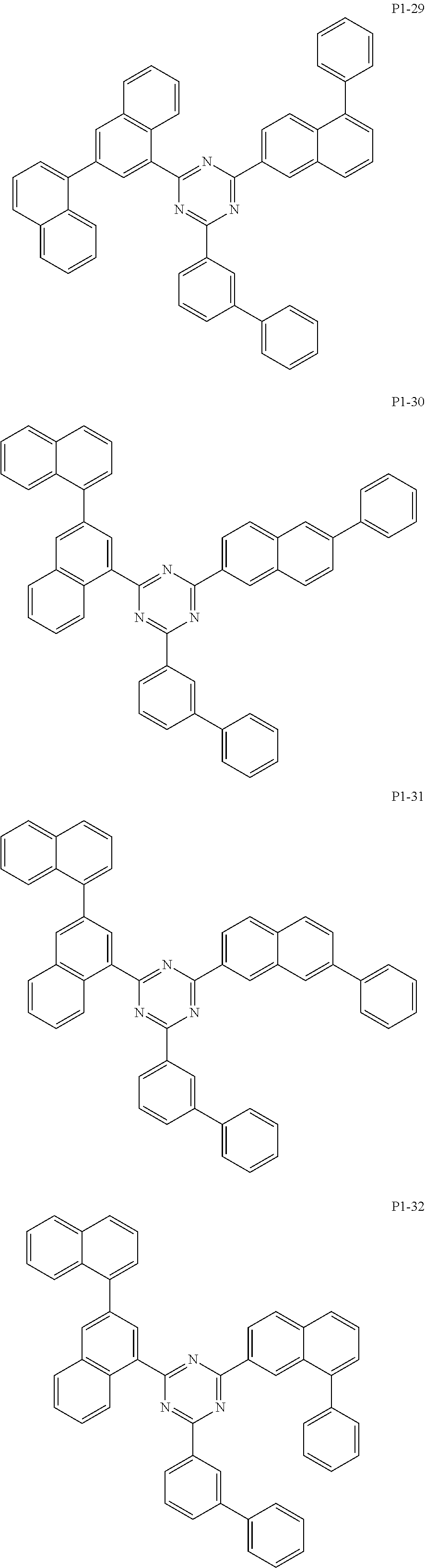 [SMILES: c1ccc(-c2cccc(-c3nc(-c4ccc5c(-c6ccccc6)cccc5c4)nc(-c4cc(-c5cccc6ccccc56)cc5ccccc45)n3)c2)cc1 ; c1ccc(-c2cccc(-c3nc(-c4ccc5cc(-c6ccccc6)ccc5c4)nc(-c4cc(-c5cccc6ccccc56)cc5ccccc45)n3)c2)cc1 ; c1ccc(-c2cccc(-c3nc(-c4ccc5ccc(-c6ccccc6)cc5c4)nc(-c4cc(-c5cccc6ccccc56)cc5ccccc45)n3)c2)cc1 ; c1ccc(-c2cccc(-c3nc(-c4ccc5cccc(-c6ccccc6)c5c4)nc(-c4cc(-c5cccc6ccccc56)cc5ccccc45)n3)c2)cc1]

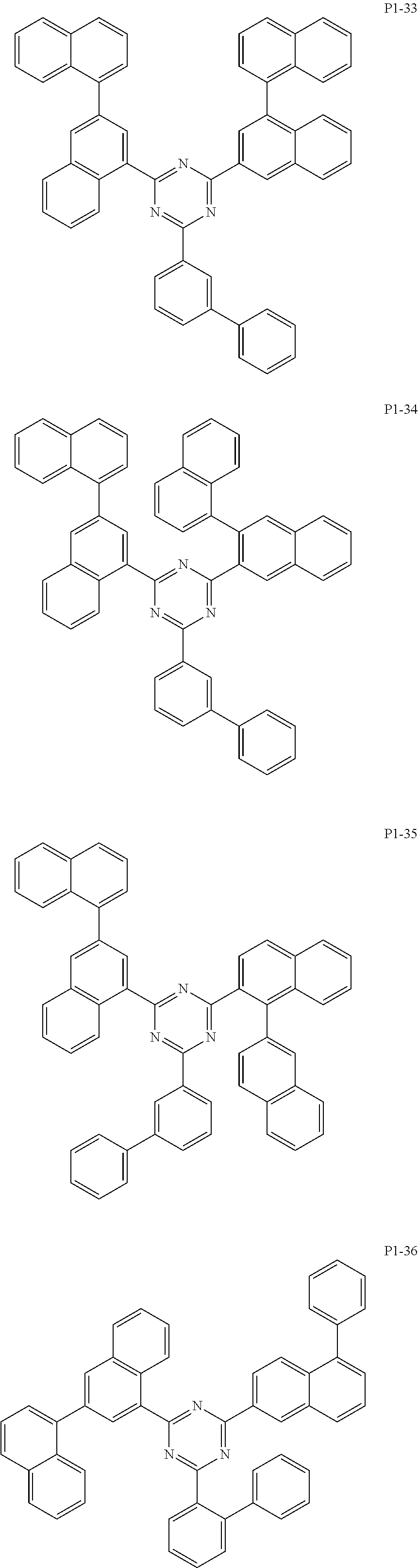 [SMILES: c1ccc(-c2cccc(-c3nc(-c4cc(-c5cccc6ccccc56)c5ccccc5c4)nc(-c4cc(-c5cccc6ccccc56)cc5ccccc45)n3)c2)cc1 ; c1ccc(-c2cccc(-c3nc(-c4cc5ccccc5cc4-c4cccc5ccccc45)nc(-c4cc(-c5cccc6ccccc56)cc5ccccc45)n3)c2)cc1 ; c1ccc(-c2cccc(-c3nc(-c4ccc5ccccc5c4-c4ccc5ccccc5c4)nc(-c4cc(-c5cccc6ccccc56)cc5ccccc45)n3)c2)cc1 ; c1ccc(-c2ccccc2-c2nc(-c3ccc4c(-c5ccccc5)cccc4c3)nc(-c3cc(-c4cccc5ccccc45)cc4ccccc34)n2)cc1]

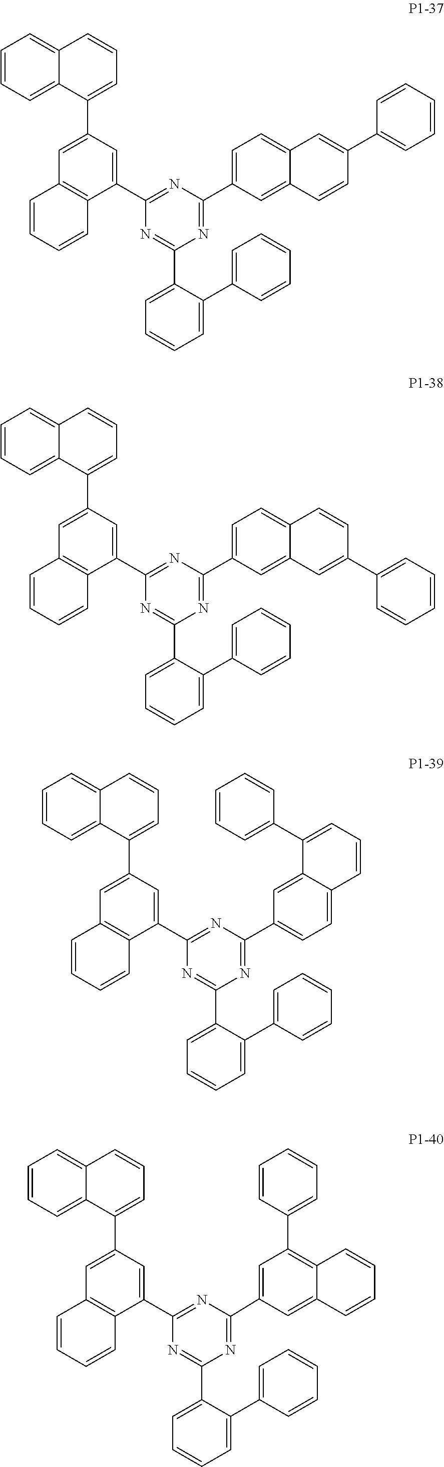 [SMILES: c1ccc(-c2ccc3cc(-c4nc(-c5ccccc5-c5ccccc5)nc(-c5cc(-c6cccc7ccccc67)cc6ccccc56)n4)ccc3c2)cc1 ; c1ccc(-c2ccc3ccc(-c4nc(-c5ccccc5-c5ccccc5)nc(-c5cc(-c6cccc7ccccc67)cc6ccccc56)n4)cc3c2)cc1 ; c1ccc(-c2ccccc2-c2nc(-c3ccc4cccc(-c5ccccc5)c4c3)nc(-c3cc(-c4cccc5ccccc45)cc4ccccc34)n2)cc1 ; c1ccc(-c2ccccc2-c2nc(-c3cc(-c4ccccc4)c4ccccc4c3)nc(-c3cc(-c4cccc5ccccc45)cc4ccccc34)n2)cc1]

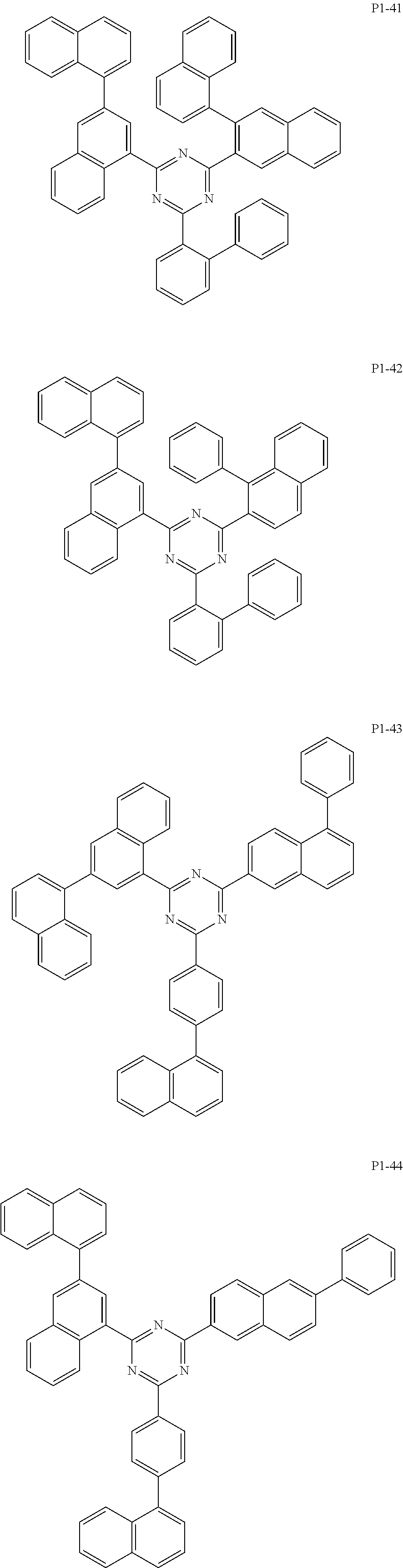 [SMILES: c1ccc(-c2ccccc2-c2nc(-c3cc4ccccc4cc3-c3cccc4ccccc34)nc(-c3cc(-c4cccc5ccccc45)cc4ccccc34)n2)cc1 ; c1ccc(-c2ccccc2-c2nc(-c3ccc4ccccc4c3-c3ccccc3)nc(-c3cc(-c4cccc5ccccc45)cc4ccccc34)n2)cc1 ; c1ccc(-c2cccc3cc(-c4nc(-c5ccc(-c6cccc7ccccc67)cc5)nc(-c5cc(-c6cccc7ccccc67)cc6ccccc56)n4)ccc23)cc1 ; c1ccc(-c2ccc3cc(-c4nc(-c5ccc(-c6cccc7ccccc67)cc5)nc(-c5cc(-c6cccc7ccccc67)cc6ccccc56)n4)ccc3c2)cc1]

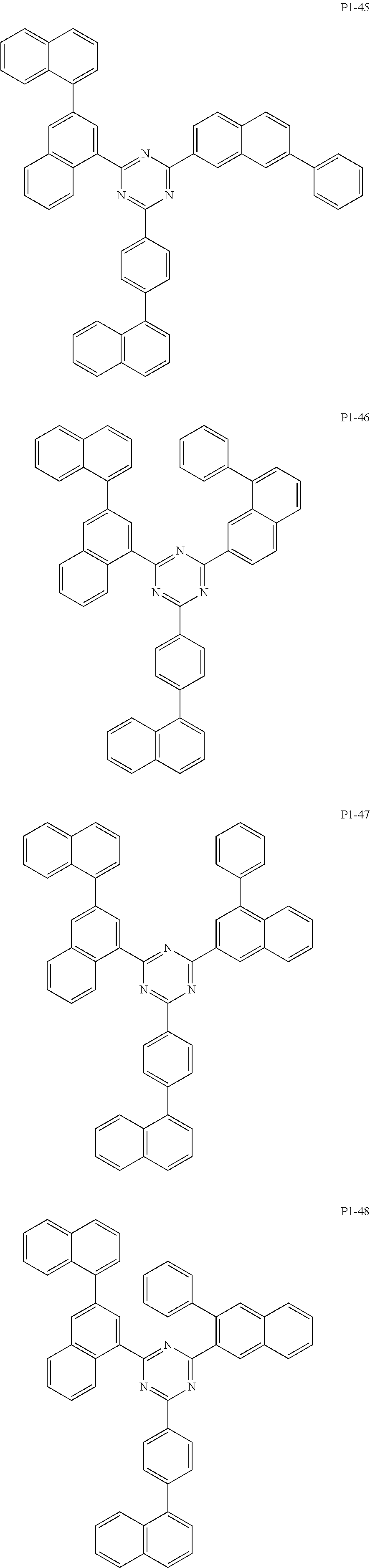 [SMILES: c1ccc(-c2ccc3ccc(-c4nc(-c5ccc(-c6cccc7ccccc67)cc5)nc(-c5cc(-c6cccc7ccccc67)cc6ccccc56)n4)cc3c2)cc1 ; c1ccc(-c2cccc3ccc(-c4nc(-c5ccc(-c6cccc7ccccc67)cc5)nc(-c5cc(-c6cccc7ccccc67)cc6ccccc56)n4)cc23)cc1 ; c1ccc(-c2cc(-c3nc(-c4ccc(-c5cccc6ccccc56)cc4)nc(-c4cc(-c5cccc6ccccc56)cc5ccccc45)n3)cc3ccccc23)cc1 ; c1ccc(-c2cc3ccccc3cc2-c2nc(-c3ccc(-c4cccc5ccccc45)cc3)nc(-c3cc(-c4cccc5ccccc45)cc4ccccc34)n2)cc1]

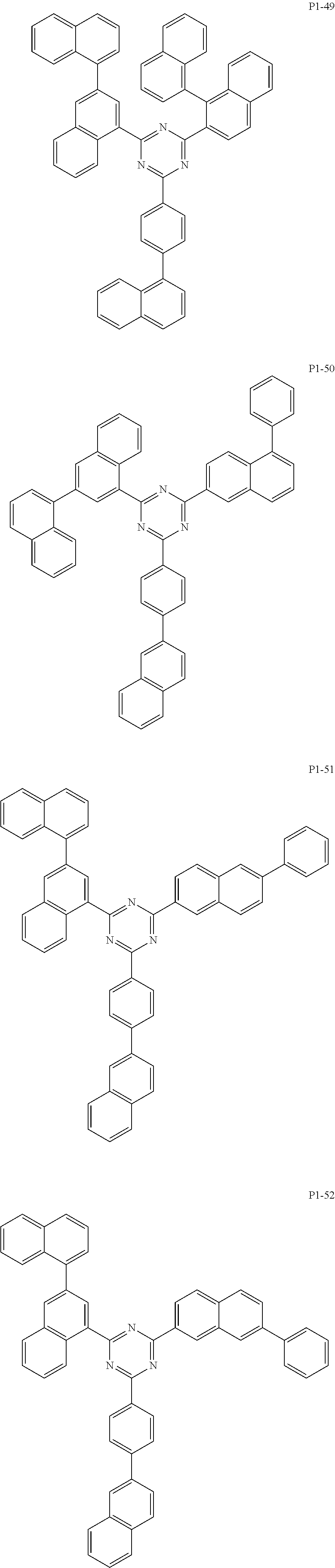 [SMILES: c1ccc2c(-c3ccc(-c4nc(-c5ccc6ccccc6c5-c5cccc6ccccc56)nc(-c5cc(-c6cccc7ccccc67)cc6ccccc56)n4)cc3)cccc2c1 ; c1ccc(-c2cccc3cc(-c4nc(-c5ccc(-c6ccc7ccccc7c6)cc5)nc(-c5cc(-c6cccc7ccccc67)cc6ccccc56)n4)ccc23)cc1 ; c1ccc(-c2ccc3cc(-c4nc(-c5ccc(-c6ccc7ccccc7c6)cc5)nc(-c5cc(-c6cccc7ccccc67)cc6ccccc56)n4)ccc3c2)cc1 ; c1ccc(-c2ccc3ccc(-c4nc(-c5ccc(-c6ccc7ccccc7c6)cc5)nc(-c5cc(-c6cccc7ccccc67)cc6ccccc56)n4)cc3c2)cc1]

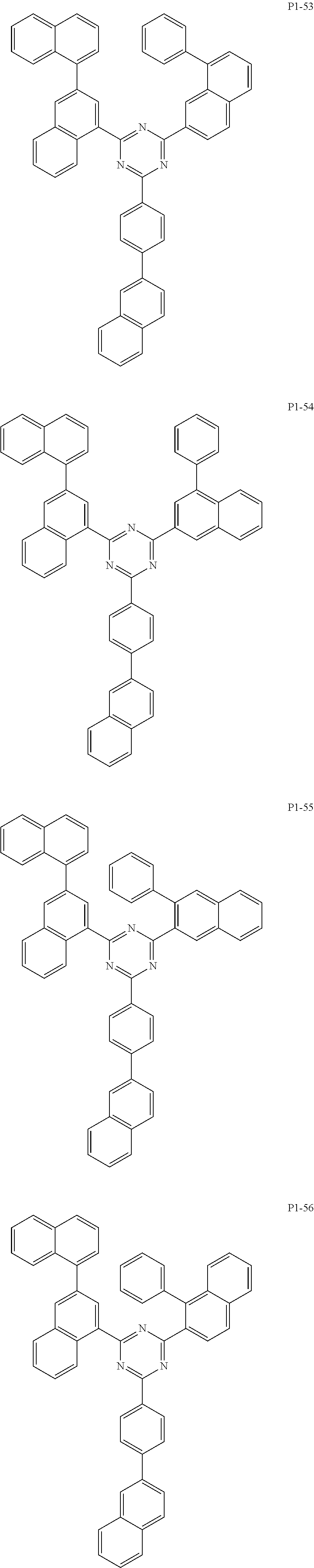 [SMILES: c1ccc(-c2cccc3ccc(-c4nc(-c5ccc(-c6ccc7ccccc7c6)cc5)nc(-c5cc(-c6cccc7ccccc67)cc6ccccc56)n4)cc23)cc1 ; c1ccc(-c2cc(-c3nc(-c4ccc(-c5ccc6ccccc6c5)cc4)nc(-c4cc(-c5cccc6ccccc56)cc5ccccc45)n3)cc3ccccc23)cc1 ; c1ccc(-c2cc3ccccc3cc2-c2nc(-c3ccc(-c4ccc5ccccc5c4)cc3)nc(-c3cc(-c4cccc5ccccc45)cc4ccccc34)n2)cc1 ; c1ccc(-c2c(-c3nc(-c4ccc(-c5ccc6ccccc6c5)cc4)nc(-c4cc(-c5cccc6ccccc56)cc5ccccc45)n3)ccc3ccccc23)cc1]

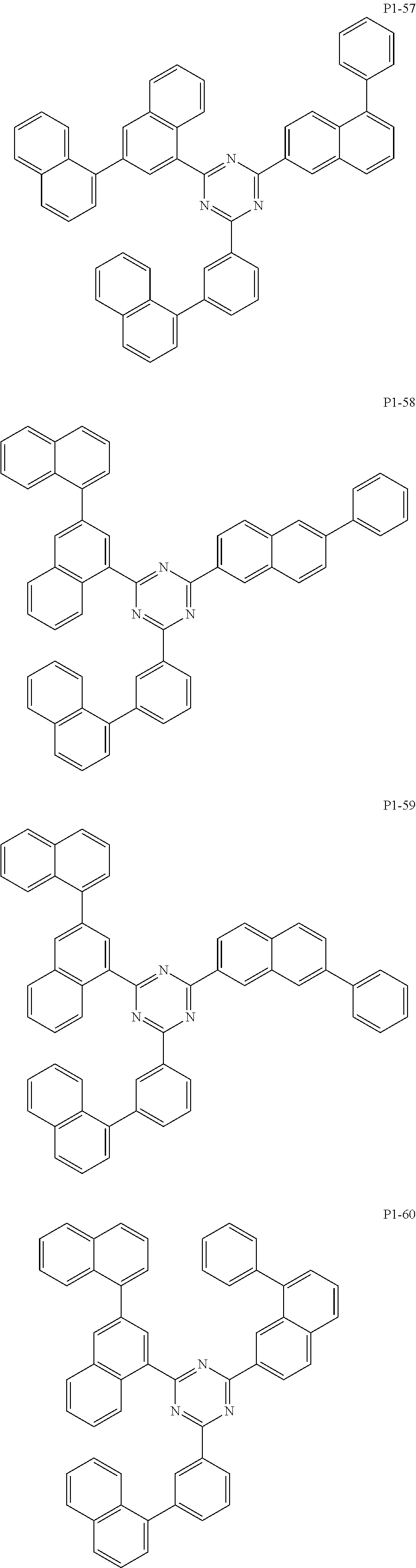 [SMILES: c1ccc(-c2cccc3cc(-c4nc(-c5cccc(-c6cccc7ccccc67)c5)nc(-c5cc(-c6cccc7ccccc67)cc6ccccc56)n4)ccc23)cc1 ; c1ccc(-c2ccc3cc(-c4nc(-c5cccc(-c6cccc7ccccc67)c5)nc(-c5cc(-c6cccc7ccccc67)cc6ccccc56)n4)ccc3c2)cc1 ; c1ccc(-c2ccc3ccc(-c4nc(-c5cccc(-c6cccc7ccccc67)c5)nc(-c5cc(-c6cccc7ccccc67)cc6ccccc56)n4)cc3c2)cc1 ; c1ccc(-c2cccc3ccc(-c4nc(-c5cccc(-c6cccc7ccccc67)c5)nc(-c5cc(-c6cccc7ccccc67)cc6ccccc56)n4)cc23)cc1]

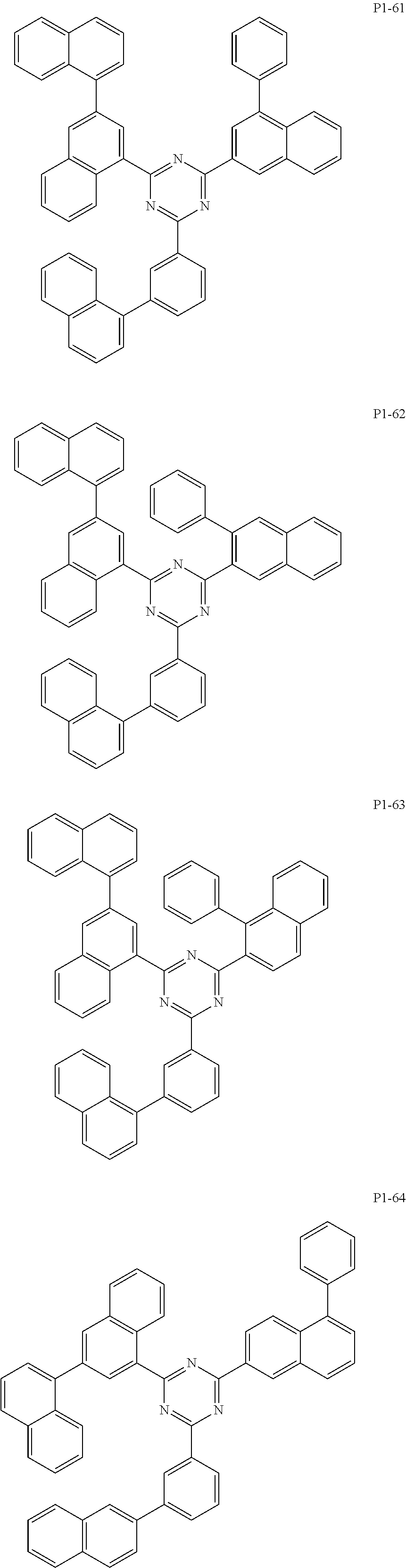 [SMILES: c1ccc(-c2cc(-c3nc(-c4cccc(-c5cccc6ccccc56)c4)nc(-c4cc(-c5cccc6ccccc56)cc5ccccc45)n3)cc3ccccc23)cc1 ; c1ccc(-c2cc3ccccc3cc2-c2nc(-c3cccc(-c4cccc5ccccc45)c3)nc(-c3cc(-c4cccc5ccccc45)cc4ccccc34)n2)cc1 ; c1ccc(-c2c(-c3nc(-c4cccc(-c5cccc6ccccc56)c4)nc(-c4cc(-c5cccc6ccccc56)cc5ccccc45)n3)ccc3ccccc23)cc1 ; c1ccc(-c2cccc3cc(-c4nc(-c5cccc(-c6ccc7ccccc7c6)c5)nc(-c5cc(-c6cccc7ccccc67)cc6ccccc56)n4)ccc23)cc1]

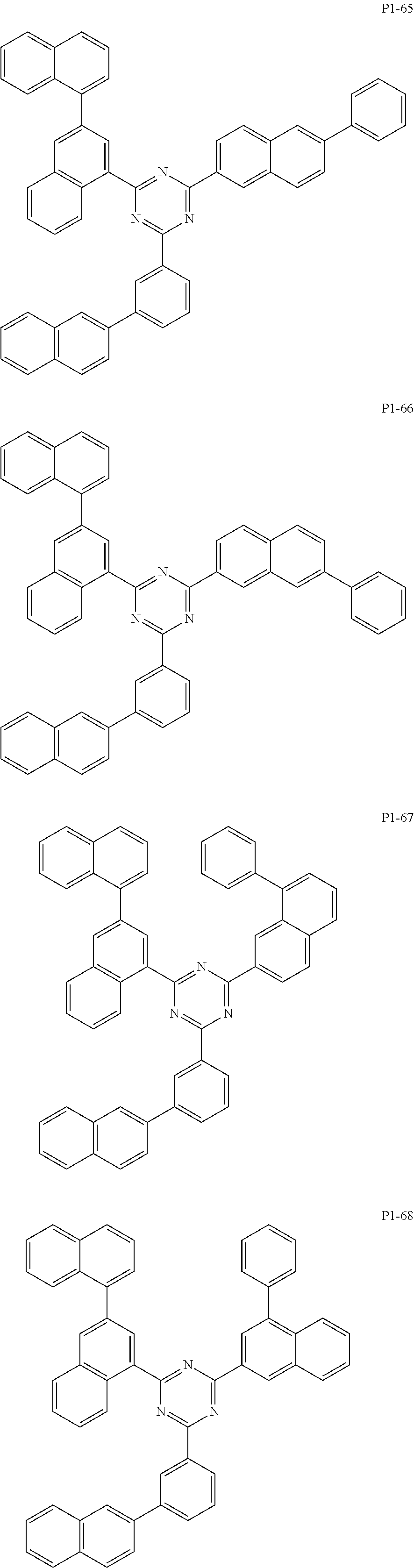 [SMILES: c1ccc(-c2ccc3cc(-c4nc(-c5cccc(-c6ccc7ccccc7c6)c5)nc(-c5cc(-c6cccc7ccccc67)cc6ccccc56)n4)ccc3c2)cc1 ; c1ccc(-c2ccc3ccc(-c4nc(-c5cccc(-c6ccc7ccccc7c6)c5)nc(-c5cc(-c6cccc7ccccc67)cc6ccccc56)n4)cc3c2)cc1 ; c1ccc(-c2cccc3ccc(-c4nc(-c5cccc(-c6ccc7ccccc7c6)c5)nc(-c5cc(-c6cccc7ccccc67)cc6ccccc56)n4)cc23)cc1 ; c1ccc(-c2cc(-c3nc(-c4cccc(-c5ccc6ccccc6c5)c4)nc(-c4cc(-c5cccc6ccccc56)cc5ccccc45)n3)cc3ccccc23)cc1]

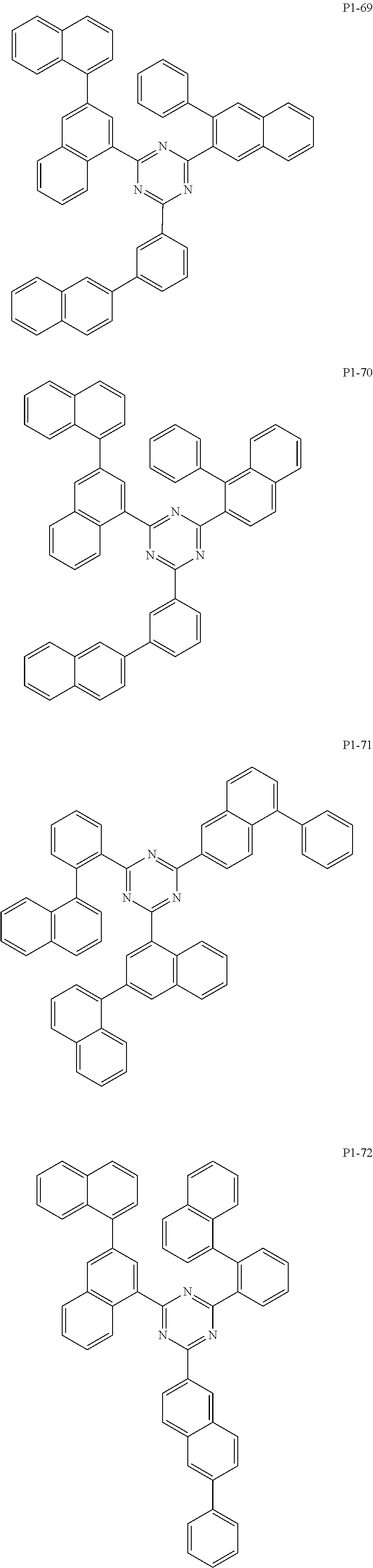 [SMILES: c1ccc(-c2cc3ccccc3cc2-c2nc(-c3cccc(-c4ccc5ccccc5c4)c3)nc(-c3cc(-c4cccc5ccccc45)cc4ccccc34)n2)cc1 ; c1ccc(-c2c(-c3nc(-c4cccc(-c5ccc6ccccc6c5)c4)nc(-c4cc(-c5cccc6ccccc56)cc5ccccc45)n3)ccc3ccccc23)cc1 ; c1ccc(-c2cccc3cc(-c4nc(-c5ccccc5-c5cccc6ccccc56)nc(-c5cc(-c6cccc7ccccc67)cc6ccccc56)n4)ccc23)cc1 ; c1ccc(-c2ccc3cc(-c4nc(-c5ccccc5-c5cccc6ccccc56)nc(-c5cc(-c6cccc7ccccc67)cc6ccccc56)n4)ccc3c2)cc1]

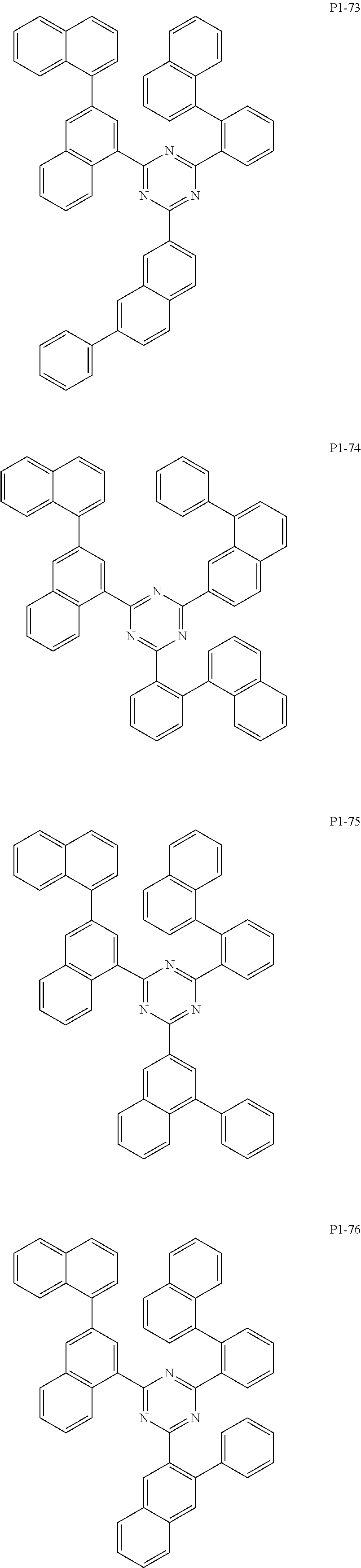 [SMILES: c1ccc(-c2ccc3ccc(-c4nc(-c5ccccc5-c5cccc6ccccc56)nc(-c5cc(-c6cccc7ccccc67)cc6ccccc56)n4)cc3c2)cc1 ; c1ccc(-c2cccc3ccc(-c4nc(-c5ccccc5-c5cccc6ccccc56)nc(-c5cc(-c6cccc7ccccc67)cc6ccccc56)n4)cc23)cc1 ; c1ccc(-c2cc(-c3nc(-c4ccccc4-c4cccc5ccccc45)nc(-c4cc(-c5cccc6ccccc56)cc5ccccc45)n3)cc3ccccc23)cc1 ; c1ccc(-c2cc3ccccc3cc2-c2nc(-c3ccccc3-c3cccc4ccccc34)nc(-c3cc(-c4cccc5ccccc45)cc4ccccc34)n2)cc1]

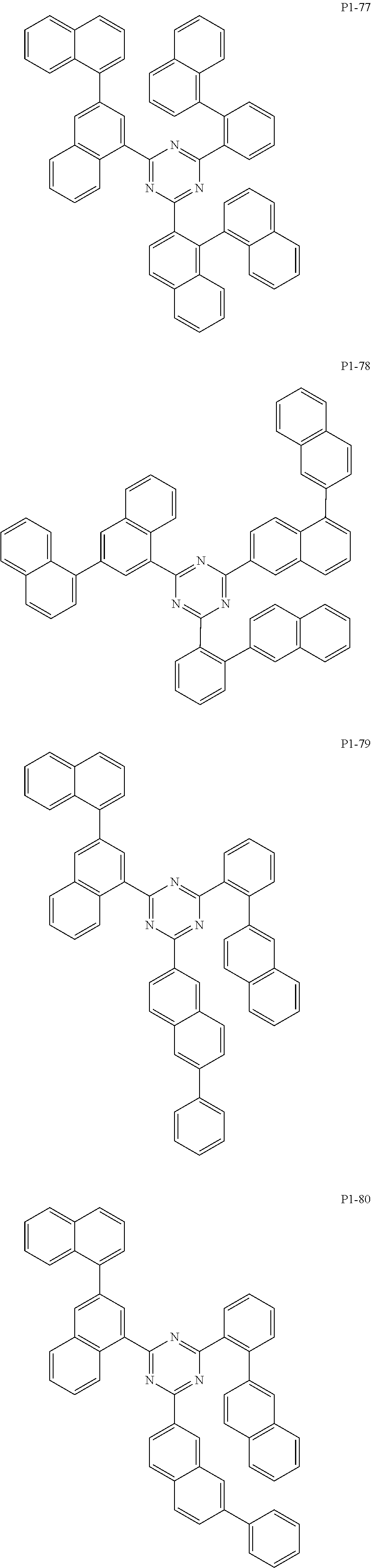 [SMILES: c1ccc(-c2cccc3ccccc23)c(-c2nc(-c3ccc4ccccc4c3-c3cccc4ccccc34)nc(-c3cc(-c4cccc5ccccc45)cc4ccccc34)n2)c1 ; c1ccc(-c2nc(-c3ccc4c(-c5ccc6ccccc6c5)cccc4c3)nc(-c3cc(-c4cccc5ccccc45)cc4ccccc34)n2)c(-c2ccc3ccccc3c2)c1 ; c1ccc(-c2ccc3cc(-c4nc(-c5ccccc5-c5ccc6ccccc6c5)nc(-c5cc(-c6cccc7ccccc67)cc6ccccc56)n4)ccc3c2)cc1 ; c1ccc(-c2ccc3ccc(-c4nc(-c5ccccc5-c5ccc6ccccc6c5)nc(-c5cc(-c6cccc7ccccc67)cc6ccccc56)n4)cc3c2)cc1]

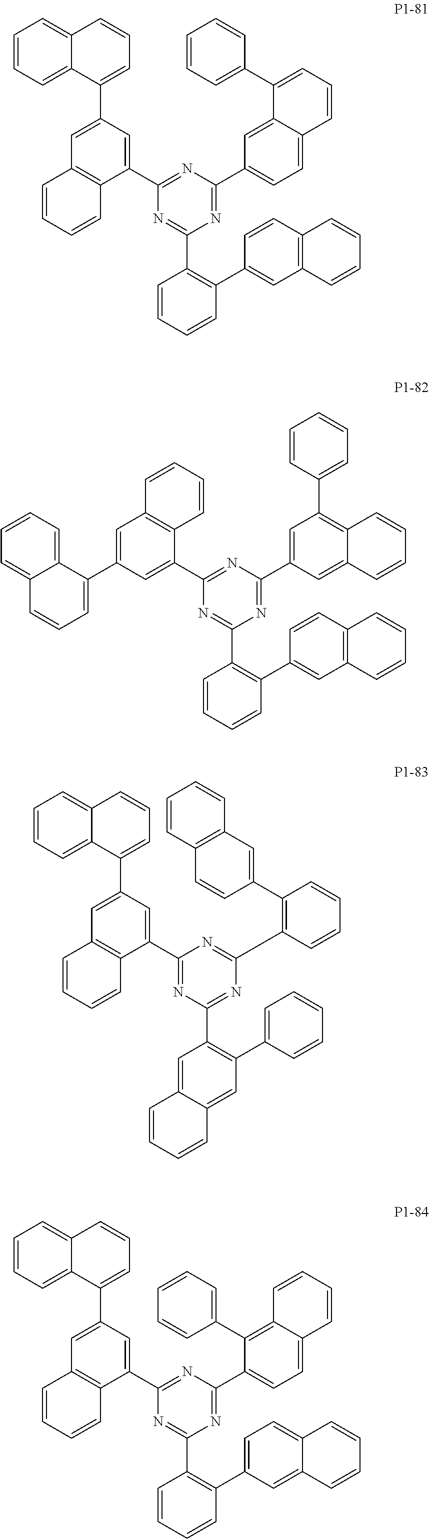 [SMILES: c1ccc(-c2cccc3ccc(-c4nc(-c5ccccc5-c5ccc6ccccc6c5)nc(-c5cc(-c6cccc7ccccc67)cc6ccccc56)n4)cc23)cc1 ; c1ccc(-c2cc(-c3nc(-c4ccccc4-c4ccc5ccccc5c4)nc(-c4cc(-c5cccc6ccccc56)cc5ccccc45)n3)cc3ccccc23)cc1 ; c1ccc(-c2cc3ccccc3cc2-c2nc(-c3ccccc3-c3ccc4ccccc4c3)nc(-c3cc(-c4cccc5ccccc45)cc4ccccc34)n2)cc1 ; c1ccc(-c2c(-c3nc(-c4ccccc4-c4ccc5ccccc5c4)nc(-c4cc(-c5cccc6ccccc56)cc5ccccc45)n3)ccc3ccccc23)cc1]

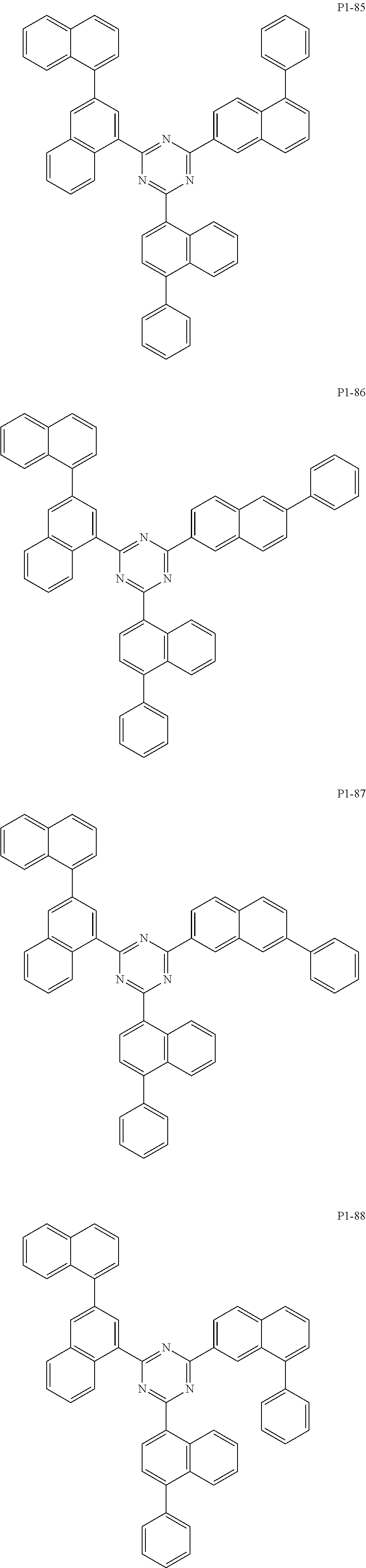 [SMILES: c1ccc(-c2cccc3cc(-c4nc(-c5cc(-c6cccc7ccccc67)cc6ccccc56)nc(-c5ccc(-c6ccccc6)c6ccccc56)n4)ccc23)cc1 ; c1ccc(-c2ccc3cc(-c4nc(-c5cc(-c6cccc7ccccc67)cc6ccccc56)nc(-c5ccc(-c6ccccc6)c6ccccc56)n4)ccc3c2)cc1 ; c1ccc(-c2ccc3ccc(-c4nc(-c5cc(-c6cccc7ccccc67)cc6ccccc56)nc(-c5ccc(-c6ccccc6)c6ccccc56)n4)cc3c2)cc1 ; c1ccc(-c2cccc3ccc(-c4nc(-c5cc(-c6cccc7ccccc67)cc6ccccc56)nc(-c5ccc(-c6ccccc6)c6ccccc56)n4)cc23)cc1]

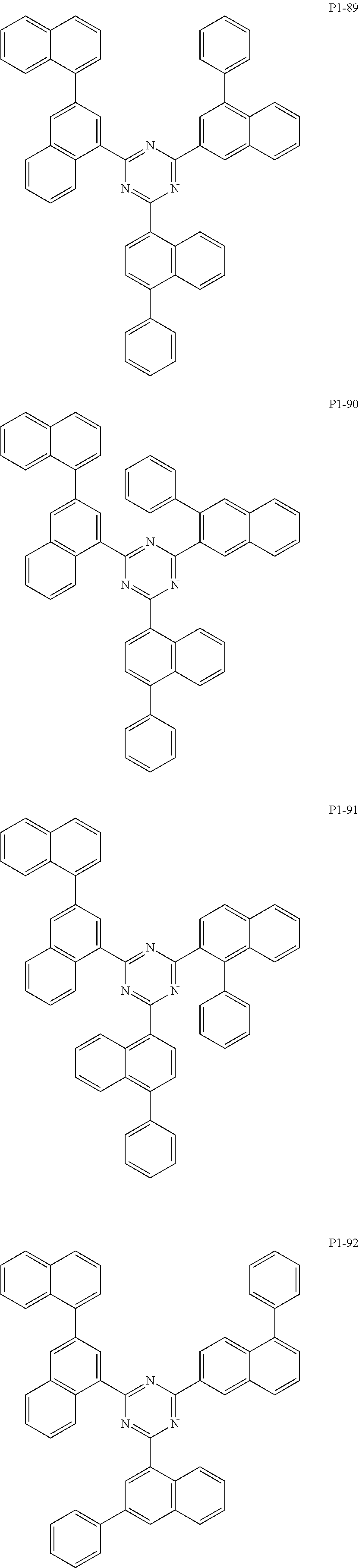 [SMILES: c1ccc(-c2cc(-c3nc(-c4cc(-c5cccc6ccccc56)cc5ccccc45)nc(-c4ccc(-c5ccccc5)c5ccccc45)n3)cc3ccccc23)cc1 ; c1ccc(-c2cc3ccccc3cc2-c2nc(-c3cc(-c4cccc5ccccc45)cc4ccccc34)nc(-c3ccc(-c4ccccc4)c4ccccc34)n2)cc1 ; c1ccc(-c2c(-c3nc(-c4cc(-c5cccc6ccccc56)cc5ccccc45)nc(-c4ccc(-c5ccccc5)c5ccccc45)n3)ccc3ccccc23)cc1 ; c1ccc(-c2cc(-c3nc(-c4ccc5c(-c6ccccc6)cccc5c4)nc(-c4cc(-c5cccc6ccccc56)cc5ccccc45)n3)c3ccccc3c2)cc1]

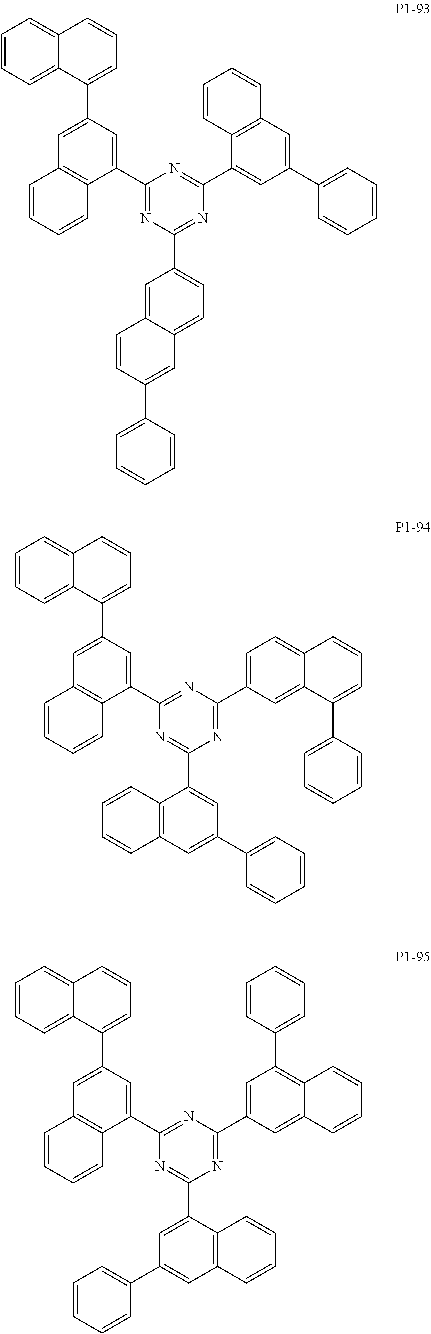 [SMILES: c1ccc(-c2ccc3cc(-c4nc(-c5cc(-c6ccccc6)cc6ccccc56)nc(-c5cc(-c6cccc7ccccc67)cc6ccccc56)n4)ccc3c2)cc1 ; c1ccc(-c2cc(-c3nc(-c4ccc5cccc(-c6ccccc6)c5c4)nc(-c4cc(-c5cccc6ccccc56)cc5ccccc45)n3)c3ccccc3c2)cc1 ; c1ccc(-c2cc(-c3nc(-c4cc(-c5ccccc5)c5ccccc5c4)nc(-c4cc(-c5cccc6ccccc56)cc5ccccc45)n3)c3ccccc3c2)cc1]

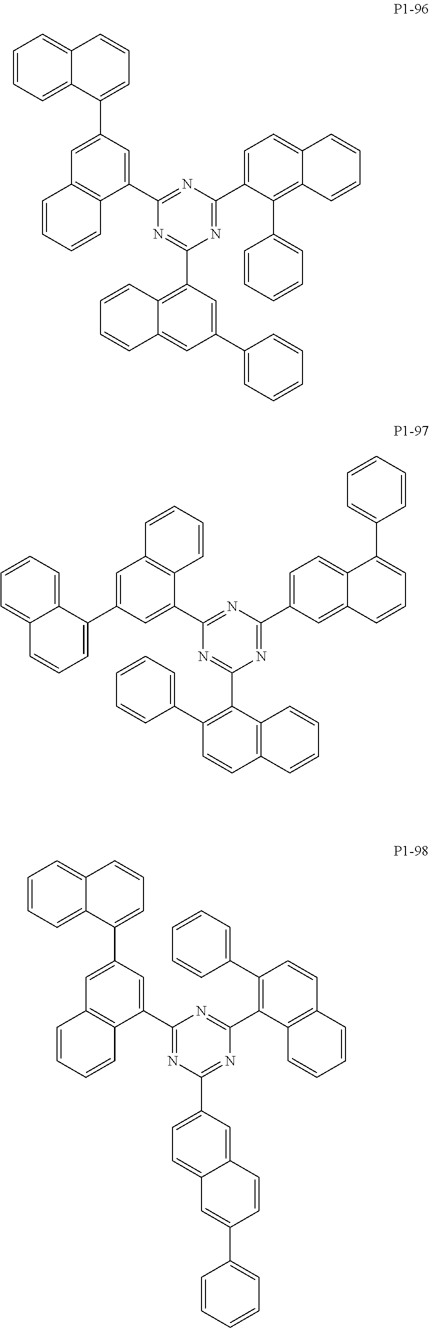 [SMILES: c1ccc(-c2cc(-c3nc(-c4ccc5ccccc5c4-c4ccccc4)nc(-c4cc(-c5cccc6ccccc56)cc5ccccc45)n3)c3ccccc3c2)cc1 ; c1ccc(-c2ccc3ccccc3c2-c2nc(-c3ccc4c(-c5ccccc5)cccc4c3)nc(-c3cc(-c4cccc5ccccc45)cc4ccccc34)n2)cc1 ; c1ccc(-c2ccc3cc(-c4nc(-c5cc(-c6cccc7ccccc67)cc6ccccc56)nc(-c5c(-c6ccccc6)ccc6ccccc56)n4)ccc3c2)cc1]

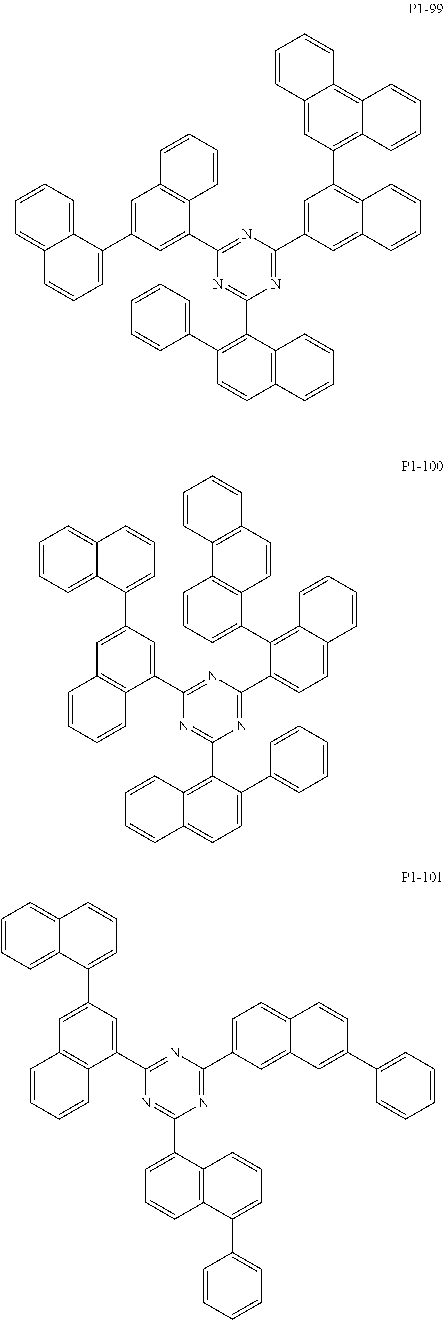 [SMILES: c1ccc(-c2ccc3ccccc3c2-c2nc(-c3cc(-c4cc5ccccc5c5ccccc45)c4ccccc4c3)nc(-c3cc(-c4cccc5ccccc45)cc4ccccc34)n2)cc1 ; c1ccc(-c2ccc3ccccc3c2-c2nc(-c3ccc4ccccc4c3-c3cccc4c3ccc3ccccc34)nc(-c3cc(-c4cccc5ccccc45)cc4ccccc34)n2)cc1 ; c1ccc(-c2ccc3ccc(-c4nc(-c5cc(-c6cccc7ccccc67)cc6ccccc56)nc(-c5cccc6c(-c7ccccc7)cccc56)n4)cc3c2)cc1]

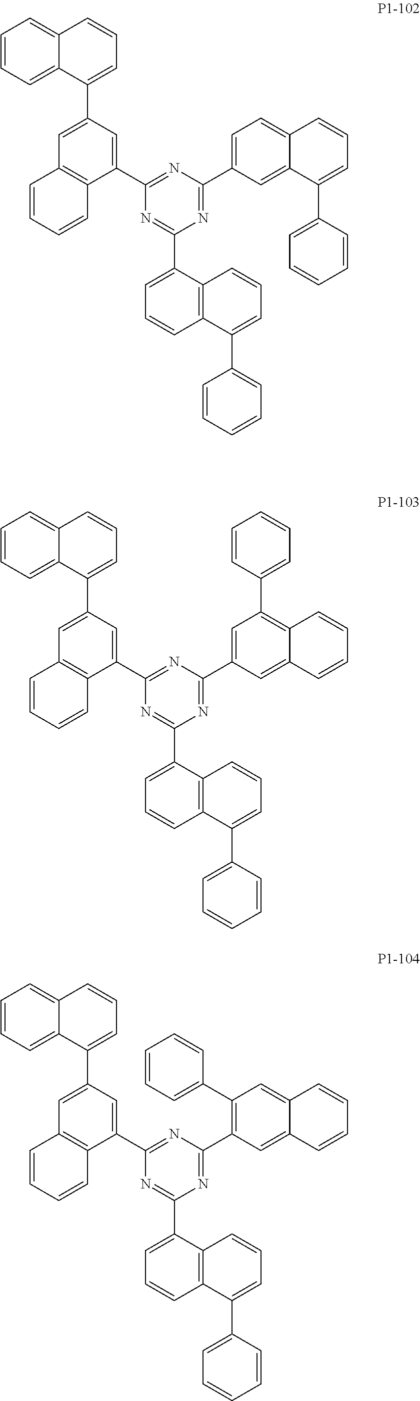 [SMILES: c1ccc(-c2cccc3ccc(-c4nc(-c5cc(-c6cccc7ccccc67)cc6ccccc56)nc(-c5cccc6c(-c7ccccc7)cccc56)n4)cc23)cc1 ; c1ccc(-c2cc(-c3nc(-c4cc(-c5cccc6ccccc56)cc5ccccc45)nc(-c4cccc5c(-c6ccccc6)cccc45)n3)cc3ccccc23)cc1 ; c1ccc(-c2cc3ccccc3cc2-c2nc(-c3cc(-c4cccc5ccccc45)cc4ccccc34)nc(-c3cccc4c(-c5ccccc5)cccc34)n2)cc1]

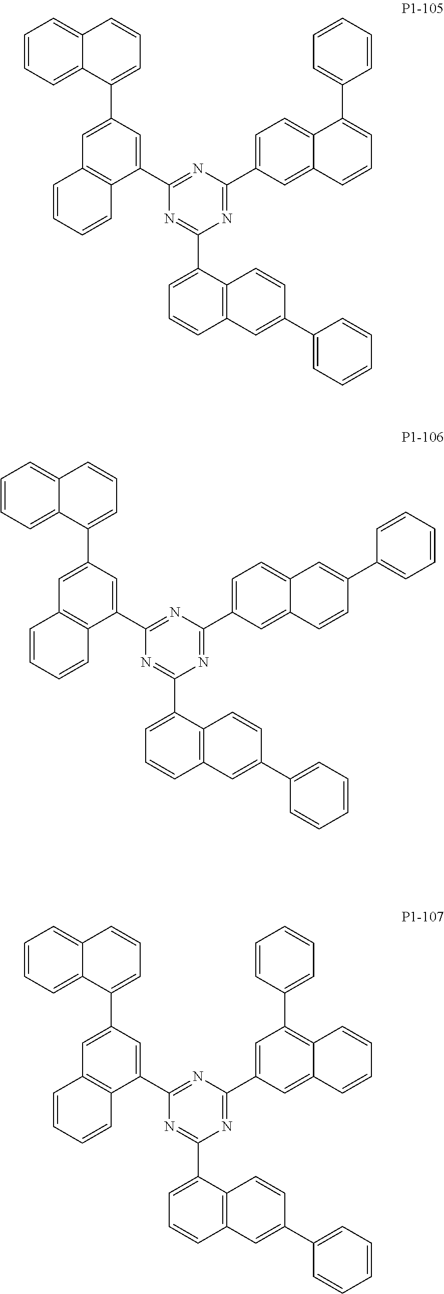 [SMILES: c1ccc(-c2ccc3c(-c4nc(-c5ccc6c(-c7ccccc7)cccc6c5)nc(-c5cc(-c6cccc7ccccc67)cc6ccccc56)n4)cccc3c2)cc1 ; c1ccc(-c2ccc3cc(-c4nc(-c5cccc6cc(-c7ccccc7)ccc56)nc(-c5cc(-c6cccc7ccccc67)cc6ccccc56)n4)ccc3c2)cc1 ; c1ccc(-c2ccc3c(-c4nc(-c5cc(-c6ccccc6)c6ccccc6c5)nc(-c5cc(-c6cccc7ccccc67)cc6ccccc56)n4)cccc3c2)cc1]

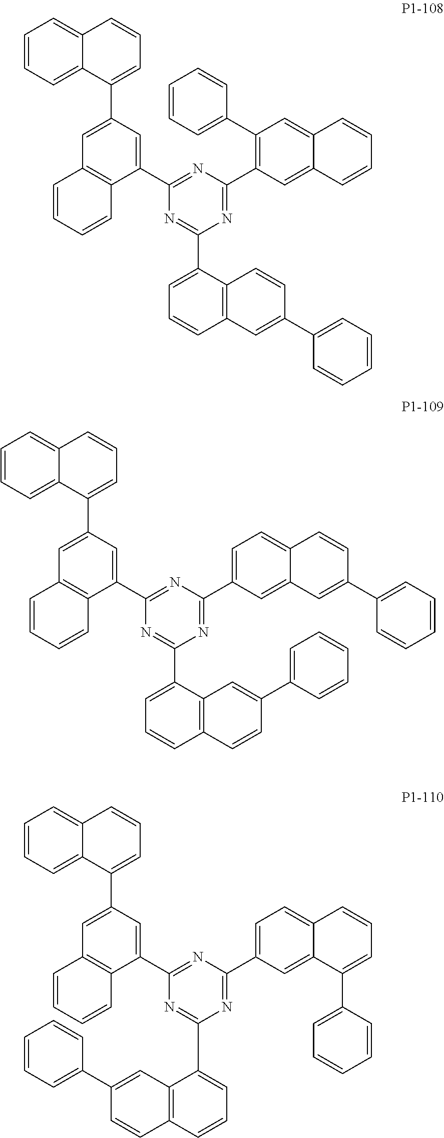 [SMILES: c1ccc(-c2ccc3c(-c4nc(-c5cc6ccccc6cc5-c5ccccc5)nc(-c5cc(-c6cccc7ccccc67)cc6ccccc56)n4)cccc3c2)cc1 ; c1ccc(-c2ccc3ccc(-c4nc(-c5cc(-c6cccc7ccccc67)cc6ccccc56)nc(-c5cccc6ccc(-c7ccccc7)cc56)n4)cc3c2)cc1 ; c1ccc(-c2ccc3cccc(-c4nc(-c5ccc6cccc(-c7ccccc7)c6c5)nc(-c5cc(-c6cccc7ccccc67)cc6ccccc56)n4)c3c2)cc1]

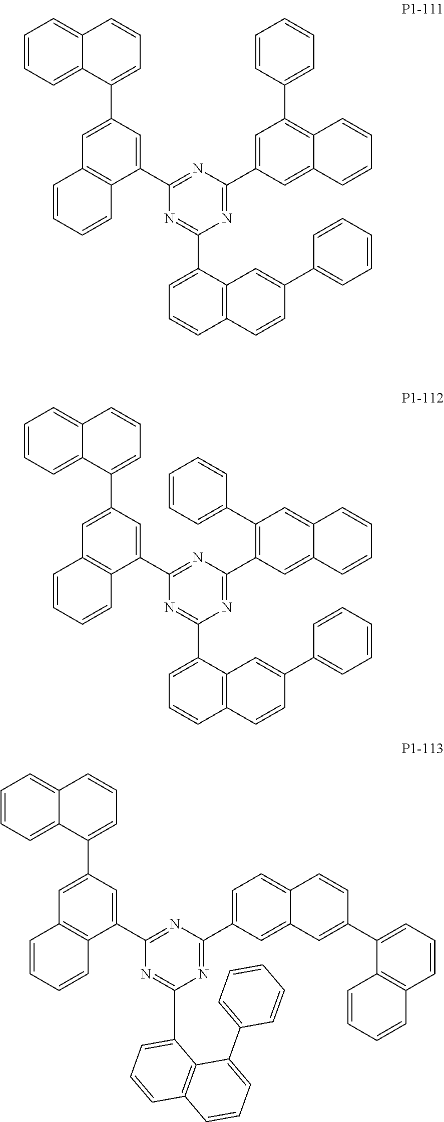 [SMILES: c1ccc(-c2ccc3cccc(-c4nc(-c5cc(-c6ccccc6)c6ccccc6c5)nc(-c5cc(-c6cccc7ccccc67)cc6ccccc56)n4)c3c2)cc1 ; c1ccc(-c2ccc3cccc(-c4nc(-c5cc6ccccc6cc5-c5ccccc5)nc(-c5cc(-c6cccc7ccccc67)cc6ccccc56)n4)c3c2)cc1 ; c1ccc(-c2cccc3cccc(-c4nc(-c5ccc6ccc(-c7cccc8ccccc78)cc6c5)nc(-c5cc(-c6cccc7ccccc67)cc6ccccc56)n4)c23)cc1]

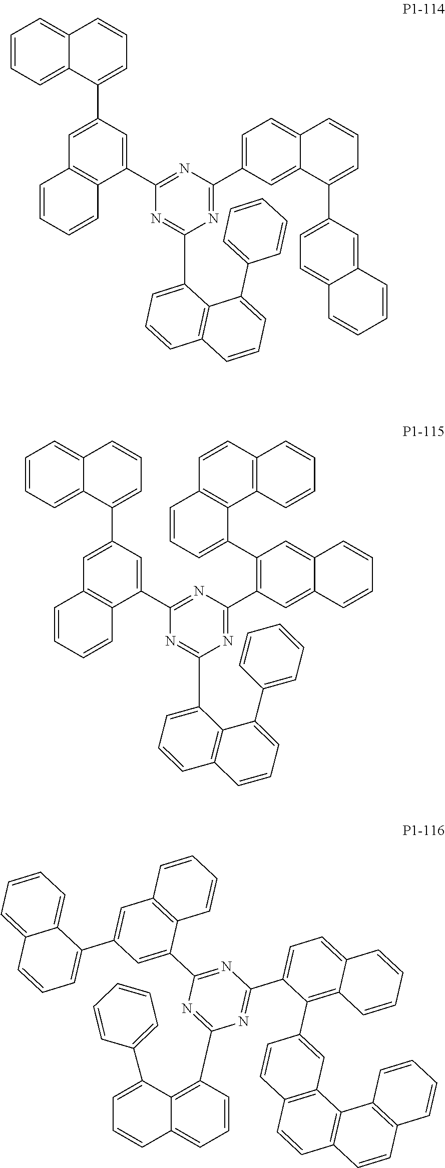 [SMILES: c1ccc(-c2cccc3cccc(-c4nc(-c5ccc6cccc(-c7ccc8ccccc8c7)c6c5)nc(-c5cc(-c6cccc7ccccc67)cc6ccccc56)n4)c23)cc1 ; c1ccc(-c2cccc3cccc(-c4nc(-c5cc6ccccc6cc5-c5cccc6ccc7ccccc7c56)nc(-c5cc(-c6cccc7ccccc67)cc6ccccc56)n4)c23)cc1 ; c1ccc(-c2cccc3cccc(-c4nc(-c5ccc6ccccc6c5-c5ccc6ccc7ccc8ccccc8c7c6c5)nc(-c5cc(-c6cccc7ccccc67)cc6ccccc56)n4)c23)cc1]

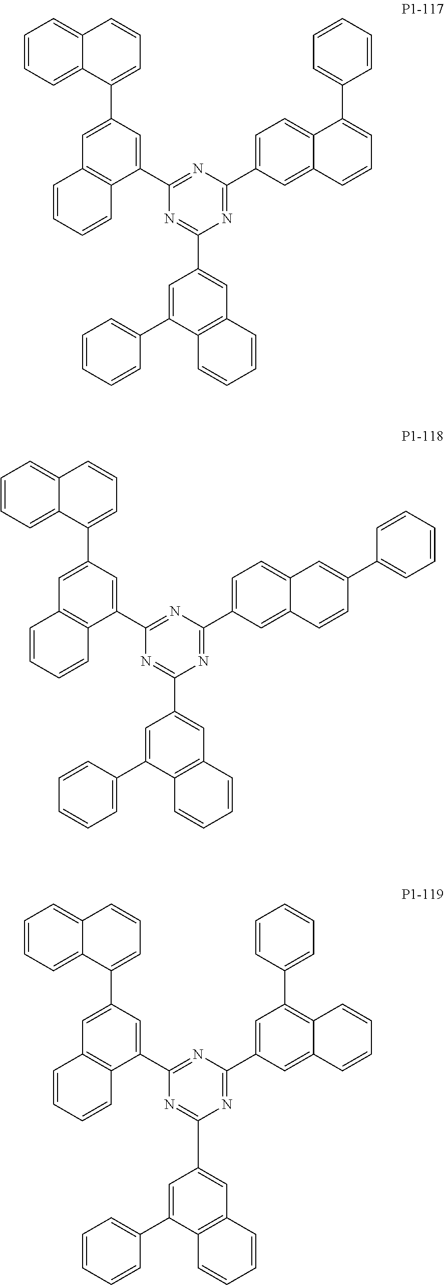 [SMILES: c1ccc(-c2cccc3cc(-c4nc(-c5cc(-c6ccccc6)c6ccccc6c5)nc(-c5cc(-c6cccc7ccccc67)cc6ccccc56)n4)ccc23)cc1 ; c1ccc(-c2ccc3cc(-c4nc(-c5cc(-c6ccccc6)c6ccccc6c5)nc(-c5cc(-c6cccc7ccccc67)cc6ccccc56)n4)ccc3c2)cc1 ; c1ccc(-c2cc(-c3nc(-c4cc(-c5ccccc5)c5ccccc5c4)nc(-c4cc(-c5cccc6ccccc56)cc5ccccc45)n3)cc3ccccc23)cc1]

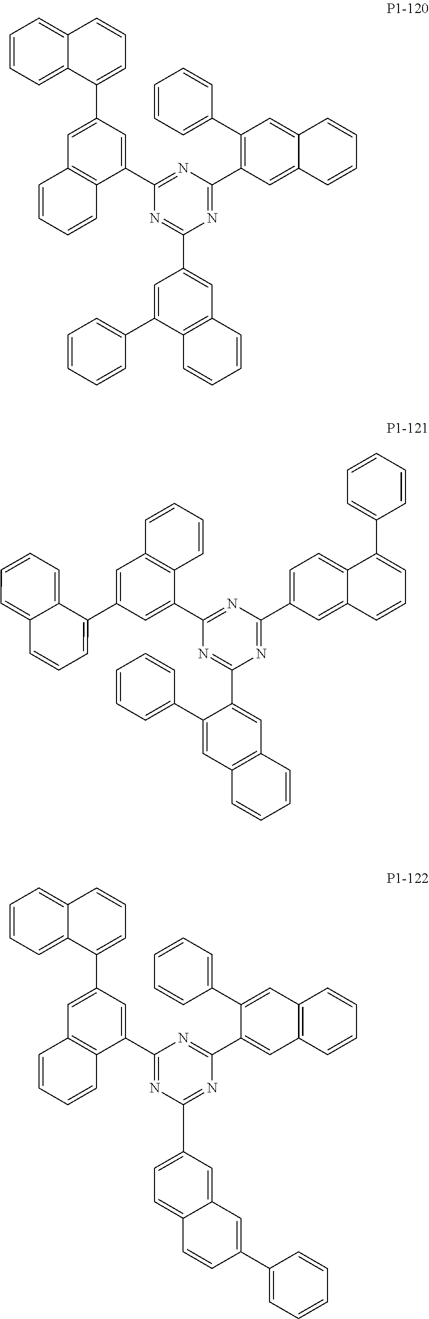 [SMILES: c1ccc(-c2cc3ccccc3cc2-c2nc(-c3cc(-c4ccccc4)c4ccccc4c3)nc(-c3cc(-c4cccc5ccccc45)cc4ccccc34)n2)cc1 ; c1ccc(-c2cc3ccccc3cc2-c2nc(-c3ccc4c(-c5ccccc5)cccc4c3)nc(-c3cc(-c4cccc5ccccc45)cc4ccccc34)n2)cc1 ; c1ccc(-c2ccc3ccc(-c4nc(-c5cc6ccccc6cc5-c5ccccc5)nc(-c5cc(-c6cccc7ccccc67)cc6ccccc56)n4)cc3c2)cc1]

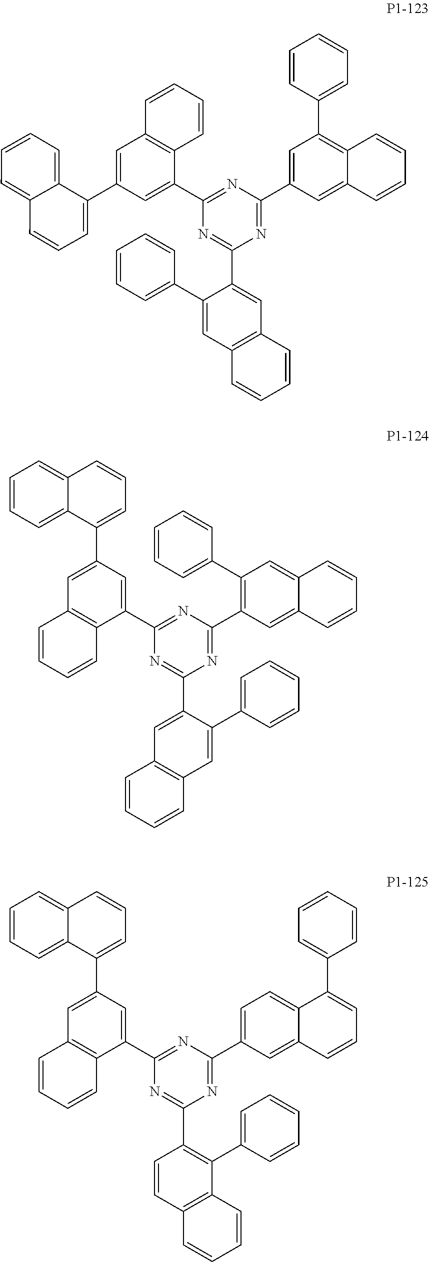 [SMILES: c1ccc(-c2cc3ccccc3cc2-c2nc(-c3cc(-c4ccccc4)c4ccccc4c3)nc(-c3cc(-c4cccc5ccccc45)cc4ccccc34)n2)cc1 ; c1ccc(-c2cc3ccccc3cc2-c2nc(-c3cc4ccccc4cc3-c3ccccc3)nc(-c3cc(-c4cccc5ccccc45)cc4ccccc34)n2)cc1 ; c1ccc(-c2cccc3cc(-c4nc(-c5ccc6ccccc6c5-c5ccccc5)nc(-c5cc(-c6cccc7ccccc67)cc6ccccc56)n4)ccc23)cc1]

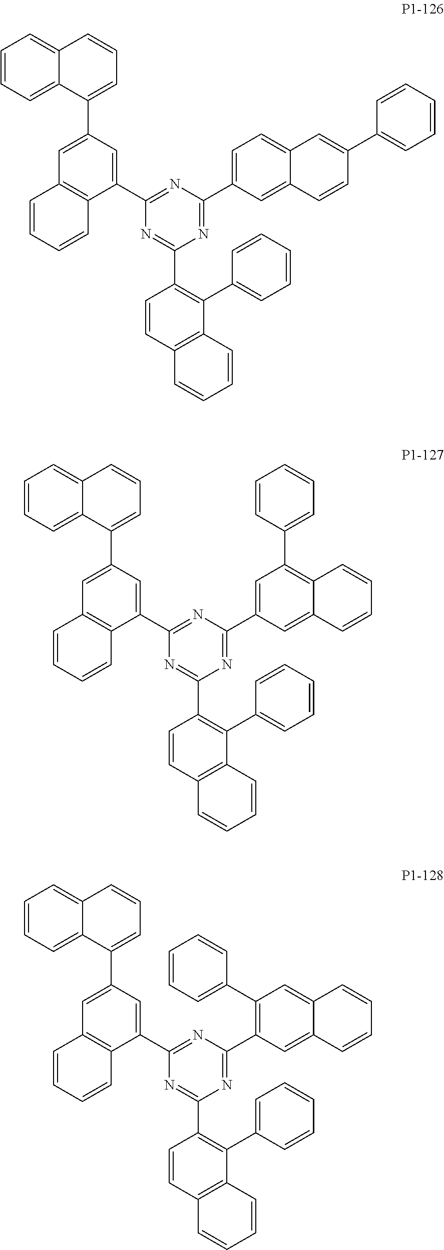 [SMILES: c1ccc(-c2ccc3cc(-c4nc(-c5ccc6ccccc6c5-c5ccccc5)nc(-c5cc(-c6cccc7ccccc67)cc6ccccc56)n4)ccc3c2)cc1 ; c1ccc(-c2cc(-c3nc(-c4ccc5ccccc5c4-c4ccccc4)nc(-c4cc(-c5cccc6ccccc56)cc5ccccc45)n3)cc3ccccc23)cc1 ; c1ccc(-c2cc3ccccc3cc2-c2nc(-c3ccc4ccccc4c3-c3ccccc3)nc(-c3cc(-c4cccc5ccccc45)cc4ccccc34)n2)cc1]

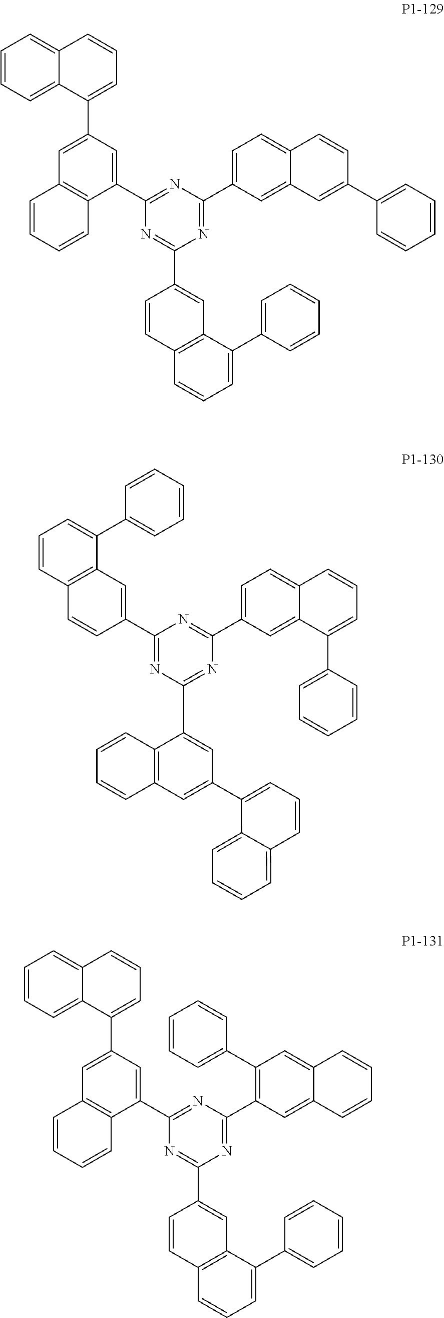 [SMILES: c1ccc(-c2ccc3ccc(-c4nc(-c5ccc6cccc(-c7ccccc7)c6c5)nc(-c5cc(-c6cccc7ccccc67)cc6ccccc56)n4)cc3c2)cc1 ; c1ccc(-c2cccc3ccc(-c4nc(-c5ccc6cccc(-c7ccccc7)c6c5)nc(-c5cc(-c6cccc7ccccc67)cc6ccccc56)n4)cc23)cc1 ; c1ccc(-c2cc3ccccc3cc2-c2nc(-c3ccc4cccc(-c5ccccc5)c4c3)nc(-c3cc(-c4cccc5ccccc45)cc4ccccc34)n2)cc1]

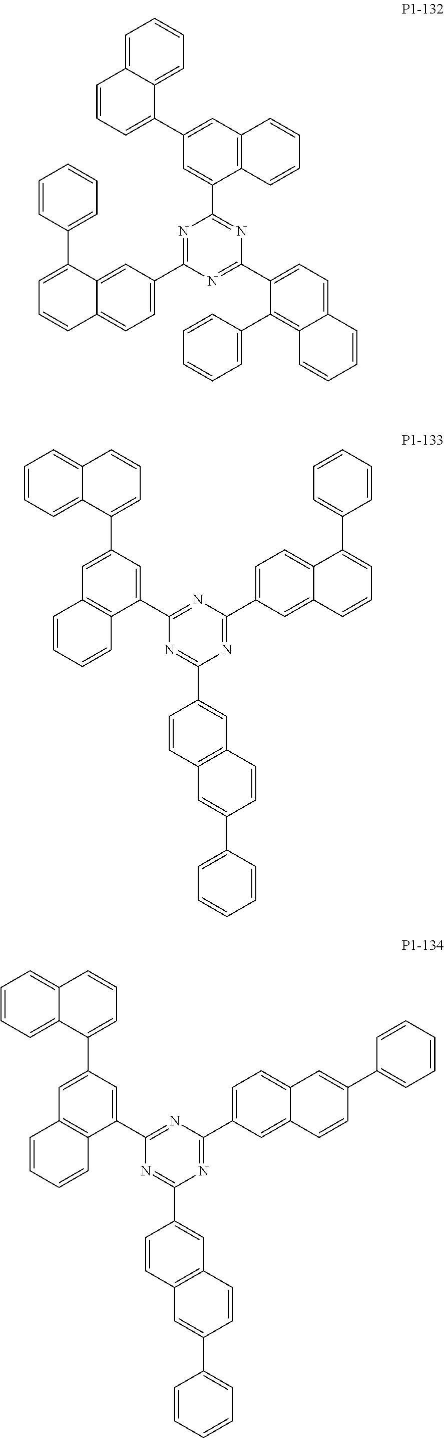 [SMILES: c1ccc(-c2cccc3ccc(-c4nc(-c5ccc6ccccc6c5-c5ccccc5)nc(-c5cc(-c6cccc7ccccc67)cc6ccccc56)n4)cc23)cc1 ; c1ccc(-c2ccc3cc(-c4nc(-c5ccc6c(-c7ccccc7)cccc6c5)nc(-c5cc(-c6cccc7ccccc67)cc6ccccc56)n4)ccc3c2)cc1 ; c1ccc(-c2ccc3cc(-c4nc(-c5ccc6cc(-c7ccccc7)ccc6c5)nc(-c5cc(-c6cccc7ccccc67)cc6ccccc56)n4)ccc3c2)cc1]

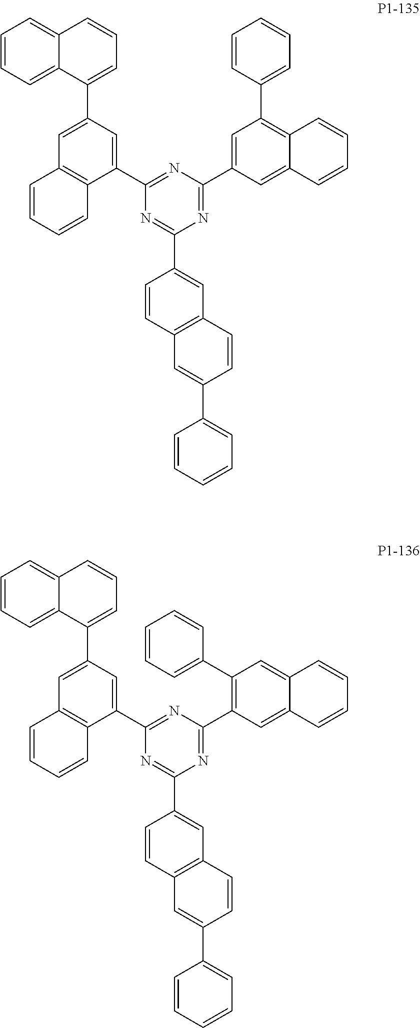 [SMILES: c1ccc(-c2ccc3cc(-c4nc(-c5cc(-c6ccccc6)c6ccccc6c5)nc(-c5cc(-c6cccc7ccccc67)cc6ccccc56)n4)ccc3c2)cc1 ; c1ccc(-c2ccc3cc(-c4nc(-c5cc6ccccc6cc5-c5ccccc5)nc(-c5cc(-c6cccc7ccccc67)cc6ccccc56)n4)ccc3c2)cc1]

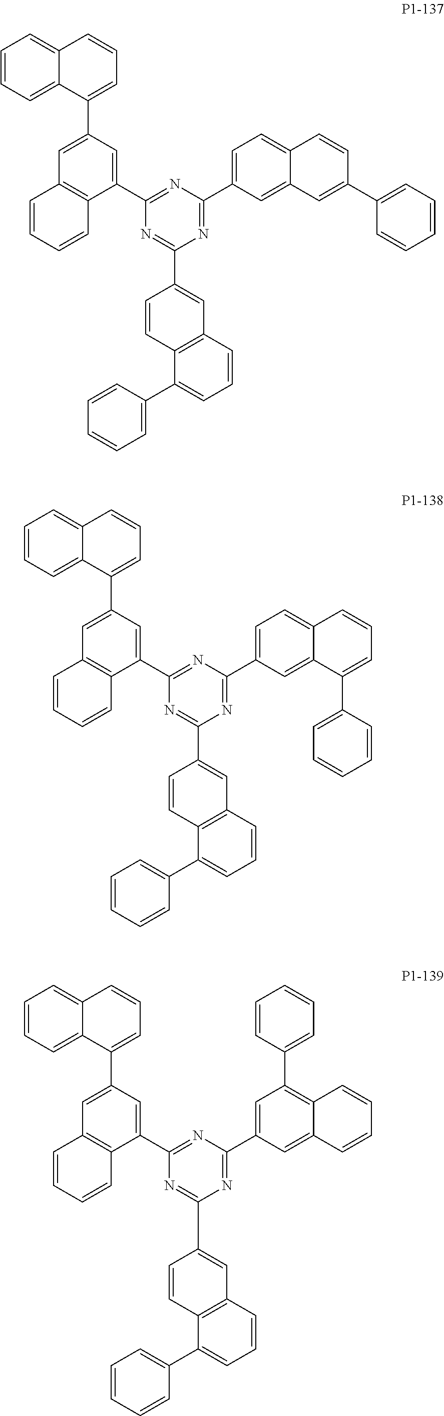 [SMILES: c1ccc(-c2ccc3ccc(-c4nc(-c5ccc6c(-c7ccccc7)cccc6c5)nc(-c5cc(-c6cccc7ccccc67)cc6ccccc56)n4)cc3c2)cc1 ; c1ccc(-c2cccc3cc(-c4nc(-c5ccc6cccc(-c7ccccc7)c6c5)nc(-c5cc(-c6cccc7ccccc67)cc6ccccc56)n4)ccc23)cc1 ; c1ccc(-c2cccc3cc(-c4nc(-c5cc(-c6ccccc6)c6ccccc6c5)nc(-c5cc(-c6cccc7ccccc67)cc6ccccc56)n4)ccc23)cc1]

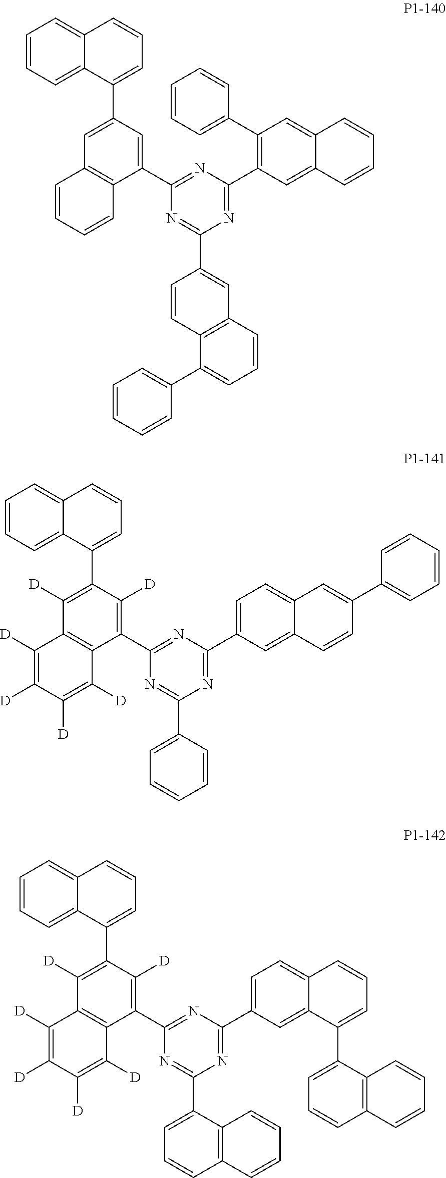 [SMILES: c1ccc(-c2cc3ccccc3cc2-c2nc(-c3ccc4c(-c5ccccc5)cccc4c3)nc(-c3cc(-c4cccc5ccccc45)cc4ccccc34)n2)cc1 ; [2H]c1c([2H])c([2H])c2c(-c3nc(-c4ccccc4)nc(-c4ccc5cc(-c6ccccc6)ccc5c4)n3)c([2H])c(-c3cccc4ccccc34)c([2H])c2c1[2H] ; [2H]c1c([2H])c([2H])c2c(-c3nc(-c4ccc5cccc(-c6cccc7ccccc67)c5c4)nc(-c4cccc5ccccc45)n3)c([2H])c(-c3cccc4ccccc34)c([2H])c2c1[2H]]

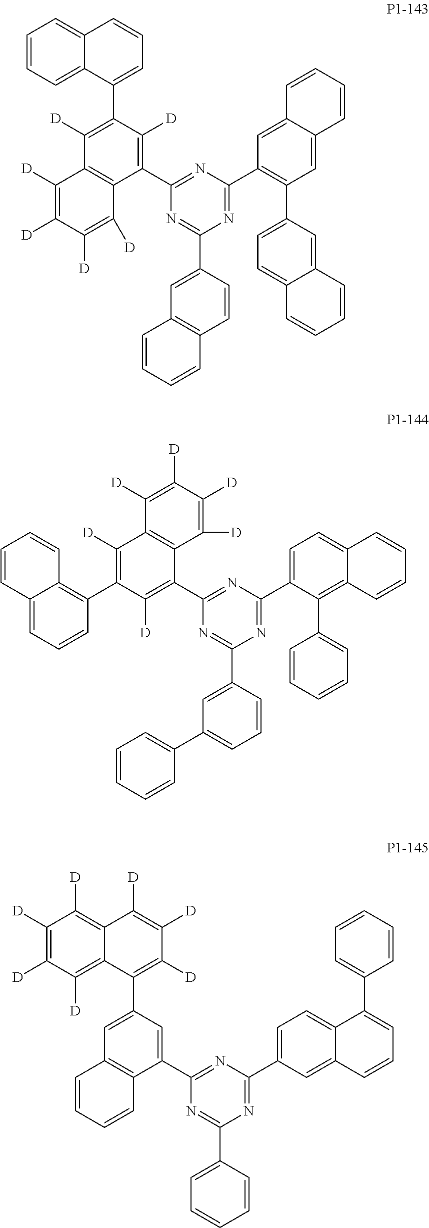 [SMILES: [2H]c1c([2H])c([2H])c2c(-c3nc(-c4ccc5ccccc5c4)nc(-c4cc5ccccc5cc4-c4ccc5ccccc5c4)n3)c([2H])c(-c3cccc4ccccc34)c([2H])c2c1[2H] ; [2H]c1c([2H])c([2H])c2c(-c3nc(-c4cccc(-c5ccccc5)c4)nc(-c4ccc5ccccc5c4-c4ccccc4)n3)c([2H])c(-c3cccc4ccccc34)c([2H])c2c1[2H] ; [2H]c1c([2H])c([2H])c2c(-c3cc(-c4nc(-c5ccccc5)nc(-c5ccc6c(-c7ccccc7)cccc6c5)n4)c4ccccc4c3)c([2H])c([2H])c([2H])c2c1[2H]]

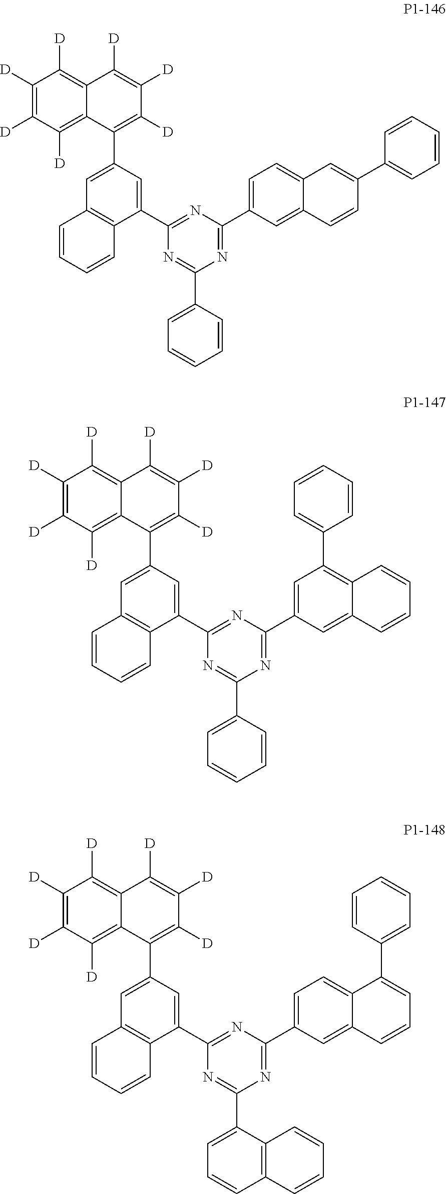 [SMILES: [2H]c1c([2H])c([2H])c2c(-c3cc(-c4nc(-c5ccccc5)nc(-c5ccc6cc(-c7ccccc7)ccc6c5)n4)c4ccccc4c3)c([2H])c([2H])c([2H])c2c1[2H] ; [2H]c1c([2H])c([2H])c2c(-c3cc(-c4nc(-c5ccccc5)nc(-c5cc(-c6ccccc6)c6ccccc6c5)n4)c4ccccc4c3)c([2H])c([2H])c([2H])c2c1[2H] ; [2H]c1c([2H])c([2H])c2c(-c3cc(-c4nc(-c5ccc6c(-c7ccccc7)cccc6c5)nc(-c5cccc6ccccc56)n4)c4ccccc4c3)c([2H])c([2H])c([2H])c2c1[2H]]

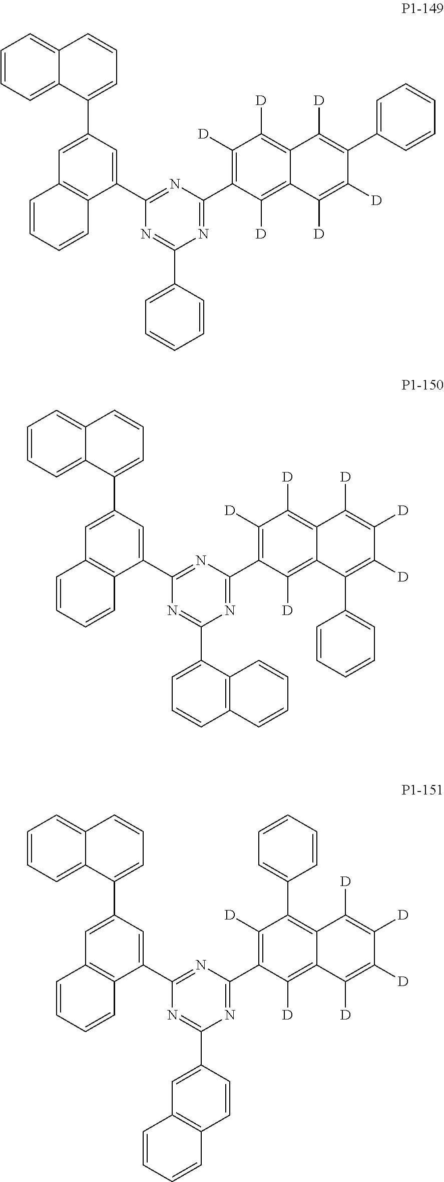 [SMILES: [2H]c1c(-c2nc(-c3ccccc3)nc(-c3cc(-c4cccc5ccccc45)cc4ccccc34)n2)c([2H])c2c([2H])c([2H])c(-c3ccccc3)c([2H])c2c1[2H] ; [2H]c1c([2H])c(-c2ccccc2)c2c([2H])c(-c3nc(-c4cccc5ccccc45)nc(-c4cc(-c5cccc6ccccc56)cc5ccccc45)n3)c([2H])c([2H])c2c1[2H] ; [2H]c1c([2H])c([2H])c2c(-c3ccccc3)c([2H])c(-c3nc(-c4ccc5ccccc5c4)nc(-c4cc(-c5cccc6ccccc56)cc5ccccc45)n3)c([2H])c2c1[2H]]

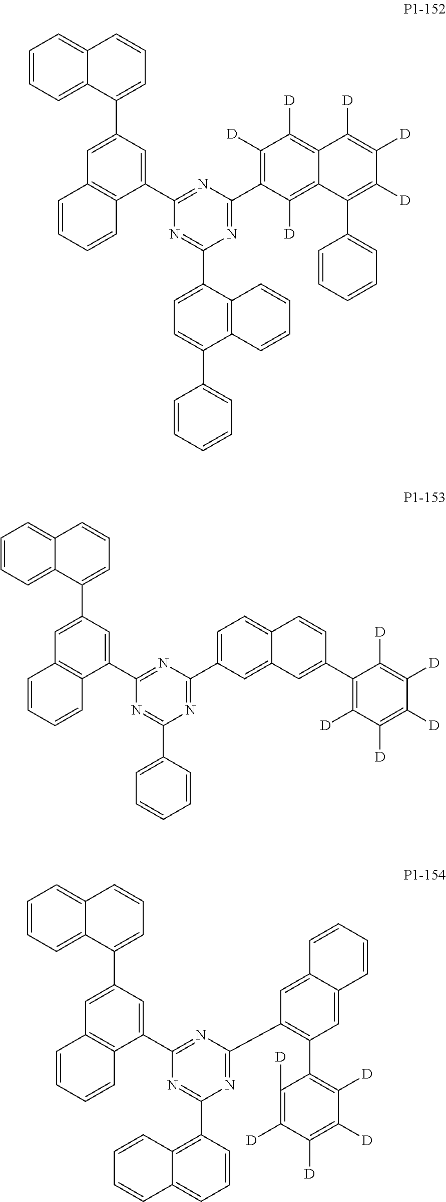 [SMILES: [2H]c1c([2H])c(-c2ccccc2)c2c([2H])c(-c3nc(-c4cc(-c5cccc6ccccc56)cc5ccccc45)nc(-c4ccc(-c5ccccc5)c5ccccc45)n3)c([2H])c([2H])c2c1[2H] ; [2H]c1c([2H])c([2H])c(-c2ccc3ccc(-c4nc(-c5ccccc5)nc(-c5cc(-c6cccc7ccccc67)cc6ccccc56)n4)cc3c2)c([2H])c1[2H] ; [2H]c1c([2H])c([2H])c(-c2cc3ccccc3cc2-c2nc(-c3cccc4ccccc34)nc(-c3cc(-c4cccc5ccccc45)cc4ccccc34)n2)c([2H])c1[2H]]

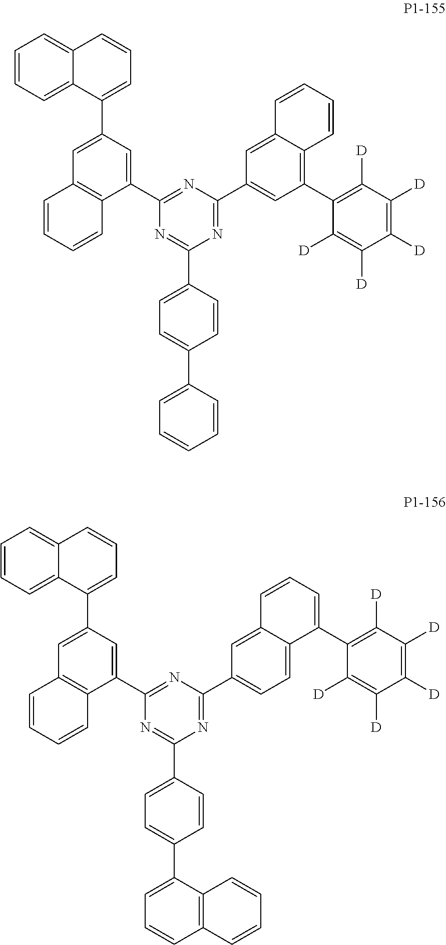 [SMILES: [2H]c1c([2H])c([2H])c(-c2cc(-c3nc(-c4ccc(-c5ccccc5)cc4)nc(-c4cc(-c5cccc6ccccc56)cc5ccccc45)n3)cc3ccccc23)c([2H])c1[2H] ; [2H]c1c([2H])c([2H])c(-c2cccc3cc(-c4nc(-c5ccc(-c6cccc7ccccc67)cc5)nc(-c5cc(-c6cccc7ccccc67)cc6ccccc56)n4)ccc23)c([2H])c1[2H]]

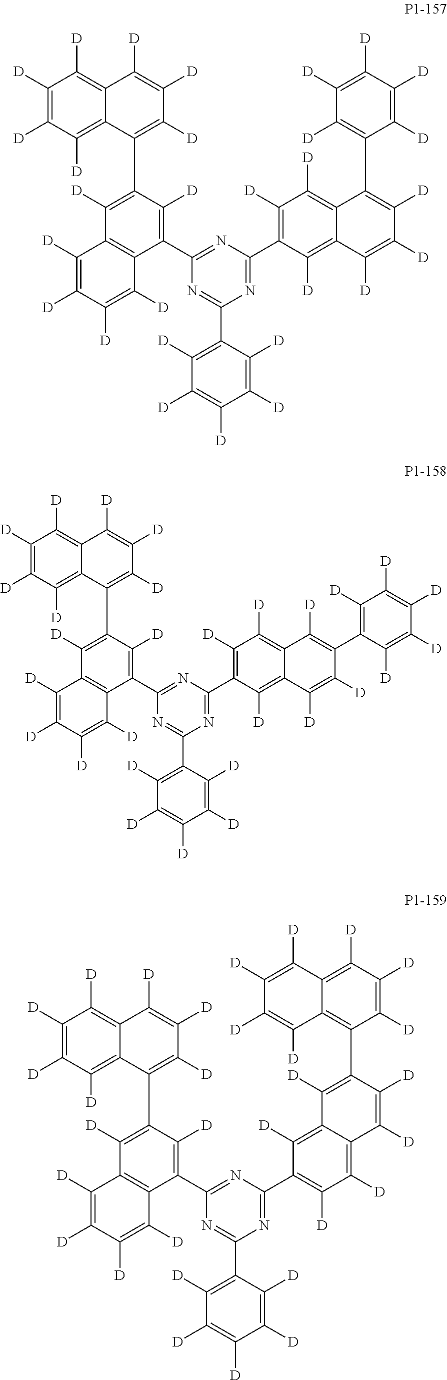

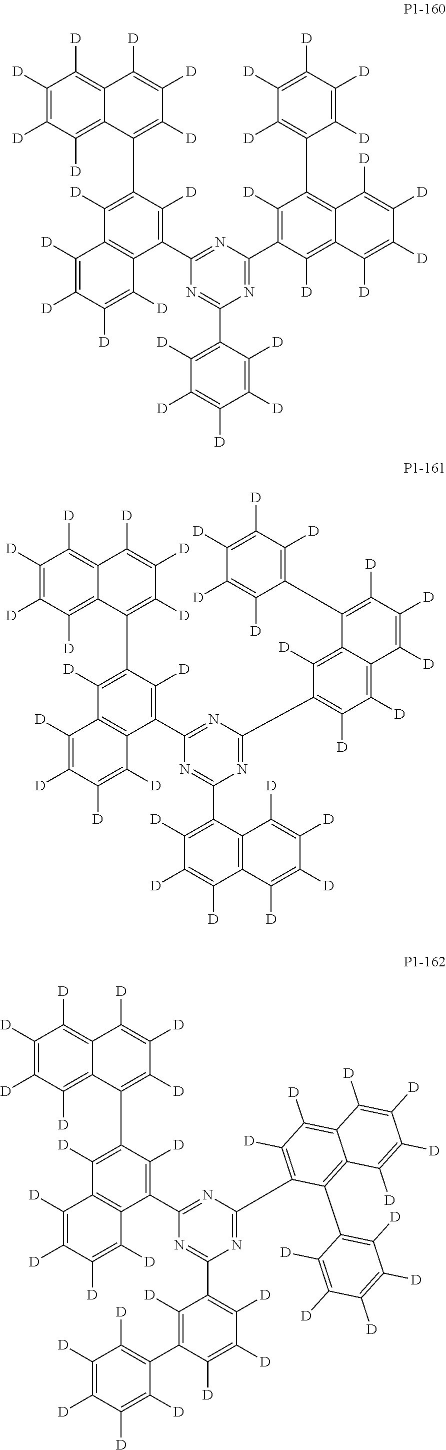

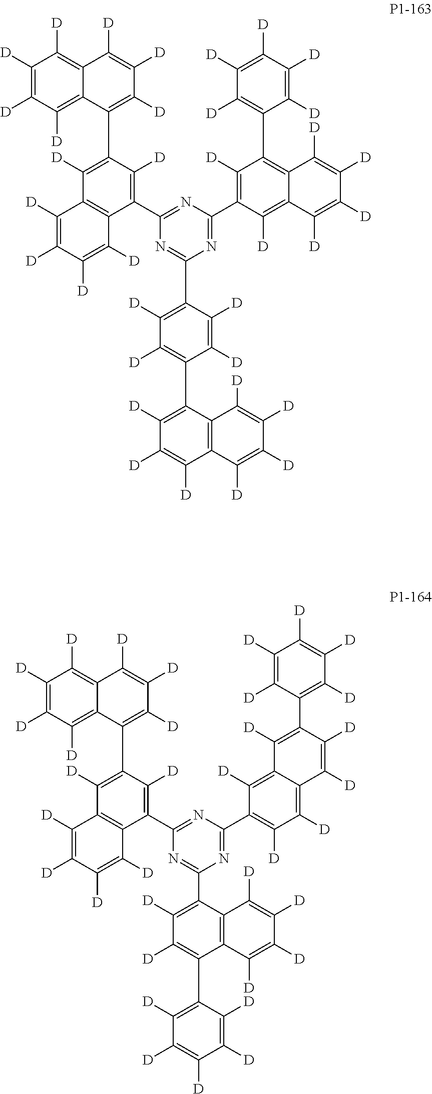

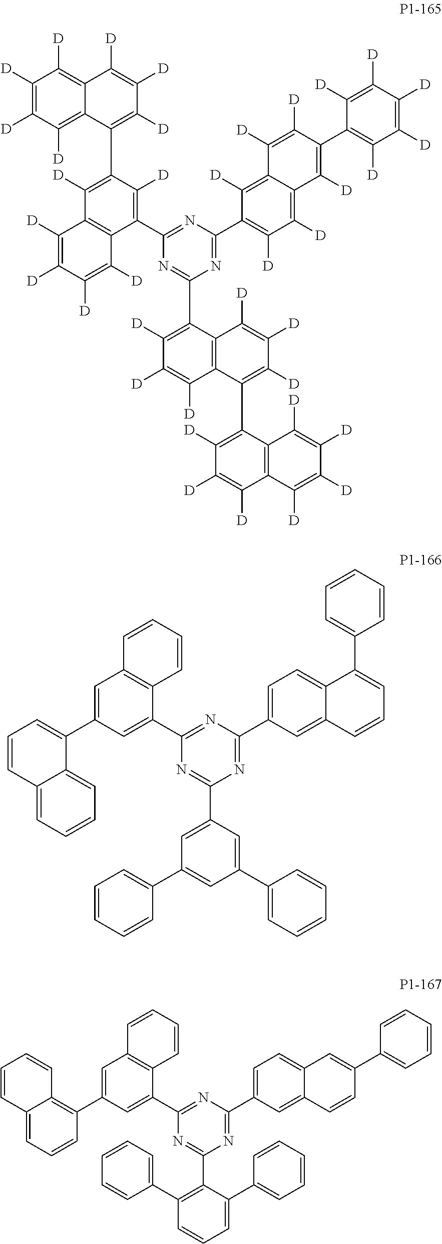

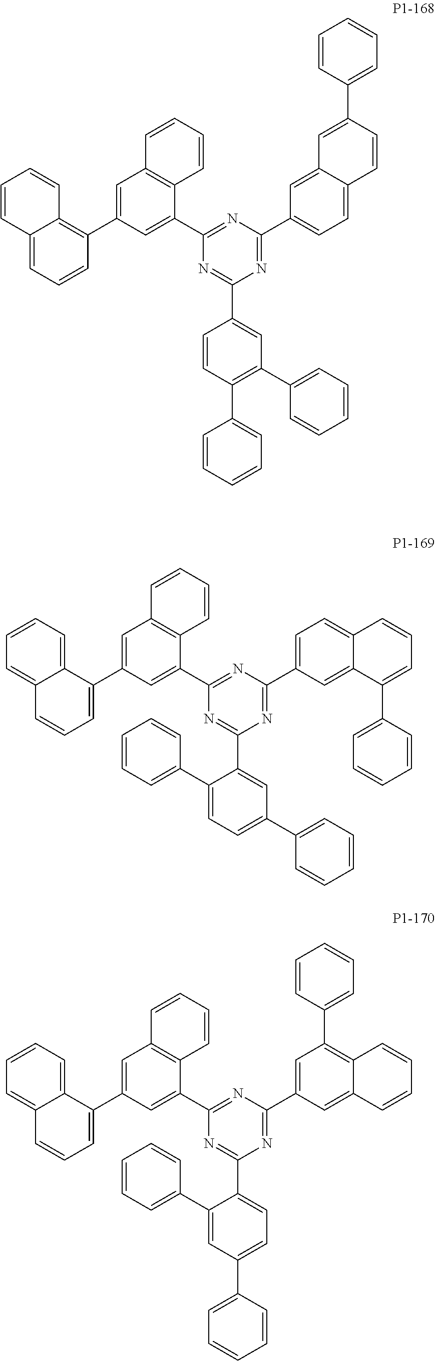 [SMILES: c1ccc(-c2ccc3ccc(-c4nc(-c5ccc(-c6ccccc6)c(-c6ccccc6)c5)nc(-c5cc(-c6cccc7ccccc67)cc6ccccc56)n4)cc3c2)cc1 ; c1ccc(-c2ccc(-c3ccccc3)c(-c3nc(-c4ccc5cccc(-c6ccccc6)c5c4)nc(-c4cc(-c5cccc6ccccc56)cc5ccccc45)n3)c2)cc1 ; c1ccc(-c2ccc(-c3nc(-c4cc(-c5ccccc5)c5ccccc5c4)nc(-c4cc(-c5cccc6ccccc56)cc5ccccc45)n3)c(-c3ccccc3)c2)cc1]

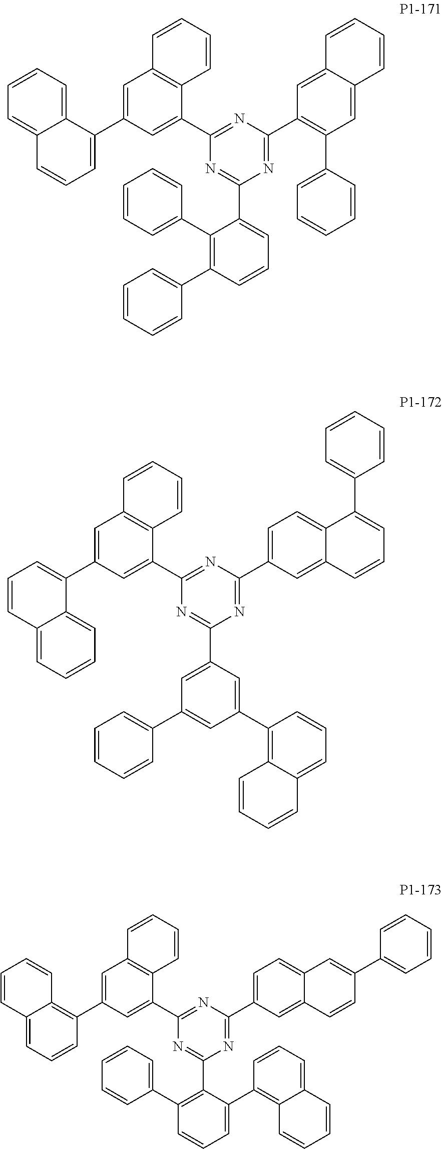 [SMILES: c1ccc(-c2cc3ccccc3cc2-c2nc(-c3cccc(-c4ccccc4)c3-c3ccccc3)nc(-c3cc(-c4cccc5ccccc45)cc4ccccc34)n2)cc1 ; c1ccc(-c2cc(-c3nc(-c4ccc5c(-c6ccccc6)cccc5c4)nc(-c4cc(-c5cccc6ccccc56)cc5ccccc45)n3)cc(-c3cccc4ccccc34)c2)cc1 ; c1ccc(-c2ccc3cc(-c4nc(-c5c(-c6ccccc6)cccc5-c5cccc6ccccc56)nc(-c5cc(-c6cccc7ccccc67)cc6ccccc56)n4)ccc3c2)cc1]

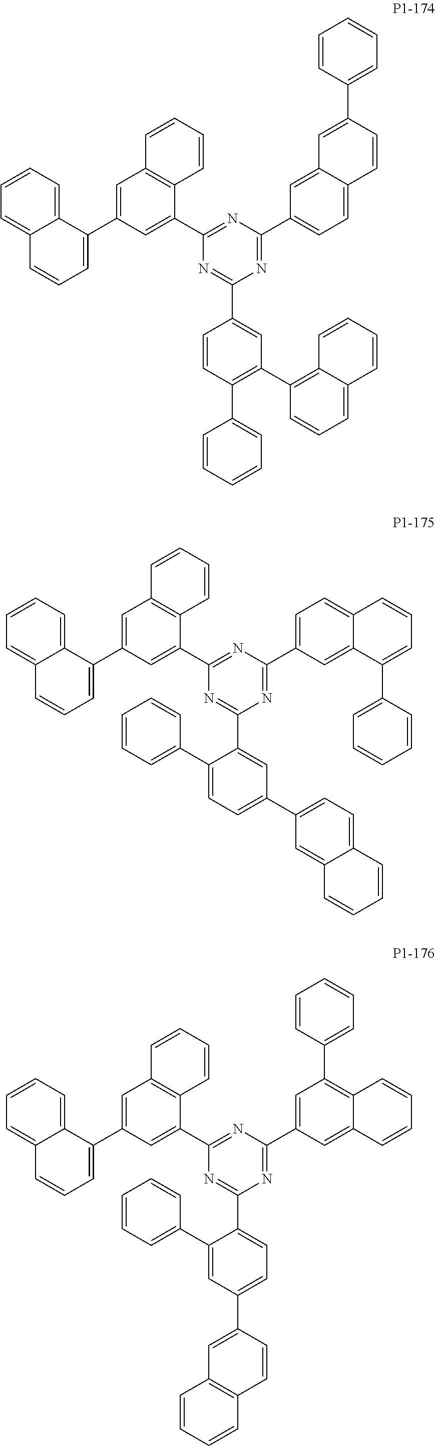 [SMILES: c1ccc(-c2ccc3ccc(-c4nc(-c5ccc(-c6ccccc6)c(-c6cccc7ccccc67)c5)nc(-c5cc(-c6cccc7ccccc67)cc6ccccc56)n4)cc3c2)cc1 ; c1ccc(-c2ccc(-c3ccc4ccccc4c3)cc2-c2nc(-c3ccc4cccc(-c5ccccc5)c4c3)nc(-c3cc(-c4cccc5ccccc45)cc4ccccc34)n2)cc1 ; c1ccc(-c2cc(-c3ccc4ccccc4c3)ccc2-c2nc(-c3cc(-c4ccccc4)c4ccccc4c3)nc(-c3cc(-c4cccc5ccccc45)cc4ccccc34)n2)cc1]

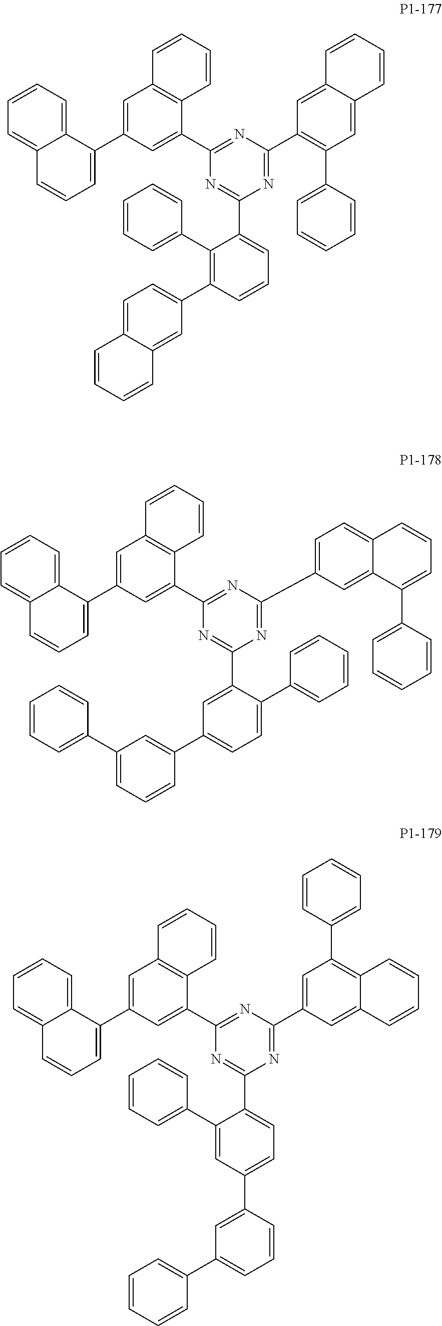 [SMILES: c1ccc(-c2cc3ccccc3cc2-c2nc(-c3cccc(-c4ccc5ccccc5c4)c3-c3ccccc3)nc(-c3cc(-c4cccc5ccccc45)cc4ccccc34)n2)cc1 ; c1ccc(-c2cccc(-c3ccc(-c4ccccc4)c(-c4nc(-c5ccc6cccc(-c7ccccc7)c6c5)nc(-c5cc(-c6cccc7ccccc67)cc6ccccc56)n4)c3)c2)cc1 ; c1ccc(-c2cccc(-c3ccc(-c4nc(-c5cc(-c6ccccc6)c6ccccc6c5)nc(-c5cc(-c6cccc7ccccc67)cc6ccccc56)n4)c(-c4ccccc4)c3)c2)cc1]

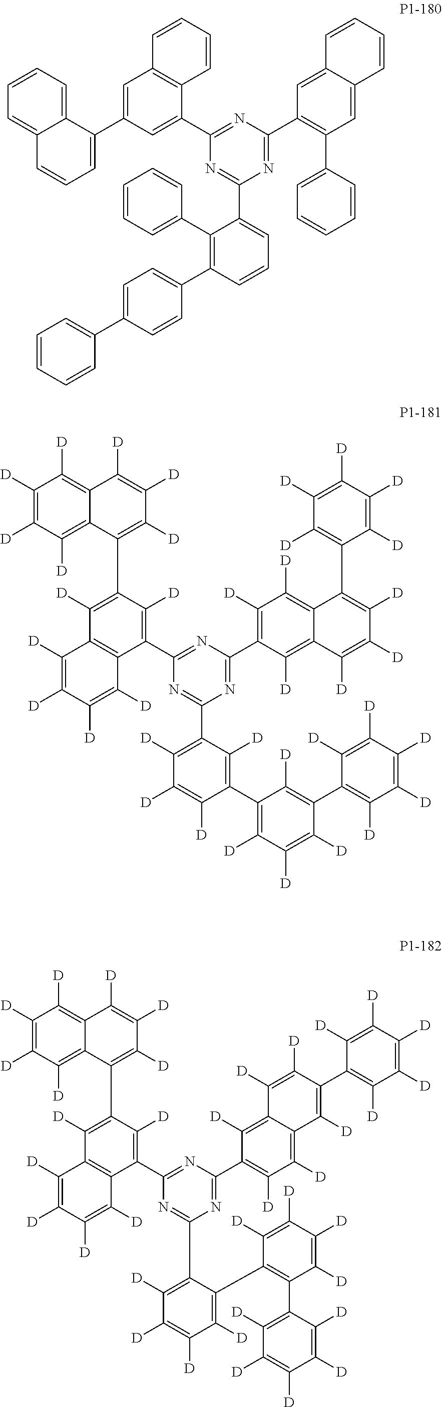

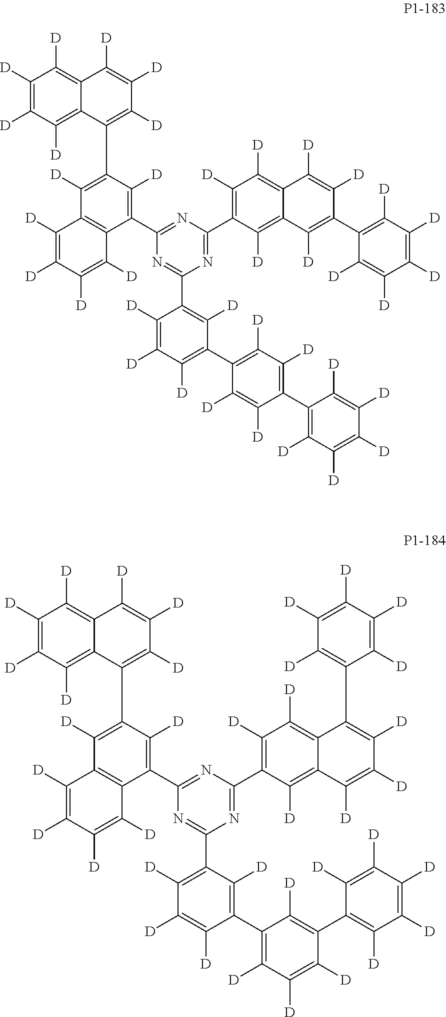

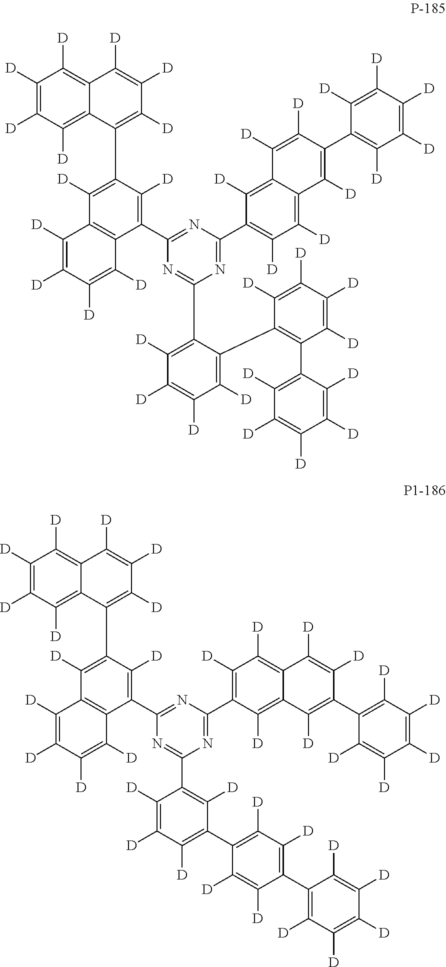

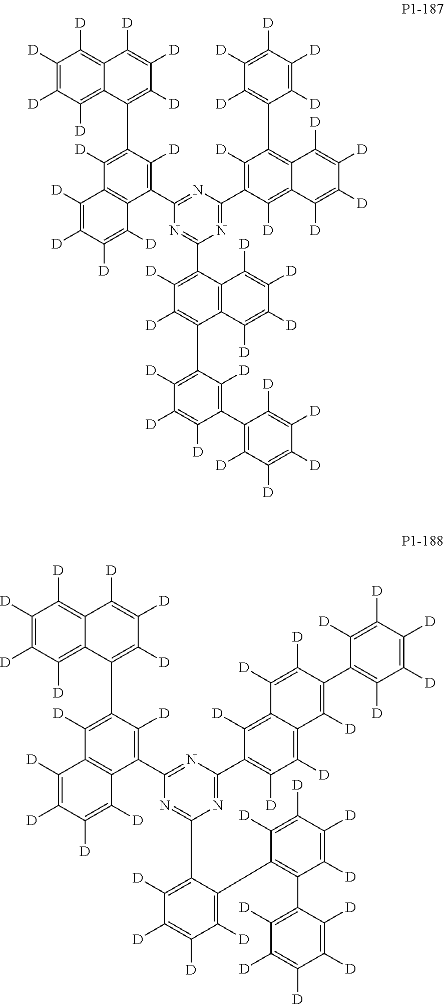

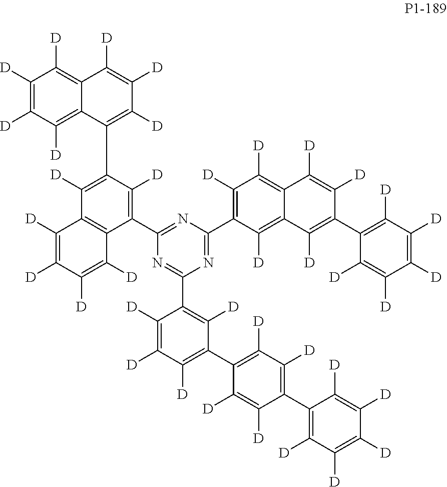 [SMILES: [2H]c1c([2H])c([2H])c(-c2c([2H])c([2H])c(-c3c([2H])c([2H])c([2H])c(-c4nc(-c5c([2H])c([2H])c6c([2H])c([2H])c(-c7c([2H])c([2H])c([2H])c([2H])c7[2H])c([2H])c6c5[2H])nc(-c5c([2H])c(-c6c([2H])c([2H])c([2H])c7c([2H])c([2H])c([2H])c([2H])c67)c([2H])c6c([2H])c([2H])c([2H])c([2H])c56)n4)c3[2H])c([2H])c2[2H])c([2H])c1[2H]]

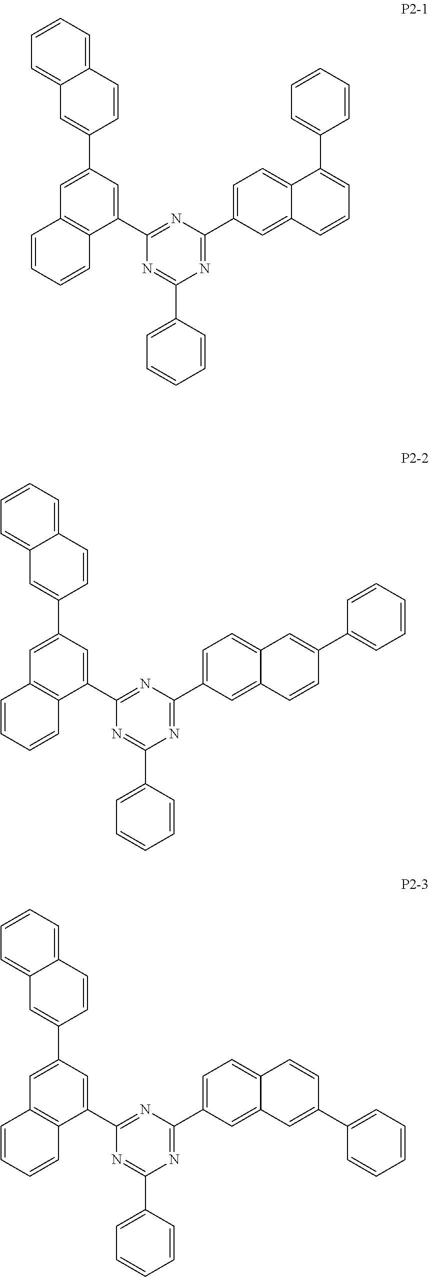 [SMILES: c1ccc(-c2nc(-c3ccc4c(-c5ccccc5)cccc4c3)nc(-c3cc(-c4ccc5ccccc5c4)cc4ccccc34)n2)cc1 ; c1ccc(-c2ccc3cc(-c4nc(-c5ccccc5)nc(-c5cc(-c6ccc7ccccc7c6)cc6ccccc56)n4)ccc3c2)cc1 ; c1ccc(-c2ccc3ccc(-c4nc(-c5ccccc5)nc(-c5cc(-c6ccc7ccccc7c6)cc6ccccc56)n4)cc3c2)cc1]

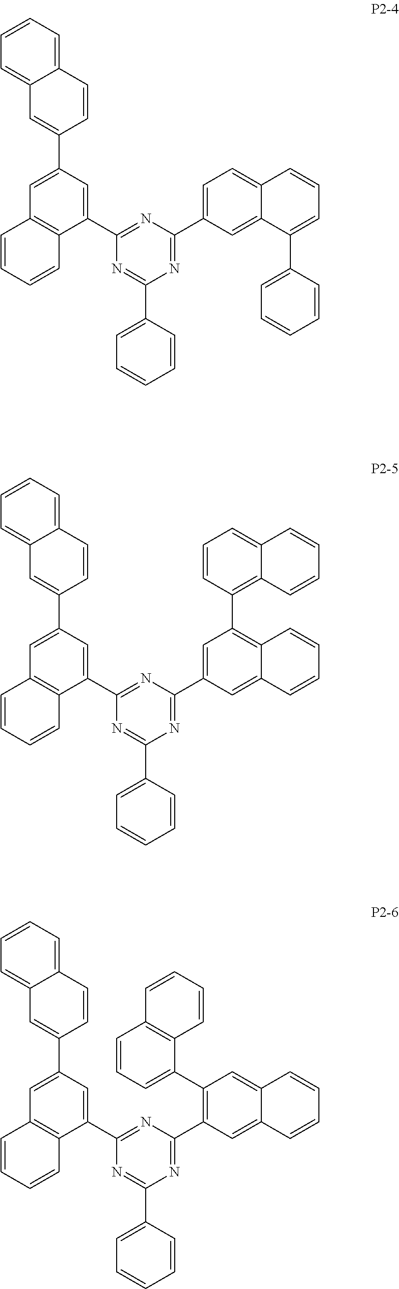 [SMILES: c1ccc(-c2nc(-c3ccc4cccc(-c5ccccc5)c4c3)nc(-c3cc(-c4ccc5ccccc5c4)cc4ccccc34)n2)cc1 ; c1ccc(-c2nc(-c3cc(-c4cccc5ccccc45)c4ccccc4c3)nc(-c3cc(-c4ccc5ccccc5c4)cc4ccccc34)n2)cc1 ; c1ccc(-c2nc(-c3cc4ccccc4cc3-c3cccc4ccccc34)nc(-c3cc(-c4ccc5ccccc5c4)cc4ccccc34)n2)cc1]

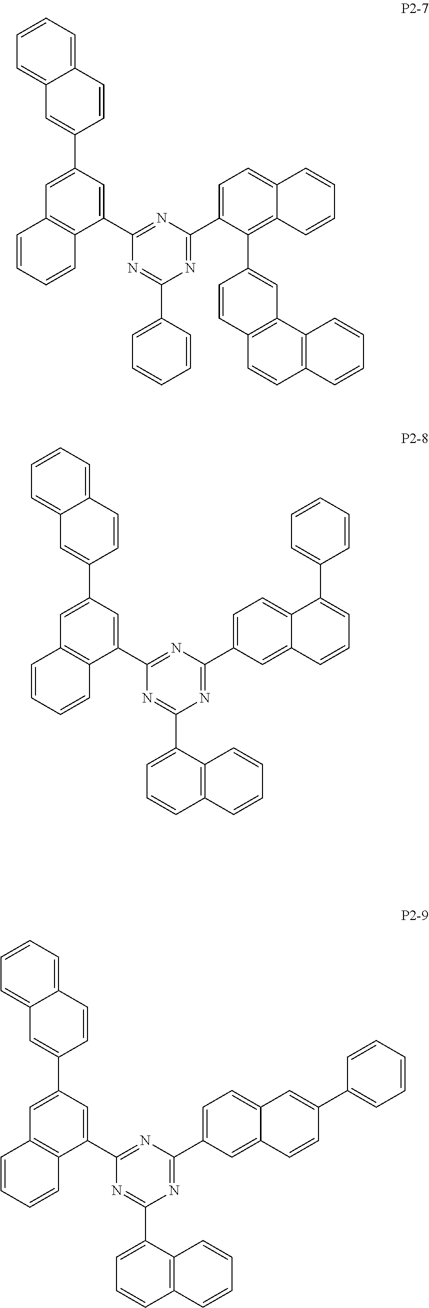 [SMILES: c1ccc(-c2nc(-c3ccc4ccccc4c3-c3ccc4ccc5ccccc5c4c3)nc(-c3cc(-c4ccc5ccccc5c4)cc4ccccc34)n2)cc1 ; c1ccc(-c2cccc3cc(-c4nc(-c5cccc6ccccc56)nc(-c5cc(-c6ccc7ccccc7c6)cc6ccccc56)n4)ccc23)cc1 ; c1ccc(-c2ccc3cc(-c4nc(-c5cccc6ccccc56)nc(-c5cc(-c6ccc7ccccc7c6)cc6ccccc56)n4)ccc3c2)cc1]

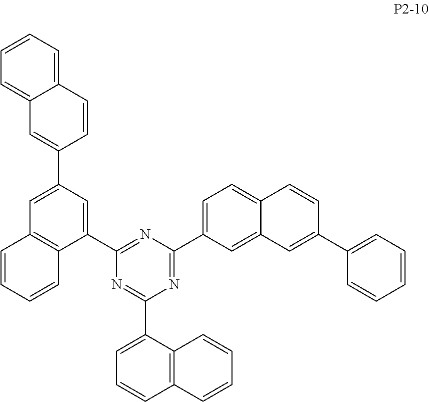 [SMILES: c1ccc(-c2ccc3ccc(-c4nc(-c5cccc6ccccc56)nc(-c5cc(-c6ccc7ccccc7c6)cc6ccccc56)n4)cc3c2)cc1]

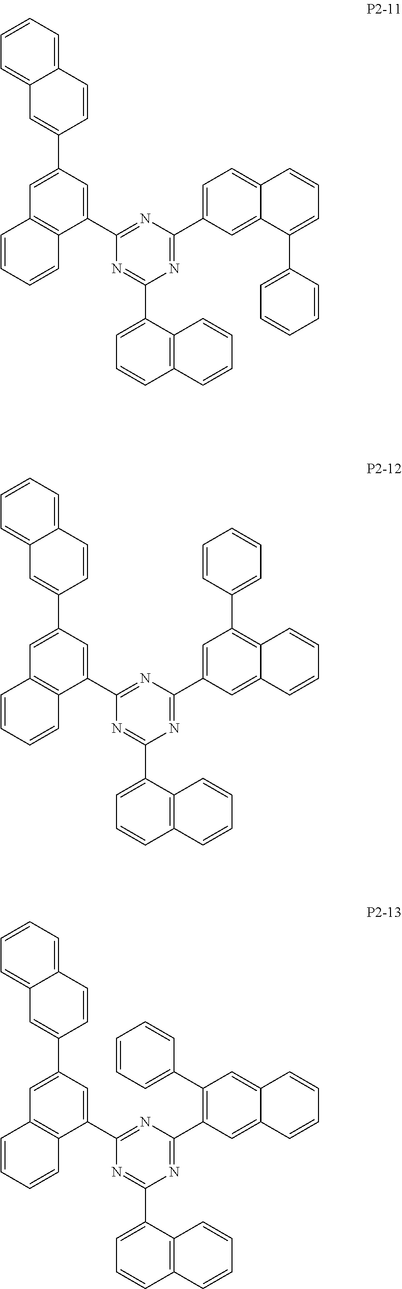 [SMILES: c1ccc(-c2cccc3ccc(-c4nc(-c5cccc6ccccc56)nc(-c5cc(-c6ccc7ccccc7c6)cc6ccccc56)n4)cc23)cc1 ; c1ccc(-c2cc(-c3nc(-c4cccc5ccccc45)nc(-c4cc(-c5ccc6ccccc6c5)cc5ccccc45)n3)cc3ccccc23)cc1 ; c1ccc(-c2cc3ccccc3cc2-c2nc(-c3cccc4ccccc34)nc(-c3cc(-c4ccc5ccccc5c4)cc4ccccc34)n2)cc1]

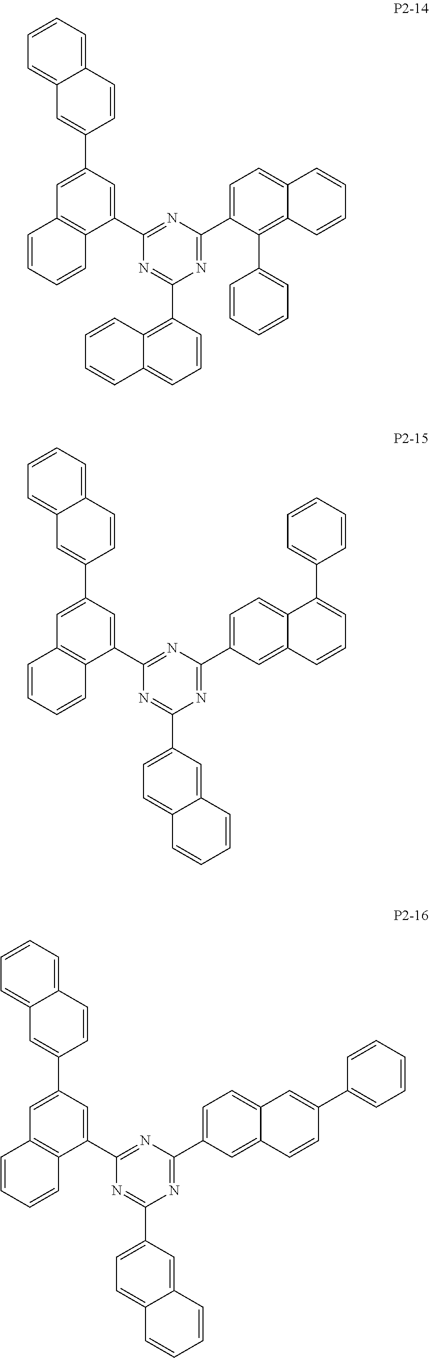 [SMILES: c1ccc(-c2c(-c3nc(-c4cccc5ccccc45)nc(-c4cc(-c5ccc6ccccc6c5)cc5ccccc45)n3)ccc3ccccc23)cc1 ; c1ccc(-c2cccc3cc(-c4nc(-c5ccc6ccccc6c5)nc(-c5cc(-c6ccc7ccccc7c6)cc6ccccc56)n4)ccc23)cc1 ; c1ccc(-c2ccc3cc(-c4nc(-c5ccc6ccccc6c5)nc(-c5cc(-c6ccc7ccccc7c6)cc6ccccc56)n4)ccc3c2)cc1]

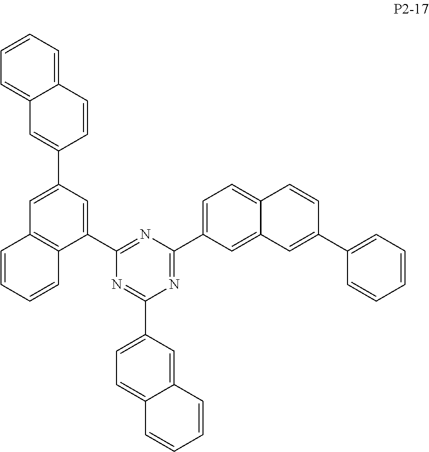 [SMILES: c1ccc(-c2ccc3ccc(-c4nc(-c5ccc6ccccc6c5)nc(-c5cc(-c6ccc7ccccc7c6)cc6ccccc56)n4)cc3c2)cc1]

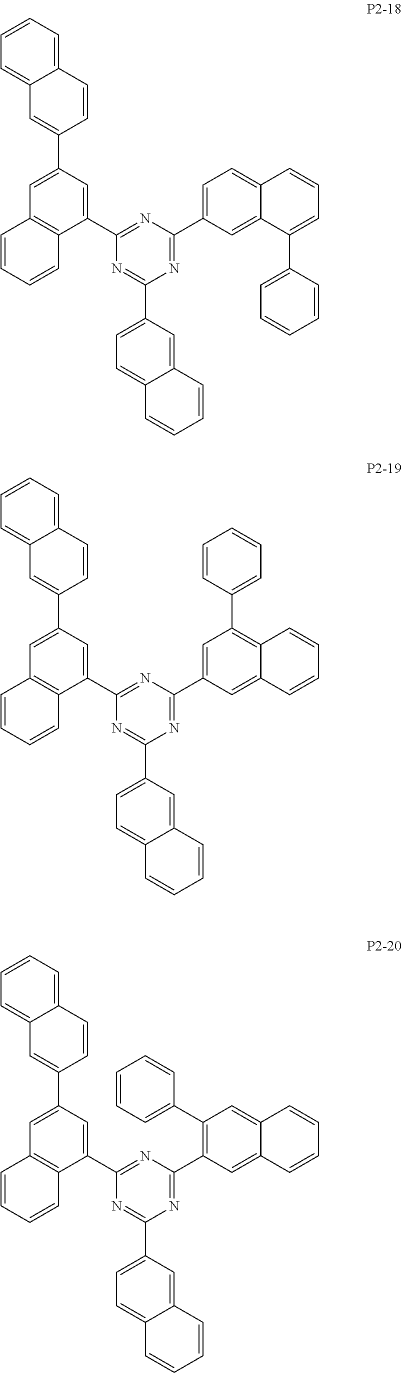 [SMILES: c1ccc(-c2cccc3ccc(-c4nc(-c5ccc6ccccc6c5)nc(-c5cc(-c6ccc7ccccc7c6)cc6ccccc56)n4)cc23)cc1 ; c1ccc(-c2cc(-c3nc(-c4ccc5ccccc5c4)nc(-c4cc(-c5ccc6ccccc6c5)cc5ccccc45)n3)cc3ccccc23)cc1 ; c1ccc(-c2cc3ccccc3cc2-c2nc(-c3ccc4ccccc4c3)nc(-c3cc(-c4ccc5ccccc5c4)cc4ccccc34)n2)cc1]

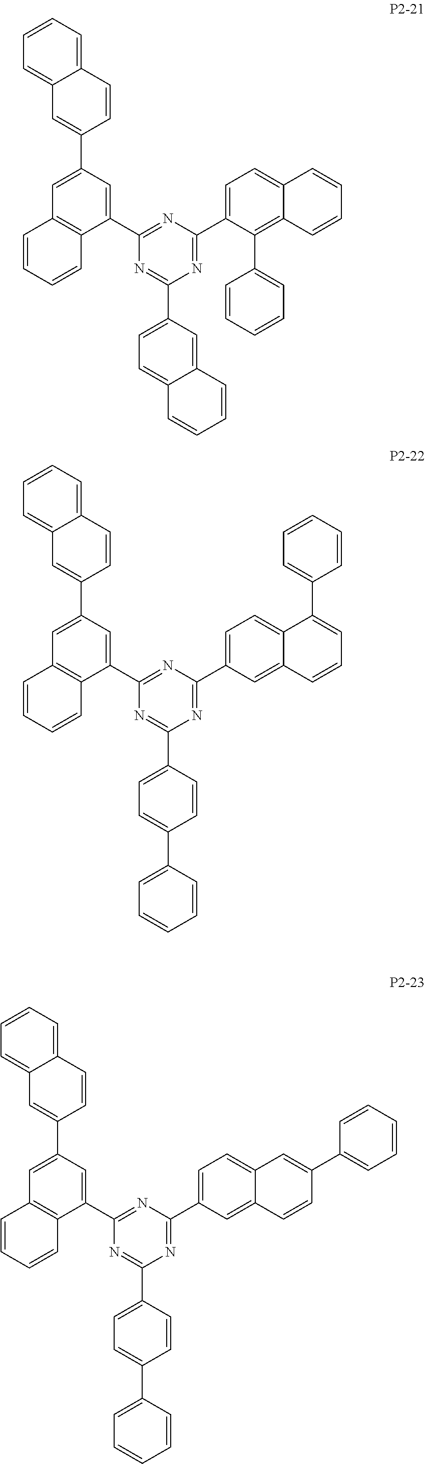 [SMILES: c1ccc(-c2c(-c3nc(-c4ccc5ccccc5c4)nc(-c4cc(-c5ccc6ccccc6c5)cc5ccccc45)n3)ccc3ccccc23)cc1 ; c1ccc(-c2ccc(-c3nc(-c4ccc5c(-c6ccccc6)cccc5c4)nc(-c4cc(-c5ccc6ccccc6c5)cc5ccccc45)n3)cc2)cc1 ; c1ccc(-c2ccc(-c3nc(-c4ccc5cc(-c6ccccc6)ccc5c4)nc(-c4cc(-c5ccc6ccccc6c5)cc5ccccc45)n3)cc2)cc1]

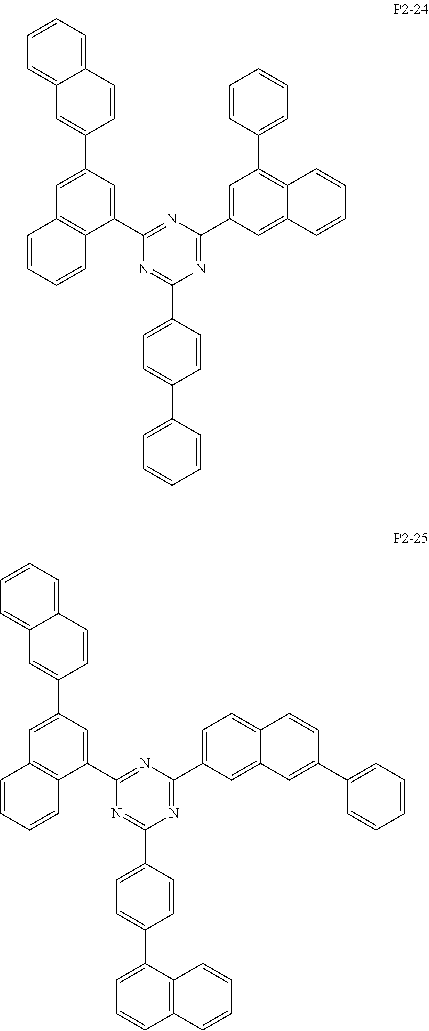 [SMILES: c1ccc(-c2ccc(-c3nc(-c4cc(-c5ccccc5)c5ccccc5c4)nc(-c4cc(-c5ccc6ccccc6c5)cc5ccccc45)n3)cc2)cc1 ; c1ccc(-c2ccc3ccc(-c4nc(-c5ccc(-c6cccc7ccccc67)cc5)nc(-c5cc(-c6ccc7ccccc7c6)cc6ccccc56)n4)cc3c2)cc1]

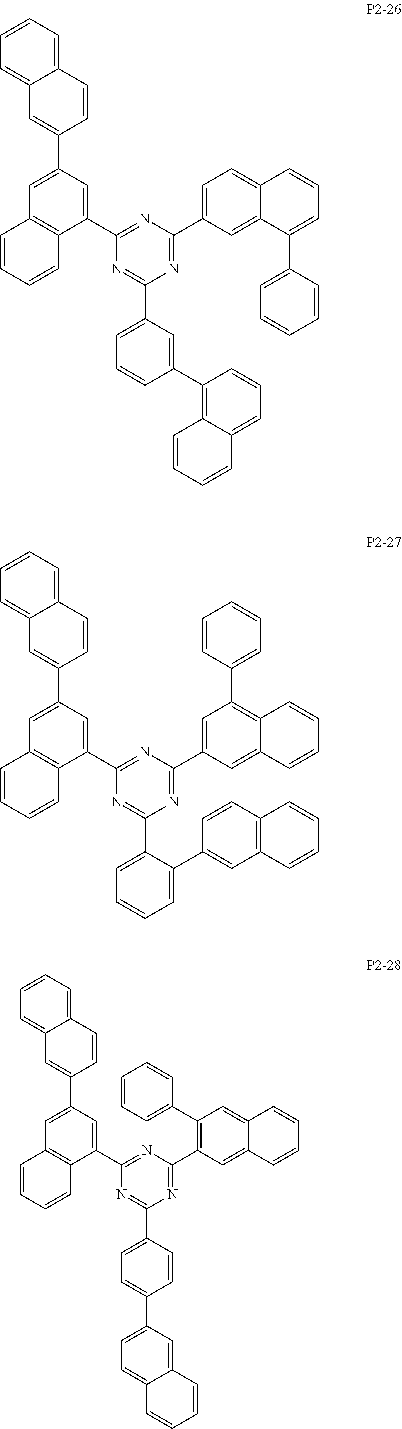 [SMILES: c1ccc(-c2cccc3ccc(-c4nc(-c5cccc(-c6cccc7ccccc67)c5)nc(-c5cc(-c6ccc7ccccc7c6)cc6ccccc56)n4)cc23)cc1 ; c1ccc(-c2cc(-c3nc(-c4ccccc4-c4ccc5ccccc5c4)nc(-c4cc(-c5ccc6ccccc6c5)cc5ccccc45)n3)cc3ccccc23)cc1 ; c1ccc(-c2cc3ccccc3cc2-c2nc(-c3ccc(-c4ccc5ccccc5c4)cc3)nc(-c3cc(-c4ccc5ccccc5c4)cc4ccccc34)n2)cc1]

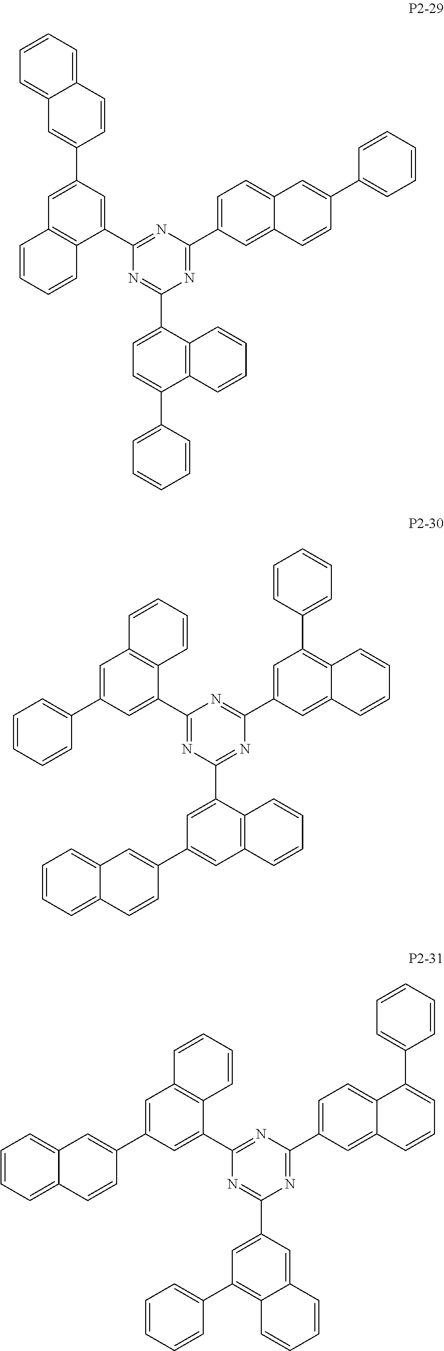 [SMILES: c1ccc(-c2ccc3cc(-c4nc(-c5cc(-c6ccc7ccccc7c6)cc6ccccc56)nc(-c5ccc(-c6ccccc6)c6ccccc56)n4)ccc3c2)cc1 ; c1ccc(-c2cc(-c3nc(-c4cc(-c5ccccc5)c5ccccc5c4)nc(-c4cc(-c5ccc6ccccc6c5)cc5ccccc45)n3)c3ccccc3c2)cc1 ; c1ccc(-c2cccc3cc(-c4nc(-c5cc(-c6ccccc6)c6ccccc6c5)nc(-c5cc(-c6ccc7ccccc7c6)cc6ccccc56)n4)ccc23)cc1]

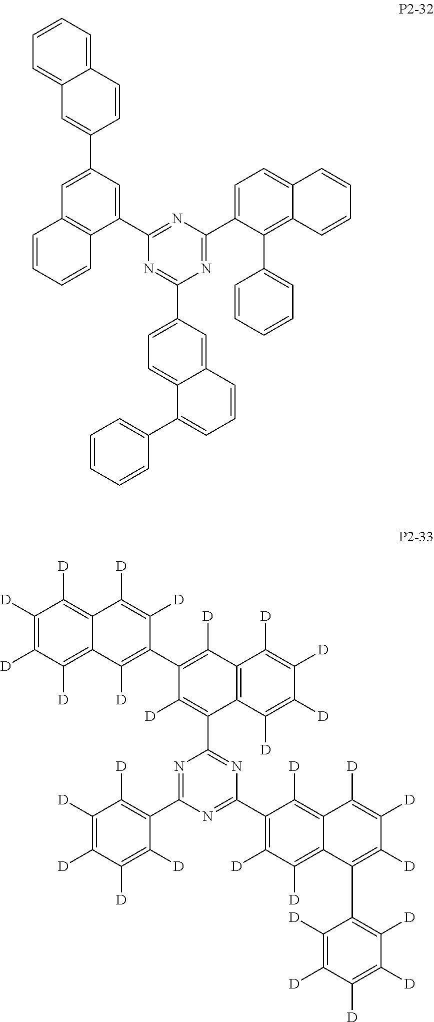 [SMILES: c1ccc(-c2cccc3cc(-c4nc(-c5ccc6ccccc6c5-c5ccccc5)nc(-c5cc(-c6ccc7ccccc7c6)cc6ccccc56)n4)ccc23)cc1 ; [2H]c1c([2H])c([2H])c(-c2nc(-c3c([2H])c([2H])c4c(-c5c([2H])c([2H])c([2H])c([2H])c5[2H])c([2H])c([2H])c([2H])c4c3[2H])nc(-c3c([2H])c(-c4c([2H])c([2H])c5c([2H])c([2H])c([2H])c([2H])c5c4[2H])c([2H])c4c([2H])c([2H])c([2H])c([2H])c34)n2)c([2H])c1[2H]]

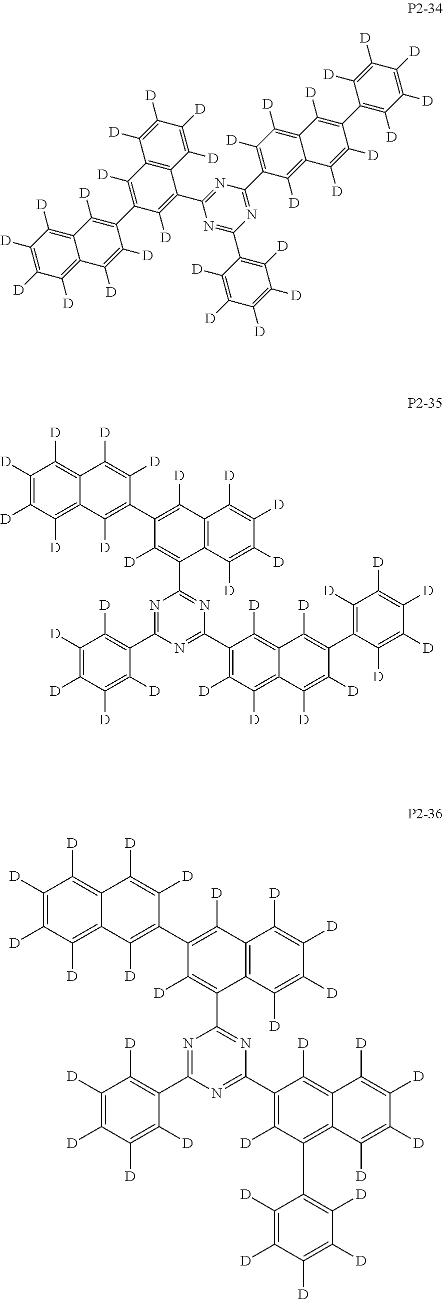

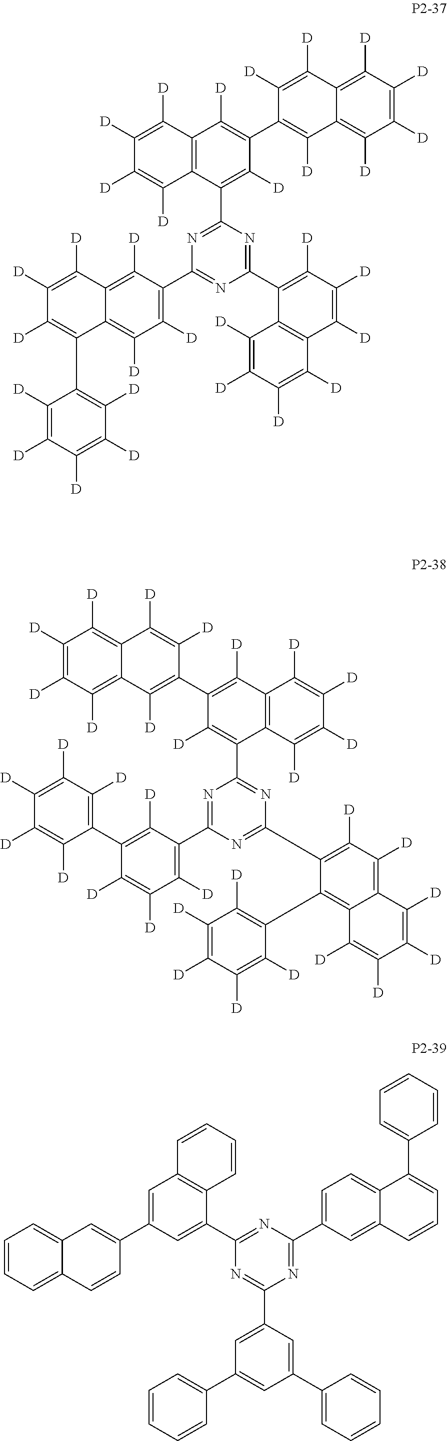

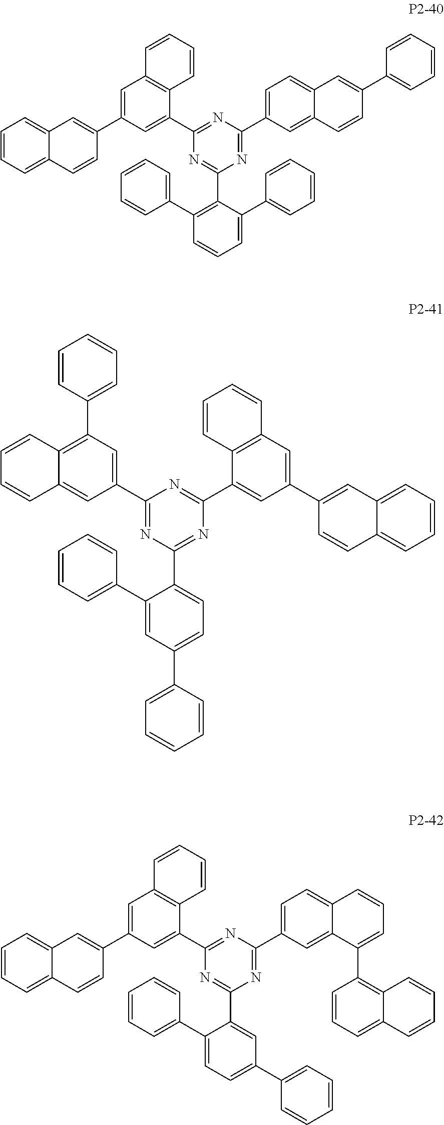 [SMILES: c1ccc(-c2ccc3cc(-c4nc(-c5c(-c6ccccc6)cccc5-c5ccccc5)nc(-c5cc(-c6ccc7ccccc7c6)cc6ccccc56)n4)ccc3c2)cc1 ; c1ccc(-c2ccc(-c3nc(-c4cc(-c5ccccc5)c5ccccc5c4)nc(-c4cc(-c5ccc6ccccc6c5)cc5ccccc45)n3)c(-c3ccccc3)c2)cc1 ; c1ccc(-c2ccc(-c3ccccc3)c(-c3nc(-c4ccc5cccc(-c6cccc7ccccc67)c5c4)nc(-c4cc(-c5ccc6ccccc6c5)cc5ccccc45)n3)c2)cc1]

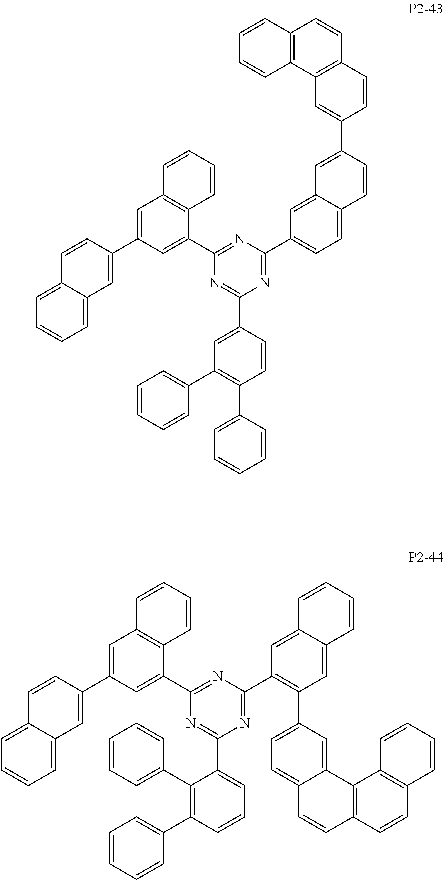 [SMILES: c1ccc(-c2ccc(-c3nc(-c4ccc5ccc(-c6ccc7ccc8ccccc8c7c6)cc5c4)nc(-c4cc(-c5ccc6ccccc6c5)cc5ccccc45)n3)cc2-c2ccccc2)cc1 ; c1ccc(-c2cccc(-c3nc(-c4cc5ccccc5cc4-c4ccc5ccc6ccc7ccccc7c6c5c4)nc(-c4cc(-c5ccc6ccccc6c5)cc5ccccc45)n3)c2-c2ccccc2)cc1]

Also, in another aspect, the present invention provides a composition for an organic electronic element comprising a first compound represented by the Formula 1; and a second compound represented by Formula 4 or Formula 5.

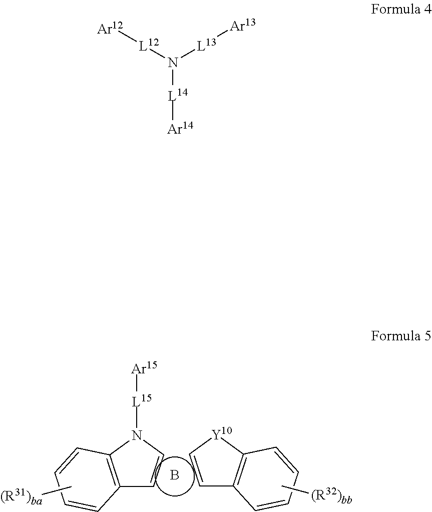

wherein:
L¹², L¹³, L¹⁴ and L¹⁵ are independently selected from the group consisting of a single bond; a C₆-C₆₀ arylene group; a fluorenylene group; a C₂-C₆₀ heterocyclic group including at least one heteroatom of O, N, S, Si or P; and a fused ring group of a C₃-C₆₀ aliphatic ring and a C₆-C₆₀ aromatic ring,

Wherein when L¹², L¹³, L¹⁴ and L¹⁵ are an arylene group, preferably a C₆-C₃₀ arylene group, more preferably a C₆-C₂₅ arylene group, a C₆-C₁₈ arylene group or a C₆-C₁₂ arylene group, such as phenylene, biphenylene, naphthylene, terphenylene, anthracenylene, etc.

Wherein when L¹², L¹³, L¹⁴ and L¹⁵ are a heterocyclic group, preferably a C₂-C₃₀ heterocyclic group, more preferably a C₂-C₂₅ heterocyclic group, a C₂-C₁₈ heterocyclic group, or a C₂-C₁₂ heterocyclic group, such as pyrazine, thiophene, pyridine, pyrimidoindole, 5-phenyl-5H-pyrimido[5,4-b]indole, quinazoline, benzoquinazoline, carbazole, dibenzoquinazoline, benzofuran, benzothiophene, benzothienopyrimidine, benzofuropyrimidine, phenothiazine, phenylphenothiazine, naphthobenzofuran, naphthobenzothiophene, etc.

Wherein when L¹², L¹³, L¹⁴ and L¹⁵ are a fused ring group, preferably a fused ring group of a C₃-C₃₀ aliphatic ring and a C₆-C₃₀ aromatic ring, more preferably a fused ring group of a C₃-C₂₅ aliphatic ring and a C₆-C₂₅ aromatic ring.

Ar¹², Ar¹³ and Ar¹⁴ are independently selected from the group consisting of a C₆-C₆₀ aryl group; a fluorenyl group; a C₂-C₆₀ heterocyclic group including at least one heteroatom of O, N, S, Si or P; a fused ring group of a C₃-C₆₀ aliphatic ring and a C₆-C₆₀ aromatic ring; a C₃-C₆₀ aliphatic ring; and -L′-N(R^{c})(R^{d});

Ar¹⁵ is independently selected from the group consisting of a C₆-C₆₀ aryl group; a fluorenyl group; a C₂-C₆₀ heterocyclic group including at least one heteroatom of O, N, S, Si or P; a fused ring group of a C₃-C₆₀ aliphatic ring and a C₆-C₆₀ aromatic ring; and a C₃-C₆₀ aliphatic ring;

Wherein when Ar¹², Ar¹³, Ar¹⁴ and Ar¹⁵ are an aryl group, preferably a C₆-C₃₀ aryl group, more preferably a C₆-C₂₅ aryl group, a C₆-C₁₈ aryl group or a C₆-C₁₂ aryl group, such as phenyl, biphenyl, terphenyl, naphthalene, phenanthrene, etc.

Wherein when Ar¹², Ar¹³, Ar¹⁴ and Ar¹⁵ are a heterocyclic group, preferably a C₂-C₃₀ heterocyclic group, more preferably a C₂-C₂₅ heterocyclic group, a C₂-C₁₈ heterocyclic group, or a C₂-C₁₂ heterocyclic group, such as pyrazine, thiophene, pyridine, pyrimidoindole, 5-phenyl-5H-pyrimido[5,4-b]indole, quinazoline, benzoquinazoline, carbazole, dibenzoquinazoline, dibenzofuran, dibenzothiophene, benzothienopyrimidine, benzofuropyrimidine, phenothiazine, phenylphenothiazine, naphthobenzofuran, naphthobenzothiophene, etc.

Wherein when Ar¹², Ar¹³, Ar¹⁴ and Ar¹⁵ are a fused ring group, preferably a fused ring group of a C₃-C₃₀ aliphatic ring and a C₆-C₃₀ aromatic ring, more preferably a fused ring group of a C₃-C₂₅ aliphatic ring and a C₆-C₂₅ aromatic ring.

Wherein when Ar¹², Ar¹³, Ar¹⁴ and Ar¹⁵ are an aliphatic ring group, preferably a C₃-C₃₀ aliphatic ring group, more preferably a C₃-C₂₅ aliphatic ring group, a C₃-C₁₈ aliphatic ring group, and a C₃-C₁₂ aliphatic ring group, and specifically, cyclobutane, cyclopentane, cyclohexane, bicycloheptane, adamantyl, etc.

Y¹⁰ is O, S, C(R⁵¹)(R⁵²) or NR⁵³,

Ring B is an C₆-C₂₀ aryl,

L′ is selected from the group consisting of a single bond; a C₆-C₆₀ arylene group; a fluorenylene group; a C₂-C₆₀ heterocyclic group including at least one heteroatom of O, N, S, Si or P; and a C₃-C₆₀ aliphatic ring;

Wherein when L′ is an arylene group, preferably a C₆-C₃₀ arylene group, more preferably a C₆-C₂₅ arylene group, a C₆-C₁₈ arylene group or a C₆-C₁₂ arylene group, such as phenylene, biphenylene, naphthylene, terphenylene, anthracenylene, etc.

Wherein when L′ is a heterocyclic group, preferably a C₂-C₃₀ heterocyclic group, more preferably a C₂-C₂₅ heterocyclic group, a C₂-C₁₈ heterocyclic group, or a C₂-C₁₂ heterocyclic group, such as pyridine, pyrimidine, quinoline, quinazoline, quinoxaline, dibenzofuran, dibenzothiophene, naphthobenzothiophene, naphthobenzofuran, benzofuran, benzothiophene, etc.

Wherein when L′ is an aliphatic ring group, preferably a C₃-C₃₀ aliphatic ring group, more preferably a C₃-C₂₅ aliphatic ring group, a C₃-C₁₈ aliphatic ring group, and a C₃-C₁₂ aliphatic ring group, and specifically, cyclobutane, cyclopentane, cyclohexane, bicycloheptane, adamantyl, etc.

R³¹ and R³² are independently the same or different from each other and are independently selected from the group consisting of deuterium; a C₆-C₆₀ aryl group; a fluorenyl group; a C₂-C₆₀ heterocyclic group including at least one heteroatom of O, N, S, Si or P; a fused ring group of a C₃-C₆₀ aliphatic ring and a C₆-C₆₀ aromatic ring; a C₃-C₆₀ aliphatic ring; a C₁-C₅₀ alkyl group; a C₂-C₂₀ alkenyl group; a C₂-C₂₀ alkynyl group; a C₁-C₃₀ alkoxy group; and a C₆-C₃₀ aryloxy group; and a plurality of adjacent groups thereof may be bonded to each other to form a ring,

R⁵¹, R⁵², R⁵³, R^{c} and R^{d} are independently the same or different from each other and are independently selected from the group consisting of a C₆-C₆₀ aryl group; a fluorenyl group; a C₂-C₆₀ heterocyclic group including at least one heteroatom of O, N, S, Si or P; a fused ring group of a C₃-C₆₀ aliphatic ring and a C₆-C₆₀ aromatic ring; a C₃-C₆₀ aliphatic ring; a C₁-C₅₀ alkyl group; a C₂-C₂₀ alkenyl group; a C₂-C₂₀ alkynyl group; a C₁-C₃₀ alkoxy group; and a C₆-C₃₀ aryloxy group; and a plurality of adjacent groups thereof may be bonded to each other to form a spiro,

Wherein when R³¹, R³², R⁵¹, R⁵², R⁵³, R^{c} and R^{d} are an aryl group, preferably a C₆-C₃₀ aryl group, more preferably a C₆-C₂₅ aryl group, a C₆-C₁₈ aryl group or a C₆-C₁₂ aryl group, such as phenyl, biphenyl, terphenyl, naphthalene, phenanthrene, etc.

Wherein when R³¹, R³², R⁵¹, R⁵², R⁵³, R^{c} and R^{d} are a heterocyclic group, preferably a C₂-C₃₀ heterocyclic group, more preferably a C₂-C₂₅ heterocyclic group, a C₂-C₁₈ heterocyclic group, or a C₂-C₁₂ heterocyclic group, such as pyrazine, thiophene, pyridine, pyrimidoindole, 5-phenyl-5H-pyrimido[5,4-b]indole, quinazoline, benzoquinazoline, carbazole, dibenzoquinazoline, dibenzofuran, dibenzothiophene, benzothienopyrimidine, benzofuropyrimidine, phenothiazine, phenylphenothiazine, naphthobenzofuran, naphthobenzothiophene, etc.

Wherein when R³¹, R³², R⁵¹, R⁵², R⁵³, R^{c} and R^{d} are a fused ring group, preferably a fused ring group of a C₃-C₃₀ aliphatic ring and a C₆-C₃₀ aromatic ring, more preferably a fused ring group of a C₃-C₂₅ aliphatic ring and a C₆-C₂₅ aromatic ring.

Wherein when R³¹, R³², R⁵¹, R⁵², R⁵³, R^{c} and R^{d} are an alkyl group, preferably a C₁-C₃₀ alkyl group, more preferably a C₁-C₂₅ alkyl group, a C₁-C₁₈ alkyl group or a C₁-C₁₂ alkyl group, such as a methyl group, ethyl group, propyl group, isopropyl group, butyl group, t-butyl group, pentyl group, etc.

Wherein when R³¹, R³², R⁵¹, R⁵², R⁵³, R^{c} and R^{d} are an alkoxyl group, preferably a C₁-C₂₅ alkoxyl group, more preferably a C₁-C₁₈ alkoxyl group, or a C₁-C₁₂ alkoxyl group,

Wherein when R³¹, R³², R⁵¹, R⁵², R⁵³, R^{c} and R^{d} are an aryloxy group, preferably an C₆-C₂₅ aryloxy group, more preferably an C₆-C₁₈ aryloxy group, or an C₆-C₁₂ aryloxy group.

ba and bb are independently an integer from 0 to 4.

wherein the aryl group, arylene group, heterocyclic group, fluorenyl group, fluorenylene group, aliphatic ring group, fused ring group, alkyl group, alkenyl group, alkynyl group, alkoxy group and aryloxy group may be substituted with one or more substituents selected from the group consisting of deuterium; halogen; silane group; silane group substituted with an C₆-C₂₀ aryl group; siloxane group; boron group; germanium group; a cyano group; a nitro group; a C₁-C₂₀ alkylthio group; a C₁-C₂₀ alkoxyl group; a C₁-C₂₀ alkyl group; a C₂-C₂₀ alkenyl group; a C₂-C₂₀ alkynyl group; a C₆-C₂₀ aryl group; a C₆-C₂₀ aryl group substituted with deuterium; a fluorenyl group; a C₂-C₂₀ heterocyclic group; a C₃-C₂₀ aliphatic ring; a C₇-C₂₀ arylalkyl group; a C₈-C₂₀ arylalkenyl group; and a C₇-C₂₀ alkylaryl group; and -L′-N(R^{c})(R^{d}); and the hydrogen of these substituents may be further substituted with one or more deuterium, and the substituents may be bonded to each other to form a saturated or unsaturated ring, wherein the term ‘ring’ means a C₃-C₆₀ aliphatic ring or a C₆-C₆₀ aromatic ring or a C₂-C₆₀ heterocyclic group or a fused ring formed by the combination thereof.

Formula 4 is represented by any one of Formulas 4-1 to 4-3.

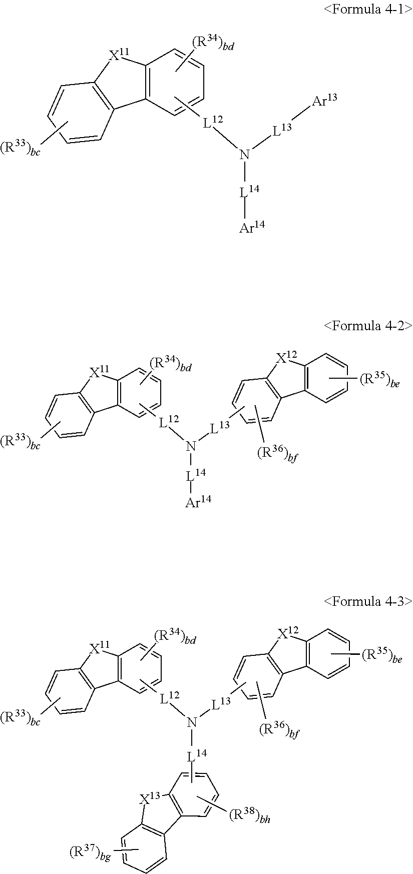

wherein:
Ar¹³, Ar¹⁴, L¹², L¹³ and L¹⁴ are the same as defined in Formula 4,
X¹¹, X¹² and X¹³ are independently O, S, C(R⁶¹)(R⁶²) or NR⁶³,
R³³, R³⁴, R³⁵, R³⁶, R³⁷ and R³⁸ are independently the same or different from each other and are independently selected from the group consisting of deuterium; halogen; a cyano group; a nitro; a C₁-C₂₀ alkyl group; a C₂-C₂₀ alkenyl group; a C₂-C₂₀ alkynyl group; a C₁-C₂₀ alkoxyl group; a C₆-C₂₀ aryl group; a C₆-C₂₀ aryl group substituted with deuterium; a fluorenyl group; a C₂-C₂₀ heterocyclic group including at least one heteroatom of O, N, S, Si or P; a C₃-C₂₀ aliphatic ring; and -L′-N(R^{c})(R^{d}); or a plurality of adjacent groups thereof may be bonded to each other to form a ring,
L′, R^{c} and R^{d} are the same as defined in Formula 4,
R⁶¹, R⁶² and R⁶³ are independently selected from the group consisting of a C₁-C₂₀ alkyl group; a C₂-C₂₀ alkenyl group; a C₂-C₂₀ alkynyl group; a C₁-C₂₀ alkoxyl group; a C₆-C₂₀ aryl group; a C₆-C₂₀ aryl group substituted with deuterium; a fluorenyl group; a C₂-C₂₀ heterocyclic group including at least one heteroatom of O, N, S, Si or P; a C₃-C₂₀ aliphatic ring; or a plurality of adjacent groups thereof may be bonded to each other to form a spiro,
bc, be and bg are independently an integer from 0 to 4, bd, bf and bh are independently an integer of 0 to 3.

Formula 5 is represented by any one of Formulas 5-1 to 5-6.

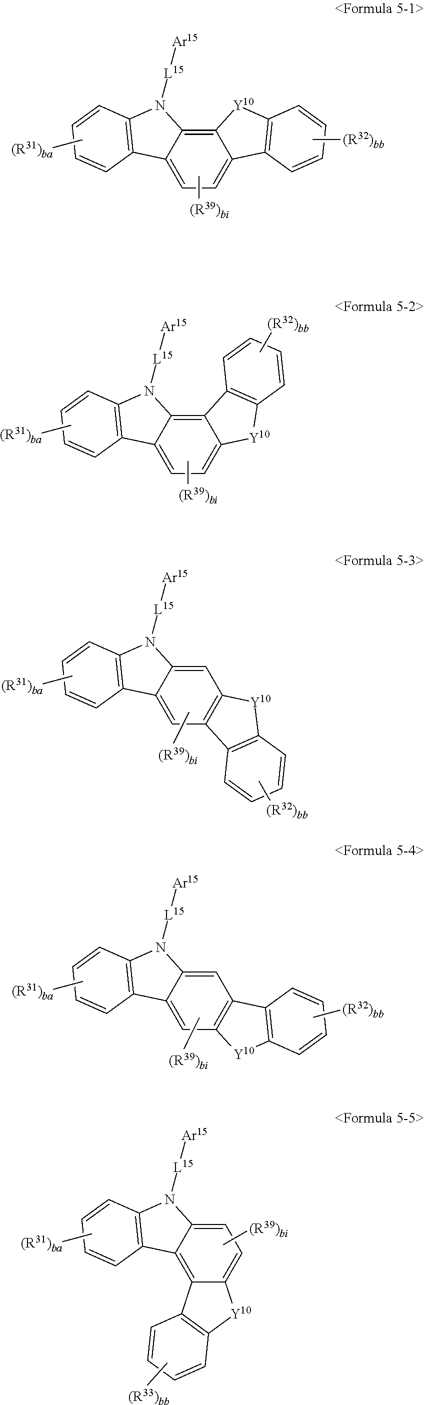

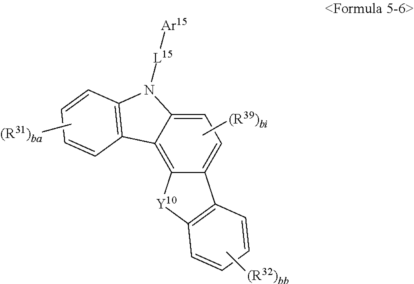

wherein:
Y¹⁰, R³¹, R³², Ar¹⁵, L¹⁵, ba and bb are the same as defined in Formula 5,
R³⁹ is the same as the definition of R³¹, or a plurality of adjacent groups thereof may be bonded to each other to form a ring,
bi is an integer from 0 to 2.

Formula 5 is represented by any one of Formulas 5-7 to 5-9.

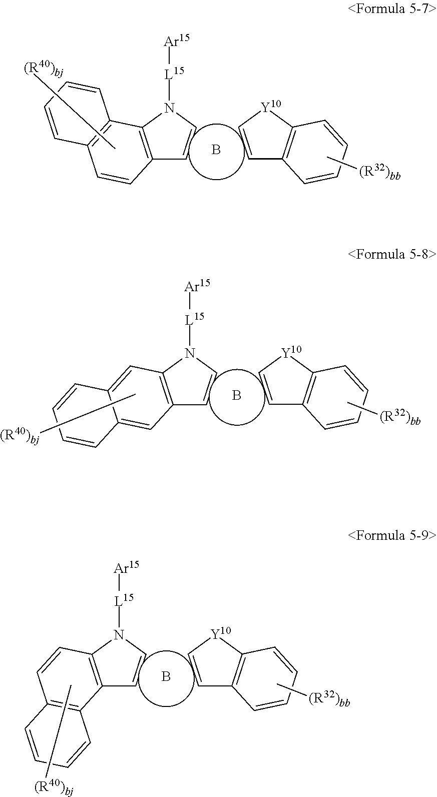

wherein:
Y¹⁰, Ring B, Ar¹⁵, L¹⁵, R³² and bb are the same as defined in Formula 5,
R⁴⁰ is the same as the definition of R³¹, or a plurality of adjacent groups thereof may be bonded to each other to form a ring,
bj is an integer from 0 to 6.

Formula 5 is represented by any one of Formulas 5-10 to 5-12.

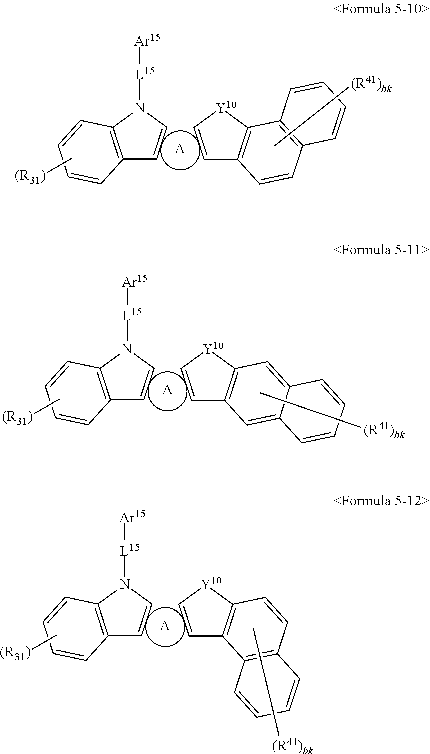

wherein:
Y¹⁰, Ring B, Ar¹⁵, L¹⁵, R³¹ and ba are the same as defined in Formula 5,
R⁴¹ is the same as the definition of R³¹, or a plurality of adjacent groups thereof may be bonded to each other to form a ring,
bk is an integer from 0 to 6.

Formula 5 is represented by any one of Formulas 5-13 to 5-18.

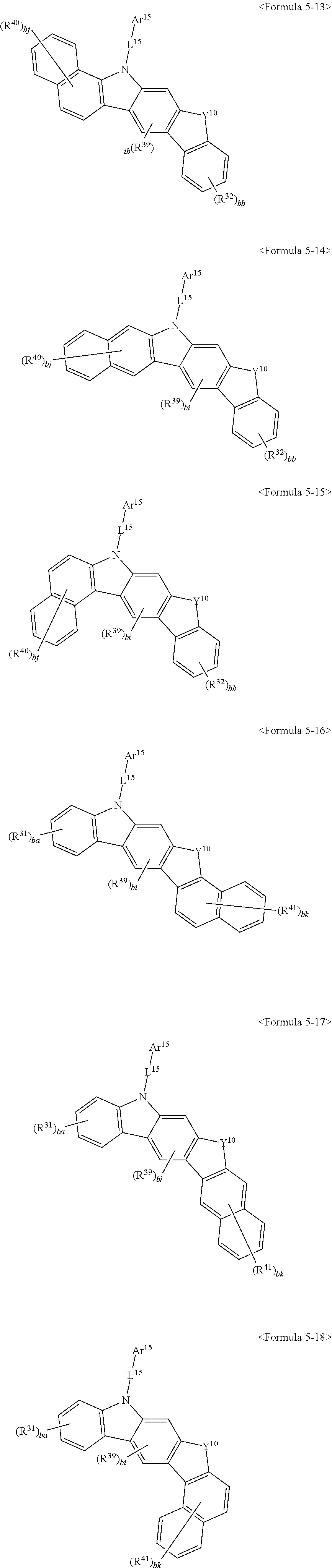

wherein:
Y¹⁰, Ar¹⁵, L¹⁵, R³¹, R³², ba and bb are the same as defined in Formula 5,
R³⁹, R⁴⁰ and R⁴¹ are the same as the definition of R³¹, or a plurality of adjacent groups thereof may be bonded to each other to form a ring,
bi is an integer from 0 to 2, bj and bk are independently an integer from 0 to 6.

Formula 5 is represented by Formula 5-19.

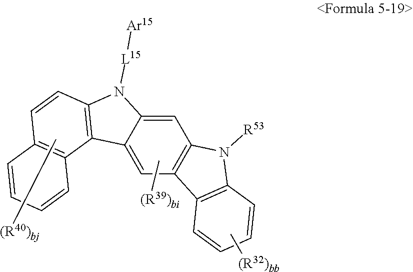

wherein:
Y¹⁰, Ar¹⁵, L¹⁵, R³¹, R³², ba and bb are the same as defined in Formula 5,
R³⁹, R⁴⁰ and R⁴¹ are the same as the definition of R³¹, or a plurality of adjacent groups thereof may be bonded to each other to form a ring,
bi is an integer from 0 to 2, bj is an integer from 0 to 6.

Specifically, the compound represented by Formula 4 may be any one of the following compounds H-1 to H-129, but is not limited thereto.

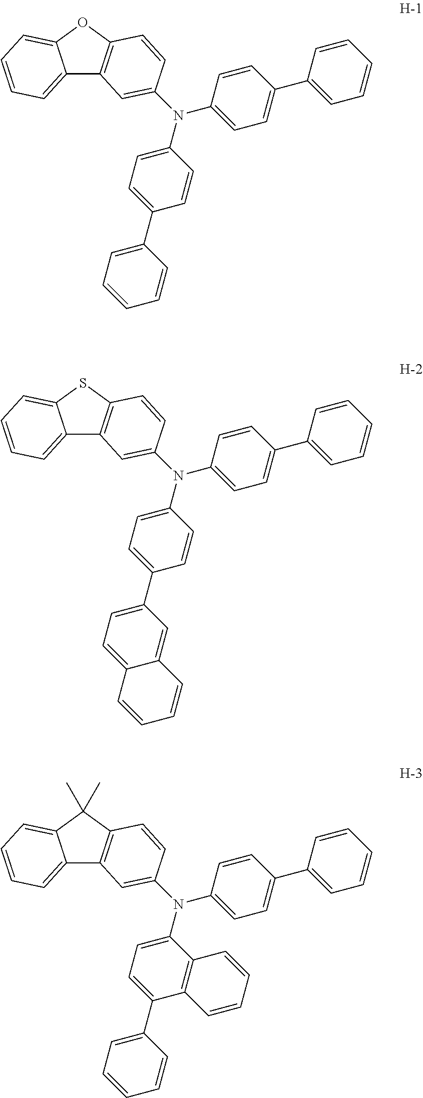 [SMILES: c1ccc(-c2ccc(N(c3ccc(-c4ccccc4)cc3)c3ccc4oc5ccccc5c4c3)cc2)cc1 ; c1ccc(-c2ccc(N(c3ccc(-c4ccc5ccccc5c4)cc3)c3ccc4sc5ccccc5c4c3)cc2)cc1 ; CC1(C)c2ccccc2-c2cc(N(c3ccc(-c4ccccc4)cc3)c3ccc(-c4ccccc4)c4ccccc34)ccc21]

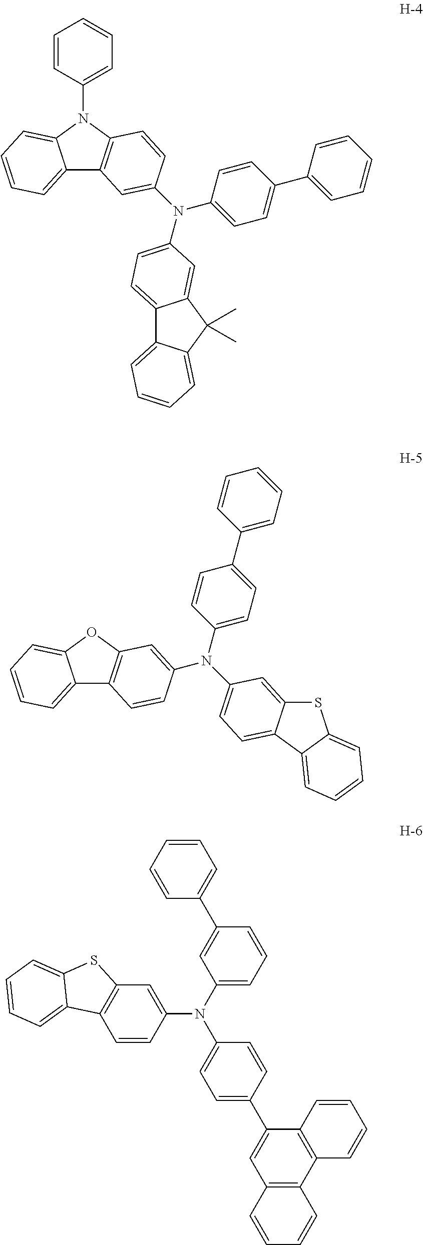 [SMILES: CC1(C)c2ccccc2-c2ccc(N(c3ccc(-c4ccccc4)cc3)c3ccc4c(c3)c3ccccc3n4-c3ccccc3)cc21 ; c1ccc(-c2ccc(N(c3ccc4c(c3)oc3ccccc34)c3ccc4c(c3)sc3ccccc34)cc2)cc1 ; c1ccc(-c2cccc(N(c3ccc(-c4cc5ccccc5c5ccccc45)cc3)c3ccc4c(c3)sc3ccccc34)c2)cc1]

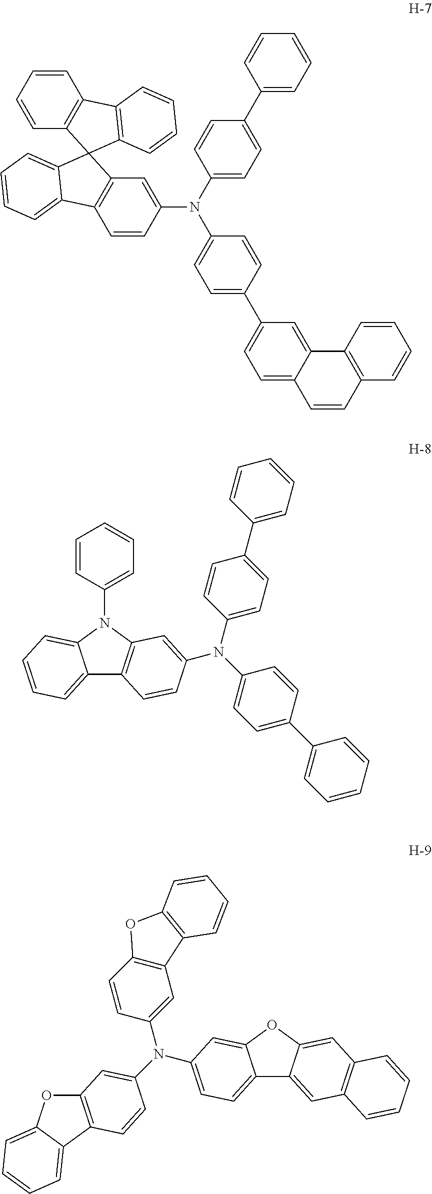 [SMILES: c1ccc(-c2ccc(N(c3ccc(-c4ccc5ccc6ccccc6c5c4)cc3)c3ccc4c(c3)C3(c5ccccc5-c5ccccc53)c3ccccc3-4)cc2)cc1 ; c1ccc(-c2ccc(N(c3ccc(-c4ccccc4)cc3)c3ccc4c5ccccc5n(-c5ccccc5)c4c3)cc2)cc1 ; c1ccc2cc3c(cc2c1)oc1cc(N(c2ccc4c(c2)oc2ccccc24)c2ccc4oc5ccccc5c4c2)ccc13]

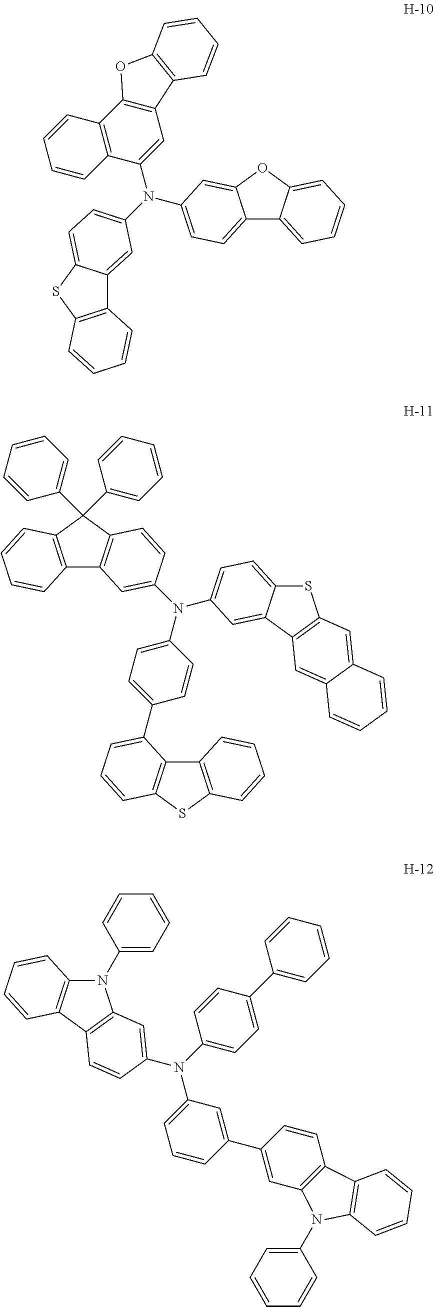 [SMILES: c1ccc2c(c1)oc1cc(N(c3ccc4sc5ccccc5c4c3)c3cc4c5ccccc5oc4c4ccccc34)ccc12 ; c1ccc(C2(c3ccccc3)c3ccccc3-c3cc(N(c4ccc(-c5cccc6sc7ccccc7c56)cc4)c4ccc5sc6cc7ccccc7cc6c5c4)ccc32)cc1 ; c1ccc(-c2ccc(N(c3cccc(-c4ccc5c6ccccc6n(-c6ccccc6)c5c4)c3)c3ccc4c5ccccc5n(-c5ccccc5)c4c3)cc2)cc1]

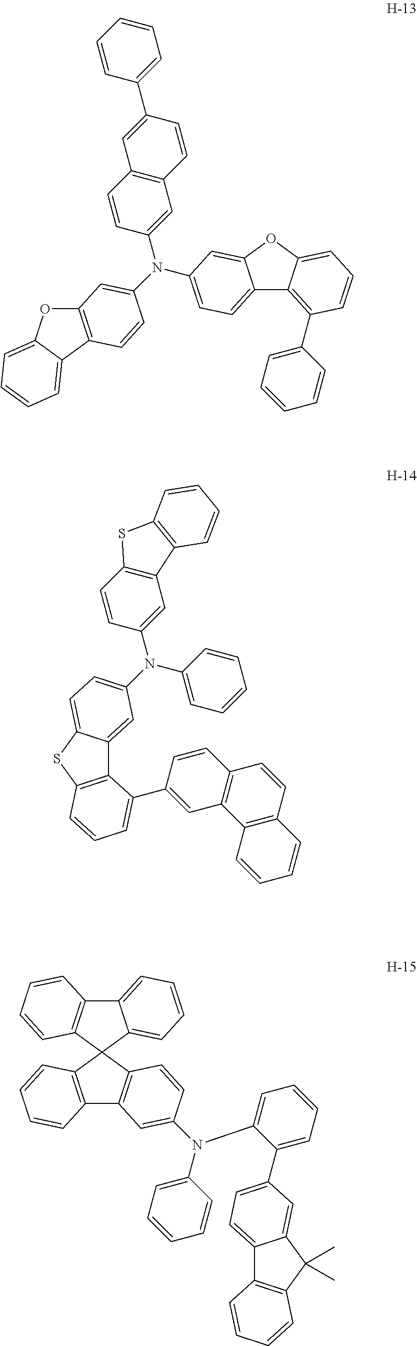 [SMILES: c1ccc(-c2ccc3cc(N(c4ccc5c(c4)oc4ccccc45)c4ccc5c(c4)oc4cccc(-c6ccccc6)c45)ccc3c2)cc1 ; c1ccc(N(c2ccc3sc4ccccc4c3c2)c2ccc3sc4cccc(-c5ccc6ccc7ccccc7c6c5)c4c3c2)cc1 ; CC1(C)c2ccccc2-c2ccc(-c3ccccc3N(c3ccccc3)c3ccc4c(c3)-c3ccccc3C43c4ccccc4-c4ccccc43)cc21]

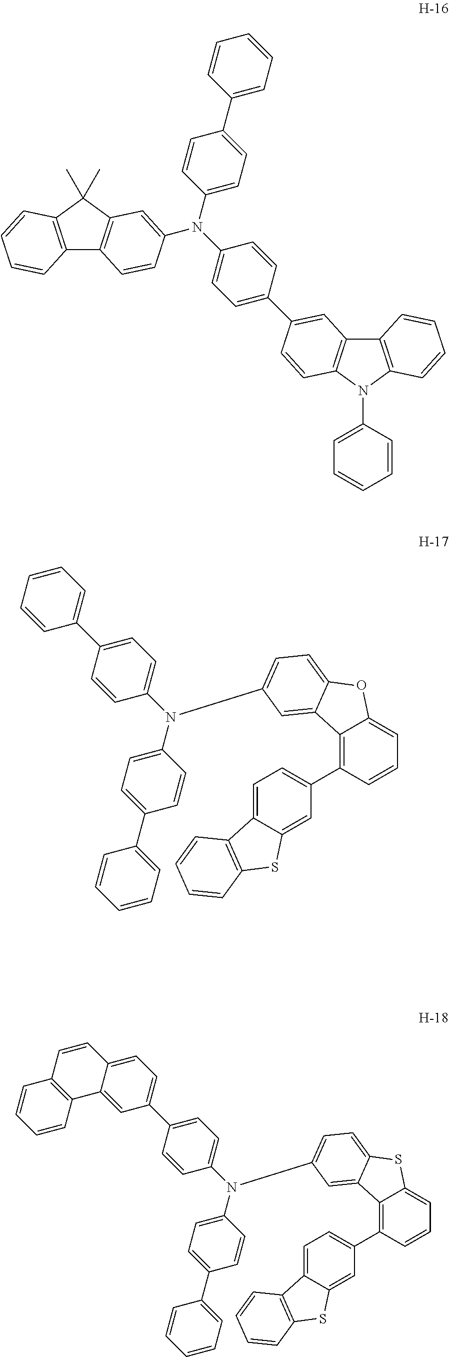 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(-c2ccc(N(c3ccc(-c4ccccc4)cc3)c3ccc4oc5cccc(-c6ccc7c(c6)sc6ccccc67)c5c4c3)cc2)cc1 ; c1ccc(-c2ccc(N(c3ccc(-c4ccc5ccc6ccccc6c5c4)cc3)c3ccc4sc5cccc(-c6ccc7c(c6)sc6ccccc67)c5c4c3)cc2)cc1]

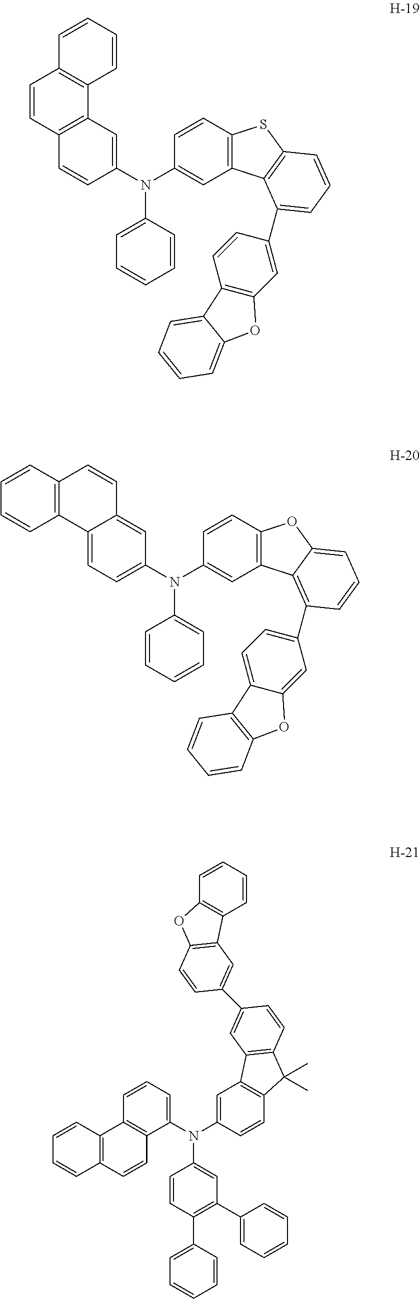 [SMILES: c1ccc(N(c2ccc3ccc4ccccc4c3c2)c2ccc3sc4cccc(-c5ccc6c(c5)oc5ccccc56)c4c3c2)cc1 ; c1ccc(N(c2ccc3c(ccc4ccccc43)c2)c2ccc3oc4cccc(-c5ccc6c(c5)oc5ccccc56)c4c3c2)cc1 ; CC1(C)c2ccc(-c3ccc4oc5ccccc5c4c3)cc2-c2cc(N(c3ccc(-c4ccccc4)c(-c4ccccc4)c3)c3cccc4c3ccc3ccccc34)ccc21]

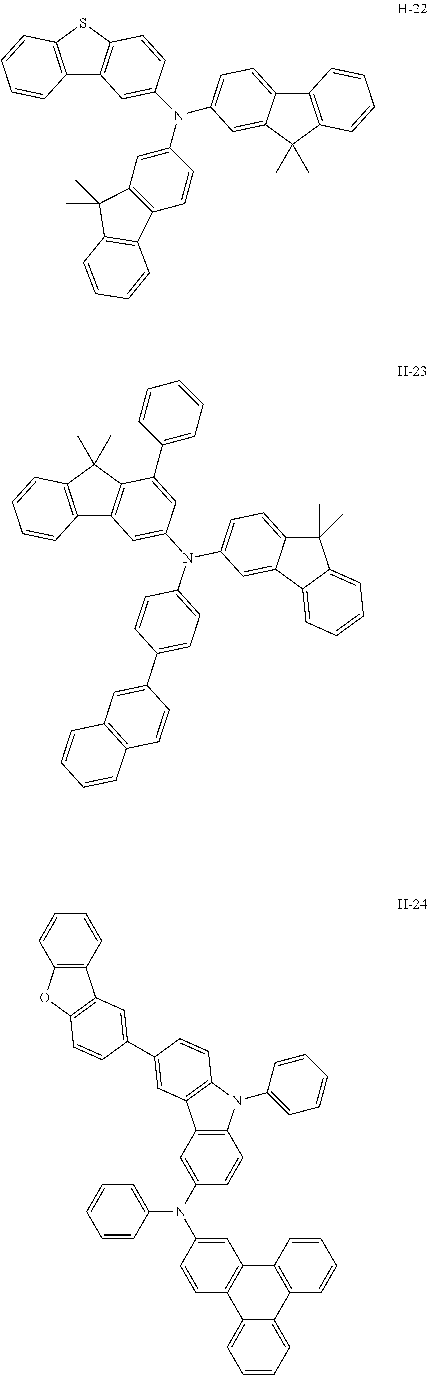 [SMILES: CC1(C)c2ccccc2-c2ccc(N(c3ccc4c(c3)C(C)(C)c3ccccc3-4)c3ccc4sc5ccccc5c4c3)cc21 ; CC1(C)c2ccccc2-c2cc(N(c3ccc(-c4ccc5ccccc5c4)cc3)c3cc(-c4ccccc4)c4c(c3)-c3ccccc3C4(C)C)ccc21 ; c1ccc(N(c2ccc3c4ccccc4c4ccccc4c3c2)c2ccc3c(c2)c2cc(-c4ccc5oc6ccccc6c5c4)ccc2n3-c2ccccc2)cc1]

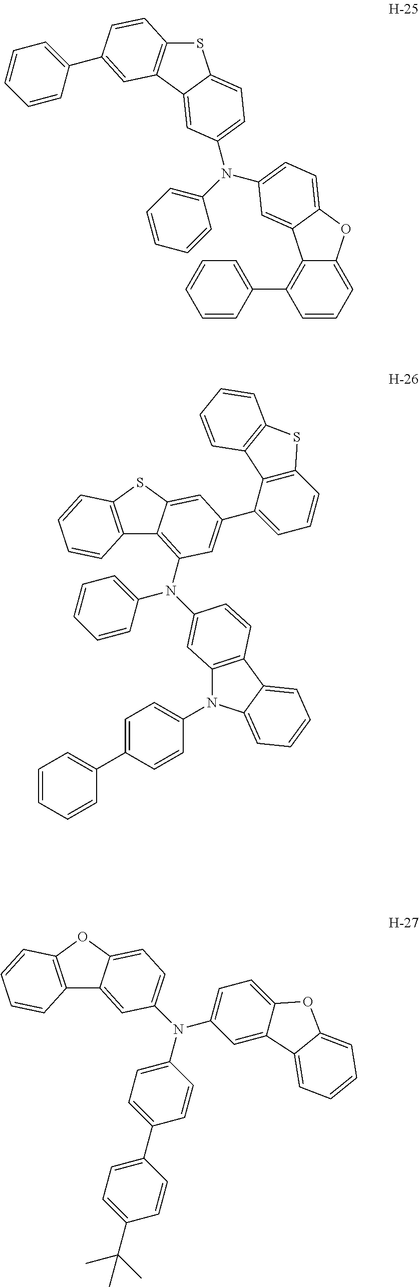 [SMILES: c1ccc(-c2ccc3sc4ccc(N(c5ccccc5)c5ccc6oc7cccc(-c8ccccc8)c7c6c5)cc4c3c2)cc1 ; c1ccc(-c2ccc(-n3c4ccccc4c4ccc(N(c5ccccc5)c5cc(-c6cccc7sc8ccccc8c67)cc6sc7ccccc7c56)cc43)cc2)cc1 ; CC(C)(C)c1ccc(-c2ccc(N(c3ccc4oc5ccccc5c4c3)c3ccc4oc5ccccc5c4c3)cc2)cc1]

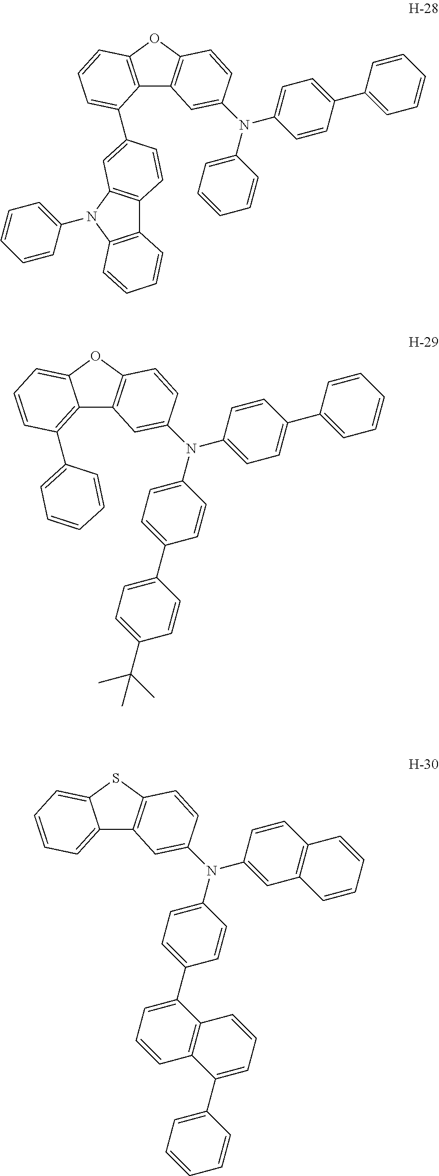 [SMILES: c1ccc(-c2ccc(N(c3ccccc3)c3ccc4oc5cccc(-c6ccc7c8ccccc8n(-c8ccccc8)c7c6)c5c4c3)cc2)cc1 ; CC(C)(C)c1ccc(-c2ccc(N(c3ccc(-c4ccccc4)cc3)c3ccc4oc5cccc(-c6ccccc6)c5c4c3)cc2)cc1 ; c1ccc(-c2cccc3c(-c4ccc(N(c5ccc6ccccc6c5)c5ccc6sc7ccccc7c6c5)cc4)cccc23)cc1]

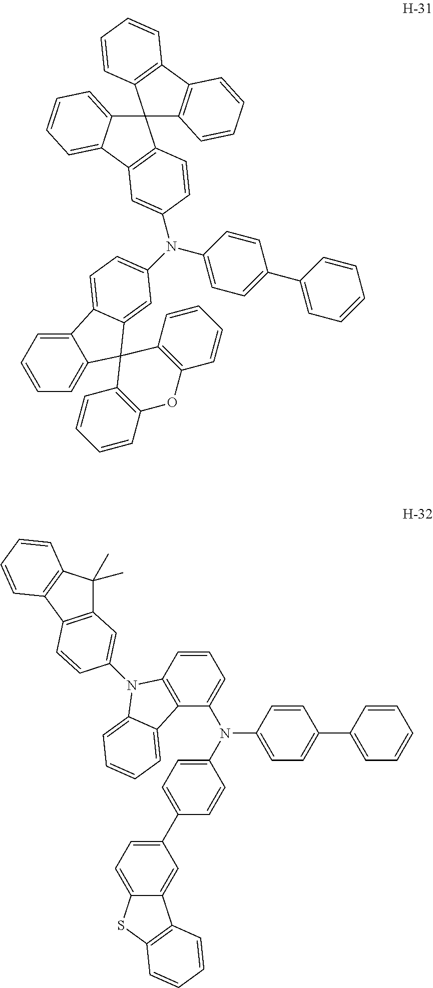 [SMILES: c1ccc(-c2ccc(N(c3ccc4c(c3)-c3ccccc3C43c4ccccc4-c4ccccc43)c3ccc4c(c3)C3(c5ccccc5Oc5ccccc53)c3ccccc3-4)cc2)cc1 ; CC1(C)c2ccccc2-c2ccc(-n3c4ccccc4c4c(N(c5ccc(-c6ccccc6)cc5)c5ccc(-c6ccc7sc8ccccc8c7c6)cc5)cccc43)cc21]

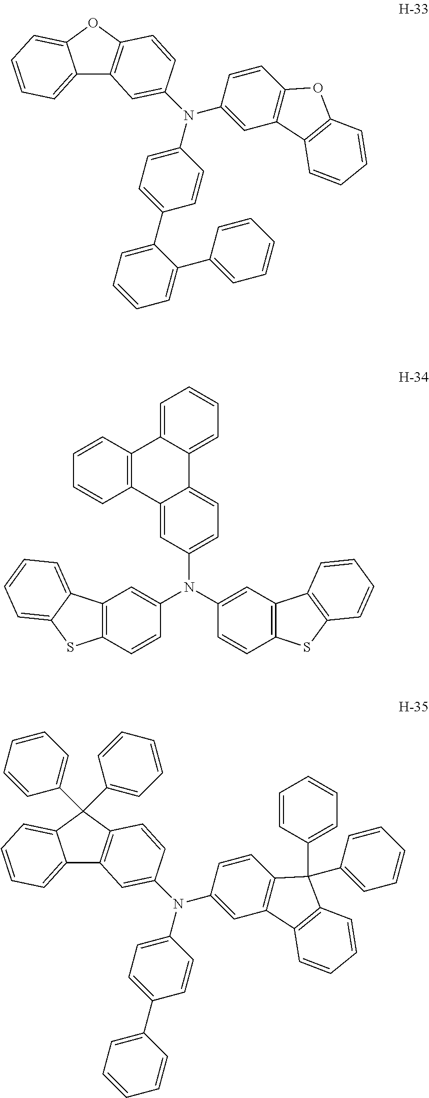 [SMILES: c1ccc(-c2ccccc2-c2ccc(N(c3ccc4oc5ccccc5c4c3)c3ccc4oc5ccccc5c4c3)cc2)cc1 ; c1ccc2c(c1)sc1ccc(N(c3ccc4sc5ccccc5c4c3)c3ccc4c5ccccc5c5ccccc5c4c3)cc12 ; c1ccc(-c2ccc(N(c3ccc4c(c3)-c3ccccc3C4(c3ccccc3)c3ccccc3)c3ccc4c(c3)-c3ccccc3C4(c3ccccc3)c3ccccc3)cc2)cc1]

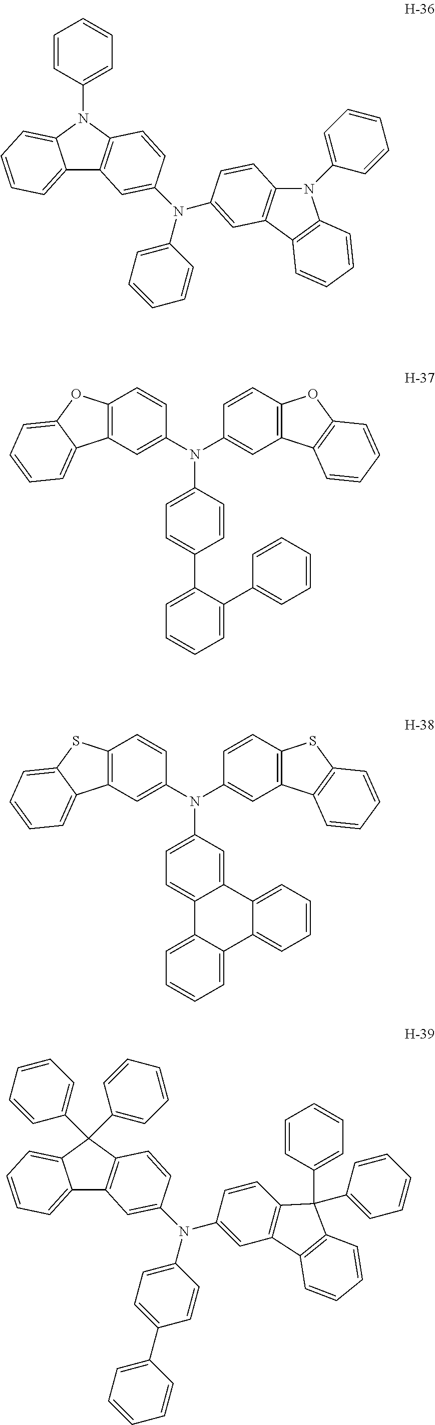 [SMILES: c1ccc(N(c2ccc3c(c2)c2ccccc2n3-c2ccccc2)c2ccc3c(c2)c2ccccc2n3-c2ccccc2)cc1 ; c1ccc(-c2ccccc2-c2ccc(N(c3ccc4oc5ccccc5c4c3)c3ccc4oc5ccccc5c4c3)cc2)cc1 ; c1ccc2c(c1)sc1ccc(N(c3ccc4sc5ccccc5c4c3)c3ccc4c5ccccc5c5ccccc5c4c3)cc12 ; c1ccc(-c2ccc(N(c3ccc4c(c3)-c3ccccc3C4(c3ccccc3)c3ccccc3)c3ccc4c(c3)-c3ccccc3C4(c3ccccc3)c3ccccc3)cc2)cc1]

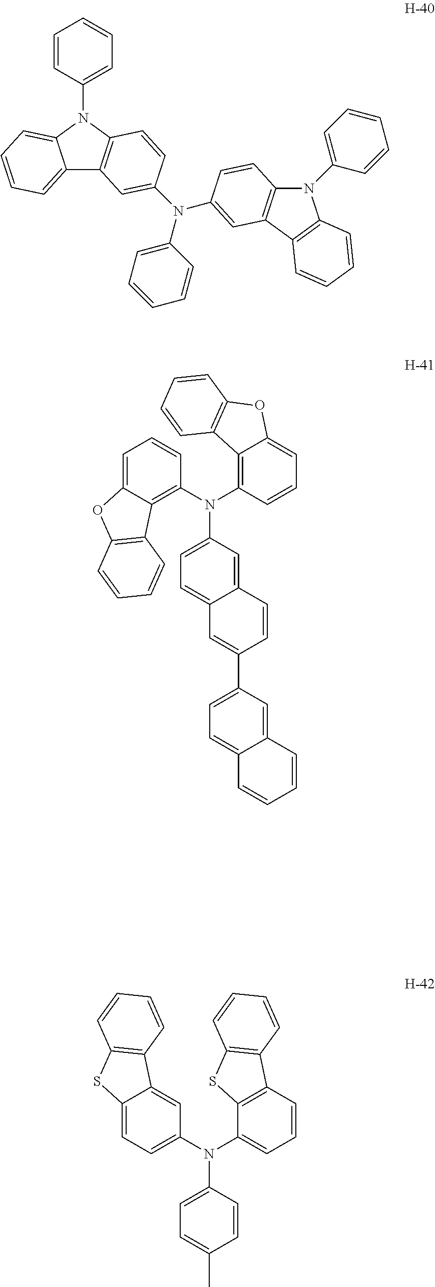 [SMILES: c1ccc(N(c2ccc3c(c2)c2ccccc2n3-c2ccccc2)c2ccc3c(c2)c2ccccc2n3-c2ccccc2)cc1 ; c1ccc2cc(-c3ccc4cc(N(c5cccc6oc7ccccc7c56)c5cccc6oc7ccccc7c56)ccc4c3)ccc2c1 ; Cc1ccc(N(c2ccc3sc4ccccc4c3c2)c2cccc3c2sc2ccccc23)cc1]

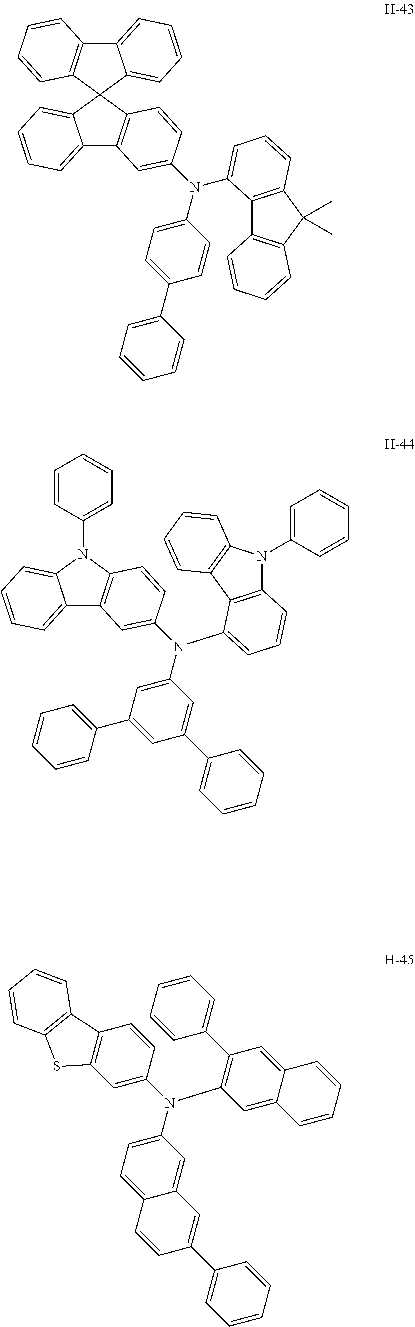 [SMILES: CC1(C)c2ccccc2-c2c(N(c3ccc(-c4ccccc4)cc3)c3ccc4c(c3)-c3ccccc3C43c4ccccc4-c4ccccc43)cccc21 ; c1ccc(-c2cc(-c3ccccc3)cc(N(c3ccc4c(c3)c3ccccc3n4-c3ccccc3)c3cccc4c3c3ccccc3n4-c3ccccc3)c2)cc1 ; c1ccc(-c2ccc3ccc(N(c4ccc5c(c4)sc4ccccc45)c4cc5ccccc5cc4-c4ccccc4)cc3c2)cc1]

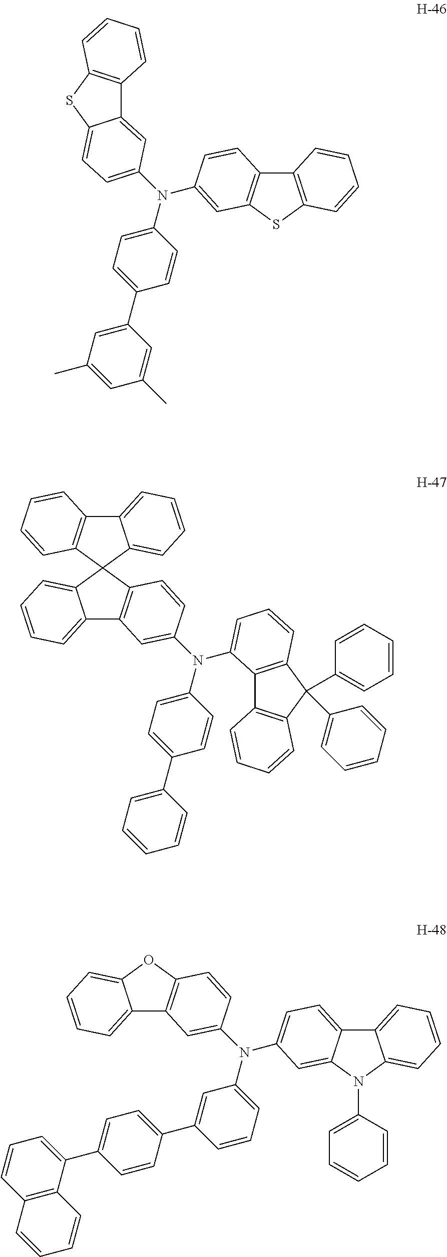 [SMILES: Cc1cc(C)cc(-c2ccc(N(c3ccc4c(c3)sc3ccccc34)c3ccc4sc5ccccc5c4c3)cc2)c1 ; c1ccc(-c2ccc(N(c3ccc4c(c3)-c3ccccc3C43c4ccccc4-c4ccccc43)c3cccc4c3-c3ccccc3C4(c3ccccc3)c3ccccc3)cc2)cc1 ; c1ccc(-n2c3ccccc3c3ccc(N(c4cccc(-c5ccc(-c6cccc7ccccc67)cc5)c4)c4ccc5oc6ccccc6c5c4)cc32)cc1]

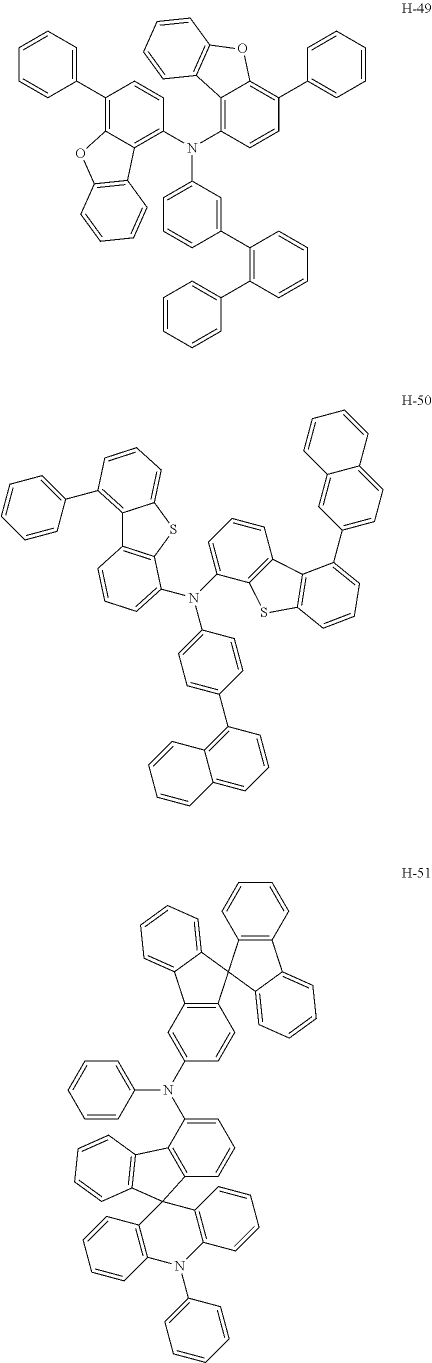 [SMILES: c1ccc(-c2ccccc2-c2cccc(N(c3ccc(-c4ccccc4)c4oc5ccccc5c34)c3ccc(-c4ccccc4)c4oc5ccccc5c34)c2)cc1 ; c1ccc(-c2cccc3sc4c(N(c5ccc(-c6cccc7ccccc67)cc5)c5cccc6c5sc5cccc(-c7ccc8ccccc8c7)c56)cccc4c23)cc1 ; c1ccc(N(c2ccc3c(c2)-c2ccccc2C32c3ccccc3-c3ccccc32)c2cccc3c2-c2ccccc2C32c3ccccc3N(c3ccccc3)c3ccccc32)cc1]

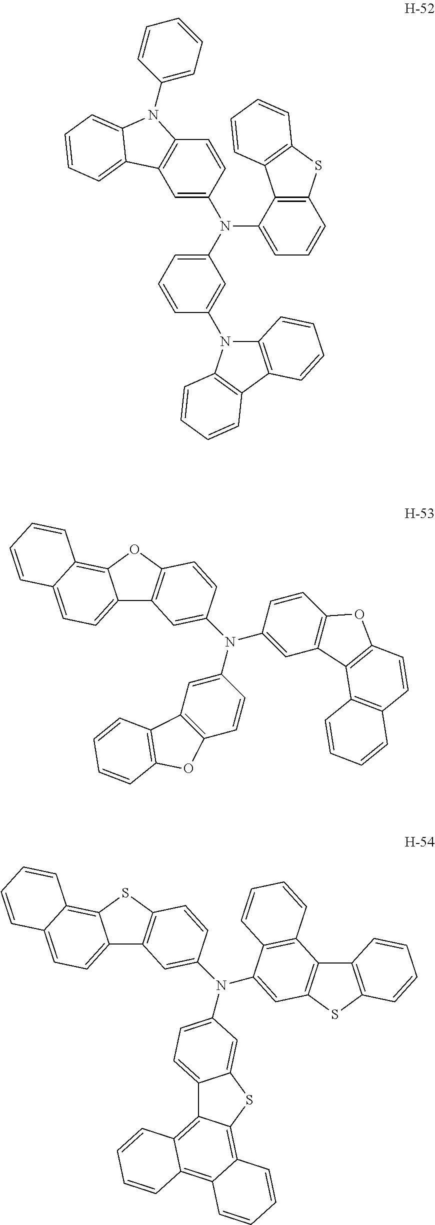 [SMILES: c1ccc(-n2c3ccccc3c3cc(N(c4cccc(-n5c6ccccc6c6ccccc65)c4)c4cccc5sc6ccccc6c45)ccc32)cc1 ; c1ccc2c(c1)ccc1c3cc(N(c4ccc5oc6ccccc6c5c4)c4ccc5oc6ccc7ccccc7c6c5c4)ccc3oc21 ; c1ccc2c(c1)ccc1c3cc(N(c4ccc5c(c4)sc4c6ccccc6c6ccccc6c54)c4cc5sc6ccccc6c5c5ccccc45)ccc3sc21]

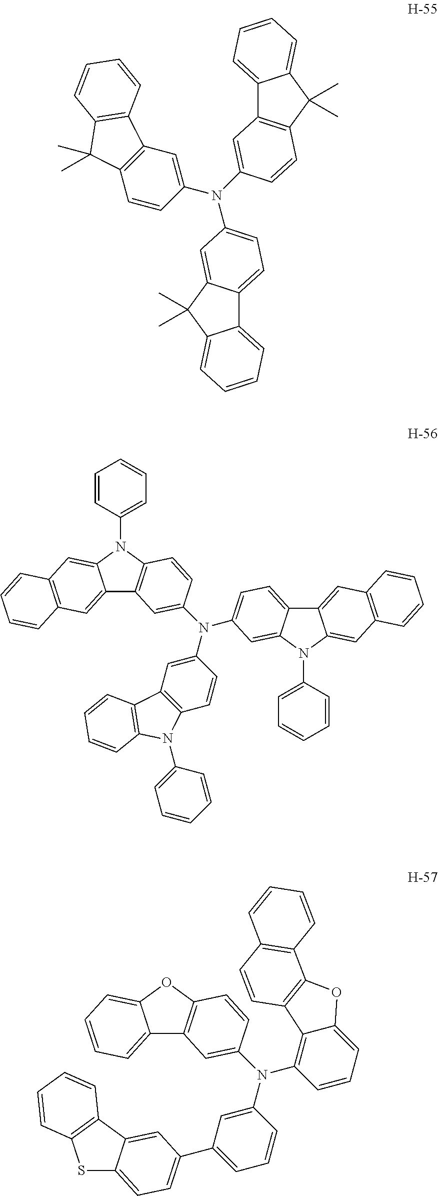

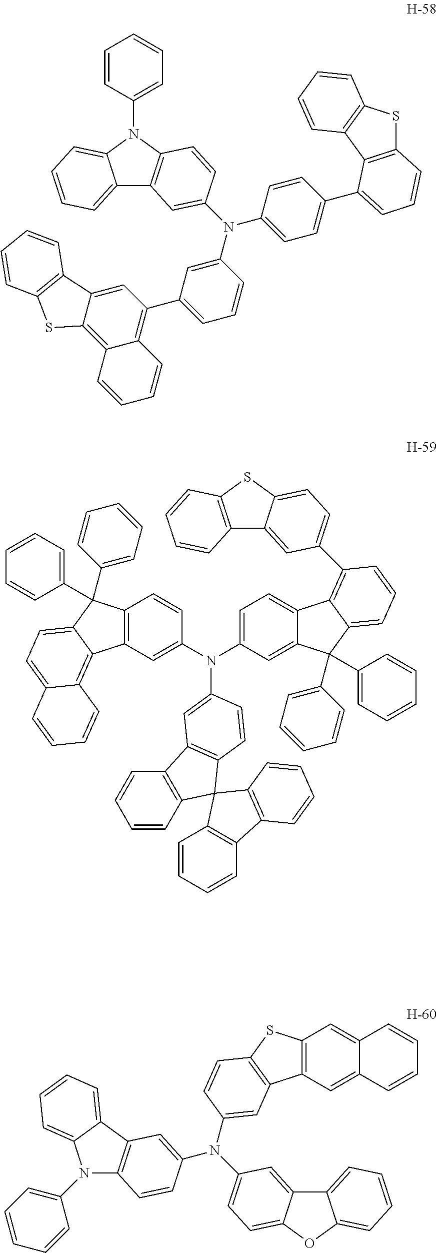 [SMILES: c1ccc(-n2c3ccccc3c3cc(N(c4ccc(-c5cccc6sc7ccccc7c56)cc4)c4cccc(-c5cc6c7ccccc7sc6c6ccccc56)c4)ccc32)cc1 ; c1ccc(C2(c3ccccc3)c3cc(N(c4ccc5c(c4)-c4c(ccc6ccccc46)C5(c4ccccc4)c4ccccc4)c4ccc5c(c4)-c4ccccc4C54c5ccccc5-c5ccccc54)ccc3-c3c(-c4ccc5sc6ccccc6c5c4)cccc32)cc1 ; c1ccc(-n2c3ccccc3c3cc(N(c4ccc5oc6ccccc6c5c4)c4ccc5sc6cc7ccccc7cc6c5c4)ccc32)cc1]

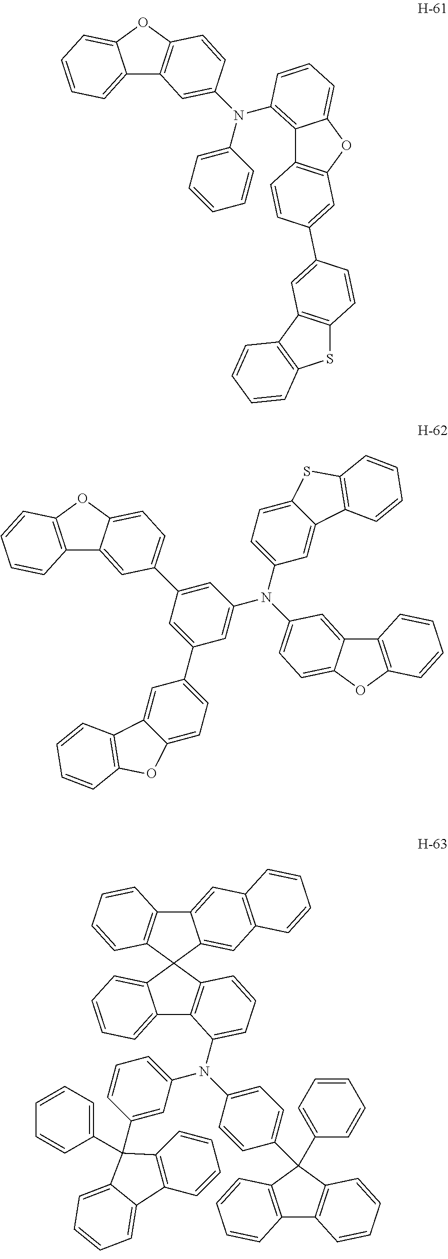

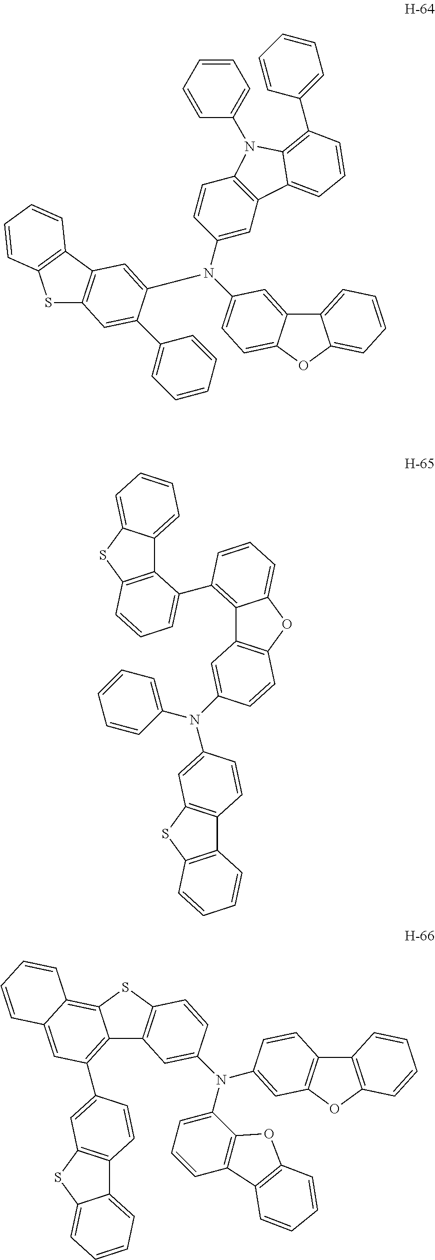 [SMILES: c1ccc(-c2cc3sc4ccccc4c3cc2N(c2ccc3oc4ccccc4c3c2)c2ccc3c(c2)c2cccc(-c4ccccc4)c2n3-c2ccccc2)cc1 ; c1ccc(N(c2ccc3c(c2)sc2ccccc23)c2ccc3oc4cccc(-c5cccc6sc7ccccc7c56)c4c3c2)cc1 ; c1ccc2c(c1)cc(-c1ccc3c(c1)sc1ccccc13)c1c3cc(N(c4ccc5c(c4)oc4ccccc45)c4cccc5c4oc4ccccc45)ccc3sc21]

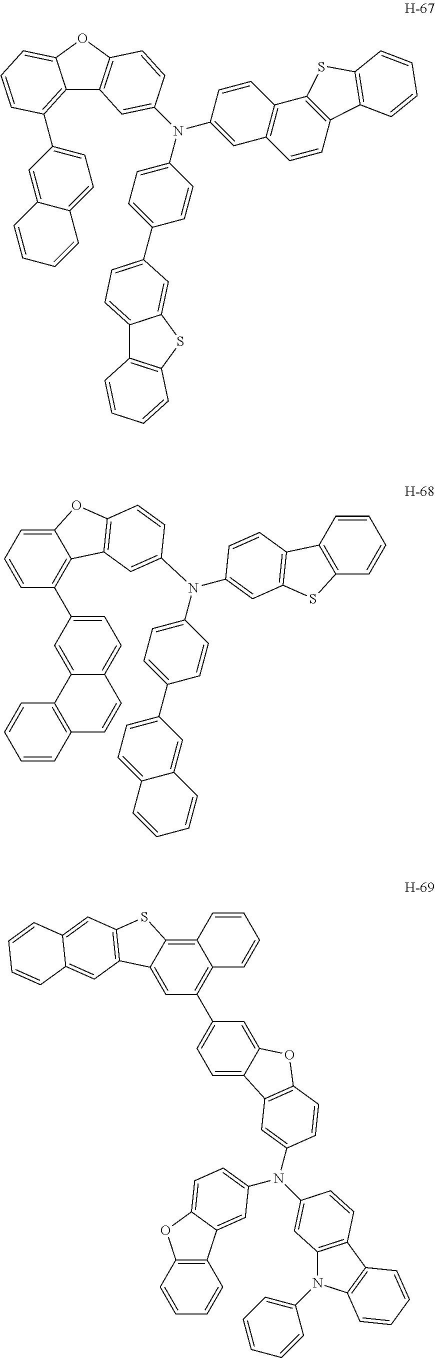 [SMILES: c1ccc2cc(-c3cccc4oc5ccc(N(c6ccc(-c7ccc8c(c7)sc7ccccc78)cc6)c6ccc7c(ccc8c9ccccc9sc78)c6)cc5c34)ccc2c1 ; c1ccc2cc(-c3ccc(N(c4ccc5c(c4)sc4ccccc45)c4ccc5oc6cccc(-c7ccc8ccc9ccccc9c8c7)c6c5c4)cc3)ccc2c1 ; c1ccc(-n2c3ccccc3c3ccc(N(c4ccc5oc6ccccc6c5c4)c4ccc5oc6cc(-c7cc8c9cc%10ccccc%10cc9sc8c8ccccc78)ccc6c5c4)cc32)cc1]

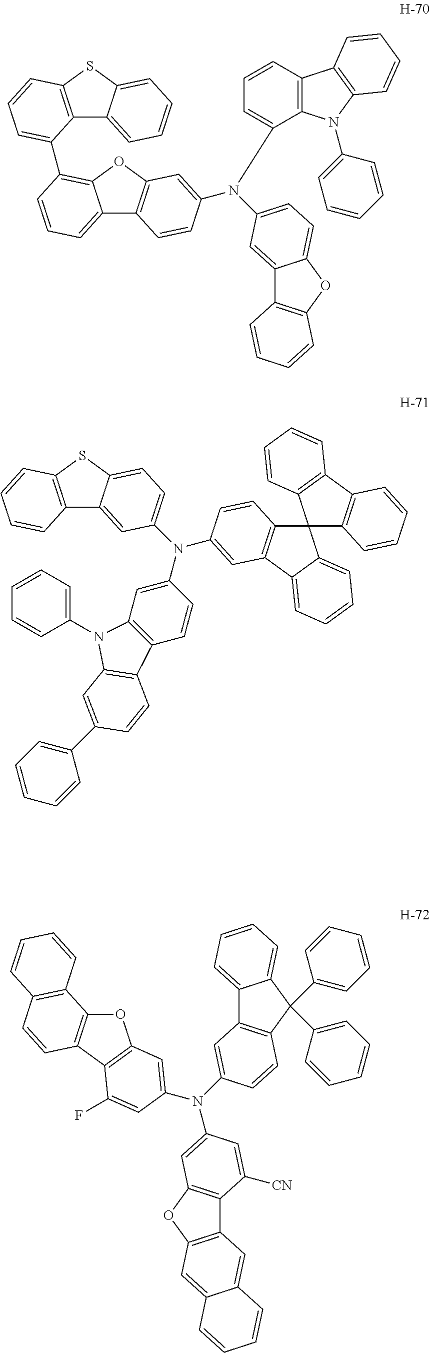 [SMILES: c1ccc(-n2c3ccccc3c3cccc(N(c4ccc5c(c4)oc4c(-c6cccc7sc8ccccc8c67)cccc45)c4ccc5oc6ccccc6c5c4)c32)cc1 ; c1ccc(-c2ccc3c4ccc(N(c5ccc6c(c5)-c5ccccc5C65c6ccccc6-c6ccccc65)c5ccc6sc7ccccc7c6c5)cc4n(-c4ccccc4)c3c2)cc1 ; N#Cc1cc(N(c2ccc3c(c2)-c2ccccc2C3(c2ccccc2)c2ccccc2)c2cc(F)c3c(c2)oc2c4ccccc4ccc23)cc2oc3cc4ccccc4cc3c12]

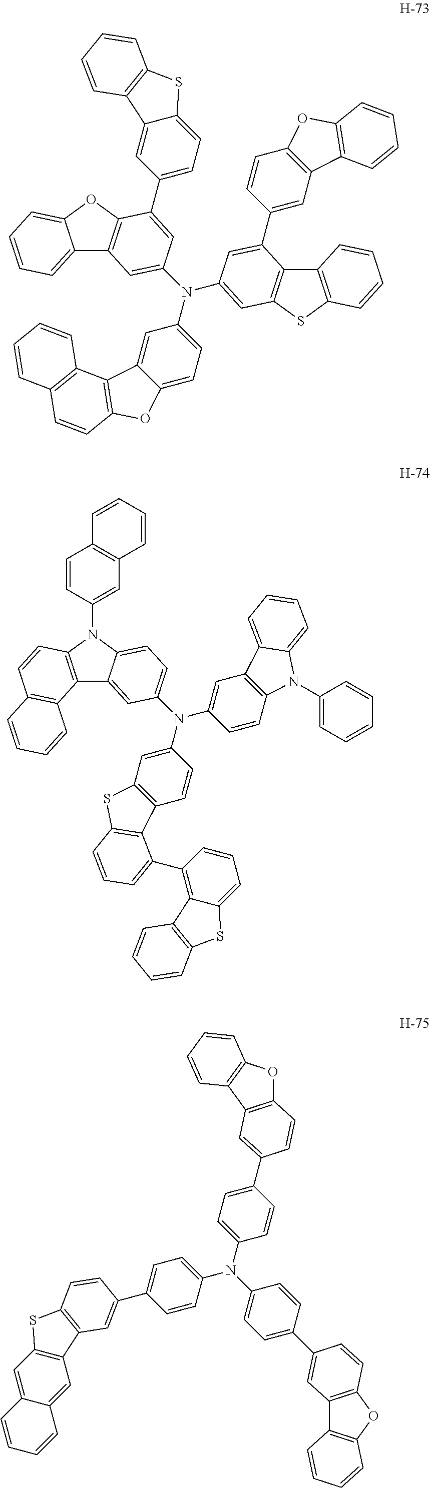 [SMILES: c1ccc2c(c1)ccc1oc3ccc(N(c4cc(-c5ccc6oc7ccccc7c6c5)c5c(c4)sc4ccccc45)c4cc(-c5ccc6sc7ccccc7c6c5)c5oc6ccccc6c5c4)cc3c12 ; c1ccc(-n2c3ccccc3c3cc(N(c4ccc5c(c4)sc4cccc(-c6cccc7sc8ccccc8c67)c45)c4ccc5c(c4)c4c6ccccc6ccc4n5-c4ccc5ccccc5c4)ccc32)cc1 ; c1ccc2cc3c(cc2c1)sc1ccc(-c2ccc(N(c4ccc(-c5ccc6oc7ccccc7c6c5)cc4)c4ccc(-c5ccc6oc7ccccc7c6c5)cc4)cc2)cc13]

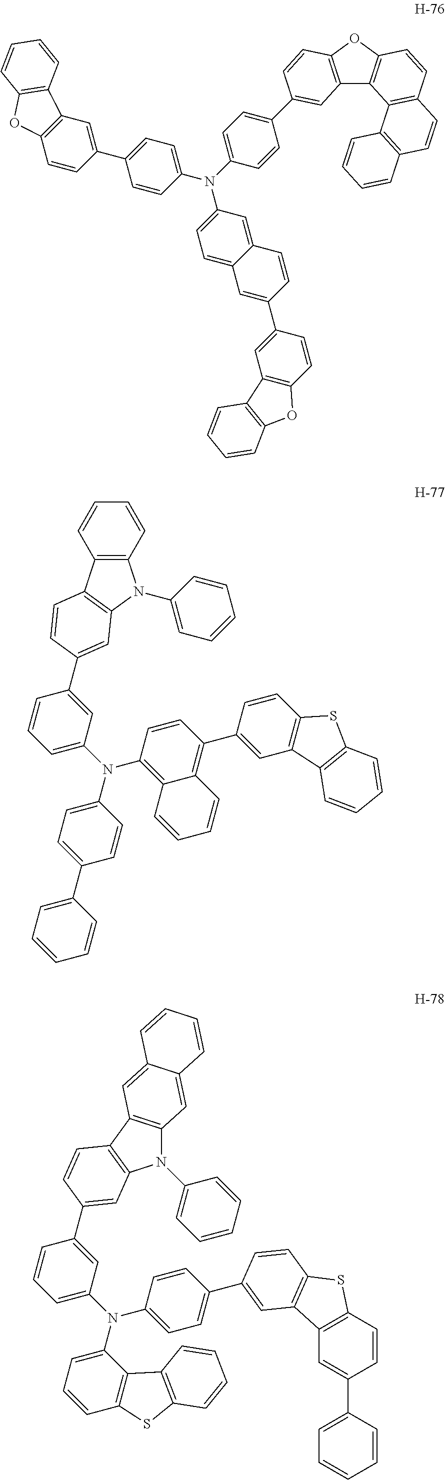 [SMILES: c1ccc2c(c1)ccc1ccc3oc4ccc(-c5ccc(N(c6ccc(-c7ccc8oc9ccccc9c8c7)cc6)c6ccc7cc(-c8ccc9oc%10ccccc%10c9c8)ccc7c6)cc5)cc4c3c12 ; c1ccc(-c2ccc(N(c3cccc(-c4ccc5c6ccccc6n(-c6ccccc6)c5c4)c3)c3ccc(-c4ccc5sc6ccccc6c5c4)c4ccccc34)cc2)cc1 ; c1ccc(-c2ccc3sc4ccc(-c5ccc(N(c6cccc(-c7ccc8c9cc%10ccccc%10cc9n(-c9ccccc9)c8c7)c6)c6cccc7sc8ccccc8c67)cc5)cc4c3c2)cc1]

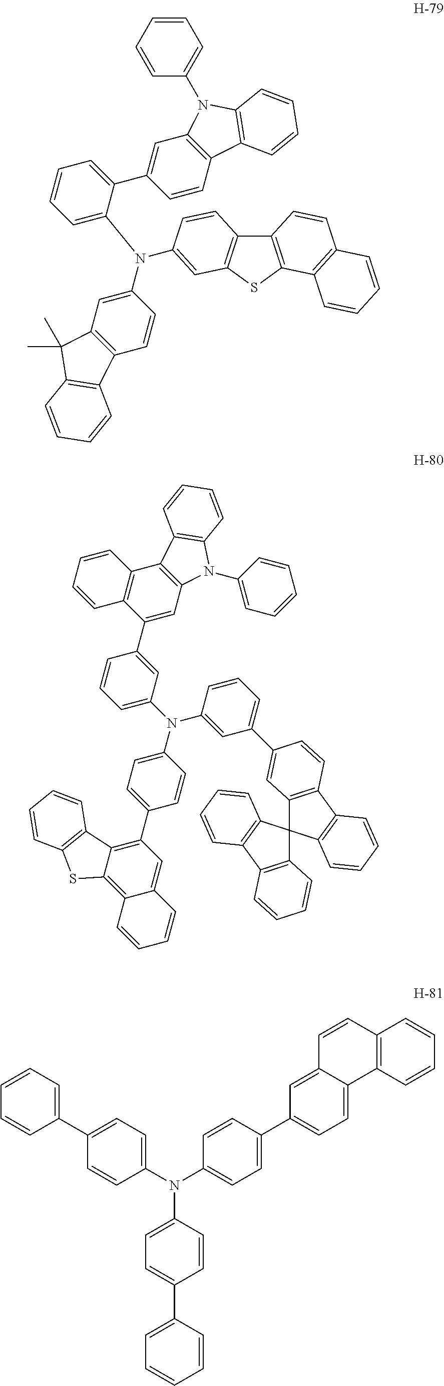 [SMILES: CC1(C)c2ccccc2-c2ccc(N(c3ccc4c(c3)sc3c5ccccc5ccc43)c3ccccc3-c3ccc4c5ccccc5n(-c5ccccc5)c4c3)cc21 ; c1ccc(-n2c3ccccc3c3c4ccccc4c(-c4cccc(N(c5ccc(-c6cc7ccccc7c7sc8ccccc8c67)cc5)c5cccc(-c6ccc7c(c6)C6(c8ccccc8-c8ccccc86)c6ccccc6-7)c5)c4)cc32)cc1 ; c1ccc(-c2ccc(N(c3ccc(-c4ccccc4)cc3)c3ccc(-c4ccc5c(ccc6ccccc65)c4)cc3)cc2)cc1]

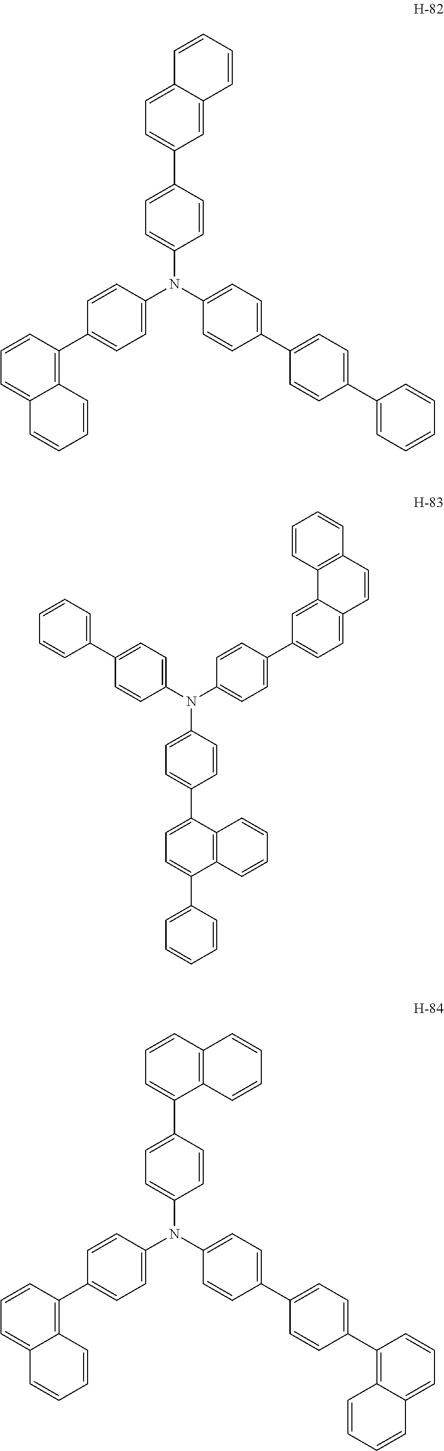 [SMILES: c1ccc(-c2ccc(-c3ccc(N(c4ccc(-c5ccc6ccccc6c5)cc4)c4ccc(-c5cccc6ccccc56)cc4)cc3)cc2)cc1 ; c1ccc(-c2ccc(N(c3ccc(-c4ccc5ccc6ccccc6c5c4)cc3)c3ccc(-c4ccc(-c5ccccc5)c5ccccc45)cc3)cc2)cc1 ; c1ccc2c(-c3ccc(-c4ccc(N(c5ccc(-c6cccc7ccccc67)cc5)c5ccc(-c6cccc7ccccc67)cc5)cc4)cc3)cccc2c1]

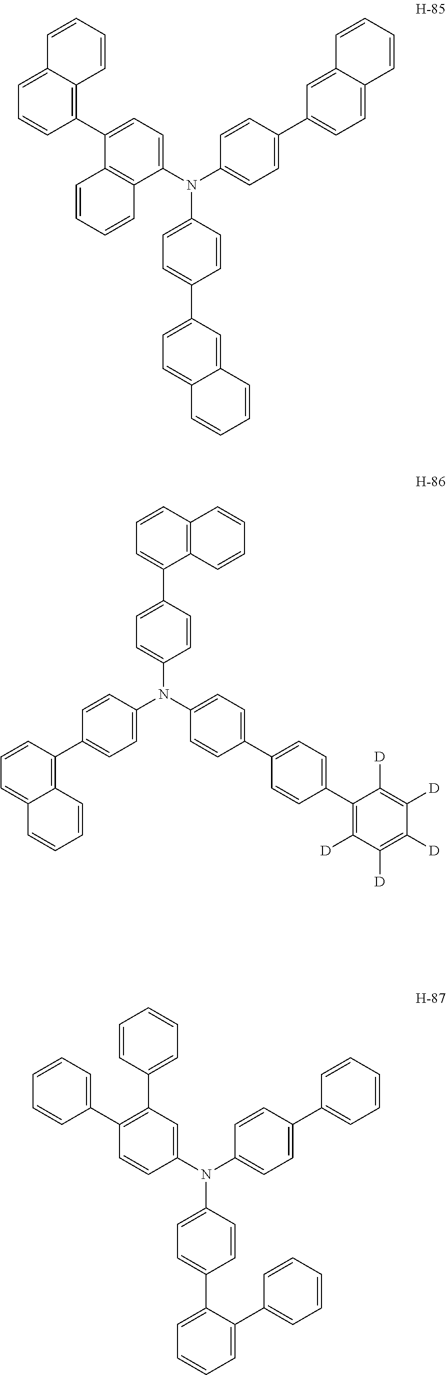 [SMILES: c1ccc2cc(-c3ccc(N(c4ccc(-c5ccc6ccccc6c5)cc4)c4ccc(-c5cccc6ccccc56)c5ccccc45)cc3)ccc2c1 ; [2H]c1c([2H])c([2H])c(-c2ccc(-c3ccc(N(c4ccc(-c5cccc6ccccc56)cc4)c4ccc(-c5cccc6ccccc56)cc4)cc3)cc2)c([2H])c1[2H] ; c1ccc(-c2ccc(N(c3ccc(-c4ccccc4-c4ccccc4)cc3)c3ccc(-c4ccccc4)c(-c4ccccc4)c3)cc2)cc1]

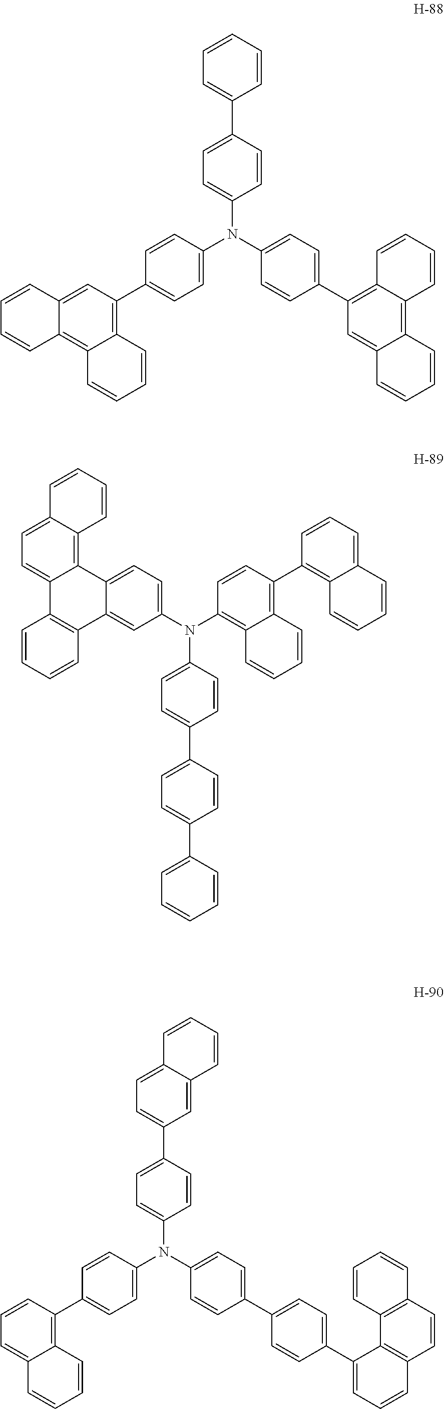 [SMILES: c1ccc(-c2ccc(N(c3ccc(-c4cc5ccccc5c5ccccc45)cc3)c3ccc(-c4cc5ccccc5c5ccccc45)cc3)cc2)cc1 ; c1ccc(-c2ccc(-c3ccc(N(c4ccc5c(c4)c4ccccc4c4ccc6ccccc6c45)c4ccc(-c5cccc6ccccc56)c5ccccc45)cc3)cc2)cc1 ; c1ccc2cc(-c3ccc(N(c4ccc(-c5ccc(-c6cccc7ccc8ccccc8c67)cc5)cc4)c4ccc(-c5cccc6ccccc56)cc4)cc3)ccc2c1]

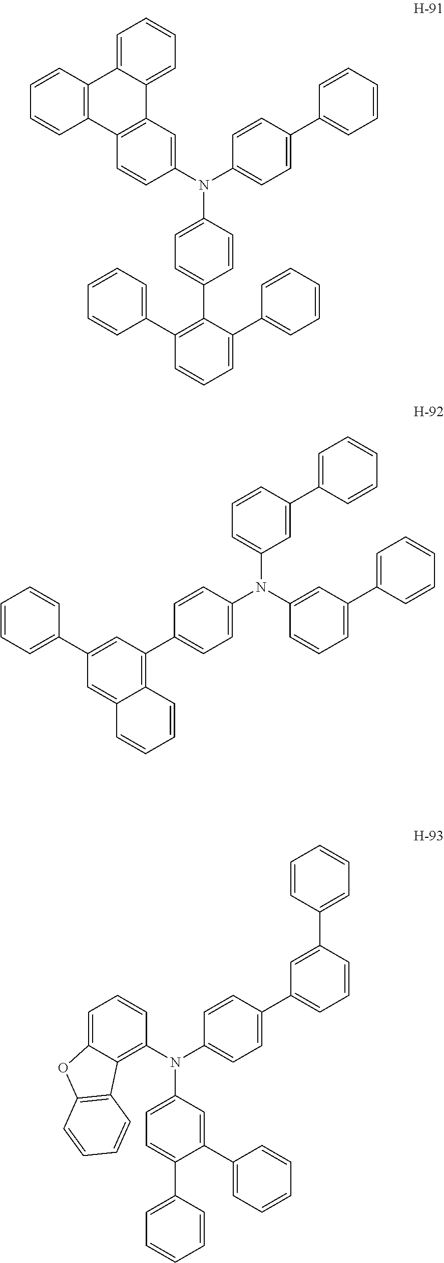 [SMILES: c1ccc(-c2ccc(N(c3ccc(-c4c(-c5ccccc5)cccc4-c4ccccc4)cc3)c3ccc4c5ccccc5c5ccccc5c4c3)cc2)cc1 ; c1ccc(-c2cccc(N(c3ccc(-c4cc(-c5ccccc5)cc5ccccc45)cc3)c3cccc(-c4ccccc4)c3)c2)cc1 ; c1ccc(-c2cccc(-c3ccc(N(c4ccc(-c5ccccc5)c(-c5ccccc5)c4)c4cccc5oc6ccccc6c45)cc3)c2)cc1]

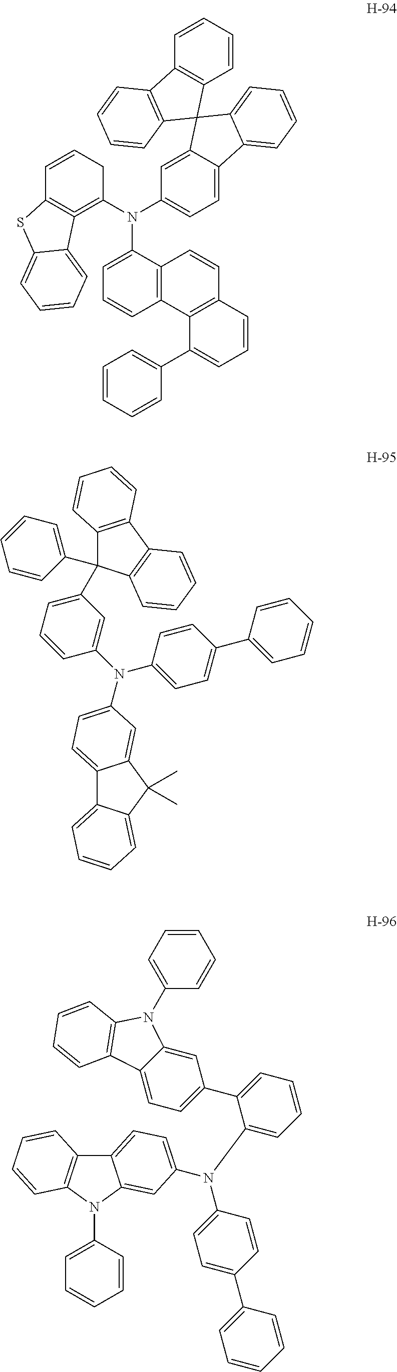

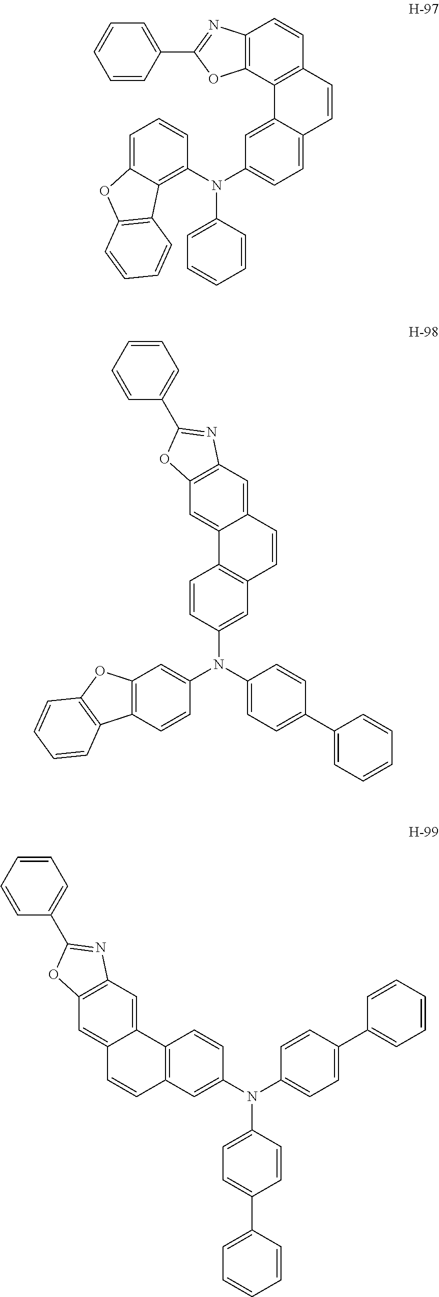 [SMILES: c1ccc(-c2nc3ccc4ccc5ccc(N(c6ccccc6)c6cccc7oc8ccccc8c67)cc5c4c3o2)cc1 ; c1ccc(-c2ccc(N(c3ccc4c(ccc5cc6nc(-c7ccccc7)oc6cc54)c3)c3ccc4c(c3)oc3ccccc34)cc2)cc1 ; c1ccc(-c2ccc(N(c3ccc(-c4ccccc4)cc3)c3ccc4c(ccc5cc6oc(-c7ccccc7)nc6cc54)c3)cc2)cc1]

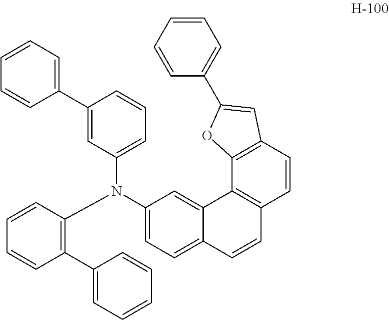 [SMILES: c1ccc(-c2cccc(N(c3ccc4ccc5ccc6cc(-c7ccccc7)oc6c5c4c3)c3ccccc3-c3ccccc3)c2)cc1]

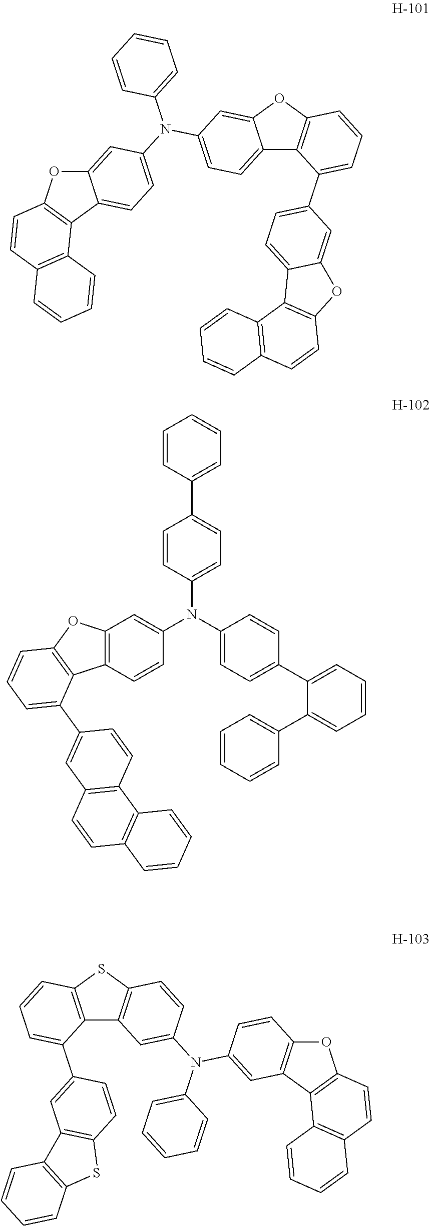 [SMILES: c1ccc(N(c2ccc3c(c2)oc2cccc(-c4ccc5c(c4)oc4ccc6ccccc6c45)c23)c2ccc3c(c2)oc2ccc4ccccc4c23)cc1 ; c1ccc(-c2ccc(N(c3ccc(-c4ccccc4-c4ccccc4)cc3)c3ccc4c(c3)oc3cccc(-c5ccc6c(ccc7ccccc76)c5)c34)cc2)cc1 ; c1ccc(N(c2ccc3sc4cccc(-c5ccc6sc7ccccc7c6c5)c4c3c2)c2ccc3oc4ccc5ccccc5c4c3c2)cc1]

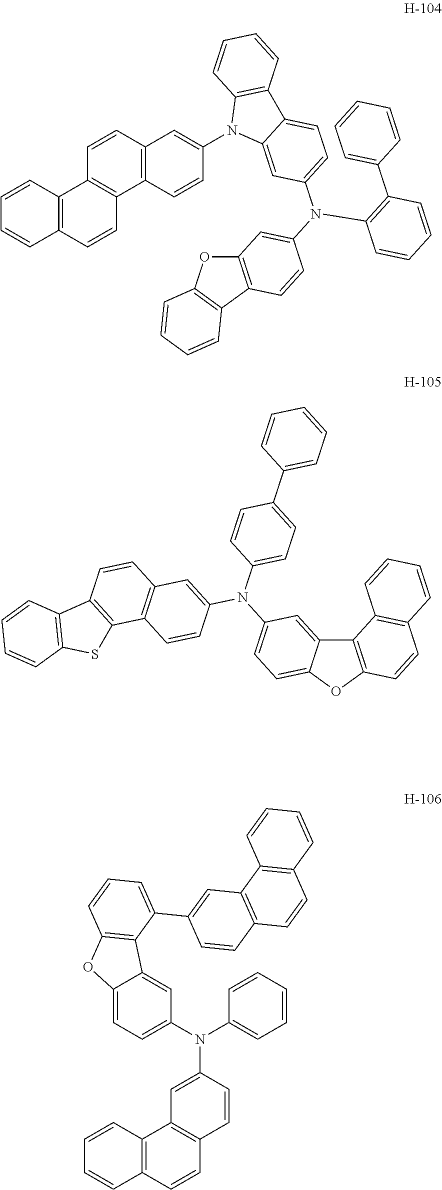 [SMILES: c1ccc(-c2ccccc2N(c2ccc3c(c2)oc2ccccc23)c2ccc3c4ccccc4n(-c4ccc5c(ccc6c7ccccc7ccc56)c4)c3c2)cc1 ; c1ccc(-c2ccc(N(c3ccc4c(ccc5c6ccccc6sc45)c3)c3ccc4oc5ccc6ccccc6c5c4c3)cc2)cc1 ; c1ccc(N(c2ccc3ccc4ccccc4c3c2)c2ccc3oc4cccc(-c5ccc6ccc7ccccc7c6c5)c4c3c2)cc1]

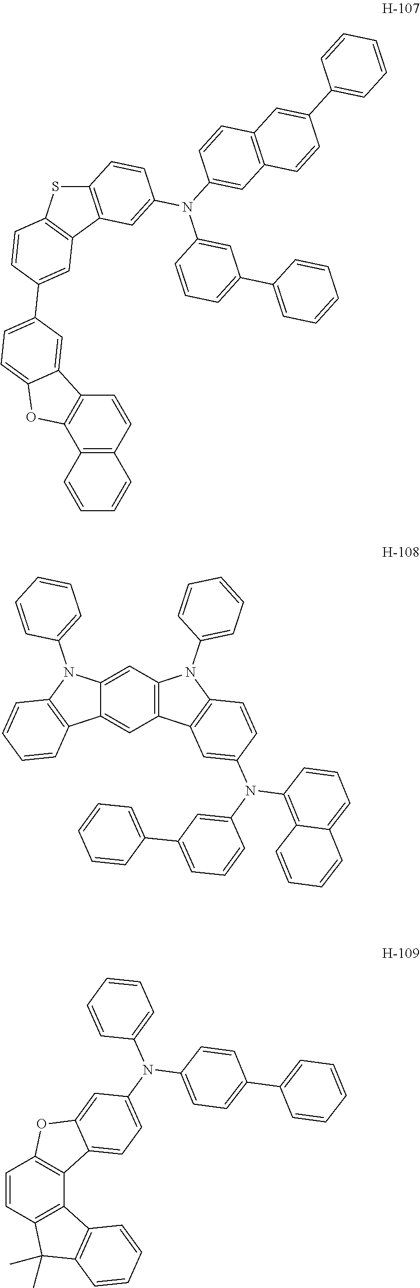 [SMILES: c1ccc(-c2cccc(N(c3ccc4cc(-c5ccccc5)ccc4c3)c3ccc4sc5ccc(-c6ccc7oc8c9ccccc9ccc8c7c6)cc5c4c3)c2)cc1 ; c1ccc(-c2cccc(N(c3ccc4c(c3)c3cc5c6ccccc6n(-c6ccccc6)c5cc3n4-c3ccccc3)c3cccc4ccccc34)c2)cc1 ; CC1(C)c2ccccc2-c2c1ccc1oc3cc(N(c4ccccc4)c4ccc(-c5ccccc5)cc4)ccc3c21]

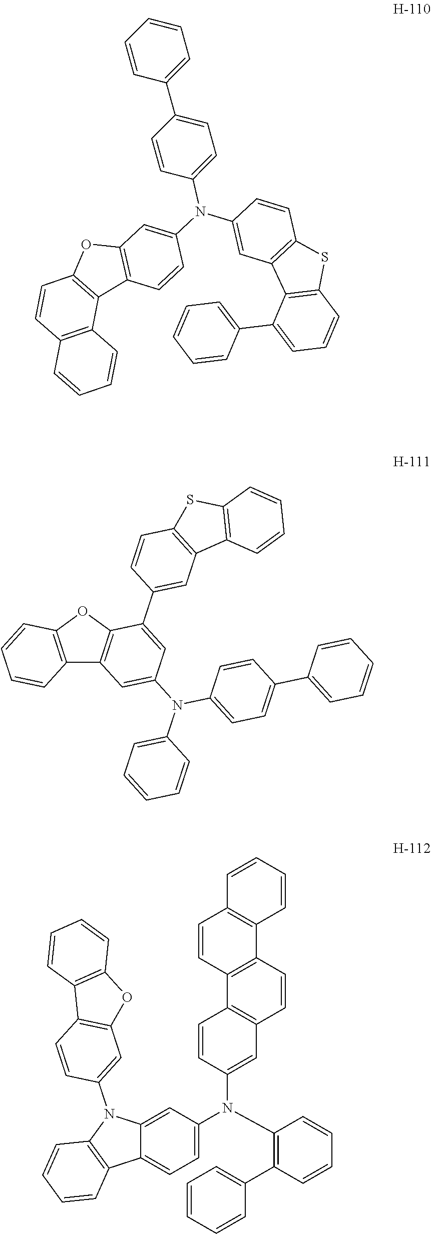 [SMILES: c1ccc(-c2ccc(N(c3ccc4c(c3)oc3ccc5ccccc5c34)c3ccc4sc5cccc(-c6ccccc6)c5c4c3)cc2)cc1 ; c1ccc(-c2ccc(N(c3ccccc3)c3cc(-c4ccc5sc6ccccc6c5c4)c4oc5ccccc5c4c3)cc2)cc1 ; c1ccc(-c2ccccc2N(c2ccc3c(ccc4c5ccccc5ccc34)c2)c2ccc3c4ccccc4n(-c4ccc5c(c4)oc4ccccc45)c3c2)cc1]

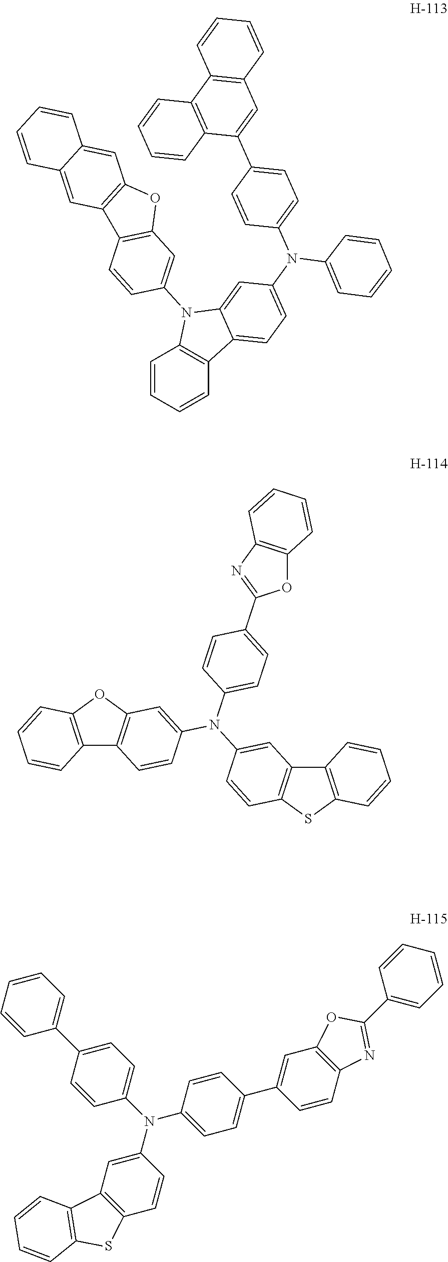 [SMILES: c1ccc(N(c2ccc(-c3cc4ccccc4c4ccccc34)cc2)c2ccc3c4ccccc4n(-c4ccc5c(c4)oc4cc6ccccc6cc45)c3c2)cc1 ; c1ccc2oc(-c3ccc(N(c4ccc5c(c4)oc4ccccc45)c4ccc5sc6ccccc6c5c4)cc3)nc2c1 ; c1ccc(-c2ccc(N(c3ccc(-c4ccc5nc(-c6ccccc6)oc5c4)cc3)c3ccc4sc5ccccc5c4c3)cc2)cc1]

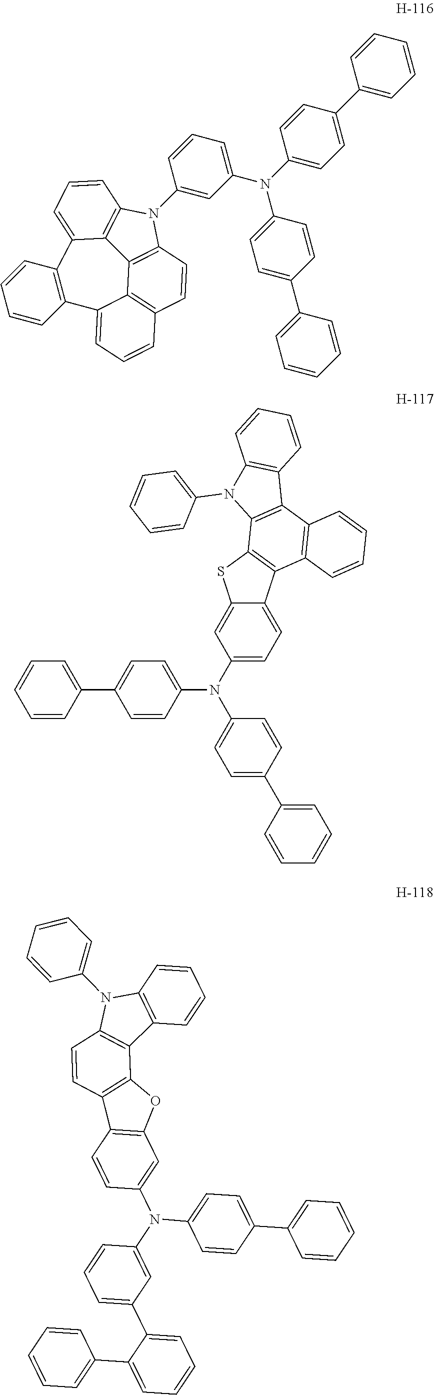 [SMILES: c1ccc(-c2ccc(N(c3ccc(-c4ccccc4)cc3)c3cccc(-n4c5cccc6c5c5c7c(cccc7ccc54)-c4ccccc4-6)c3)cc2)cc1 ; c1ccc(-c2ccc(N(c3ccc(-c4ccccc4)cc3)c3ccc4c(c3)sc3c4c4ccccc4c4c5ccccc5n(-c5ccccc5)c34)cc2)cc1 ; c1ccc(-c2ccc(N(c3cccc(-c4ccccc4-c4ccccc4)c3)c3ccc4c(c3)oc3c4ccc4c3c3ccccc3n4-c3ccccc3)cc2)cc1]

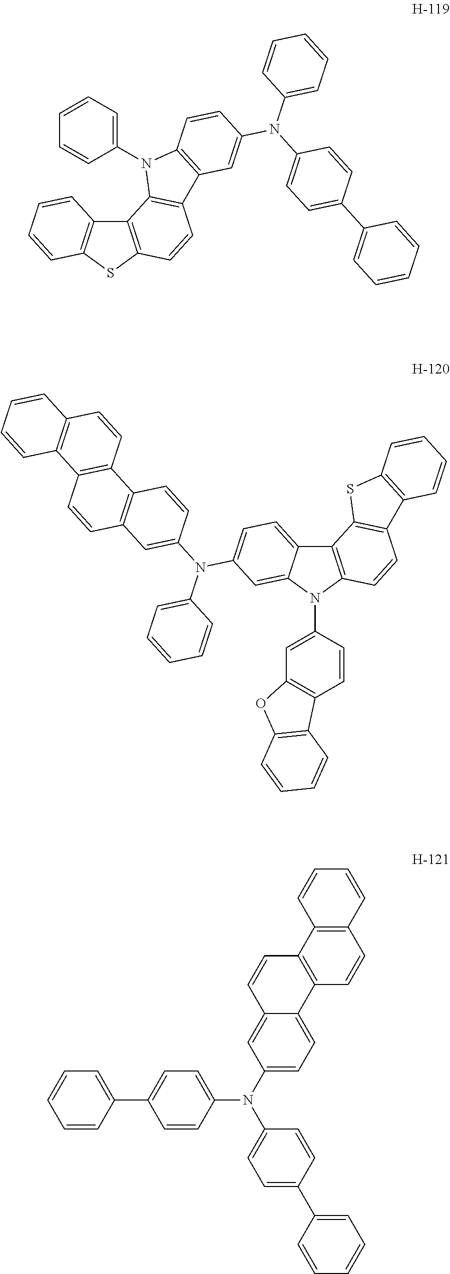 [SMILES: c1ccc(-c2ccc(N(c3ccccc3)c3ccc4c(c3)c3ccc5sc6ccccc6c5c3n4-c3ccccc3)cc2)cc1 ; c1ccc(N(c2ccc3c(ccc4c5ccccc5ccc34)c2)c2ccc3c4c5sc6ccccc6c5ccc4n(-c4ccc5c(c4)oc4ccccc45)c3c2)cc1 ; c1ccc(-c2ccc(N(c3ccc(-c4ccccc4)cc3)c3ccc4c(ccc5c6ccccc6ccc45)c3)cc2)cc1]

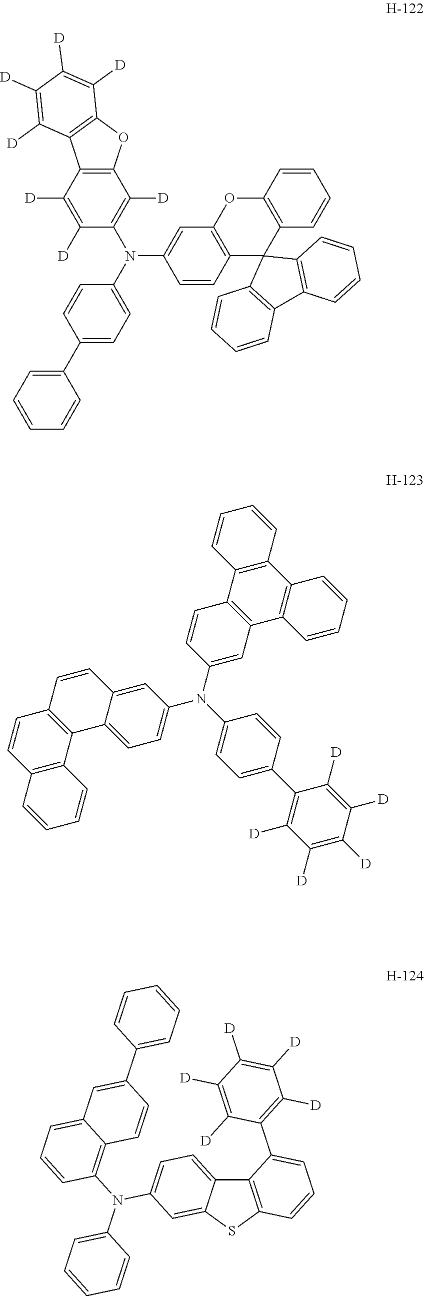 [SMILES: [2H]c1c([2H])c([2H])c2c(oc3c([2H])c(N(c4ccc(-c5ccccc5)cc4)c4ccc5c(c4)Oc4ccccc4C54c5ccccc5-c5ccccc54)c([2H])c([2H])c32)c1[2H] ; [2H]c1c([2H])c([2H])c(-c2ccc(N(c3ccc4c(ccc5ccc6ccccc6c54)c3)c3ccc4c5ccccc5c5ccccc5c4c3)cc2)c([2H])c1[2H] ; [2H]c1c([2H])c([2H])c(-c2cccc3sc4cc(N(c5ccccc5)c5cccc6cc(-c7ccccc7)ccc56)ccc4c23)c([2H])c1[2H]]

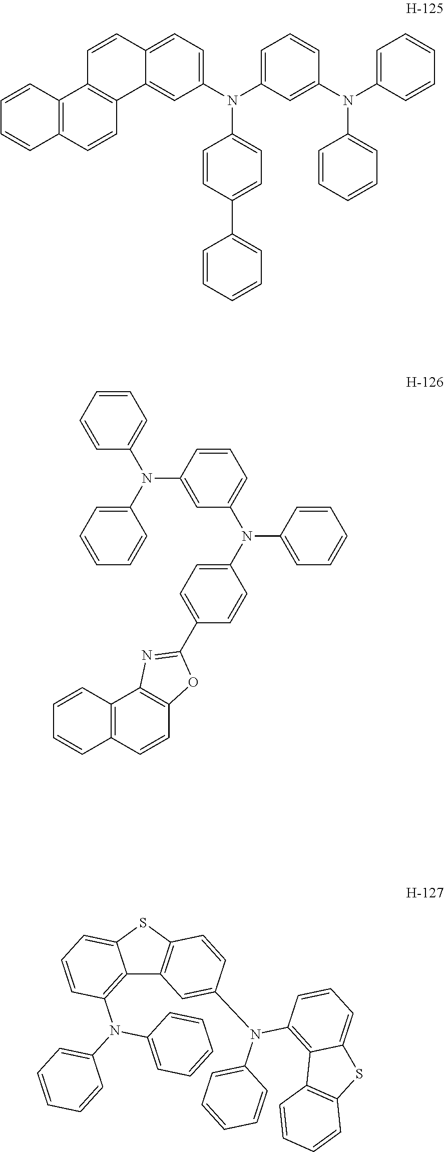 [SMILES: c1ccc(-c2ccc(N(c3cccc(N(c4ccccc4)c4ccccc4)c3)c3ccc4ccc5c6ccccc6ccc5c4c3)cc2)cc1 ; c1ccc(N(c2ccccc2)c2cccc(N(c3ccccc3)c3ccc(-c4nc5c(ccc6ccccc65)o4)cc3)c2)cc1 ; c1ccc(N(c2ccc3sc4cccc(N(c5ccccc5)c5ccccc5)c4c3c2)c2cccc3sc4ccccc4c23)cc1]

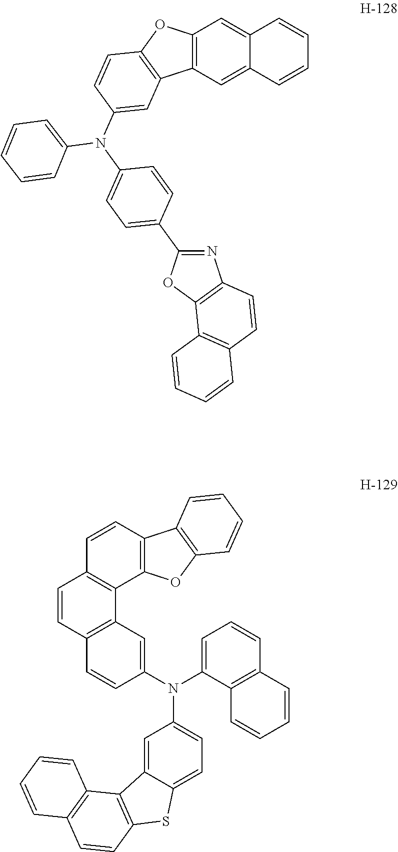 [SMILES: c1ccc(N(c2ccc(-c3nc4ccc5ccccc5c4o3)cc2)c2ccc3oc4cc5ccccc5cc4c3c2)cc1 ; c1ccc2c(N(c3ccc4sc5ccc6ccccc6c5c4c3)c3ccc4ccc5ccc6c7ccccc7oc6c5c4c3)cccc2c1]

Specifically, the compound represented by Formula 5 may be any one of the following compounds S-1 to S-116, but is not limited thereto.

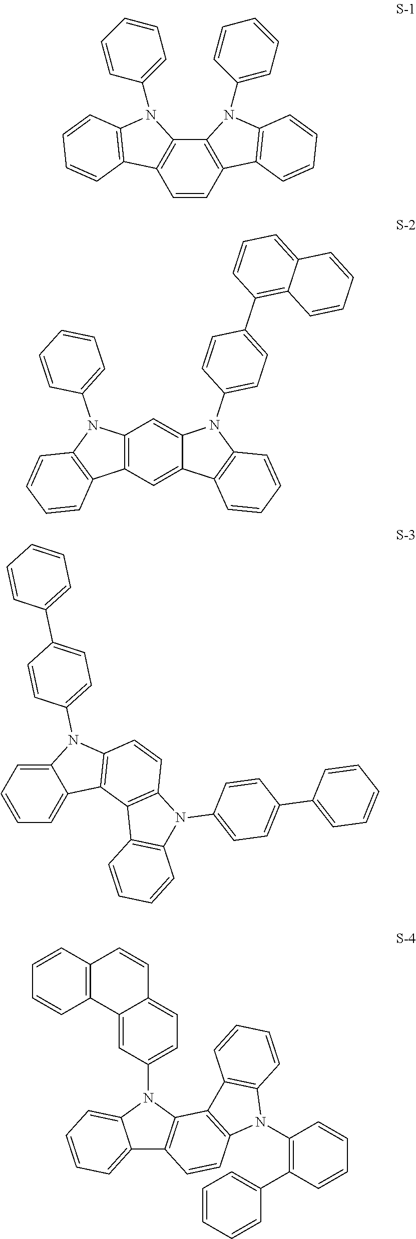

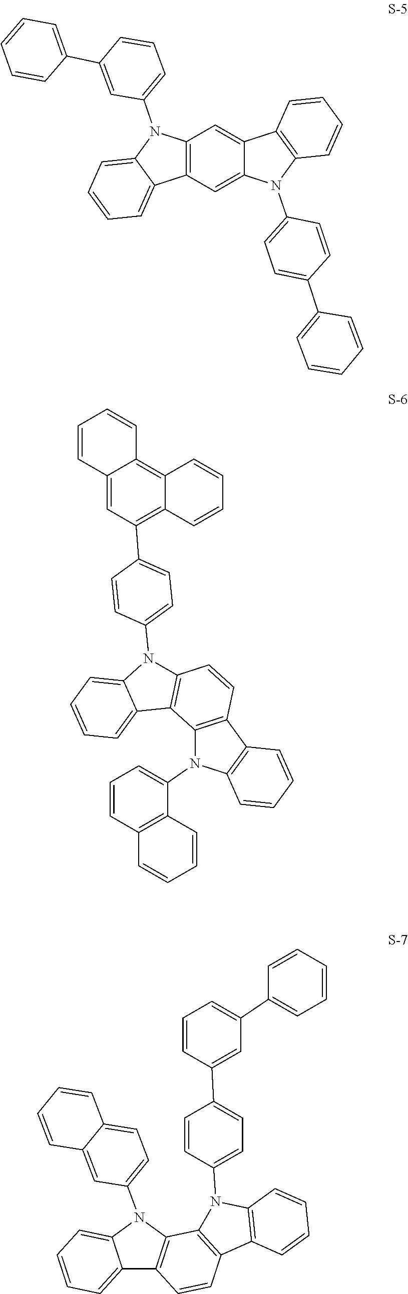 [SMILES: c1ccc(-c2ccc(-n3c4ccccc4c4cc5c(cc43)c3ccccc3n5-c3cccc(-c4ccccc4)c3)cc2)cc1 ; c1ccc2c(-n3c4ccccc4c4ccc5c(c6ccccc6n5-c5ccc(-c6cc7ccccc7c7ccccc67)cc5)c43)cccc2c1 ; c1ccc(-c2cccc(-c3ccc(-n4c5ccccc5c5ccc6c7ccccc7n(-c7ccc8ccccc8c7)c6c54)cc3)c2)cc1]

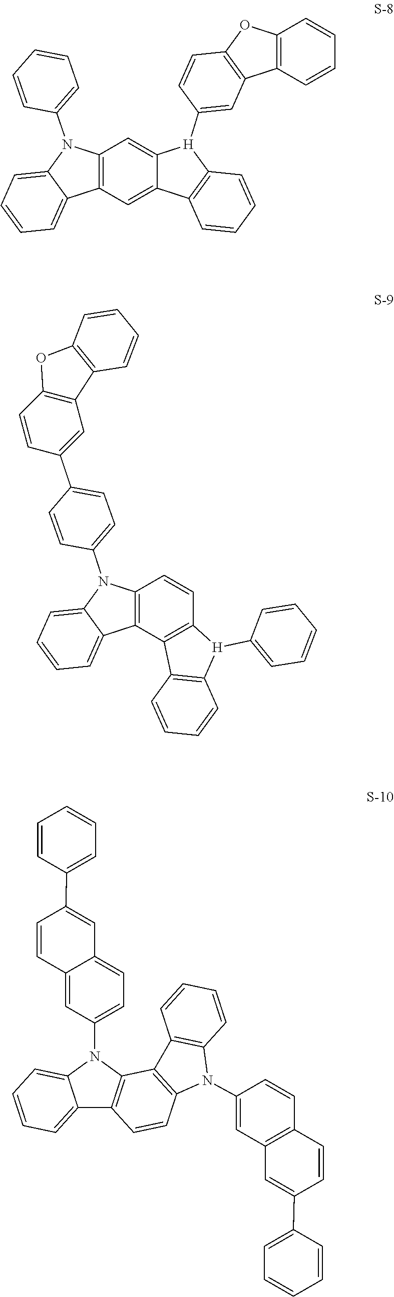

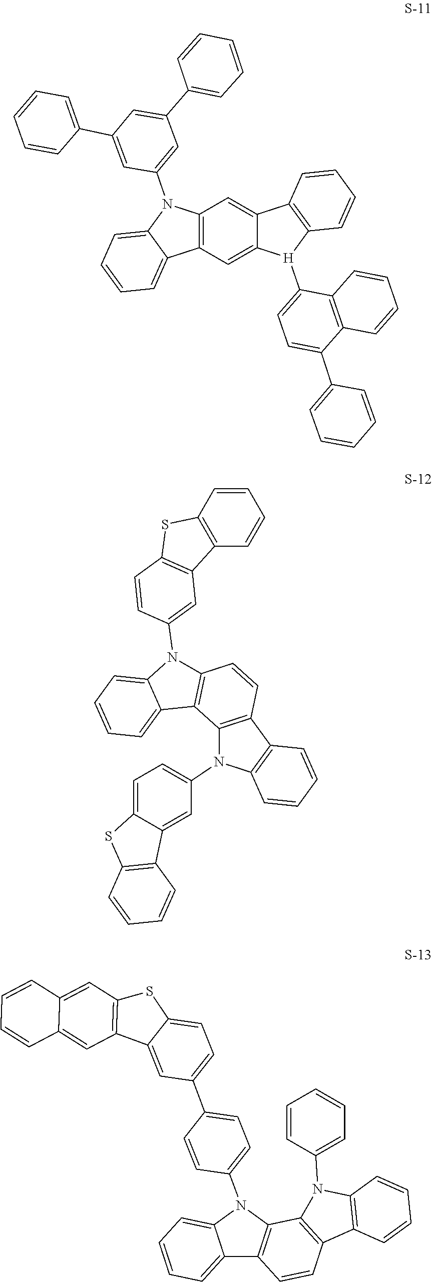

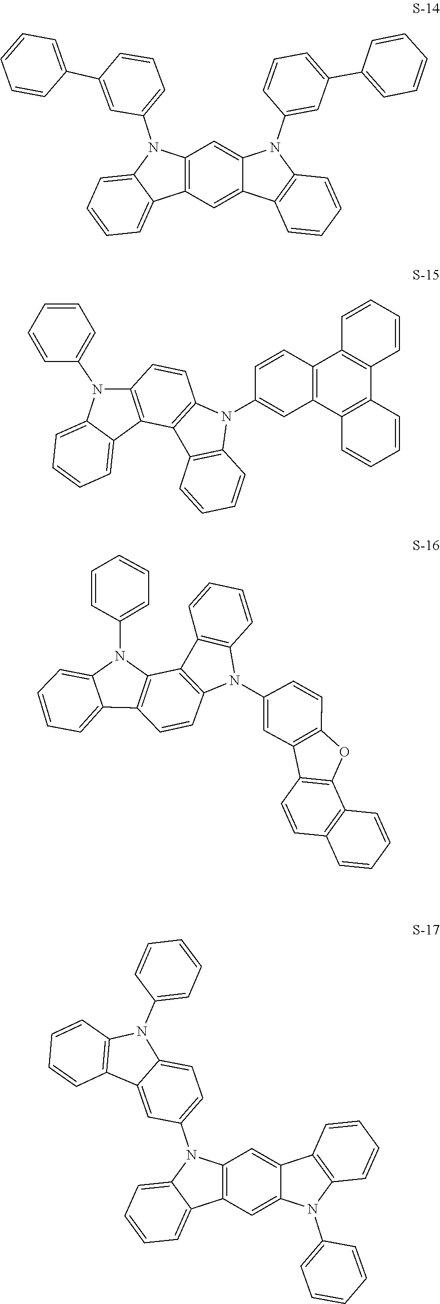 [SMILES: c1ccc(-c2cccc(-n3c4ccccc4c4cc5c6ccccc6n(-c6cccc(-c7ccccc7)c6)c5cc43)c2)cc1 ; c1ccc(-n2c3ccccc3c3c4c5ccccc5n(-c5ccc6c7ccccc7c7ccccc7c6c5)c4ccc32)cc1 ; c1ccc(-n2c3ccccc3c3ccc4c(c5ccccc5n4-c4ccc5oc6c7ccccc7ccc6c5c4)c32)cc1 ; c1ccc(-n2c3ccccc3c3cc(-n4c5ccccc5c5cc6c(cc54)c4ccccc4n6-c4ccccc4)ccc32)cc1]

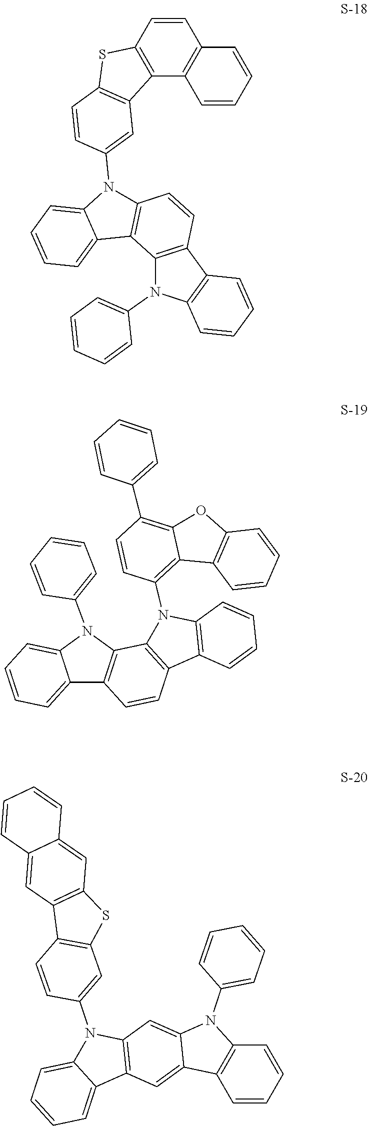 [SMILES: c1ccc(-n2c3ccccc3c3ccc4c(c5ccccc5n4-c4ccc5sc6ccc7ccccc7c6c5c4)c32)cc1 ; c1ccc(-c2ccc(-n3c4ccccc4c4ccc5c6ccccc6n(-c6ccccc6)c5c43)c3c2oc2ccccc23)cc1 ; c1ccc(-n2c3ccccc3c3cc4c5ccccc5n(-c5ccc6c(c5)sc5cc7ccccc7cc56)c4cc32)cc1]

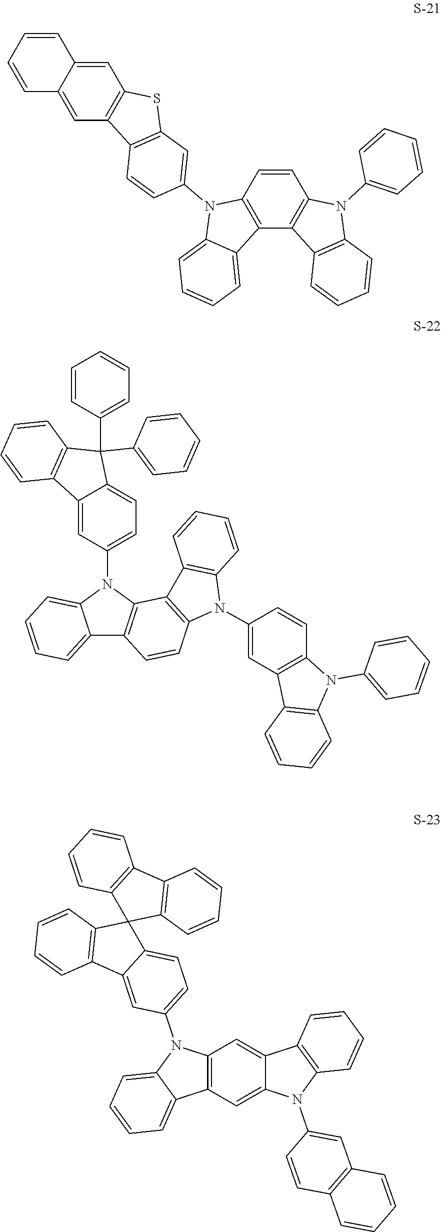 [SMILES: c1ccc(-n2c3ccccc3c3c4c5ccccc5n(-c5ccc6c(c5)sc5cc7ccccc7cc56)c4ccc32)cc1 ; c1ccc(-n2c3ccccc3c3cc(-n4c5ccccc5c5c4ccc4c6ccccc6n(-c6ccc7c(c6)-c6ccccc6C7(c6ccccc6)c6ccccc6)c45)ccc32)cc1 ; c1ccc2c(c1)-c1ccccc1C21c2ccccc2-c2cc(-n3c4ccccc4c4cc5c(cc43)c3ccccc3n5-c3ccc4ccccc4c3)ccc21]

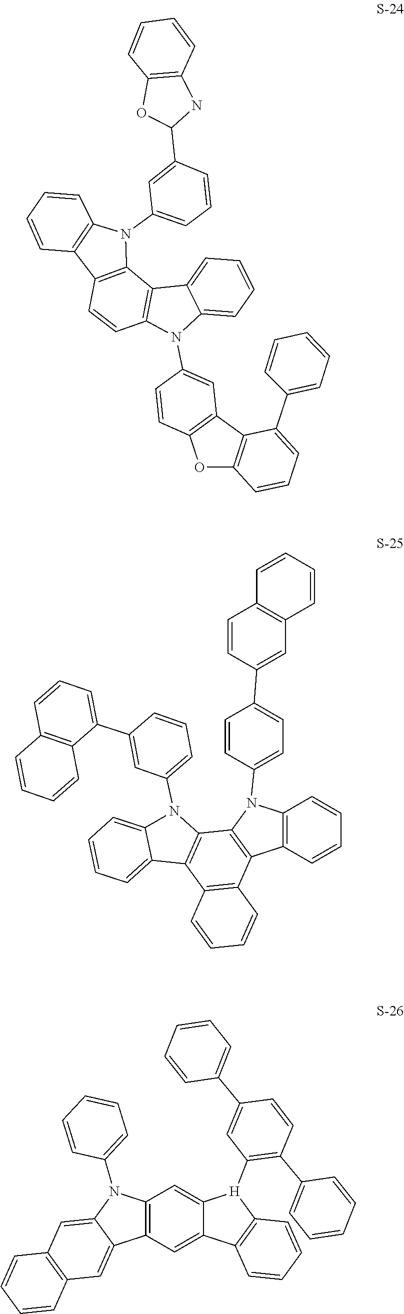

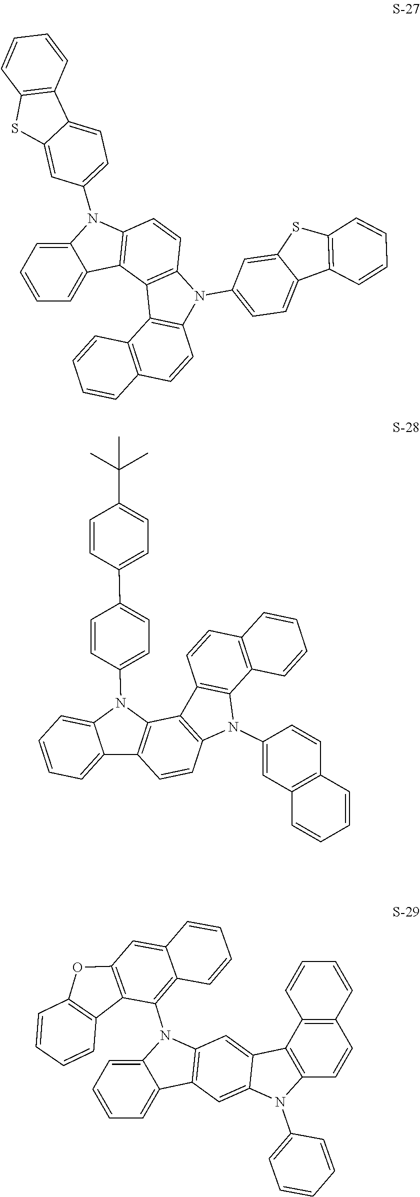

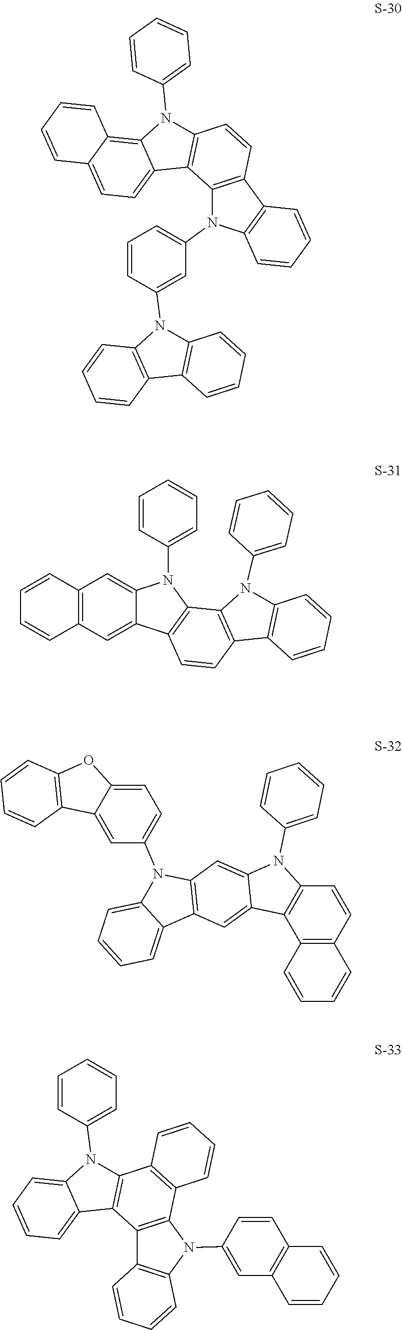 [SMILES: c1ccc(-n2c3ccc4c5ccccc5n(-c5cccc(-n6c7ccccc7c7ccccc76)c5)c4c3c3ccc4ccccc4c32)cc1 ; c1ccc(-n2c3ccccc3c3ccc4c5cc6ccccc6cc5n(-c5ccccc5)c4c32)cc1 ; c1ccc(-n2c3cc4c(cc3c3c5ccccc5ccc32)c2ccccc2n4-c2ccc3oc4ccccc4c3c2)cc1 ; c1ccc(-n2c3ccccc3c3c4c5ccccc5n(-c5ccc6ccccc6c5)c4c4ccccc4c32)cc1]

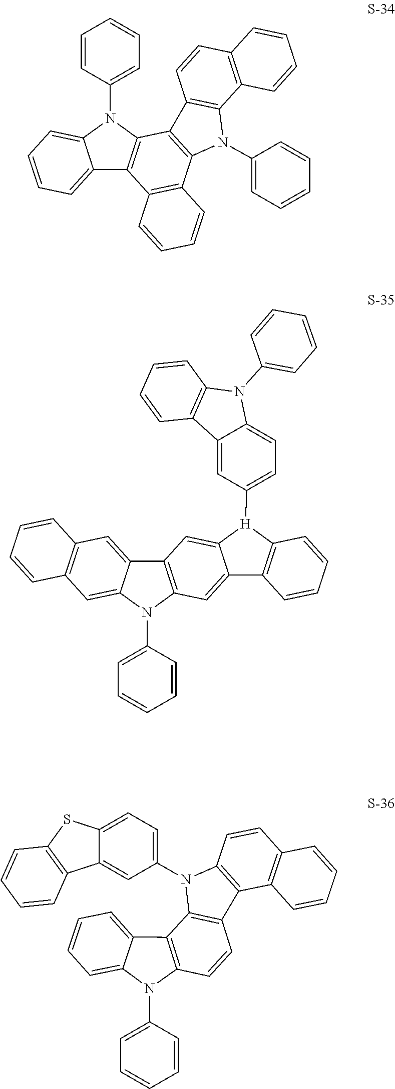

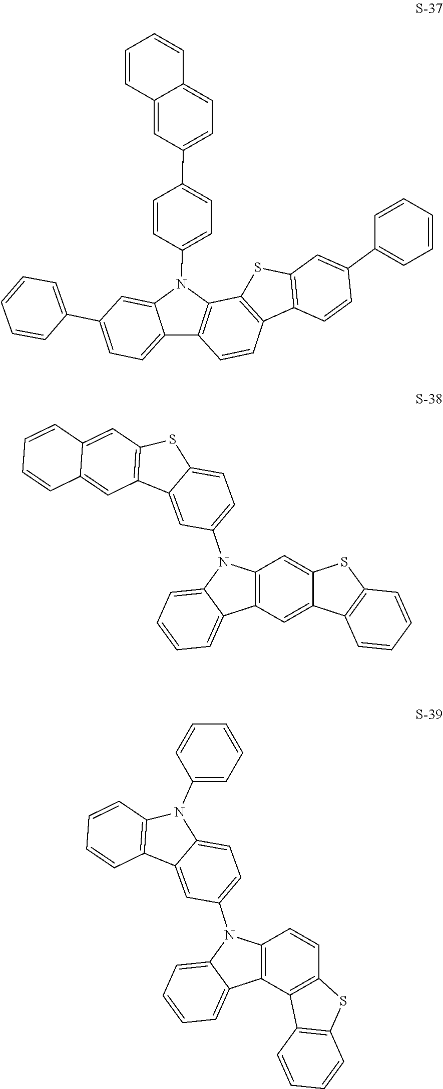 [SMILES: c1ccc(-c2ccc3c(c2)sc2c3ccc3c4ccc(-c5ccccc5)cc4n(-c4ccc(-c5ccc6ccccc6c5)cc4)c32)cc1 ; c1ccc2cc3c(cc2c1)sc1ccc(-n2c4ccccc4c4cc5c(cc42)sc2ccccc25)cc13 ; c1ccc(-n2c3ccccc3c3cc(-n4c5ccccc5c5c6c(ccc54)sc4ccccc46)ccc32)cc1]

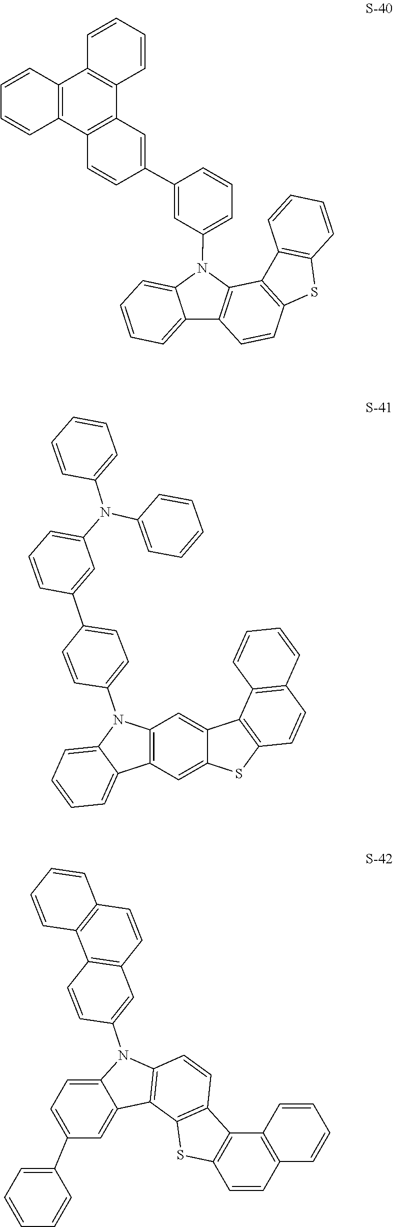 [SMILES: c1cc(-c2ccc3c4ccccc4c4ccccc4c3c2)cc(-n2c3ccccc3c3ccc4sc5ccccc5c4c32)c1 ; c1ccc(N(c2ccccc2)c2cccc(-c3ccc(-n4c5ccccc5c5cc6sc7ccc8ccccc8c7c6cc54)cc3)c2)cc1 ; c1ccc(-c2ccc3c(c2)c2c4sc5ccc6ccccc6c5c4ccc2n3-c2ccc3c(ccc4ccccc43)c2)cc1]

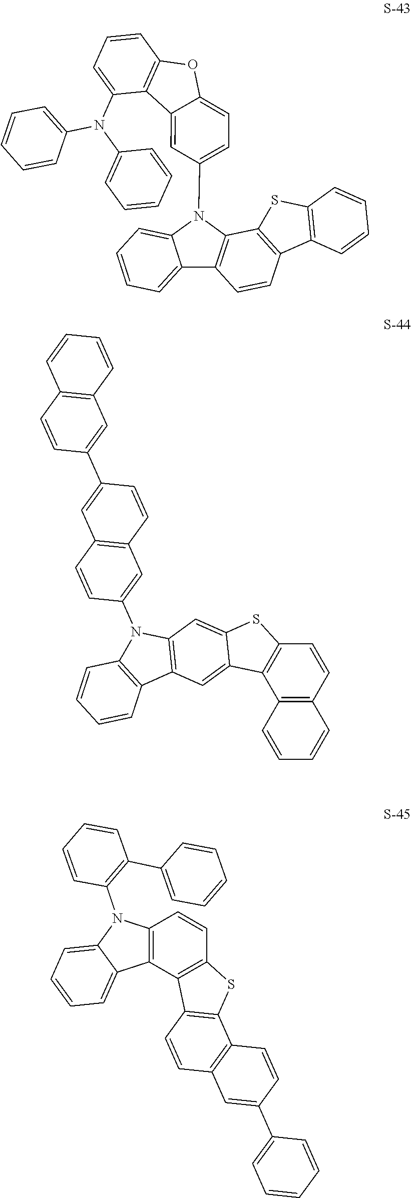 [SMILES: c1ccc(N(c2ccccc2)c2cccc3oc4ccc(-n5c6ccccc6c6ccc7c8ccccc8sc7c65)cc4c23)cc1 ; c1ccc2cc(-c3ccc4cc(-n5c6ccccc6c6cc7c(cc65)sc5ccc6ccccc6c57)ccc4c3)ccc2c1 ; c1ccc(-c2ccc3c(ccc4c3sc3ccc5c(c6ccccc6n5-c5ccccc5-c5ccccc5)c34)c2)cc1]

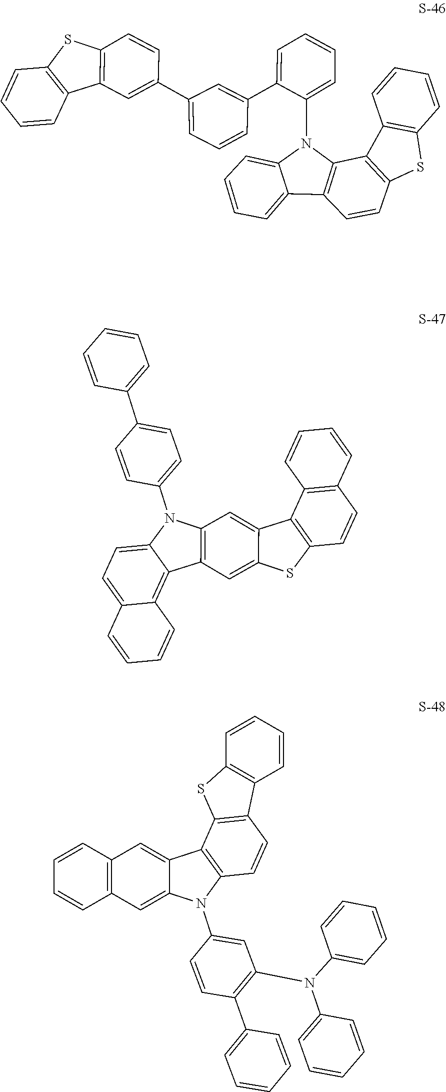 [SMILES: c1cc(-c2ccc3sc4ccccc4c3c2)cc(-c2ccccc2-n2c3ccccc3c3ccc4sc5ccccc5c4c32)c1 ; c1ccc(-c2ccc(-n3c4cc5c(cc4c4c6ccccc6ccc43)sc3ccc4ccccc4c35)cc2)cc1 ; c1ccc(-c2ccc(-n3c4cc5ccccc5cc4c4c5sc6ccccc6c5ccc43)cc2N(c2ccccc2)c2ccccc2)cc1]

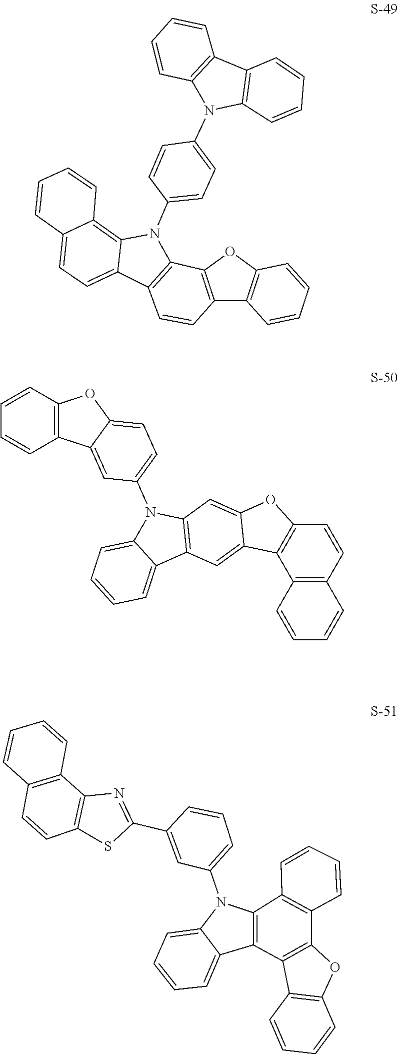 [SMILES: c1ccc2c(c1)ccc1c3ccc4c5ccccc5oc4c3n(-c3ccc(-n4c5ccccc5c5ccccc54)cc3)c21 ; c1ccc2c(c1)ccc1oc3cc4c(cc3c12)c1ccccc1n4-c1ccc2oc3ccccc3c2c1 ; c1cc(-c2nc3c(ccc4ccccc43)s2)cc(-n2c3ccccc3c3c4c5ccccc5oc4c4ccccc4c32)c1]

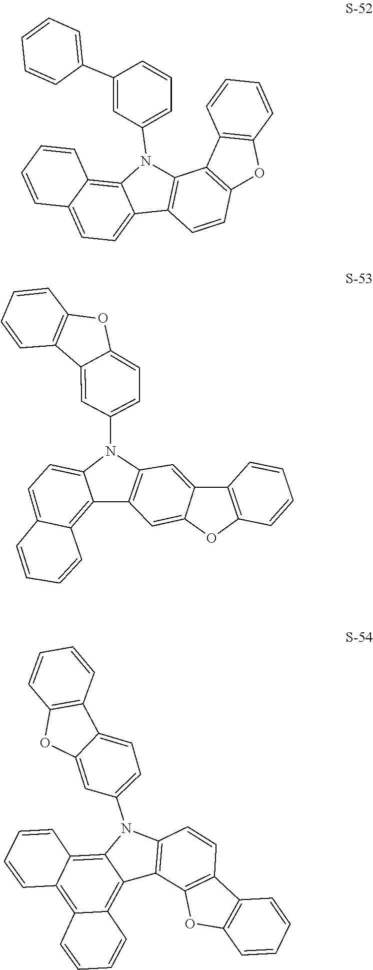 [SMILES: c1ccc(-c2cccc(-n3c4c5ccccc5ccc4c4ccc5oc6ccccc6c5c43)c2)cc1 ; c1ccc2c(c1)ccc1c2c2cc3oc4ccccc4c3cc2n1-c1ccc2oc3ccccc3c2c1 ; c1ccc2c(c1)oc1cc(-n3c4ccc5c6ccccc6oc5c4c4c5ccccc5c5ccccc5c43)ccc12]

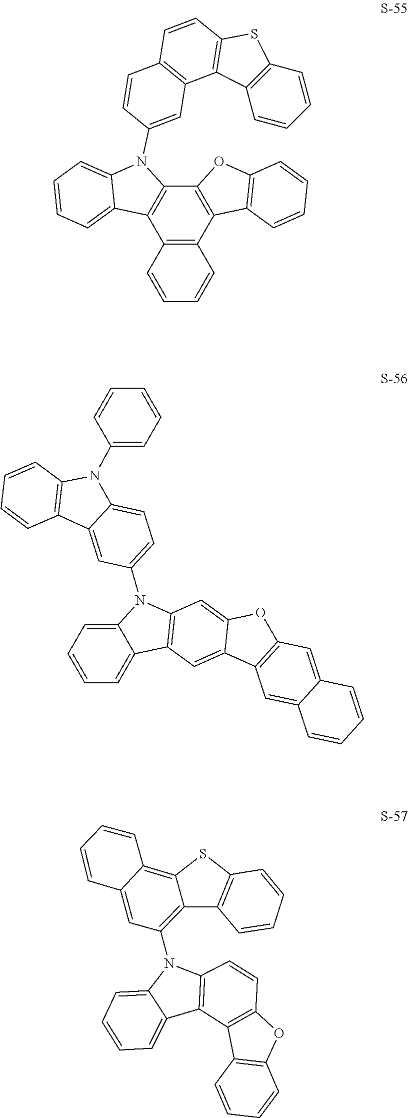 [SMILES: c1ccc2c(c1)oc1c2c2ccccc2c2c3ccccc3n(-c3ccc4ccc5sc6ccccc6c5c4c3)c12 ; c1ccc(-n2c3ccccc3c3cc(-n4c5ccccc5c5cc6c(cc54)oc4cc5ccccc5cc46)ccc32)cc1 ; c1ccc2c(c1)cc(-n1c3ccccc3c3c4c(ccc31)oc1ccccc14)c1c3ccccc3sc21]

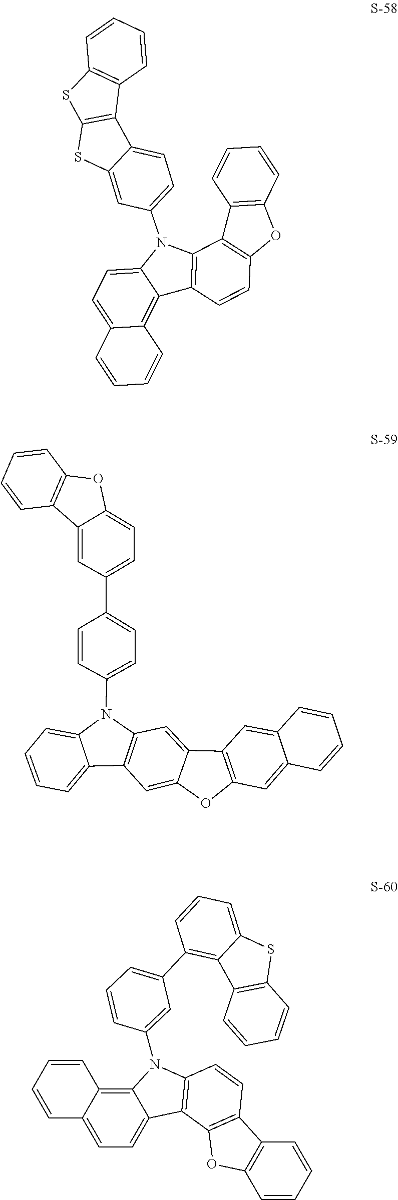 [SMILES: c1ccc2c(c1)ccc1c2c2ccc3oc4ccccc4c3c2n1-c1ccc2c(c1)sc1sc3ccccc3c12 ; c1ccc2cc3c(cc2c1)oc1cc2c4ccccc4n(-c4ccc(-c5ccc6oc7ccccc7c6c5)cc4)c2cc13 ; c1cc(-c2cccc3sc4ccccc4c23)cc(-n2c3ccc4c5ccccc5oc4c3c3ccc4ccccc4c32)c1]

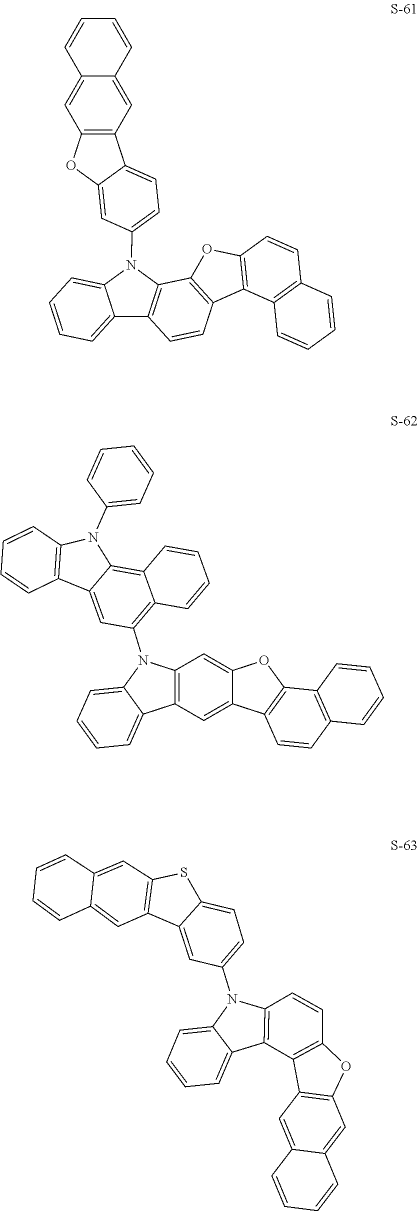 [SMILES: c1ccc2cc3c(cc2c1)oc1cc(-n2c4ccccc4c4ccc5c(oc6ccc7ccccc7c65)c42)ccc13 ; c1ccc(-n2c3ccccc3c3cc(-n4c5ccccc5c5cc6c(cc54)oc4c5ccccc5ccc64)c4ccccc4c32)cc1 ; c1ccc2cc3c(cc2c1)oc1ccc2c(c4ccccc4n2-c2ccc4sc5cc6ccccc6cc5c4c2)c13]

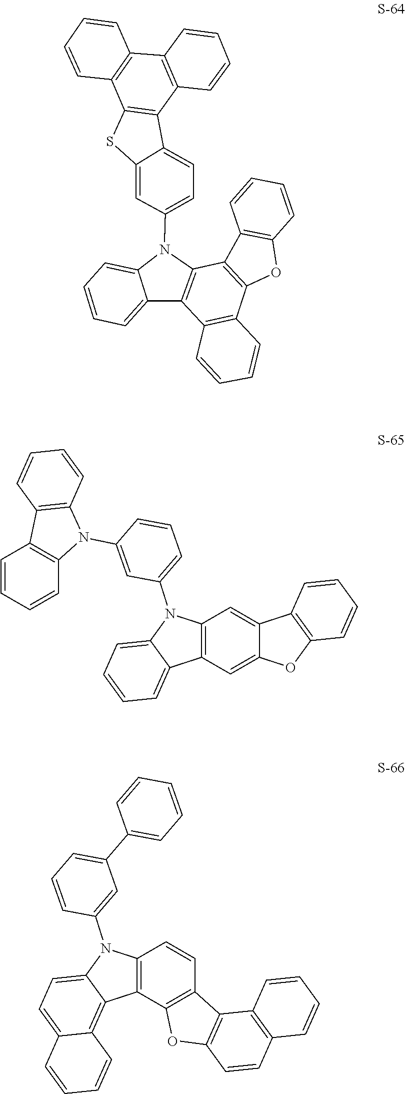 [SMILES: c1ccc2c(c1)oc1c3ccccc3c3c4ccccc4n(-c4ccc5c(c4)sc4c6ccccc6c6ccccc6c54)c3c21 ; c1cc(-n2c3ccccc3c3ccccc32)cc(-n2c3ccccc3c3cc4oc5ccccc5c4cc32)c1 ; c1ccc(-c2cccc(-n3c4ccc5ccccc5c4c4c5oc6ccc7ccccc7c6c5ccc43)c2)cc1]

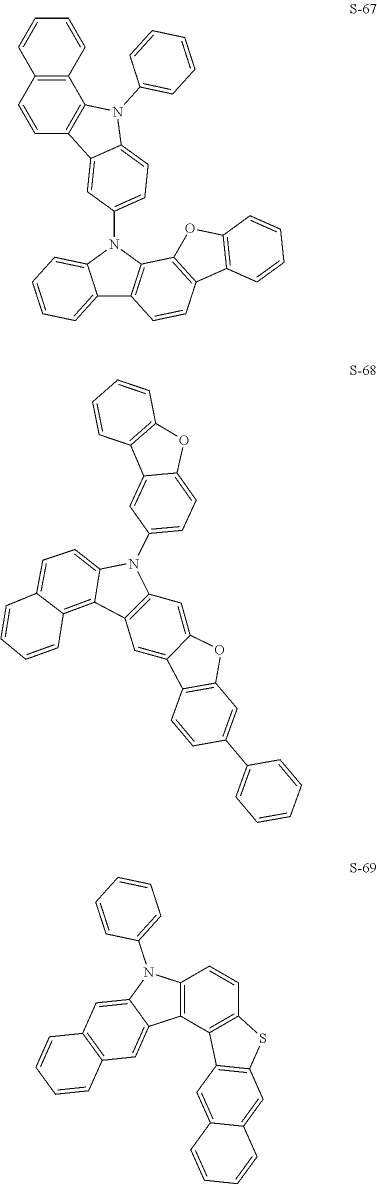 [SMILES: c1ccc(-n2c3ccc(-n4c5ccccc5c5ccc6c7ccccc7oc6c54)cc3c3ccc4ccccc4c32)cc1 ; c1ccc(-c2ccc3c(c2)oc2cc4c(cc23)c2c3ccccc3ccc2n4-c2ccc3oc4ccccc4c3c2)cc1 ; c1ccc(-n2c3cc4ccccc4cc3c3c4c(ccc32)sc2cc3ccccc3cc24)cc1]

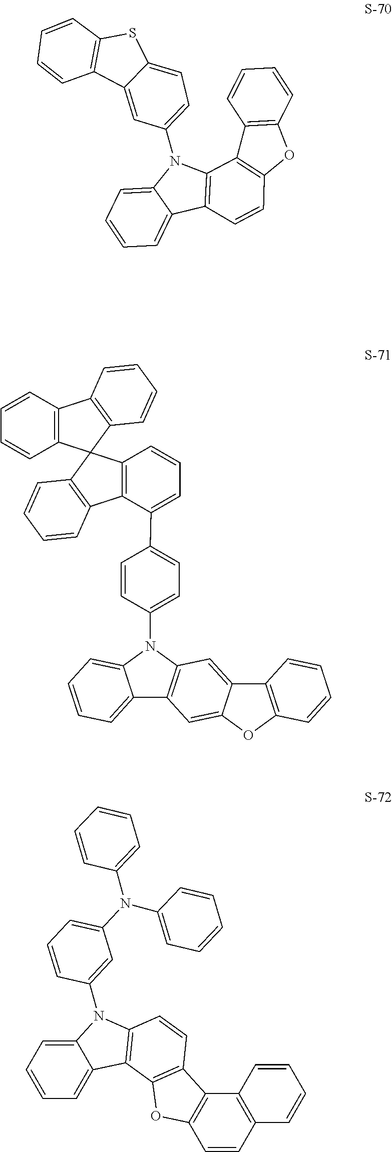 [SMILES: c1ccc2c(c1)oc1ccc3c4ccccc4n(-c4ccc5sc6ccccc6c5c4)c3c12 ; c1ccc2c(c1)-c1ccccc1C21c2ccccc2-c2c(-c3ccc(-n4c5ccccc5c5cc6oc7ccccc7c6cc54)cc3)cccc21 ; c1ccc(N(c2ccccc2)c2cccc(-n3c4ccccc4c4c5oc6ccc7ccccc7c6c5ccc43)c2)cc1]

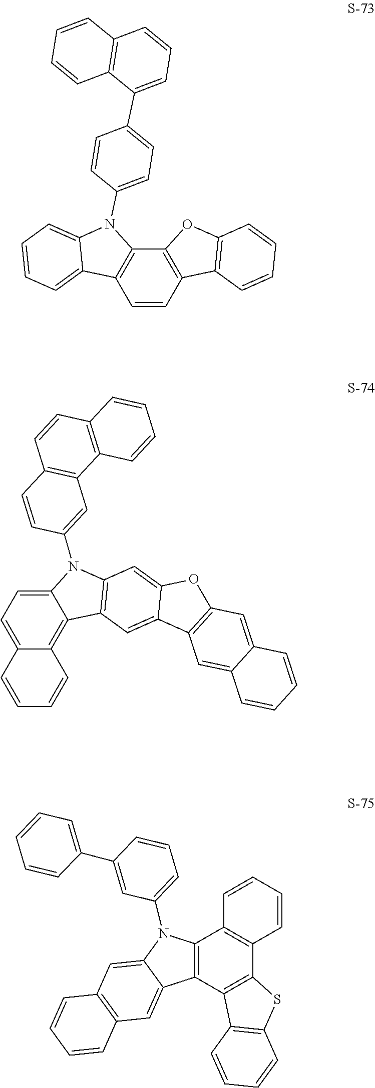 [SMILES: c1ccc2c(-c3ccc(-n4c5ccccc5c5ccc6c7ccccc7oc6c54)cc3)cccc2c1 ; c1ccc2cc3c(cc2c1)oc1cc2c(cc13)c1c3ccccc3ccc1n2-c1ccc2ccc3ccccc3c2c1 ; c1ccc(-c2cccc(-n3c4cc5ccccc5cc4c4c5c6ccccc6sc5c5ccccc5c43)c2)cc1]

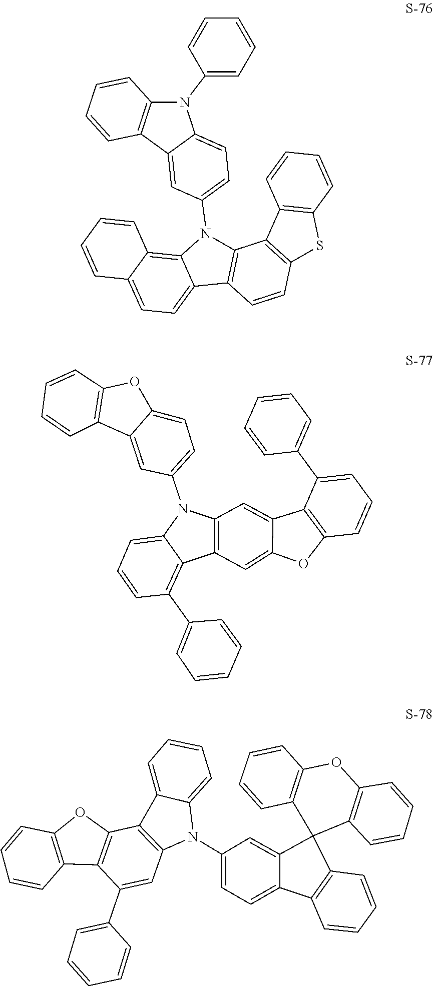 [SMILES: c1ccc(-n2c3ccccc3c3cc(-n4c5c6ccccc6ccc5c5ccc6sc7ccccc7c6c54)ccc32)cc1 ; c1ccc(-c2cccc3oc4cc5c6c(-c7ccccc7)cccc6n(-c6ccc7oc8ccccc8c7c6)c5cc4c23)cc1 ; c1ccc(-c2cc3c(c4ccccc4n3-c3ccc4c(c3)C3(c5ccccc5Oc5ccccc53)c3ccccc3-4)c3oc4ccccc4c23)cc1]

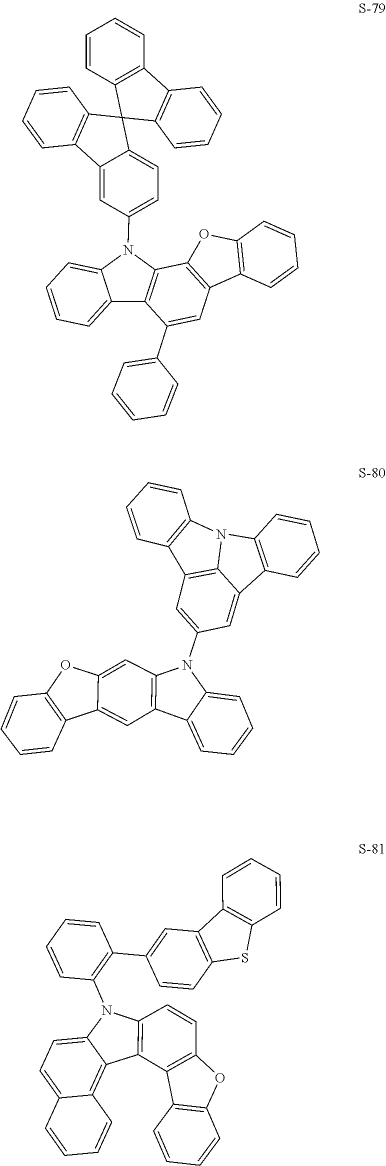 [SMILES: c1ccc(-c2cc3c4ccccc4oc3c3c2c2ccccc2n3-c2ccc3c(c2)-c2ccccc2C32c3ccccc3-c3ccccc32)cc1 ; c1ccc2c(c1)oc1cc3c(cc12)c1ccccc1n3-c1cc2c3ccccc3n3c4ccccc4c(c1)c23 ; c1ccc(-n2c3ccc4ccccc4c3c3c4c(ccc32)oc2ccccc24)c(-c2ccc3sc4ccccc4c3c2)c1]

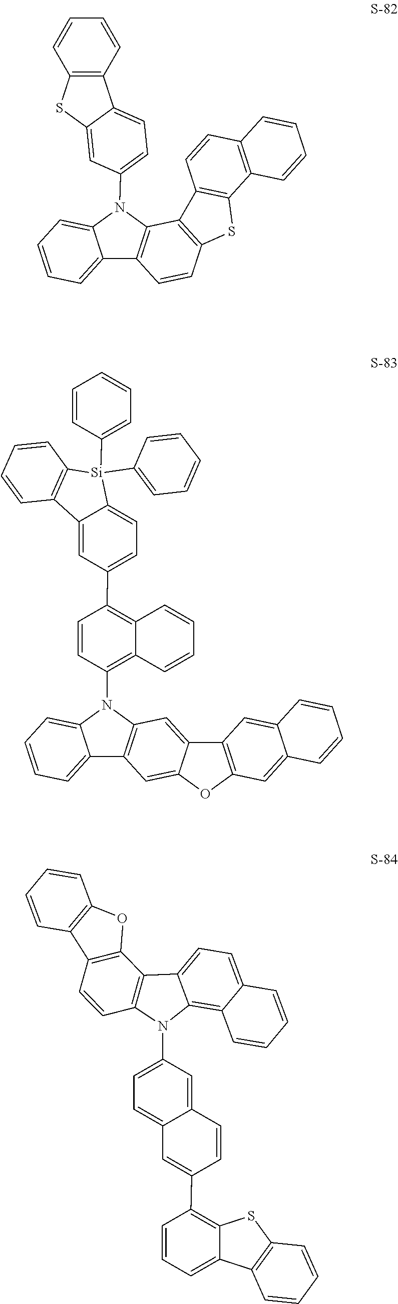 [SMILES: c1ccc2c(c1)ccc1c2sc2ccc3c4ccccc4n(-c4ccc5c(c4)sc4ccccc45)c3c21 ; c1ccc([Si]2(c3ccccc3)c3ccccc3-c3cc(-c4ccc(-n5c6ccccc6c6cc7oc8cc9ccccc9cc8c7cc65)c5ccccc45)ccc32)cc1 ; c1ccc2c(c1)ccc1c3c4oc5ccccc5c4ccc3n(-c3ccc4cc(-c5cccc6c5sc5ccccc56)ccc4c3)c21]

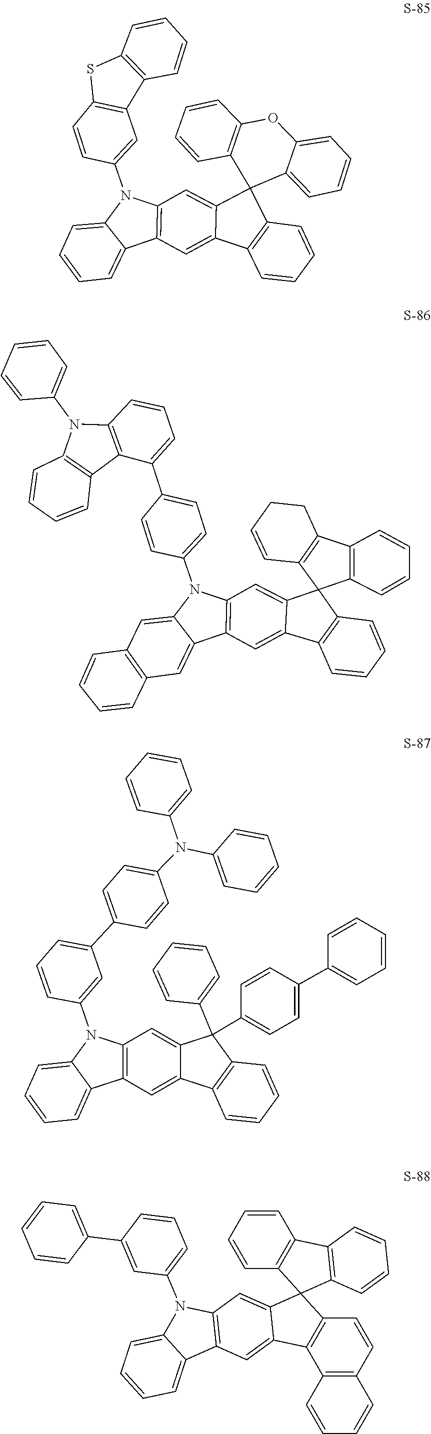 [SMILES: c1ccc2c(c1)Oc1ccccc1C21c2ccccc2-c2cc3c4ccccc4n(-c4ccc5sc6ccccc6c5c4)c3cc21 ; C=C1/C=C\C=C/CN(c2ccccc2)c2cccc(-c3ccc(-n4c5cc6c(cc5c5cc7ccccc7cc54)-c4ccccc4C64C5=C(CCC=C5)c5ccccc54)cc3)c21 ; c1ccc(-c2ccc(C3(c4ccccc4)c4ccccc4-c4cc5c6ccccc6n(-c6cccc(-c7ccc(N(c8ccccc8)c8ccccc8)cc7)c6)c5cc43)cc2)cc1 ; c1ccc(-c2cccc(-n3c4ccccc4c4cc5c(cc43)C3(c4ccccc4-c4ccccc43)c3ccc4ccccc4c3-5)c2)cc1]

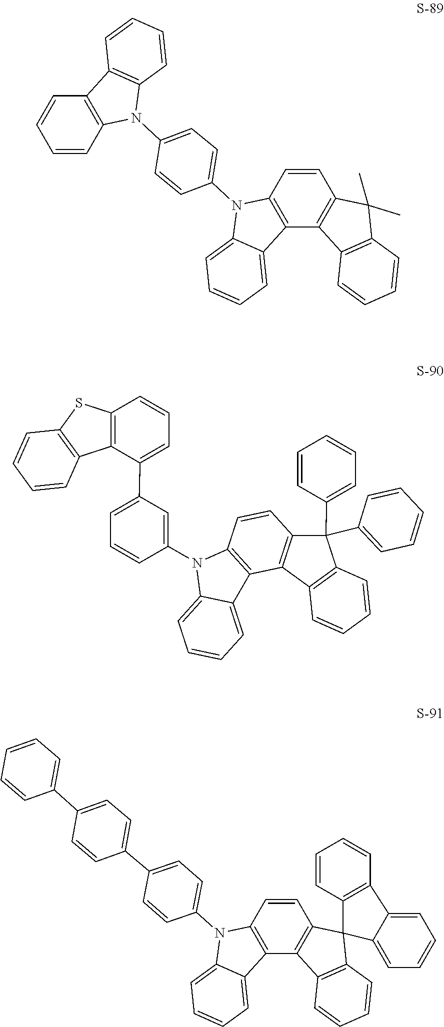 [SMILES: CC1(C)c2ccccc2-c2c1ccc1c2c2ccccc2n1-c1ccc(-n2c3ccccc3c3ccccc32)cc1 ; c1ccc(C2(c3ccccc3)c3ccccc3-c3c2ccc2c3c3ccccc3n2-c2cccc(-c3cccc4sc5ccccc5c34)c2)cc1 ; c1ccc(-c2ccc(-c3ccc(-n4c5ccccc5c5c6c(ccc54)C4(c5ccccc5-c5ccccc54)c4ccccc4-6)cc3)cc2)cc1]

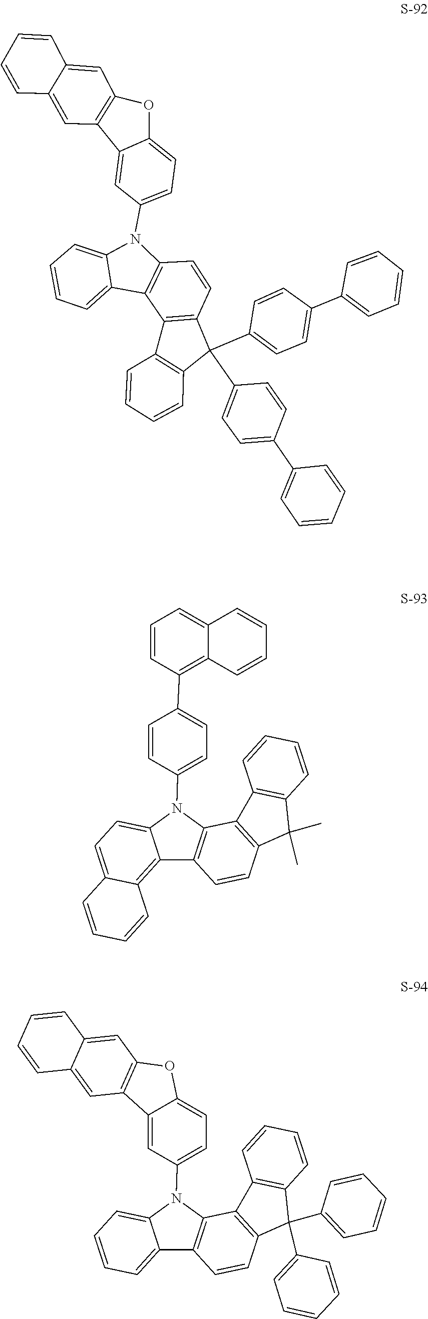 [SMILES: c1ccc(-c2ccc(C3(c4ccc(-c5ccccc5)cc4)c4ccccc4-c4c3ccc3c4c4ccccc4n3-c3ccc4oc5cc6ccccc6cc5c4c3)cc2)cc1 ; CC1(C)c2ccccc2-c2c1ccc1c3c4ccccc4ccc3n(-c3ccc(-c4cccc5ccccc45)cc3)c21 ; c1ccc(C2(c3ccccc3)c3ccccc3-c3c2ccc2c4ccccc4n(-c4ccc5oc6cc7ccccc7cc6c5c4)c32)cc1]

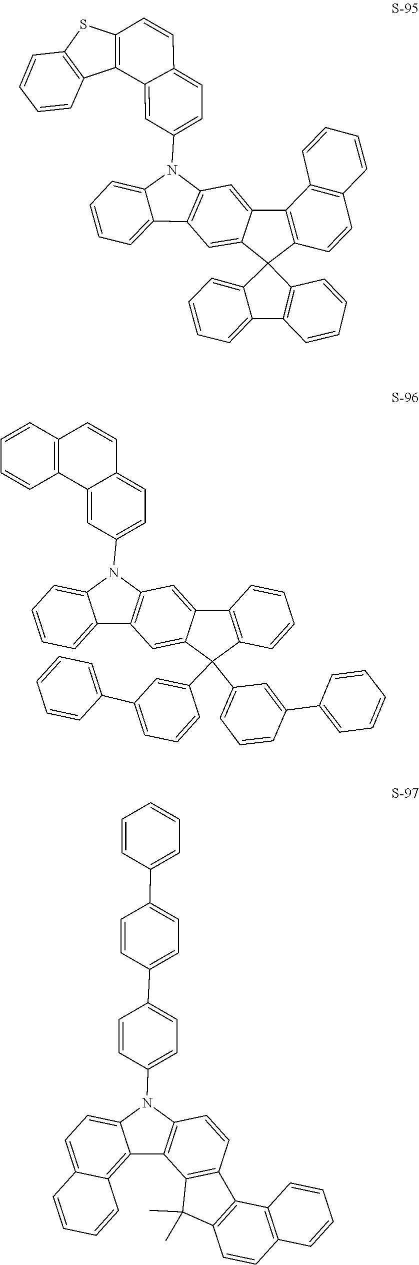 [SMILES: c1ccc2c(c1)-c1ccccc1C21c2cc3c4ccccc4n(-c4ccc5ccc6sc7ccccc7c6c5c4)c3cc2-c2c1ccc1ccccc21 ; c1ccc(-c2cccc(C3(c4cccc(-c5ccccc5)c4)c4ccccc4-c4cc5c(cc43)c3ccccc3n5-c3ccc4ccc5ccccc5c4c3)c2)cc1 ; CC1(C)c2ccc3ccccc3c2-c2ccc3c(c21)c1c2ccccc2ccc1n3-c1ccc(-c2ccc(-c3ccccc3)cc2)cc1]

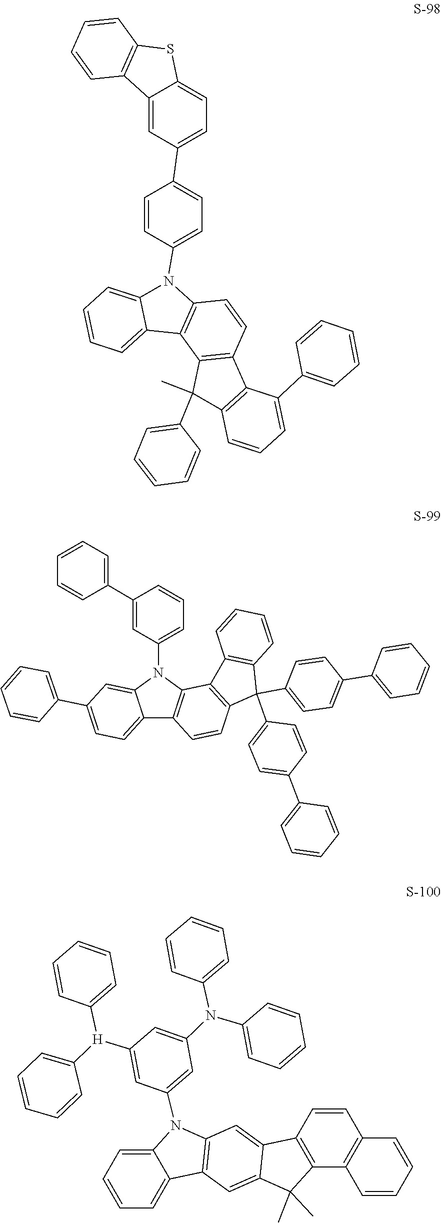

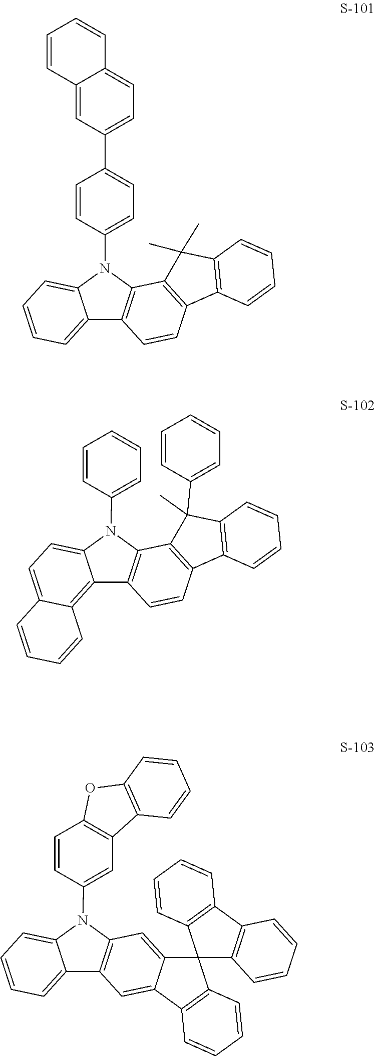 [SMILES: CC1(C)c2ccccc2-c2ccc3c4ccccc4n(-c4ccc(-c5ccc6ccccc6c5)cc4)c3c21 ; CC1(c2ccccc2)c2ccccc2-c2ccc3c4c5ccccc5ccc4n(-c4ccccc4)c3c21 ; c1ccc2c(c1)-c1ccccc1C21c2ccccc2-c2cc3c4ccccc4n(-c4ccc5oc6ccccc6c5c4)c3cc21]

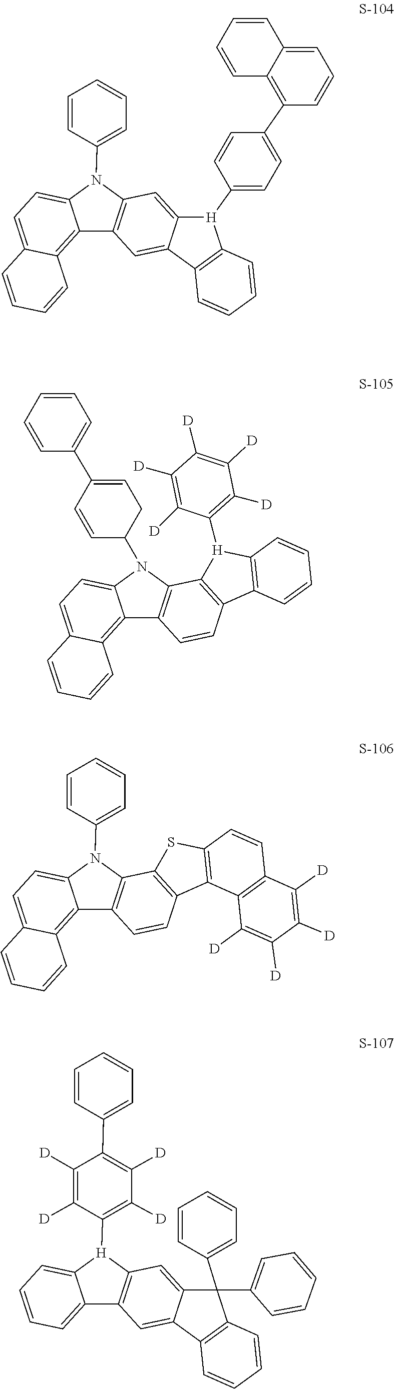

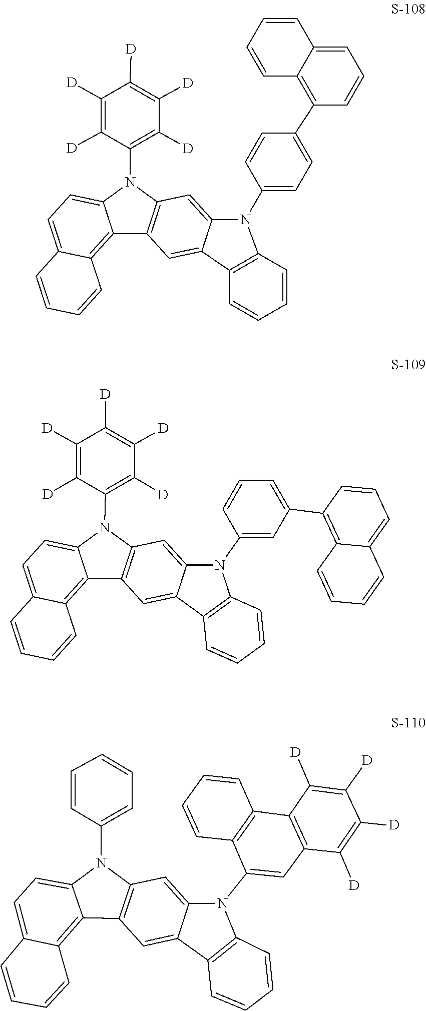 [SMILES: [2H]c1c([2H])c([2H])c(-n2c3cc4c(cc3c3c5ccccc5ccc32)c2ccccc2n4-c2ccc(-c3cccc4ccccc34)cc2)c([2H])c1[2H] ; [2H]c1c([2H])c([2H])c(-n2c3cc4c(cc3c3c5ccccc5ccc32)c2ccccc2n4-c2cccc(-c3cccc4ccccc34)c2)c([2H])c1[2H] ; [2H]c1c([2H])c([2H])c2c(cc(-n3c4ccccc4c4cc5c6c7ccccc7ccc6n(-c6ccccc6)c5cc43)c3ccccc32)c1[2H]]

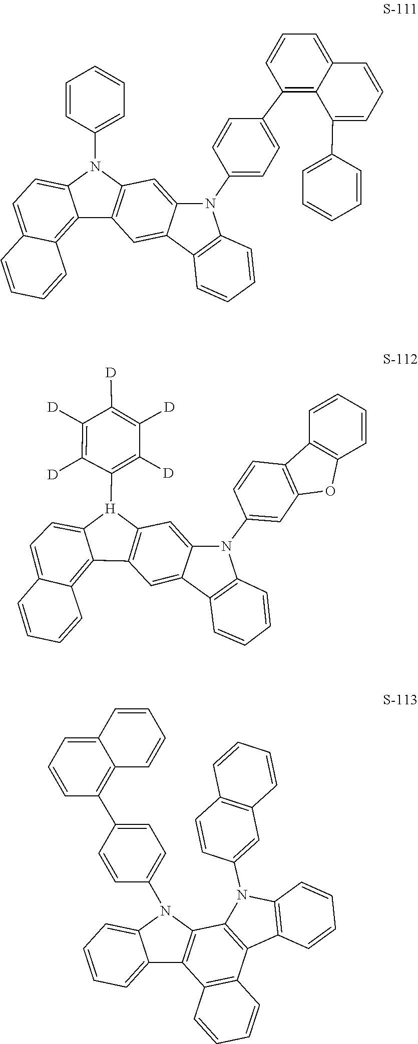

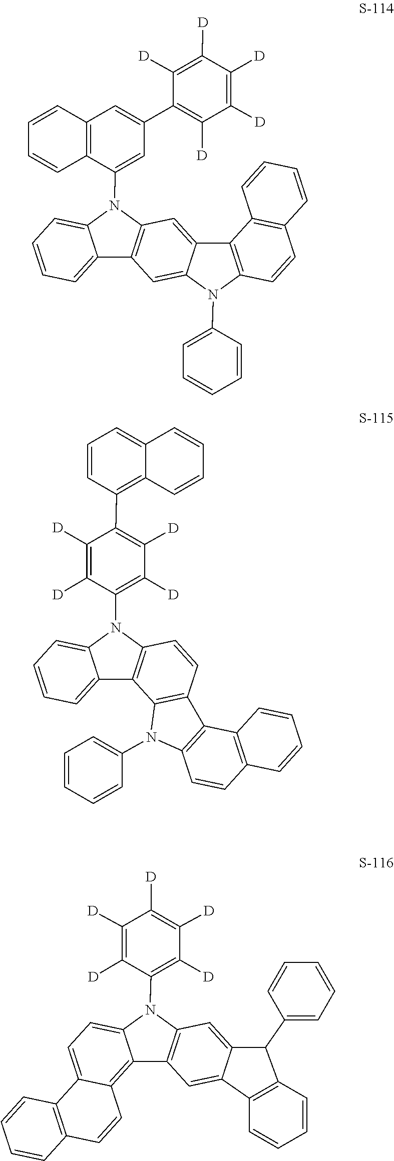 [SMILES: [2H]c1c([2H])c([2H])c(-c2cc(-n3c4ccccc4c4cc5c(cc43)c3c4ccccc4ccc3n5-c3ccccc3)c3ccccc3c2)c([2H])c1[2H] ; [2H]c1c([2H])c(-n2c3ccccc3c3c2ccc2c4c5ccccc5ccc4n(-c4ccccc4)c23)c([2H])c([2H])c1-c1cccc2ccccc12 ; [2H]c1c([2H])c([2H])c(-n2c3cc4c(cc3c3c5ccc6ccccc6c5ccc32)-c2ccccc2C4c2ccccc2)c([2H])c1[2H]]

In another aspect, the present invention provides a method for reusing a compound of Formula 1 comprising:
recovering a crude organic light emitting material comprising the compound of Formula 1 from a deposition apparatus used in the process for depositing the organic emitting material to prepare an organic light emitting device;
removing impurities from the crude organic light emitting material;
recovering the organic light emitting material after the impurities are removed; and
purifying the recovered organic light emitting material to have a purity of 99.9% or higher.

The step of removing impurities from the crude organic light emitting material recovered from the deposition apparatus may preferably comprise performing a pre-purification process to obtain a purity of 98% or more by recrystallization in a recrystallization solvent.

The recrystallization solvent may be preferably a polar solvent having a polarity index (PI) of 5.5 to 7.2.

The recrystallization solvent may be preferably a polar solvent having a polarity index (PI) of 5.5 to 7.2.

The recrystallization solvent may preferably be used by mixing a polar solvent having a polarity value of 5.5 to 7.2 and a non-polar solvent having a polarity value of 2.0 to 4.7.

When a mixture of a polar solvent and a non-polar solvent is used, the recrystallization solvent may be used in an amount of 15% (v/v) or less of the non-polar solvent compared to the polar solvent.

The recrystallization solvent is preferably a single solvent of N-Methylpyrrolidone (NMP); or a polar solvent mixed any one selected from the group consisting of 1,3-Dimethyl-2-imidazolidinone, 2-pyrrolidone, N,N-Dimethyl formamide, Dimethyl acetamide, and Dimethyl sulfoxide to the N-Methylpyrrolidone; or alone; or mixed non-polar solvents; selected from the group consisting of Toluene, Dichloromethane (DCM), Dichloroethane (DCE), Tetrahydrofuran (THF), Chloroform, Ethyl acetate and Butanone; or a mixture of a polar solvent and a non-polar solvent.

The pre-purification process may comprise a step of precipitating crystals of by cooling to 0° C. to 5° C. after dissolving the crude organic light emitting material recovered from the deposition apparatus in a polar solvent at 90° C. to 120° C.

The pre-purification process may comprise a step of precipitating crystals by cooling to 35° C. to 40° C., adding a non-polar solvent, and then cooling to 0° C. to 5° C. after dissolving the crude organic light emitting material recovered from the deposition apparatus in a polar solvent at 90° C. to 120° C.

The pre-purification process may comprise a step of precipitating crystals while concentrating the solvent and removing the non-polar solvent, after dissolving the crude organic light emitting material recovered from the deposition apparatus in a non-polar solvent.

The pre-purification process may comprise a step of recrystallizing again with a non-polar solvent after recrystallizing first with a polar solvent.

The step of purifying the recovered impurities to a purity of 99.9% or higher may comprise performing an adsorption separation process to adsorb and remove impurities by adsorbing on the adsorbent.

The adsorbent may be activated carbon, silica gel, alumina, or a material for known adsorption purposes.

The step of purifying the recovered impurities to a purity of 99.9% or higher may comprise performing sublimation purification.

Also, in another aspect, the present invention provides an organic electronic element comprising a first electrode, a second electrode, an organic material layer between the first electrode and the second electrode; wherein the organic material layer comprises a compound represented by Formula 1; or a composition comprising a first compound represented by Formula 1 and a second compound represented by Formula 4 or 5.

As another example, the organic material layer comprises a hole transport area, an emitting layer and an electron transport area.

As another example, the emitting layer comprises a compound represented by Formula 1; or a composition comprising a first compound represented by Formula 1 and a second compound represented by Formula 4 or 5.

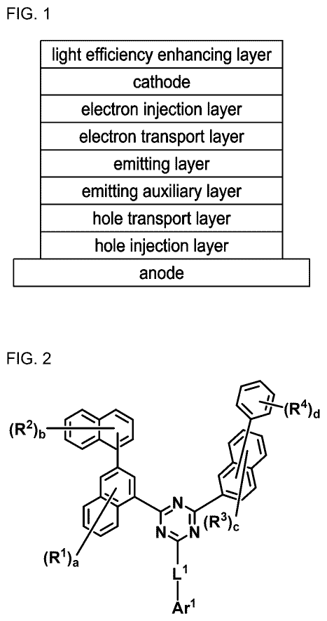

FIG. 1 is an exemplary diagram of an organic electronic element according to an embodiment of the present invention.

Referring to FIG. 1, an organic electronic element according to one embodiment of the present invention comprises a first electrode formed on a substrate (not shown), a second electrode, and an organic material layer formed between the first electrode and the second electrode.

Wherein, the first electrode may be an anode (positive electrode), and the second electrode may be a cathode (a negative electrode). In the case of an inverted organic electronic element, the first electrode may be a cathode, and the second electrode may be an anode.

Additionally, the present invention may comprise a hole transport region between the first electrode and the emitting layer, and may comprise an electron transport region between the second electrode and the emitting layer. That is, the organic material layer may comprise a hole transport area, an emitting layer, and an electron transport area.

Additionally, the hole transport area may comprise a hole injection layer (HIL), a hole transport layer (HTL), an electron blocking layer (EBL), a hole transport auxiliary layer, etc., and may comprise at least one hole transport layer.

The electron transport area may comprise an electron injection layer (EIL), an electron transport layer (ETL), a hole blocking layer (HBL), an electron transport auxiliary layer, etc., and may comprise at least one electron transport layer.

An emitting auxiliary layer may be formed between the hole transport layer and the emitting layer, and although not shown in the drawing, a buffer layer may be further formed between the hole transport layer and the emitting auxiliary layer. Specifically, a hole injection layer, a hole transport layer, a buffer layer, an emitting auxiliary layer, an emitting layer, an electron transport layer, and an electron injection layer can be sequentially formed on the first electrode.

Preferably, a light efficiency enhancing layer may be formed on one side of the first electrode or the second electrode that is not in contact with the organic material layer, and when the light efficiency enhancing layer is formed, the light efficiency of the organic electronic element may be improved.

For example, a light efficiency enhancing layer can be formed on the second electrode. In the case of a top emission organic light emitting device, the formation of the light efficiency enhancing layer can reduce optical energy loss due to SPPs (surface plasmon polaritons) at the second electrode, and in the case of a bottom emission organic light emitting device, the light efficiency enhancing layer can serve as a buffer for the second electrode.

Although not shown in FIG. 1, an electron transport auxiliary layer may be further formed between the emitting layer and the electron transport layer.

According to another embodiment of the present invention, the organic material layer may be formed in a form in which a plurality of stacks comprising a hole injection layer, a hole transport layer, an emitting auxiliary layer, an emitting layer, and an electron transport layer are formed.

In general, organic light-emitting devices can be divided into single-light-emitting structure devices (Single OLED) and multilayer light-emitting structure devices (Tandem OLED) depending on the number of light-emitting parts. The multilayer light-emitting structure device (Tandem OLED) is an OLED device composed of 2 or more light-emitting parts (stacks), and it is easy to improve the driving voltage and efficiency compared to the existing single OLED.

Specifically, an organic electronic element according to one embodiment of the present invention may include a first electrode, a first stack formed on the first electrode, a second stack formed on the first stack, and a second electrode. Wherein the stack may correspond to an organic material layer, and a light efficiency enhancing layer may be further formed on a surface of the first electrode and/or the second electrode that is not in contact with the organic material layer.

The first stack and the second stack are organic material layers comprising a hole transport layer, an emitting layer, and an electron transport layer, respectively, and the first stack and the second stack may be formed with the same or different laminated structures.

Additionally, a charge generation layer (CGL) may be formed between the first stack and the second stack. The charge generation layer (CGL) may comprise a first charge generation layer and a second charge generation layer. These charge generation layers (CGLs) are formed between the emitting layers of the first stack and the emitting layers of the second stack, and serve to increase the current efficiency generated in each emitting layer and to smoothly distribute charges.

2 or more stacks of these organic material layers can be formed. For example, when 3 stacks are formed, a charge generation layer (CGL) and a third stack can be additionally stacked on the second stack.

In this way, when multiple emitting layers are formed by a multi-layer stack structure, it is possible to manufacture an organic light-emitting device that emits white light through the mixing effect of the light emitted from each emitting layer, and it is also possible to manufacture an organic light-emitting device that emits light of various colors.

The present invention may further comprise a light efficiency enhancing layer formed on at least one surface of the first electrode and the second electrode, which is opposite to the organic material layer. Moreover, the organic material layer may comprise 2 or more stacks comprising a hole transport layer, an emitting layer, and an electron transport layer sequentially formed on the anode, and the organic layer may further comprise a charge generation layer formed between the 2 or more stacks.

The organic material layer according to the present invention can be manufactured with a smaller number of layers by using various polymer materials and a solution process or solvent process other than a deposition method, such as a spin coating process, a nozzle printing process, an inkjet printing process, a slot coating process, a dip coating process, a roll-to-roll process, a doctor blading process, a screen printing process, or a thermal transfer method. Since the organic material layer according to the present invention can be formed in various ways, the scope of the present invention is not limited by the formation method.

An organic electronic element according to one embodiment of the present invention may be a front-emitting, back-emitting, or double-sided emitting type depending on the material used.

Additionally, the organic electronic element according to one embodiment of the present invention may be selected from the group consisting of an organic light-emitting device, an organic solar cell, an organic photoconductor, an organic transistor, a monochrome lighting device, and a quantum dot display device.

Another embodiment of the present invention may include a display device comprising the organic electronic element of the present invention described above, and an electronic device comprising a control unit for driving the display device. Wherein the electronic device may be a current or future wired or wireless communication terminal, and includes all electronic devices such as mobile communication terminals such as mobile phones, PDAs, electronic dictionaries, PMPs, remote controls, navigation systems, game consoles, various TVs, and various computers, watches, tablets, and virtual reality (VR) devices.

Hereinafter, the synthesis examples of compounds represented by Formulas 1, 4 and 5 according to the present invention and the manufacturing examples of organic electronic elements according to the present invention will be described in detail with reference to examples, but the present invention is not limited to the following examples.

### Synthesis Example

The compound (final product) represented by Formula 1 according to the present invention can be synthesized by reacting Sub A and Sub B as in the Reaction Scheme 1, but is not limited thereto. (Hall is Br, I or Cl)

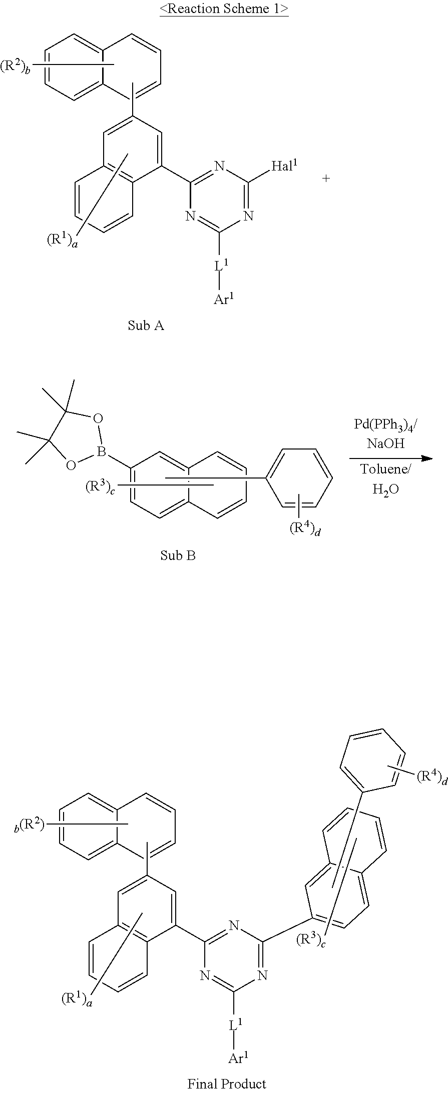

wherein: R¹, R², R³, R⁴, L¹, Ar¹, a, b, c and d are the same as defined in Formula 1.

### I. Synthesis of Sub A

Sub A of Reaction Scheme 1 can be synthesized by the reaction path of Reaction Scheme 2, but is not limited thereto. (Hal¹, Hal²=Br, I or Cl)

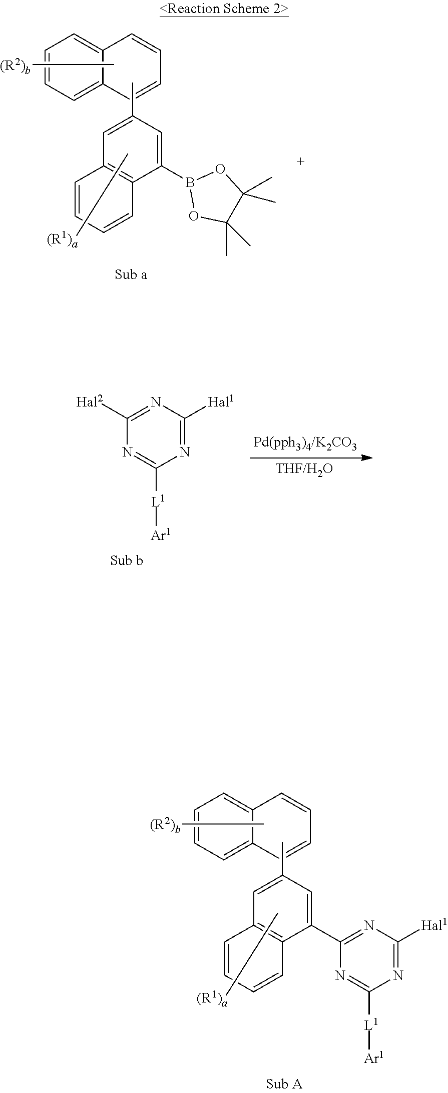

wherein: R¹, R², L¹, Ar¹, a and b are the same as defined in Formula 1.

Compounds belonging to Sub A may be, but are not limited to, the compounds below, and Table 1 shows the FD-MS (Field Desorption Mass Spectrometry) values of Compounds belonging to Sub A.

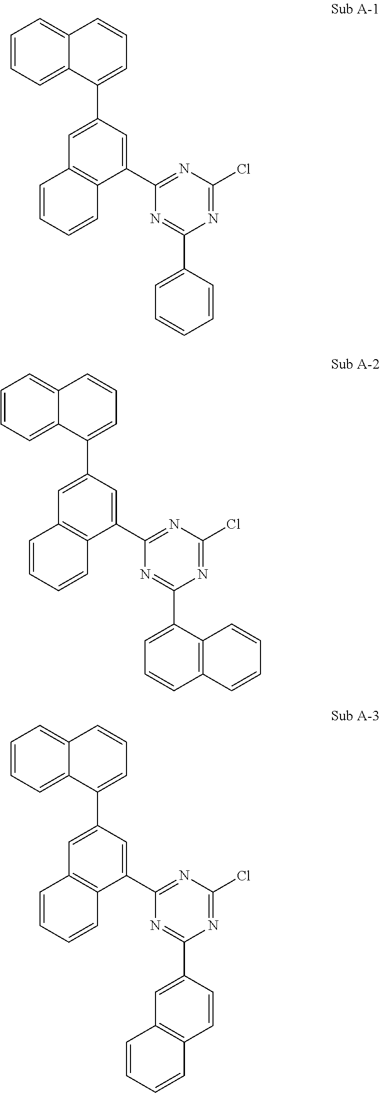 [SMILES: Clc1nc(-c2ccccc2)nc(-c2cc(-c3cccc4ccccc34)cc3ccccc23)n1 ; Clc1nc(-c2cccc3ccccc23)nc(-c2cc(-c3cccc4ccccc34)cc3ccccc23)n1 ; Clc1nc(-c2ccc3ccccc3c2)nc(-c2cc(-c3cccc4ccccc34)cc3ccccc23)n1]

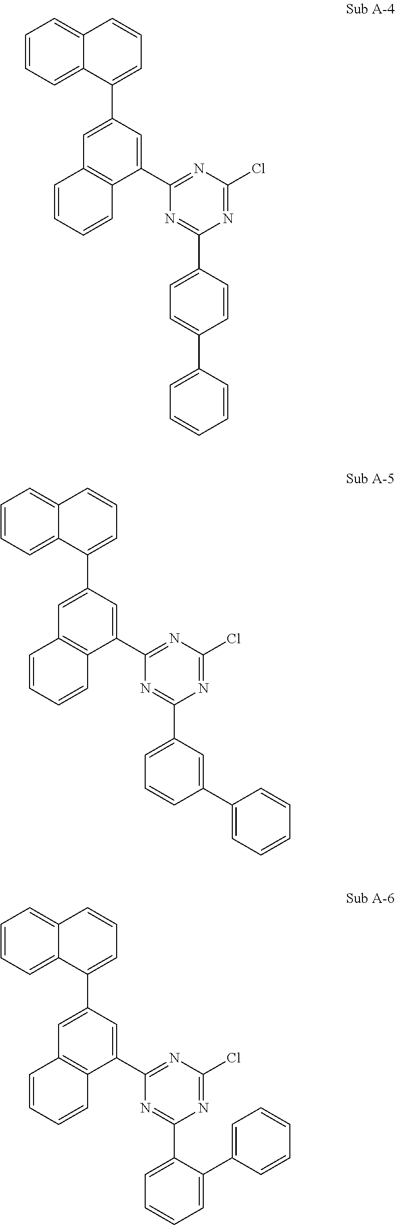 [SMILES: Clc1nc(-c2ccc(-c3ccccc3)cc2)nc(-c2cc(-c3cccc4ccccc34)cc3ccccc23)n1 ; Clc1nc(-c2cccc(-c3ccccc3)c2)nc(-c2cc(-c3cccc4ccccc34)cc3ccccc23)n1 ; Clc1nc(-c2ccccc2-c2ccccc2)nc(-c2cc(-c3cccc4ccccc34)cc3ccccc23)n1]

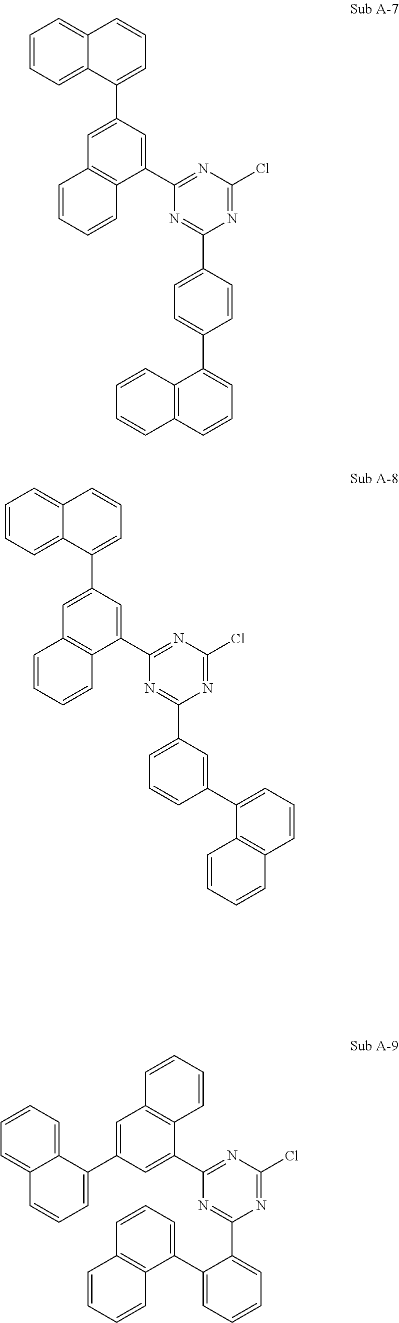 [SMILES: Clc1nc(-c2ccc(-c3cccc4ccccc34)cc2)nc(-c2cc(-c3cccc4ccccc34)cc3ccccc23)n1 ; Clc1nc(-c2cccc(-c3cccc4ccccc34)c2)nc(-c2cc(-c3cccc4ccccc34)cc3ccccc23)n1 ; Clc1nc(-c2ccccc2-c2cccc3ccccc23)nc(-c2cc(-c3cccc4ccccc34)cc3ccccc23)n1]

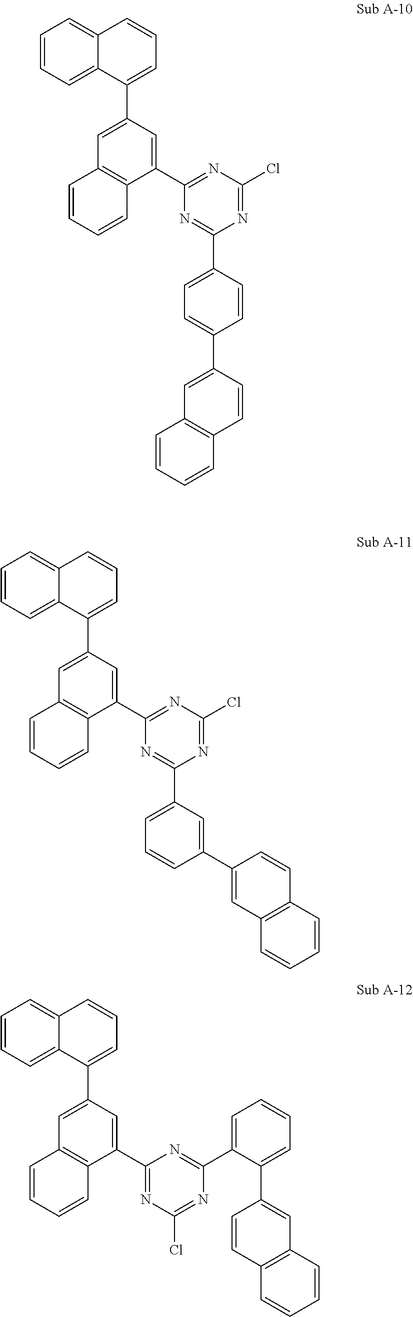 [SMILES: Clc1nc(-c2ccc(-c3ccc4ccccc4c3)cc2)nc(-c2cc(-c3cccc4ccccc34)cc3ccccc23)n1 ; Clc1nc(-c2cccc(-c3ccc4ccccc4c3)c2)nc(-c2cc(-c3cccc4ccccc34)cc3ccccc23)n1 ; Clc1nc(-c2ccccc2-c2ccc3ccccc3c2)nc(-c2cc(-c3cccc4ccccc34)cc3ccccc23)n1]

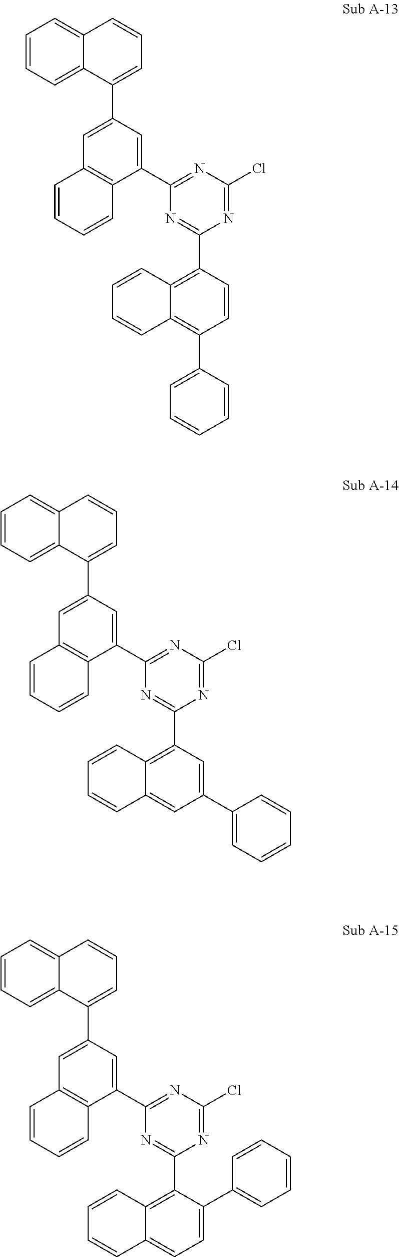 [SMILES: Clc1nc(-c2cc(-c3cccc4ccccc34)cc3ccccc23)nc(-c2ccc(-c3ccccc3)c3ccccc23)n1 ; Clc1nc(-c2cc(-c3ccccc3)cc3ccccc23)nc(-c2cc(-c3cccc4ccccc34)cc3ccccc23)n1 ; Clc1nc(-c2cc(-c3cccc4ccccc34)cc3ccccc23)nc(-c2c(-c3ccccc3)ccc3ccccc23)n1]

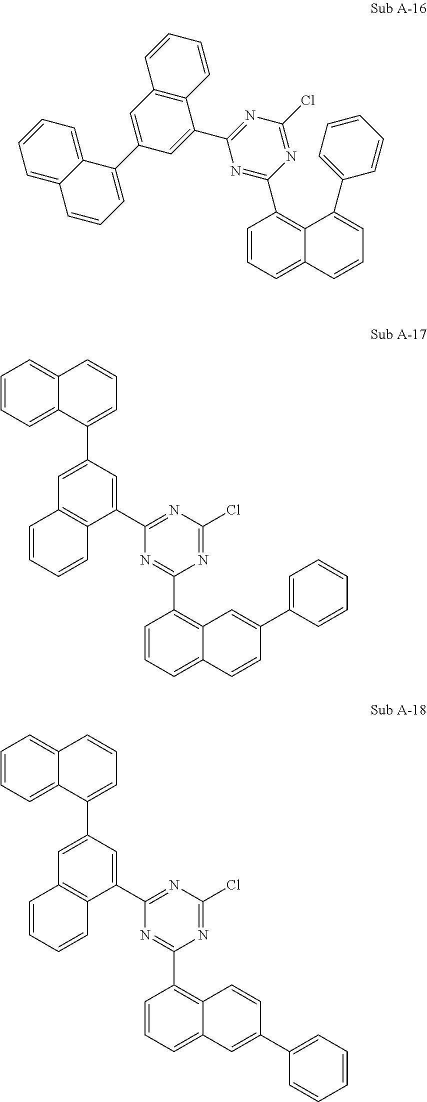 [SMILES: Clc1nc(-c2cc(-c3cccc4ccccc34)cc3ccccc23)nc(-c2cccc3cccc(-c4ccccc4)c23)n1 ; Clc1nc(-c2cc(-c3cccc4ccccc34)cc3ccccc23)nc(-c2cccc3ccc(-c4ccccc4)cc23)n1 ; Clc1nc(-c2cccc3cc(-c4ccccc4)ccc23)nc(-c2cc(-c3cccc4ccccc34)cc3ccccc23)n1]

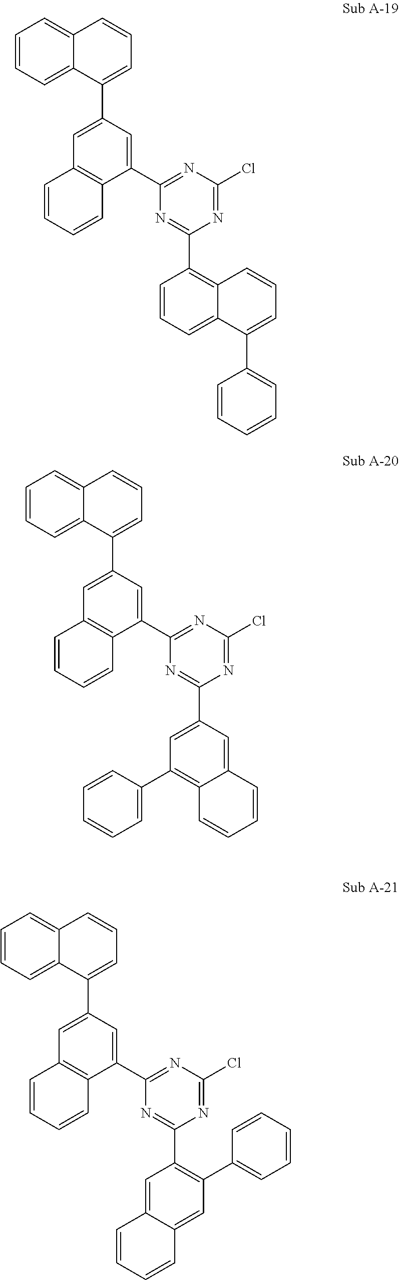 [SMILES: Clc1nc(-c2cc(-c3cccc4ccccc34)cc3ccccc23)nc(-c2cccc3c(-c4ccccc4)cccc23)n1 ; Clc1nc(-c2cc(-c3ccccc3)c3ccccc3c2)nc(-c2cc(-c3cccc4ccccc34)cc3ccccc23)n1 ; Clc1nc(-c2cc3ccccc3cc2-c2ccccc2)nc(-c2cc(-c3cccc4ccccc34)cc3ccccc23)n1]

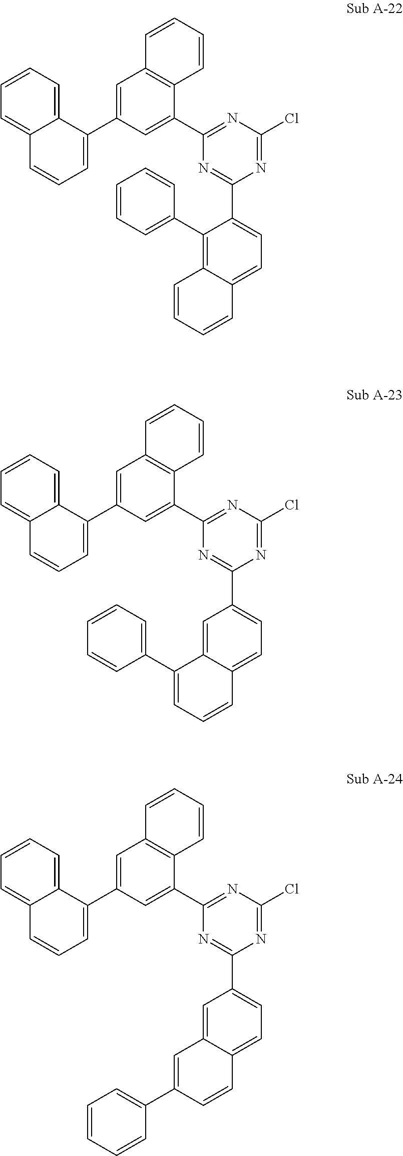

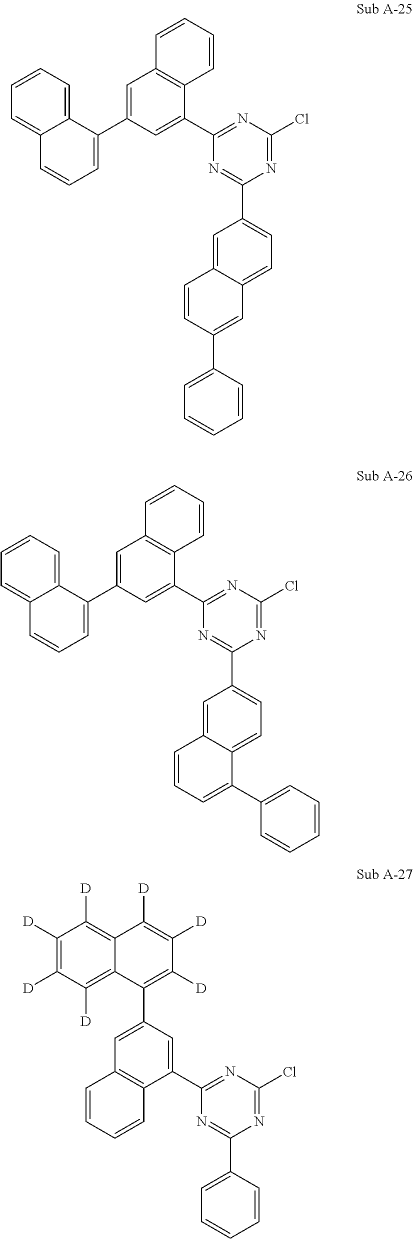

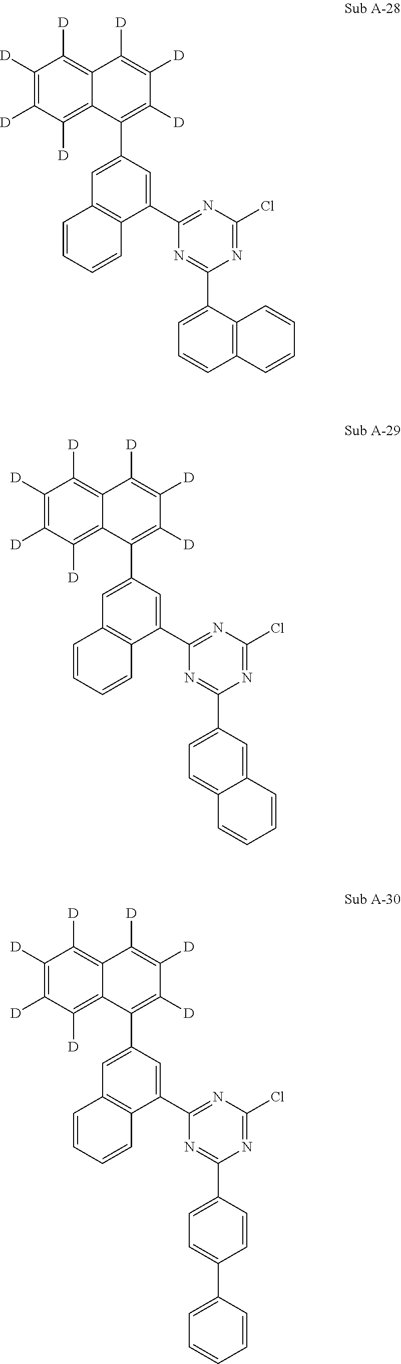

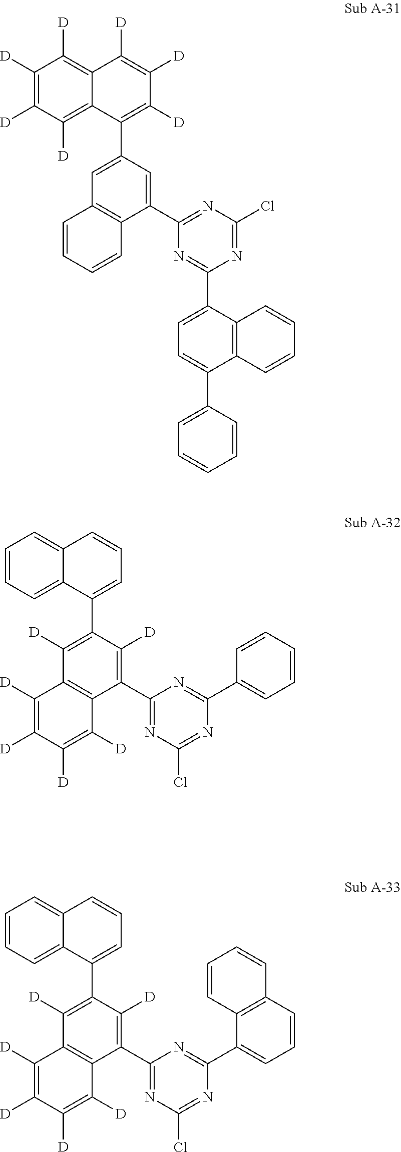

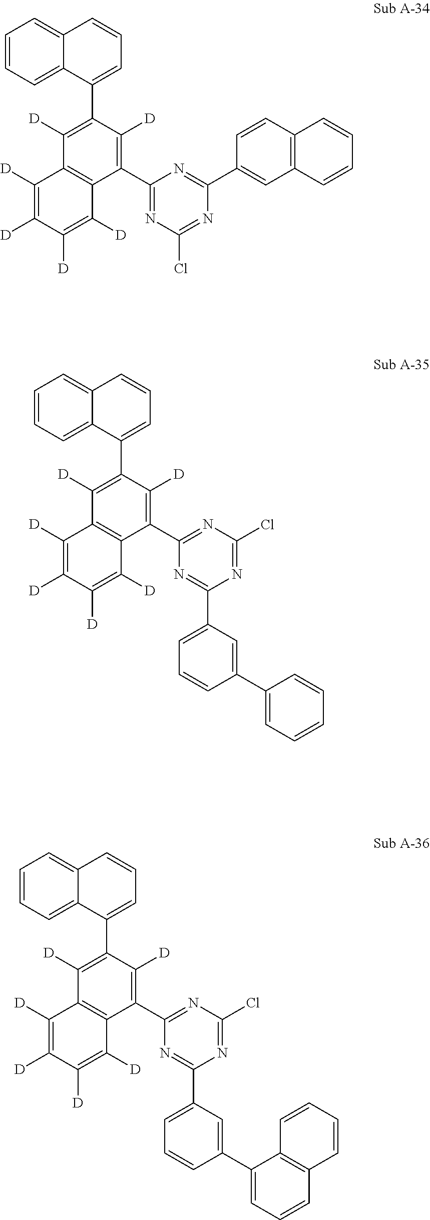 [SMILES: [2H]c1c([2H])c([2H])c2c(-c3nc(Cl)nc(-c4ccc5ccccc5c4)n3)c([2H])c(-c3cccc4ccccc34)c([2H])c2c1[2H] ; [2H]c1c([2H])c([2H])c2c(-c3nc(Cl)nc(-c4cccc(-c5ccccc5)c4)n3)c([2H])c(-c3cccc4ccccc34)c([2H])c2c1[2H] ; [2H]c1c([2H])c([2H])c2c(-c3nc(Cl)nc(-c4cccc(-c5cccc6ccccc56)c4)n3)c([2H])c(-c3cccc4ccccc34)c([2H])c2c1[2H]]

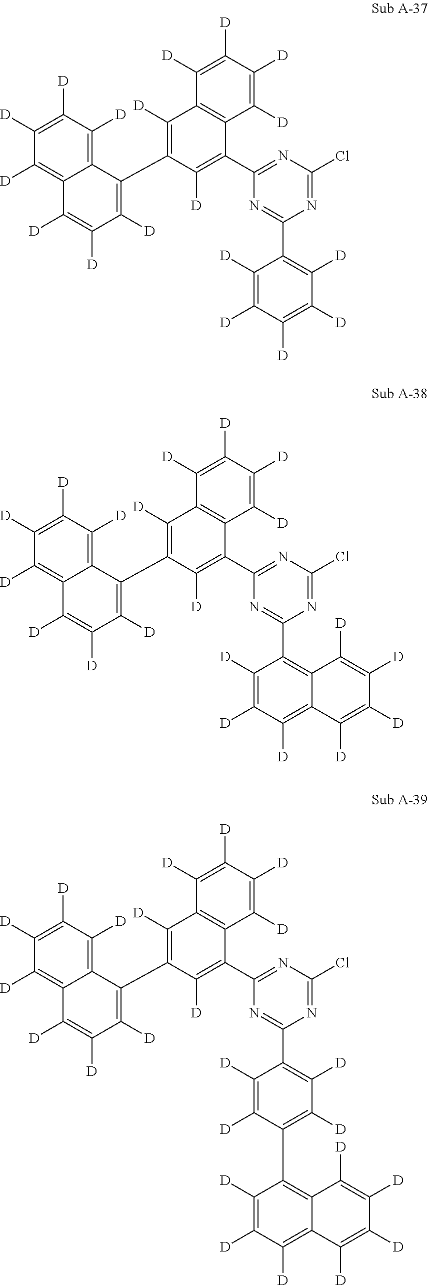

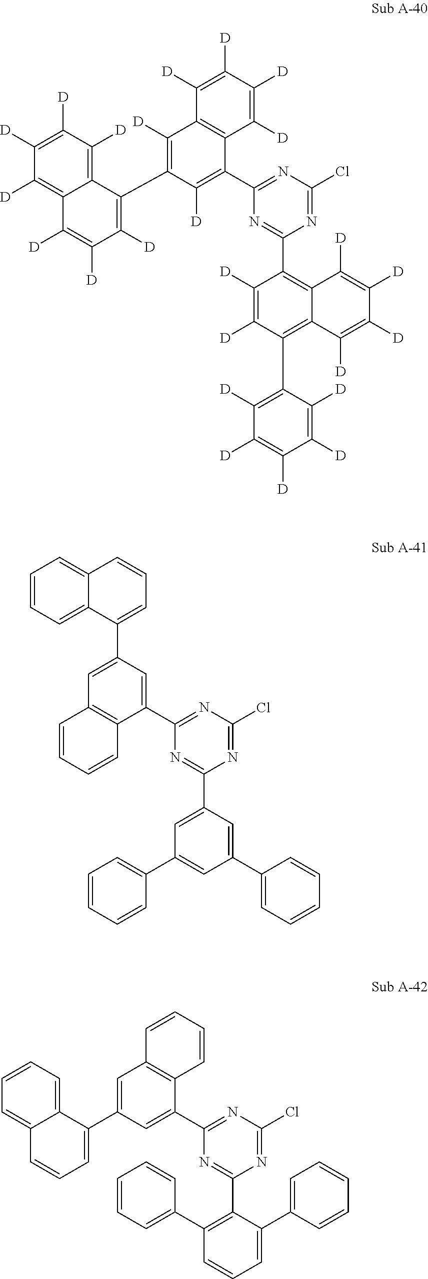 [SMILES: [2H]c1c([2H])c([2H])c(-c2c([2H])c([2H])c(-c3nc(Cl)nc(-c4c([2H])c(-c5c([2H])c([2H])c([2H])c6c([2H])c([2H])c([2H])c([2H])c56)c([2H])c5c([2H])c([2H])c([2H])c([2H])c45)n3)c3c([2H])c([2H])c([2H])c([2H])c23)c([2H])c1[2H] ; Clc1nc(-c2cc(-c3ccccc3)cc(-c3ccccc3)c2)nc(-c2cc(-c3cccc4ccccc34)cc3ccccc23)n1 ; Clc1nc(-c2c(-c3ccccc3)cccc2-c2ccccc2)nc(-c2cc(-c3cccc4ccccc34)cc3ccccc23)n1]

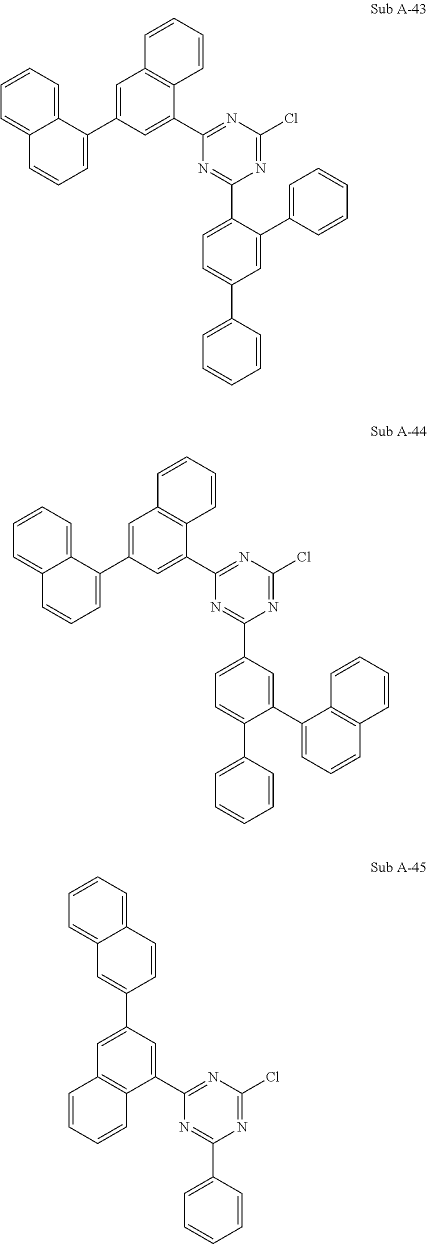 [SMILES: Clc1nc(-c2ccc(-c3ccccc3)cc2-c2ccccc2)nc(-c2cc(-c3cccc4ccccc34)cc3ccccc23)n1 ; Clc1nc(-c2ccc(-c3ccccc3)c(-c3cccc4ccccc34)c2)nc(-c2cc(-c3cccc4ccccc34)cc3ccccc23)n1 ; Clc1nc(-c2ccccc2)nc(-c2cc(-c3ccc4ccccc4c3)cc3ccccc23)n1]

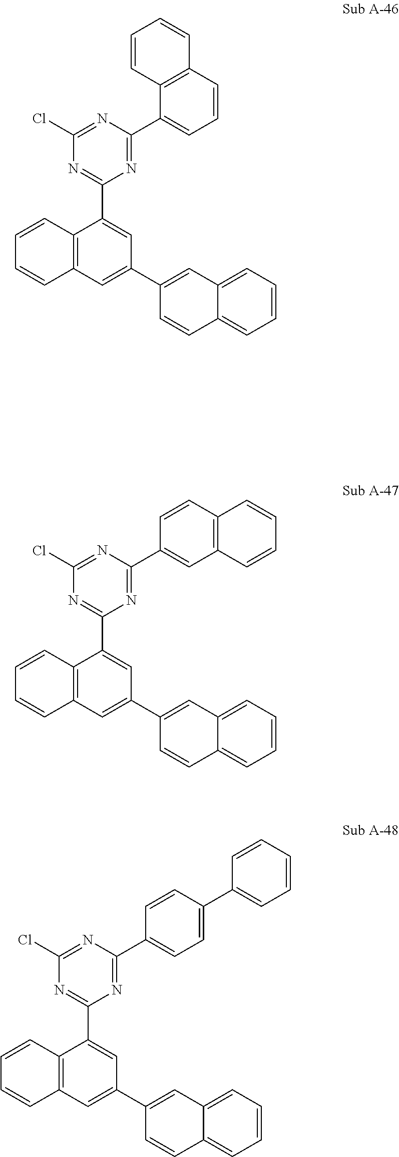 [SMILES: Clc1nc(-c2cccc3ccccc23)nc(-c2cc(-c3ccc4ccccc4c3)cc3ccccc23)n1 ; Clc1nc(-c2ccc3ccccc3c2)nc(-c2cc(-c3ccc4ccccc4c3)cc3ccccc23)n1 ; Clc1nc(-c2ccc(-c3ccccc3)cc2)nc(-c2cc(-c3ccc4ccccc4c3)cc3ccccc23)n1]

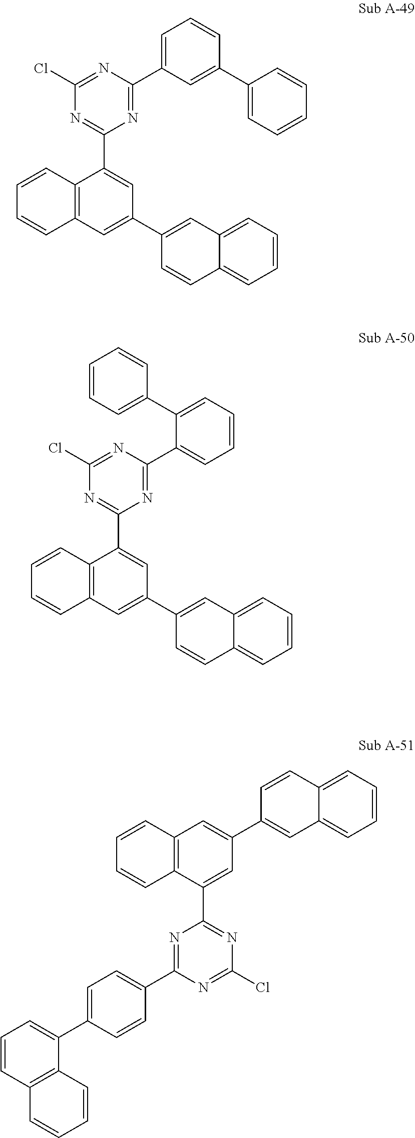 [SMILES: Clc1nc(-c2cccc(-c3ccccc3)c2)nc(-c2cc(-c3ccc4ccccc4c3)cc3ccccc23)n1 ; Clc1nc(-c2ccccc2-c2ccccc2)nc(-c2cc(-c3ccc4ccccc4c3)cc3ccccc23)n1 ; Clc1nc(-c2ccc(-c3cccc4ccccc34)cc2)nc(-c2cc(-c3ccc4ccccc4c3)cc3ccccc23)n1]

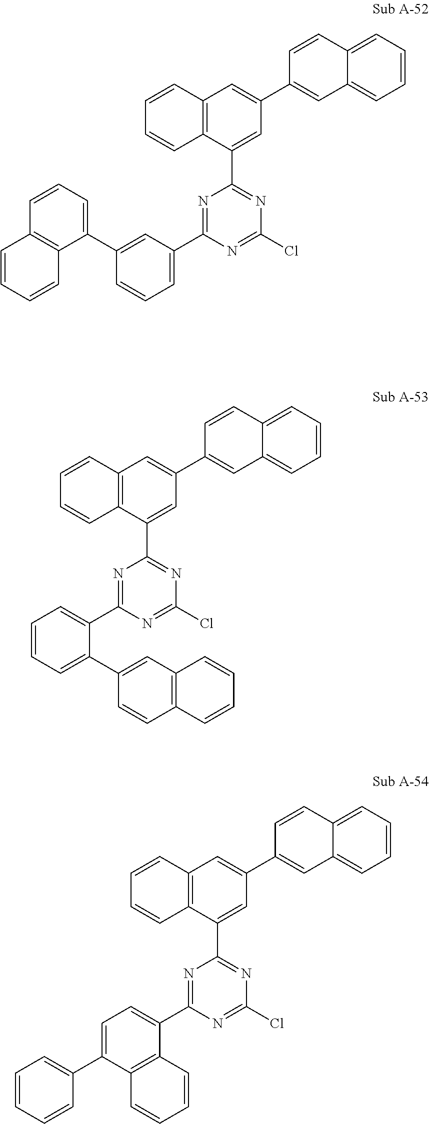 [SMILES: Clc1nc(-c2cccc(-c3cccc4ccccc34)c2)nc(-c2cc(-c3ccc4ccccc4c3)cc3ccccc23)n1 ; Clc1nc(-c2ccccc2-c2ccc3ccccc3c2)nc(-c2cc(-c3ccc4ccccc4c3)cc3ccccc23)n1 ; Clc1nc(-c2cc(-c3ccc4ccccc4c3)cc3ccccc23)nc(-c2ccc(-c3ccccc3)c3ccccc23)n1]

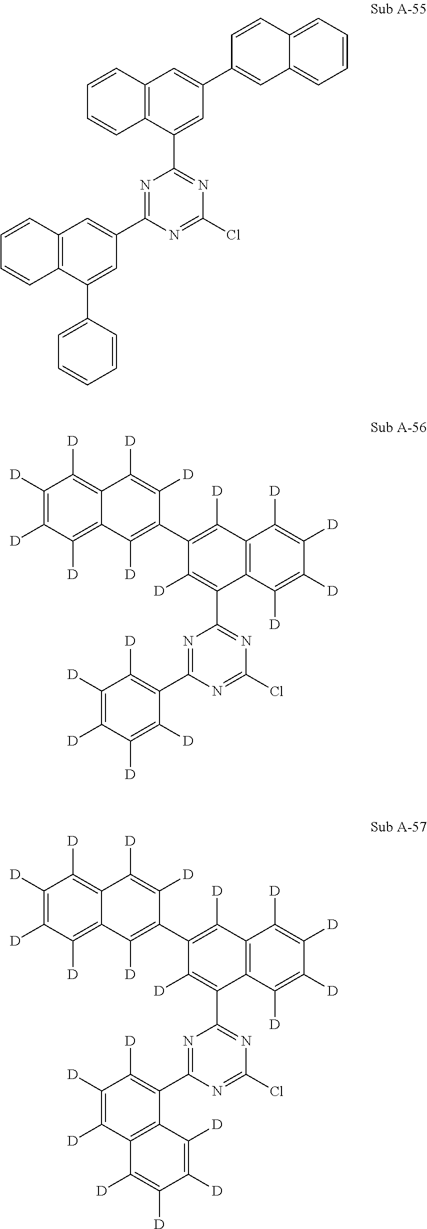 [SMILES: Clc1nc(-c2cc(-c3ccccc3)c3ccccc3c2)nc(-c2cc(-c3ccc4ccccc4c3)cc3ccccc23)n1 ; [2H]c1c([2H])c([2H])c(-c2nc(Cl)nc(-c3c([2H])c(-c4c([2H])c([2H])c5c([2H])c([2H])c([2H])c([2H])c5c4[2H])c([2H])c4c([2H])c([2H])c([2H])c([2H])c34)n2)c([2H])c1[2H] ; [2H]c1c([2H])c([2H])c2c([2H])c(-c3c([2H])c(-c4nc(Cl)nc(-c5c([2H])c([2H])c([2H])c6c([2H])c([2H])c([2H])c([2H])c56)n4)c4c([2H])c([2H])c([2H])c([2H])c4c3[2H])c([2H])c([2H])c2c1[2H]]

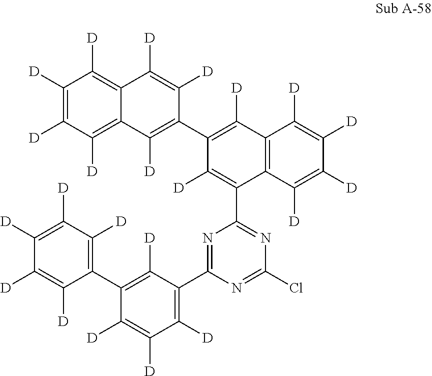 [SMILES: [2H]c1c([2H])c([2H])c(-c2c([2H])c([2H])c([2H])c(-c3nc(Cl)nc(-c4c([2H])c(-c5c([2H])c([2H])c6c([2H])c([2H])c([2H])c([2H])c6c5[2H])c([2H])c5c([2H])c([2H])c([2H])c([2H])c45)n3)c2[2H])c([2H])c1[2H]]

TABLE 1

| compound | FD-MS | compound | FD-MS |
|---|---|---|---|
| Sub A-1 | m/z = 443.12(C₂₉H₁₈ClN₃ = 443.93) | Sub A-2 | m/z = 443.12(C₂₉H₁₈ClN₃ = 443.93) |
| Sub A-3 | m/z = 443.12(C₂₉H₁₈ClN₃ = 443.93) | Sub A-4 | m/z = 519.15(C₃₅H₂₂ClN₃ = 520.03) |
| Sub A-5 | m/z = 519.15(C₃₅H₂₂ClN₃ = 520.03) | Sub A-6 | m/z = 519.15(C₃₅H₂₂ClN₃ = 520.03) |
| Sub A-7 | m/z = 569.17(C₃₉H₂₄ClN₃ = 570.09) | Sub A-8 | m/z = 569.17(C₃₉H₂₄ClN₃ = 570.09) |
| Sub A-9 | m/z = 569.17(C₃₉H₂₄ClN₃ = 570.09) | Sub A-10 | m/z = 569.17(C₃₉H₂₄ClN₃ = 570.09) |
| Sub A-11 | m/z = 569.17(C₃₉H₂₄ClN₃ = 570.09) | Sub A-12 | m/z = 569.17(C₃₉H₂₄ClN₃ = 570.09) |
| Sub A-13 | m/z = 569.17(C₃₉H₂₄ClN₃ = 570.09) | Sub A-14 | m/z = 569.17(C₃₉H₂₄ClN₃ = 570.09) |
| Sub A-15 | m/z = 569.17(C₃₉H₂₄ClN₃ = 570.09) | Sub A-16 | m/z = 569.17(C₃₉H₂₄ClN₃ = 570.09) |
| Sub A-17 | m/z = 569.17(C₃₉H₂₄ClN₃ = 570.09) | Sub A-18 | m/z = 569.17(C₃₉H₂₄ClN₃ = 570.09) |
| Sub A-19 | m/z = 569.17(C₃₉H₂₄ClN₃ = 570.09) | Sub A-20 | m/z = 569.17(C₃₉H₂₄ClN₃ = 570.09) |
| Sub A-21 | m/z = 569.17(C₃₉H₂₄ClN₃ = 570.09) | Sub A-22 | m/z = 569.17(C₃₉H₂₄ClN₃ = 570.09) |
| Sub A-23 | m/z = 569.17(C₃₉H₂₄ClN₃ = 570.09) | Sub A-24 | m/z = 569.17(C₃₉H₂₄ClN₃ = 570.09) |
| Sub A-25 | m/z = 569.17(C₃₉H₂₄ClN₃ = 570.09) | Sub A-26 | m/z = 569.17(C₃₉H₂₄ClN₃ = 570.09) |
| Sub A-27 | m/z = 450.16(C₂₉H₁₁D₇ClN₃ = 450.98) | Sub A-28 | m/z = 500.18(C₃₃H₁₃D₇ClN₃ = 501.04) |
| Sub A-29 | m/z = 500.18(C₃₃H₁₃D₇ClN₃ = 501.04) | Sub A-30 | m/z = 526.19(C₃₅H₁₅D₇ClN₃ = 527.07) |
| Sub A-31 | m/z = 576.21(C₃₉H₁₇D₇ClN₃ = 577.13) | Sub A-32 | m/z = 449.16(C₂₉H₁₂D₆ClN₃ = 449.97) |
| Sub A-33 | m/z = 499.17(C₃₃H₁₄D₆ClN₃ = 500.03) | Sub A-34 | m/z = 499.17(C₃₃H₁₄D₆ClN₃ = 500.03) |
| Sub A-35 | m/z = 525.19(C₃₅H₁₆D₆ClN₃ = 526.07) | Sub A-36 | m/z = 575.2(C₃₉H₁₈D₆ClN₃ = 576.13) |
| Sub A-37 | m/z = 461.23(C₂₉D₁₈ClN₃ = 462.04) | Sub A-38 | m/z = 513.26(C₃₃D₂₀ClN₃ = 514.12) |
| Sub A-39 | m/z = 593.32(C₃₉D₂₄ClN₃ = 594.24) | Sub A-40 | m/z = 593.32(C₃₉D₂₄ClN₃ = 594.24) |
| Sub A-41 | m/z = 595.18(C₄₁H₂₆ClN₃ = 596.13) | Sub A-42 | m/z = 595.18(C₄₁H₂₆ClN₃ = 596.13) |
| Sub A-43 | m/z = 595.18(C₄₁H₂₆ClN₃ = 596.13) | Sub A-44 | m/z = 645.2(C₄₅H₂₈ClN₃ = 646.19) |
| Sub A-45 | m/z = 443.12(C₂₉H₁₈ClN₃ = 443.93) | Sub A-46 | m/z = 493.13(C₃₃H₂₀ClN₃ = 493.99) |
| Sub A-47 | m/z = 493.13(C₃₃H₂₀ClN₃ = 493.99) | Sub A-48 | m/z = 519.15(C₃₅H₂₂ClN₃ = 520.03) |
| Sub A-49 | m/z = 519.15(C₃₅H₂₂ClN₃ = 520.03) | Sub A-50 | m/z = 519.15(C₃₅H₂₂ClN₃ = 520.03) |
| Sub A-51 | m/z = 569.17(C₃₉H₂₄ClN₃ = 570.09) | Sub A-52 | m/z = 569.17(C₃₉H₂₄ClN₃ = 570.09) |
| Sub A-53 | m/z = 569.17(C₃₉H₂₄ClN₃ = 570.09) | Sub A-54 | m/z = 569.17(C₃₉H₂₄ClN₃ = 570.09) |
| Sub A-55 | m/z = 569.17(C₃₉H₂₄ClN₃ = 570.09) | Sub A-56 | m/z = 461.23(C₂₉D₁₈ClN₃ = 462.04) |
| Sub A-57 | m/z = 513.26(C₃₃D₂₀ClN₃ = 514.12) | Sub A-58 | m/z = 541.29(C₃₅D₂₂ClN₃ = 542.17) |

### II. Synthesis of Sub 2

Sub B of Scheme 1 can be synthesized by the reaction path of Reaction Scheme 3, but is not limited thereto. (Hal³=Br, I or C₁)

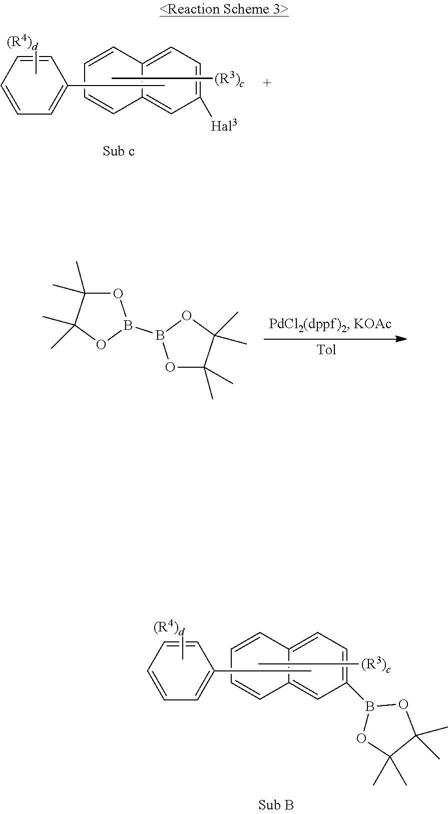

Compounds belonging to Sub B may be, but are not limited to, the compounds below, and Table 2 shows the FD-MS (Field Desorption-Mass Spectrometry) values of compounds belonging to Sub B.

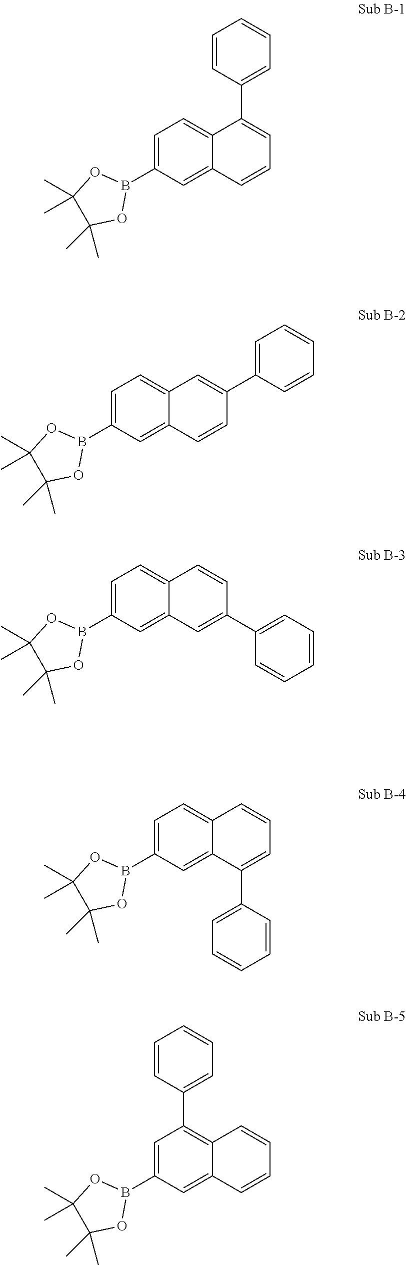 [SMILES: CC1(C)OB(c2ccc3c(-c4ccccc4)cccc3c2)OC1(C)C ; CC1(C)OB(c2ccc3cc(-c4ccccc4)ccc3c2)OC1(C)C ; CC1(C)OB(c2ccc3ccc(-c4ccccc4)cc3c2)OC1(C)C ; CC1(C)OB(c2ccc3cccc(-c4ccccc4)c3c2)OC1(C)C ; CC1(C)OB(c2cc(-c3ccccc3)c3ccccc3c2)OC1(C)C]

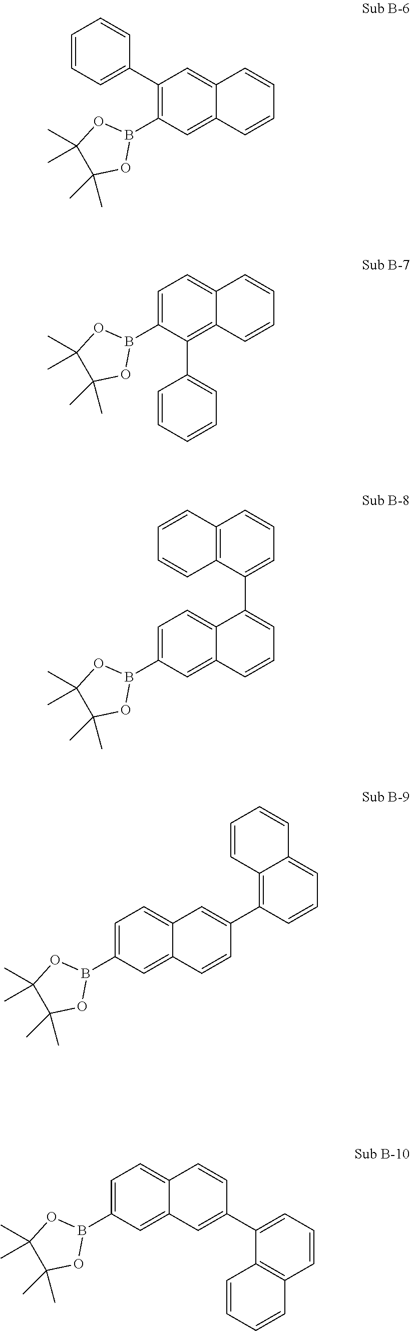 [SMILES: CC1(C)OB(c2cc3ccccc3cc2-c2ccccc2)OC1(C)C ; CC1(C)OB(c2ccc3ccccc3c2-c2ccccc2)OC1(C)C ; CC1(C)OB(c2ccc3c(-c4cccc5ccccc45)cccc3c2)OC1(C)C ; CC1(C)OB(c2ccc3cc(-c4cccc5ccccc45)ccc3c2)OC1(C)C ; CC1(C)OB(c2ccc3ccc(-c4cccc5ccccc45)cc3c2)OC1(C)C]

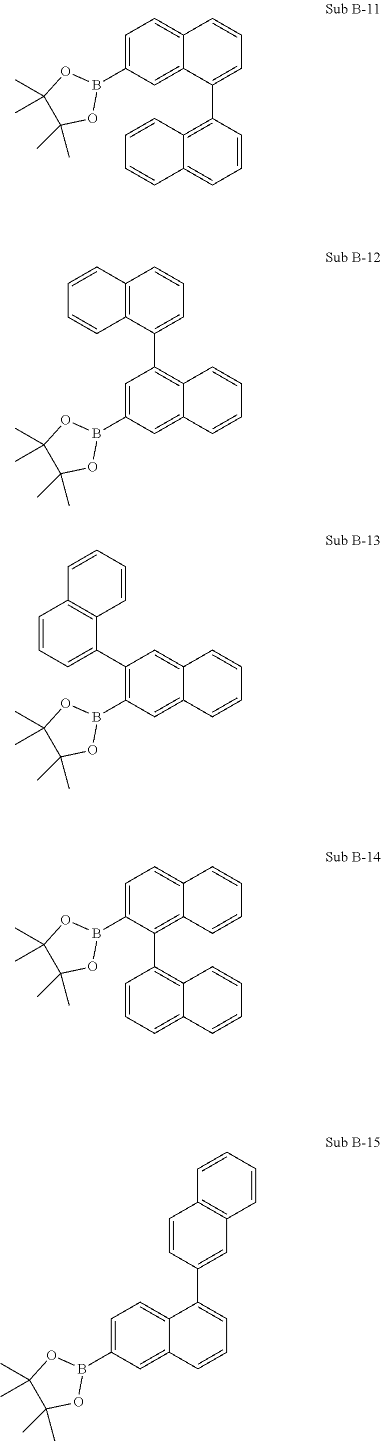 [SMILES: CC1(C)OB(c2ccc3cccc(-c4cccc5ccccc45)c3c2)OC1(C)C ; CC1(C)OB(c2cc(-c3cccc4ccccc34)c3ccccc3c2)OC1(C)C ; CC1(C)OB(c2cc3ccccc3cc2-c2cccc3ccccc23)OC1(C)C ; CC1(C)OB(c2ccc3ccccc3c2-c2cccc3ccccc23)OC1(C)C ; CC1(C)OB(c2ccc3c(-c4ccc5ccccc5c4)cccc3c2)OC1(C)C]

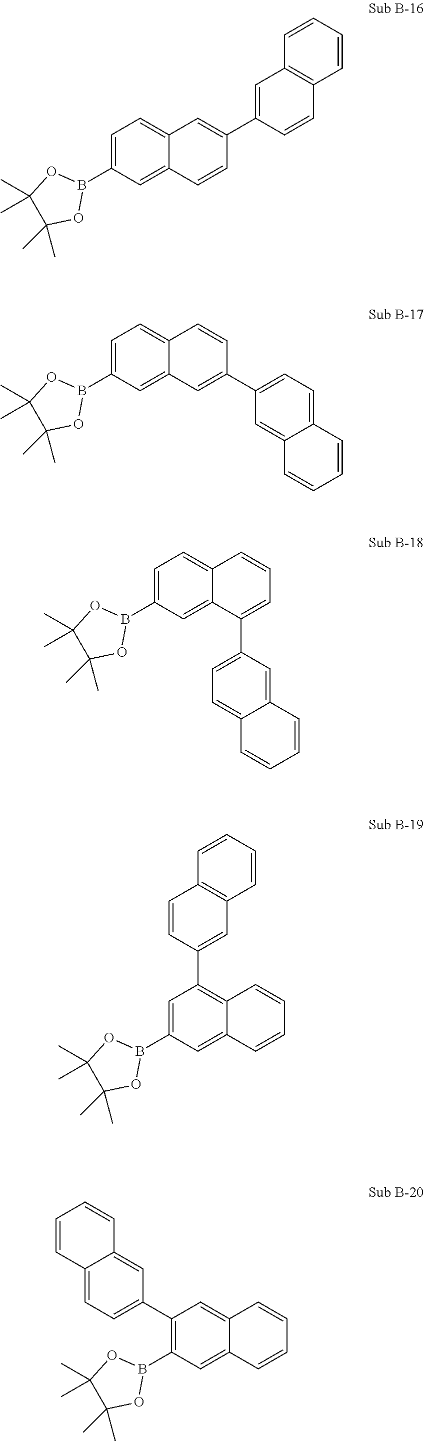 [SMILES: CC1(C)OB(c2ccc3cc(-c4ccc5ccccc5c4)ccc3c2)OC1(C)C ; CC1(C)OB(c2ccc3ccc(-c4ccc5ccccc5c4)cc3c2)OC1(C)C ; CC1(C)OB(c2ccc3cccc(-c4ccc5ccccc5c4)c3c2)OC1(C)C ; CC1(C)OB(c2cc(-c3ccc4ccccc4c3)c3ccccc3c2)OC1(C)C ; CC1(C)OB(c2cc3ccccc3cc2-c2ccc3ccccc3c2)OC1(C)C]

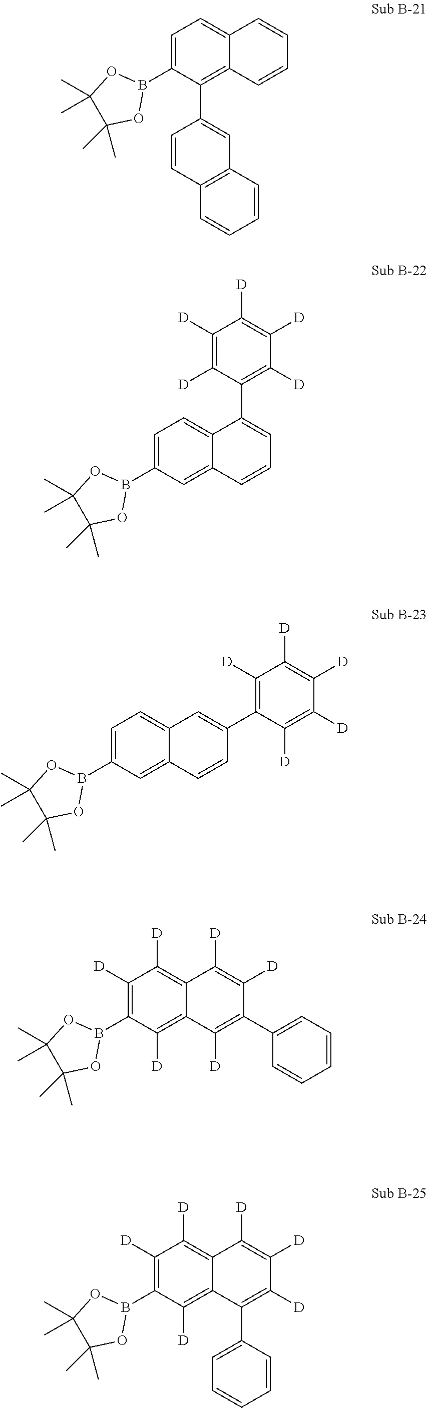 [SMILES: CC1(C)OB(c2ccc3ccccc3c2-c2ccc3ccccc3c2)OC1(C)C ; [2H]c1c([2H])c([2H])c(-c2cccc3cc(B4OC(C)(C)C(C)(C)O4)ccc23)c([2H])c1[2H] ; [2H]c1c([2H])c([2H])c(-c2ccc3cc(B4OC(C)(C)C(C)(C)O4)ccc3c2)c([2H])c1[2H] ; [2H]c1c(B2OC(C)(C)C(C)(C)O2)c([2H])c2c([2H])c(-c3ccccc3)c([2H])c([2H])c2c1[2H] ; [2H]c1c([2H])c(-c2ccccc2)c2c([2H])c(B3OC(C)(C)C(C)(C)O3)c([2H])c([2H])c2c1[2H]]

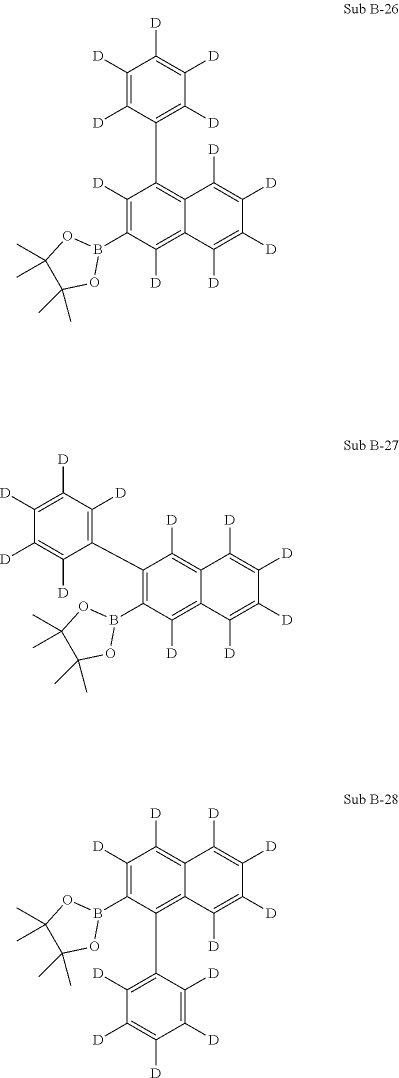 [SMILES: [2H]c1c([2H])c([2H])c(-c2c([2H])c(B3OC(C)(C)C(C)(C)O3)c([2H])c3c([2H])c([2H])c([2H])c([2H])c23)c([2H])c1[2H] ; [2H]c1c([2H])c([2H])c(-c2c(B3OC(C)(C)C(C)(C)O3)c([2H])c3c([2H])c([2H])c([2H])c([2H])c3c2[2H])c([2H])c1[2H] ; [2H]c1c([2H])c([2H])c(-c2c(B3OC(C)(C)C(C)(C)O3)c([2H])c([2H])c3c([2H])c([2H])c([2H])c([2H])c23)c([2H])c1[2H]]

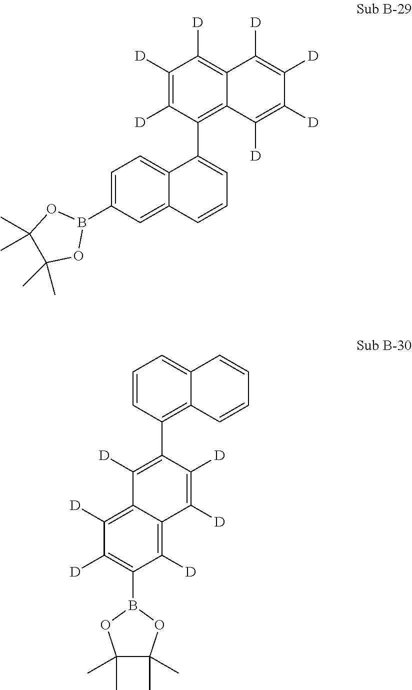 [SMILES: [2H]c1c([2H])c([2H])c2c(-c3cccc4cc(B5OC(C)(C)C(C)(C)O5)ccc34)c([2H])c([2H])c([2H])c2c1[2H] ; [2H]c1c(-c2cccc3ccccc23)c([2H])c2c([2H])c([2H])c(B3OC(C)(C)C(C)(C)O3)c([2H])c2c1[2H]]

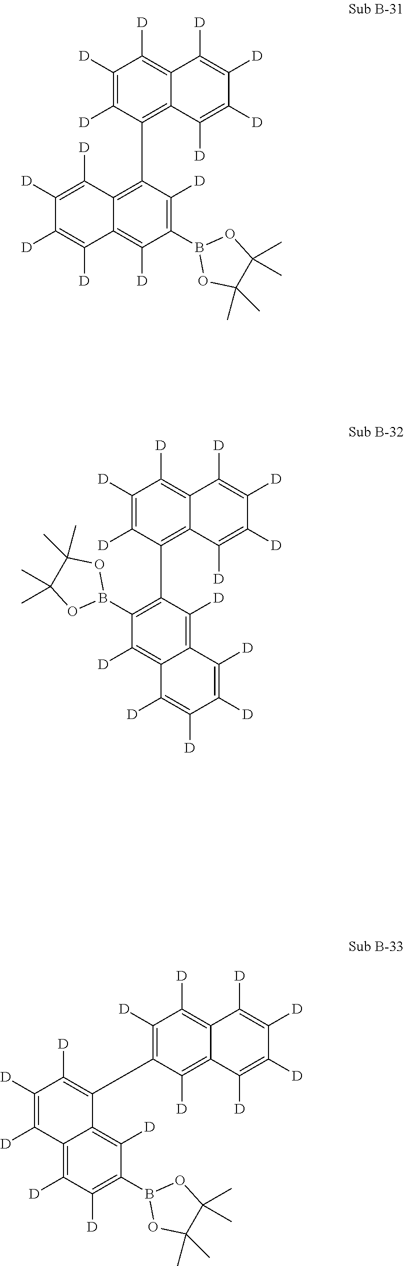

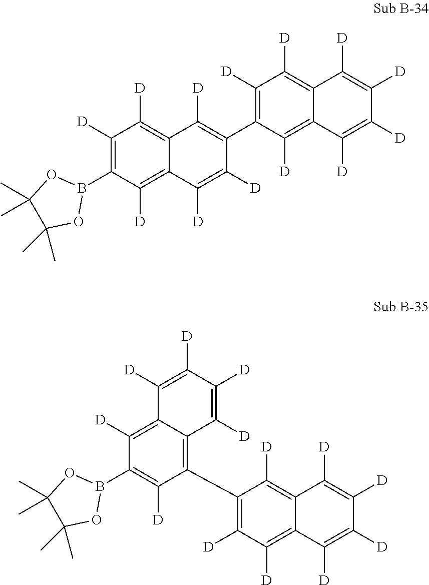 [SMILES: [2H]c1c([2H])c([2H])c2c([2H])c(-c3c([2H])c([2H])c4c([2H])c(B5OC(C)(C)C(C)(C)O5)c([2H])c([2H])c4c3[2H])c([2H])c([2H])c2c1[2H] ; [2H]c1c([2H])c([2H])c2c([2H])c(-c3c([2H])c(B4OC(C)(C)C(C)(C)O4)c([2H])c4c([2H])c([2H])c([2H])c([2H])c34)c([2H])c([2H])c2c1[2H]]

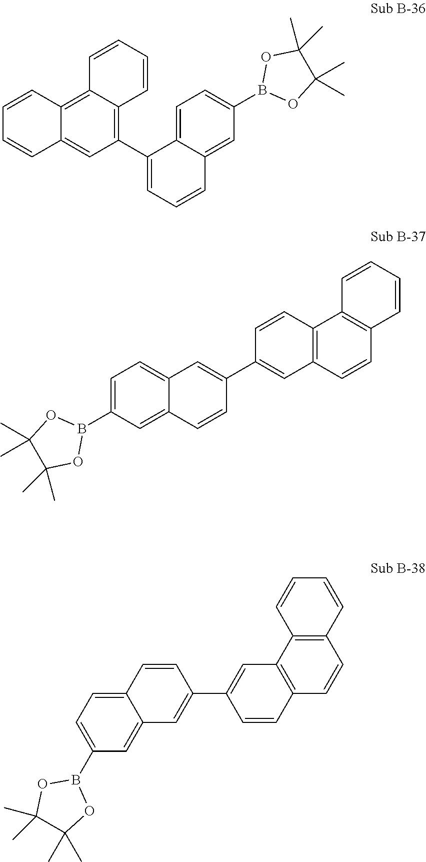

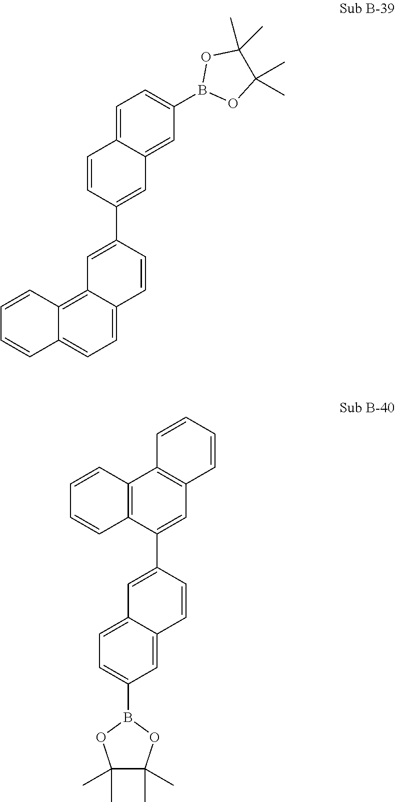 [SMILES: CC1(C)OB(c2ccc3ccc(-c4ccc5ccc6ccccc6c5c4)cc3c2)OC1(C)C ; CC1(C)OB(c2ccc3cc(-c4cc5ccccc5c5ccccc45)ccc3c2)OC1(C)C]

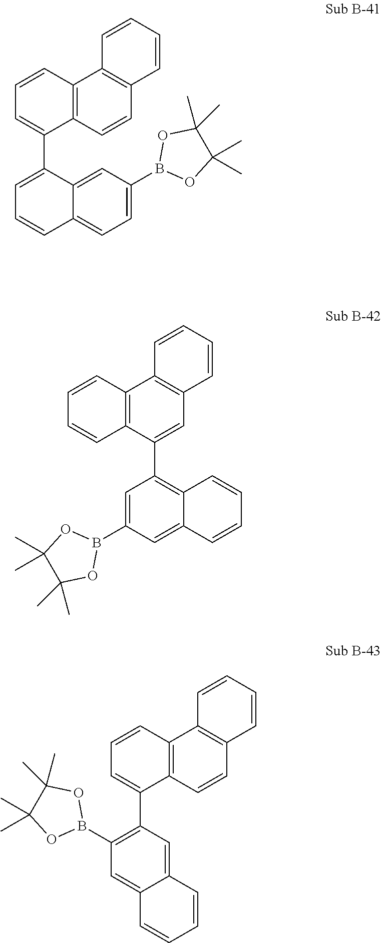

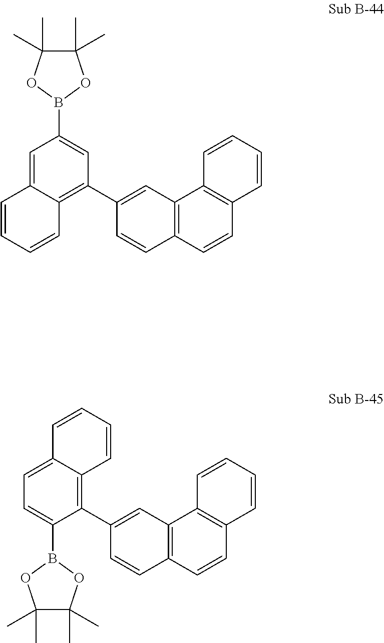 [SMILES: CC1(C)OB(c2cc(-c3ccc4ccc5ccccc5c4c3)c3ccccc3c2)OC1(C)C ; CC1(C)OB(c2ccc3ccccc3c2-c2ccc3ccc4ccccc4c3c2)OC1(C)C]

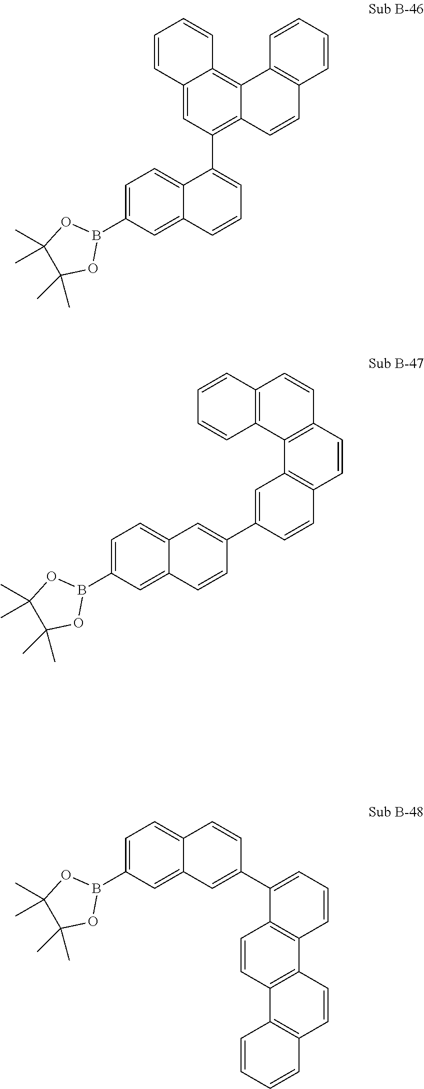 [SMILES: CC1(C)OB(c2ccc3c(-c4cc5ccccc5c5c4ccc4ccccc45)cccc3c2)OC1(C)C ; CC1(C)OB(c2ccc3cc(-c4ccc5ccc6ccc7ccccc7c6c5c4)ccc3c2)OC1(C)C ; CC1(C)OB(c2ccc3ccc(-c4cccc5c4ccc4c6ccccc6ccc54)cc3c2)OC1(C)C]

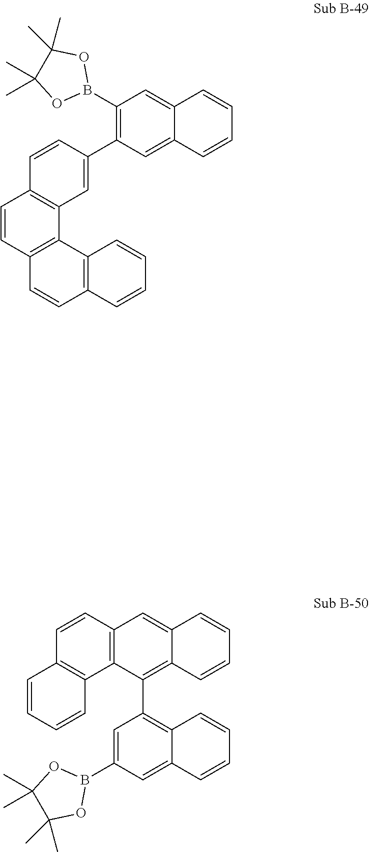 [SMILES: CC1(C)OB(c2cc3ccccc3cc2-c2ccc3ccc4ccc5ccccc5c4c3c2)OC1(C)C ; CC1(C)OB(c2cc(-c3c4ccccc4cc4ccc5ccccc5c34)c3ccccc3c2)OC1(C)C]

TABLE 2

| Compound | FD-MS | Compound | FD-MS |
|---|---|---|---|
| Sub B-1 | m/z = 330.18(C₂₂H₂₃BO₂ = 330.23) | Sub B-2 | m/z = 330.18(C₂₂H₂₃BO₂ = 330.23) |
| Sub B-3 | m/z = 330.18(C₂₂H₂₃BO₂ = 330.23) | Sub B-4 | m/z = 330.18(C₂₂H₂₃BO₂ = 330.23) |
| Sub B-5 | m/z = 330.18(C₂₂H₂₃BO₂ = 330.23) | Sub B-6 | m/z = 330.18(C₂₂H₂₃BO₂ = 330.23) |
| Sub B-7 | m/z = 330.18(C₂₂H₂₃BO₂ = 330.23) | Sub B-8 | m/z = 330.18(C₂₂H₂₃BO₂ = 330.23) |
| Sub B-9 | m/z = 380.19(C₂₆H₂₅BO₂ = 380.29) | Sub B-10 | m/z = 380.19(C₂₆H₂₅BO₂ = 380.29) |
| Sub B-11 | m/z = 380.19(C₂₆H₂₅BO₂ = 380.29) | Sub B-12 | m/z = 380.19(C₂₆H₂₅BO₂ = 380.29) |
| Sub B-13 | m/z = 380.19(C₂₆H₂₅BO₂ = 380.29) | Sub B-14 | m/z = 380.19(C₂₆H₂₅BO₂ = 380.29) |
| Sub B-15 | m/z = 380.19(C₂₆H₂₅BO₂ = 380.29) | Sub B-16 | m/z = 380.19(C₂₆H₂₅BO₂ = 380.29) |
| Sub B-17 | m/z = 380.19(C₂₆H₂₅BO₂ = 380.29) | Sub B-18 | m/z = 380.19(C₂₆H₂₅BO₂ = 380.29) |
| Sub B-19 | m/z = 380.19(C₂₆H₂₅BO₂ = 380.29) | Sub B-20 | m/z = 380.19(C₂₆H₂₅BO₂ = 380.29) |
| Sub B-21 | m/z = 380.19(C₂₆H₂₅BO₂ = 380.29) | Sub B-22 | m/z = 380.19(C₂₆H₂₅BO₂ = 380.29) |
| Sub B-23 | m/z = 335.21(C₂₂H₁₈D₅BO₂ = 335.26) | Sub B-24 | m/z = 336.22(C₂₂H₁₇D₆BO₂ = 336.27) |
| Sub B-25 | m/z = 336.22(C₂₂H₁₇D₆BO₂ = 336.27) | Sub B-26 | m/z = 341.25(C₂₂H₁₂D₁₁BO₂ = 341.3) |
| Sub B-27 | m/z = 341.25(C₂₂H₁₂D₁₁BO₂ = 341.3) | Sub B-28 | m/z = 341.25(C₂₂H₁₂D₁₁BO₂ = 341.3) |
| Sub B-29 | m/z = 387.24(C₂₆H₁₈D₇BO₂ = 387.34) | Sub B-30 | m/z = 386.23(C₂₆H₁₉D₆BO₂ = 386.33) |
| Sub B-31 | m/z = 393.28(C₂₆H₁₂D₁₃BO₂ = 393.37) | Sub B-32 | m/z = 393.28(C₂₆H₁₂D₁₃BO₂ = 393.37) |
| Sub B-33 | m/z = 393.28(C₂₆H₁₂D₁₃BO₂ = 393.37) | Sub B-34 | m/z = 393.28(C₂₆H₁₂D₁₃BO₂ = 393.37) |
| Sub B-35 | m/z = 393.28(C₂₆H₁₂D₁₃BO₂ = 393.37) | Sub B-36 | m/z = 430.21(C₃₀H₂₇BO₂ = 430.35) |
| Sub B-37 | m/z = 430.21(C₃₀H₂₇BO₂ = 430.35) | Sub B-38 | m/z = 430.21(C₃₀H₂₇BO₂ = 430.35) |
| Sub B-39 | m/z = 430.21(C₃₀H₂₇BO₂ = 430.35) | Sub B-40 | m/z = 430.21(C₃₀H₂₇BO₂ = 430.35) |
| Sub B-41 | m/z = 430.21(C₃₀H₂₇BO₂ = 430.35) | Sub B-42 | m/z = 430.21(C₃₀H₂₇BO₂ = 430.35) |
| Sub B-43 | m/z = 430.21(C₃₀H₂₇BO₂ = 430.35) | Sub B-44 | m/z = 430.21(C₃₀H₂₇BO₂ = 430.35) |
| Sub B-45 | m/z = 430.21(C₃₀H₂₇BO₂ = 430.35) | Sub B-46 | m/z = 480.23(C₃₄H₂₉BO₂ = 480.41) |
| Sub B-47 | m/z = 480.23(C₃₄H₂₉BO₂ = 480.41) | Sub B-48 | m/z = 480.23(C₃₄H₂₉BO₂ = 480.41) |
| Sub B-49 | m/z = 480.23(C₃₄H₂₉BO₂ = 480.41) | Sub B-50 | m/z = 480.23(C₃₄H₂₉BO₂ = 480.41) |

### III. Synthesis of Final Product

### 1. Synthesis Example of P1-2

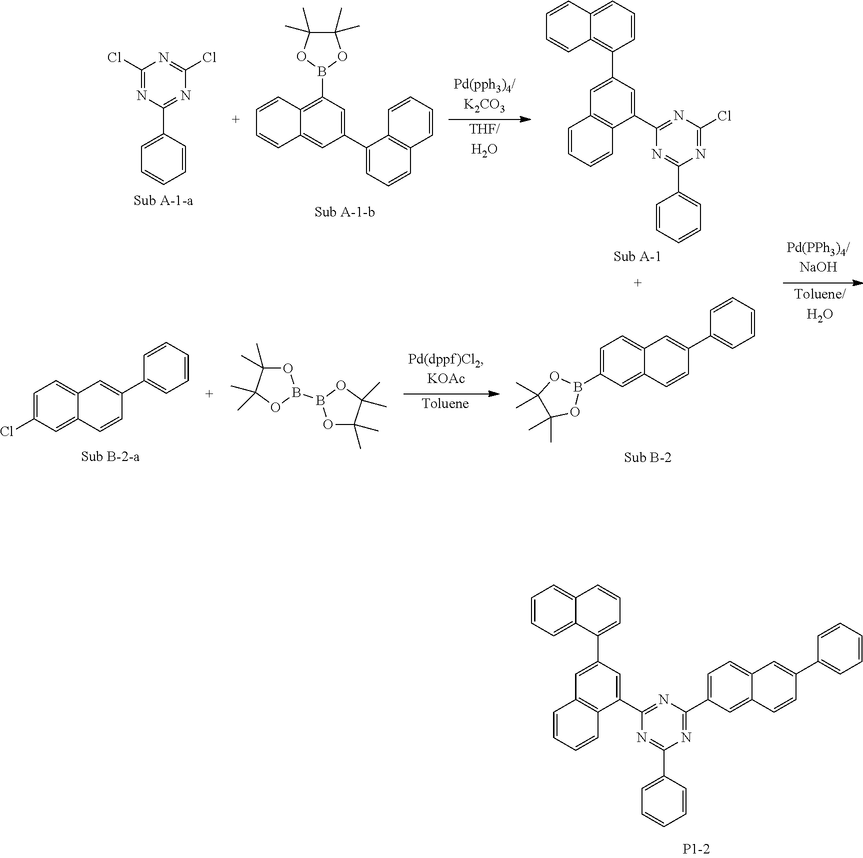

### (1) Synthesis of Sub A-1

Sub A-1-a (33.6 g, 148.6 mmol), Sub A-1-b (28.2 g, 74.3 mmol), Pd(pph₃)₄ (2.5 g, 2.2 mmol) were dissolved in THF (250 mL) in a round bottom flask, K₂CO₃ (20.5 g, 148.6 mmol) was dissolved in H₂O (80 ml) and added and stirred at 50° C. When the reaction was complete, the reaction product was extracted with CH₂Cl₂ and water, and then the organic layer was dried over MgSO₄, concentrated, and the resulting compound was recrystallized using a silica gel column to obtain 21.4 g of the product (65%).

### (2) Synthesis of Sub B-2

Sub B-2-a (35.0 g, 146.6 mmol), Bis(pinacolato)diboron (55.8 g, 219.9 mmol), Pd(dppf)Cl₂ (3.2 g, 4.4 mmol), KOAc (43.1 g, 439.8 mmol) were dissolved in Toluene (500 ml) and added and stirred at 110° C. for 3 hours. When the reaction was complete, the reaction product was extracted with CH₂Cl₂ and water, and then the organic layer was dried over MgSO₄, concentrated, and the resulting compound was recrystallized using a silica gel column to obtain 34.8 g of the product (72%).

### (3) Synthesis of P1-2

Sub A-1 (10.0 g, 22.5 mmol), Sub B-2 (7.4 g, 22.5 mmol), Pd(PPh₃)₄ (0.8 g, 0.6 mmol) were dissolved in Toluene (80 ml) in a round bottom flask, NaOH (1.8 g, 45.0 mmol) was dissolved in H₂O (25 ml), added, and refluxed for 3 hours. When the reaction was complete, the reaction product was extracted with CH₂Cl₂ and water, and then the organic layer was dried over MgSO₄, concentrated, and the resulting compound was recrystallized using a silica gel column to obtain 9.9 g of the product (72%).

### 2. Synthesis Example of P1-5

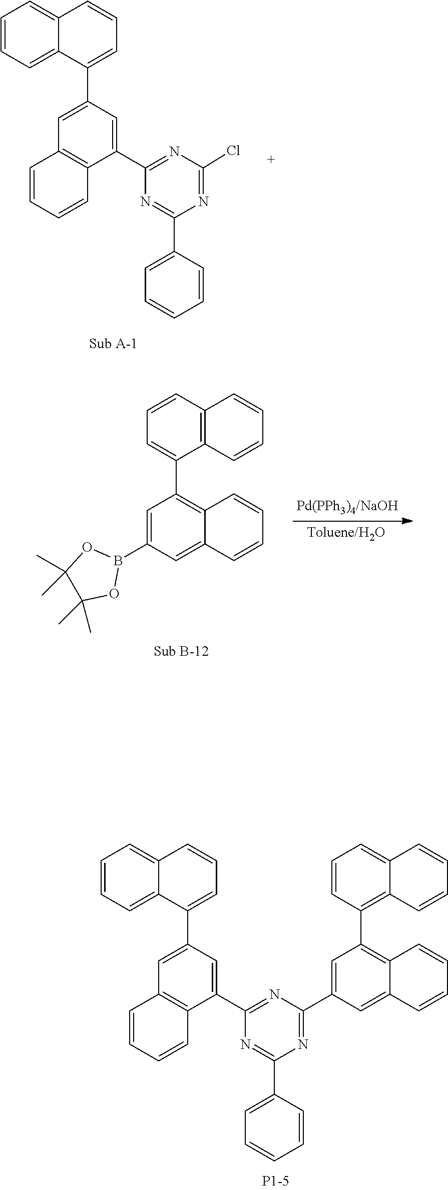 [SMILES: Clc1nc(-c2ccccc2)nc(-c2cc(-c3cccc4ccccc34)cc3ccccc23)n1 ; CC1(C)OB(c2cc(-c3cccc4ccccc34)c3ccccc3c2)OC1(C)C ; c1ccc(-c2nc(-c3cc(-c4cccc5ccccc45)c4ccccc4c3)nc(-c3cc(-c4cccc5ccccc45)cc4ccccc34)n2)cc1]

Sub A-1 (10.0 g, 22.5 mmol), Sub B-12 (8.5 g, 22.5 mmol), Pd(PPh₃)₄ (0.8 g, 0.6 mmol), NaOH (1.8 g, 45.0 mmol) were added, and 11.0 g of the product (yield 74%) was obtained using the synthesis method of P1-2.

### 3. Synthesis Example of P1-11

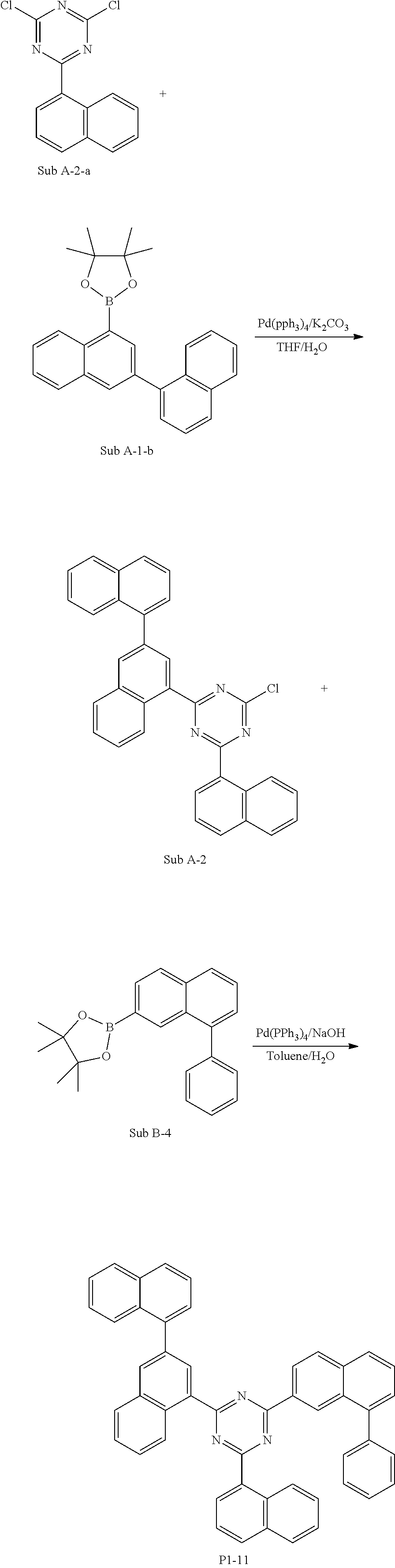 [SMILES: Clc1nc(Cl)nc(-c2cccc3ccccc23)n1 ; CC1(C)OB(c2cc(-c3cccc4ccccc34)cc3ccccc23)OC1(C)C ; Clc1nc(-c2cccc3ccccc23)nc(-c2cc(-c3cccc4ccccc34)cc3ccccc23)n1 ; CC1(C)OB(c2ccc3cccc(-c4ccccc4)c3c2)OC1(C)C ; c1ccc(-c2cccc3ccc(-c4nc(-c5cccc6ccccc56)nc(-c5cc(-c6cccc7ccccc67)cc6ccccc56)n4)cc23)cc1]

### (1) Synthesis of SubA-2

Sub A-2-a (41.0 g, 148.6 mmol), Sub A-1-b (28.2 g, 74.3 mmol), Pd(pph₃)₄ (2.5 g, 2.2 mmol), K₂CO₃ (20.5 g, 148.6 mmol) were added, and 24.9 g of the product (yield 68%) was obtained using the synthesis method of Sub A-1.

### (2) Synthesis of P1-11

Sub A-2 (15.0 g, 30.3 mmol), Sub B-4 (10 g, 30.3 mmol), Pd(PPh₃)₄ (1.1 g, 0.9 mmol), NaOH (2.4 g, 60.6 mmol) were added, and 15.6 g of the product (yield 78%) was obtained using the synthesis method of P1-2.

### 4. Synthesis Example of P1-12

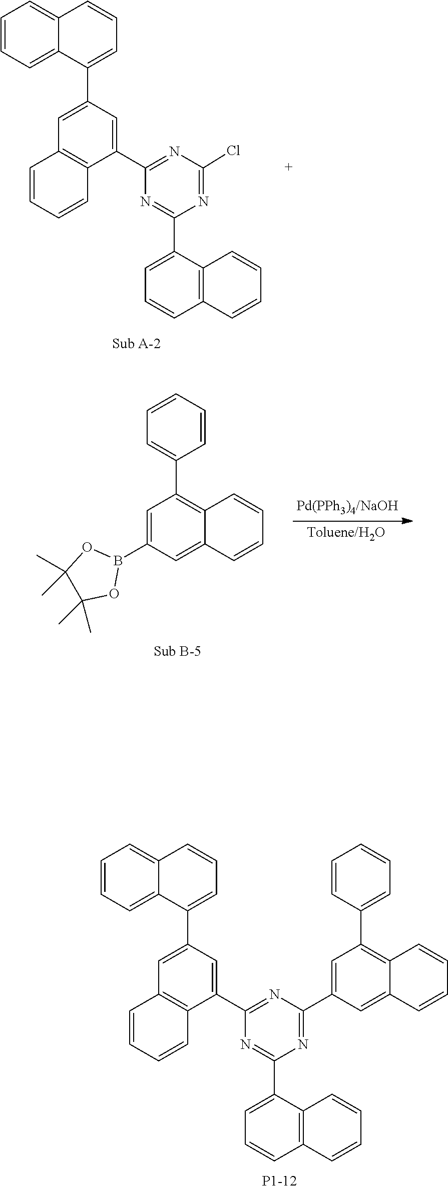 [SMILES: Clc1nc(-c2cccc3ccccc23)nc(-c2cc(-c3cccc4ccccc34)cc3ccccc23)n1 ; CC1(C)OB(c2cc(-c3ccccc3)c3ccccc3c2)OC1(C)C ; c1ccc(-c2cc(-c3nc(-c4cccc5ccccc45)nc(-c4cc(-c5cccc6ccccc56)cc5ccccc45)n3)cc3ccccc23)cc1]

Sub A-2 (15.0 g, 30.3 mmol), Sub B-5 (10 g, 30.3 mmol), Pd(PPh₃)₄ (1.1 g, 0.9 mmol), NaOH (2.4 g, 60.6 mmol) were added, and 14.4 g of the product (yield 72%) was obtained using the synthesis method of P1-2.

### 5. Synthesis Example of P1-15

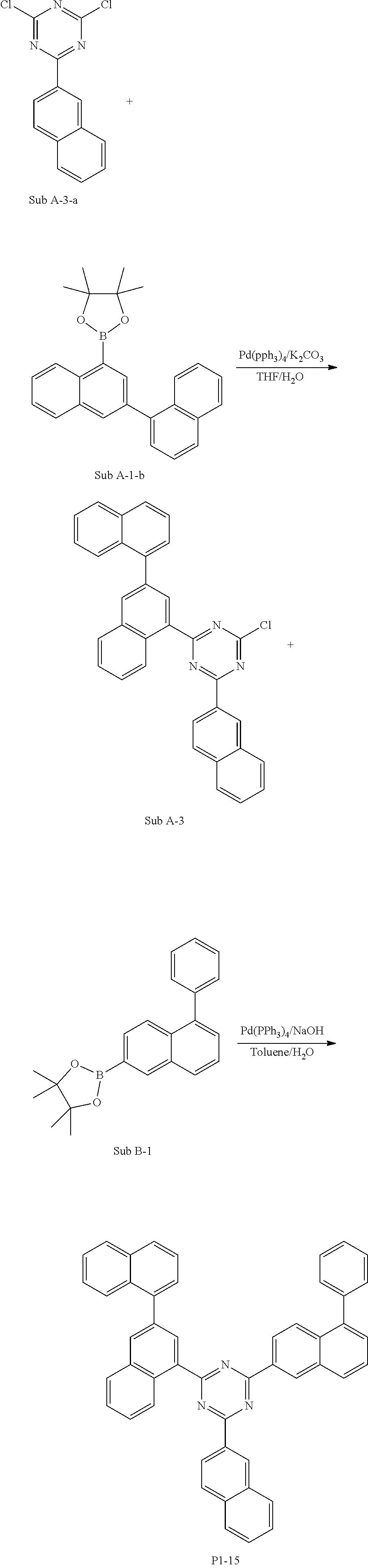 [SMILES: Clc1nc(Cl)nc(-c2ccc3ccccc3c2)n1 ; CC1(C)OB(c2cc(-c3cccc4ccccc34)cc3ccccc23)OC1(C)C ; Clc1nc(-c2ccc3ccccc3c2)nc(-c2cc(-c3cccc4ccccc34)cc3ccccc23)n1 ; CC1(C)OB(c2ccc3c(-c4ccccc4)cccc3c2)OC1(C)C ; c1ccc(-c2cccc3cc(-c4nc(-c5ccc6ccccc6c5)nc(-c5cc(-c6cccc7ccccc67)cc6ccccc56)n4)ccc23)cc1]

### (1) Synthesis of SubA-3

Sub A-3-a (41.0 g, 148.6 mmol), Sub A-1-b (25.0 g, 74.3 mmol), Pd(pph₃)₄ (2.5 g, 2.2 mmol), K₂CO₃ (20.5 g, 148.6 mmol) were added, and 27.5 g of the product (yield 75%) was obtained using the synthesis method of Sub A-1.

### (2) Synthesis of P1-15

Sub A-3 (15.0 g, 30.3 mmol), Sub B-1 (10 g, 30.3 mmol), Pd(PPh₃)₄ (1.1 g, 0.9 mmol), NaOH (2.4 g, 60.6 mmol) were added, and 15.0 g of the product (yield 75%) was obtained using the synthesis method of P1-2.

### 6. Synthesis Example of P1-24

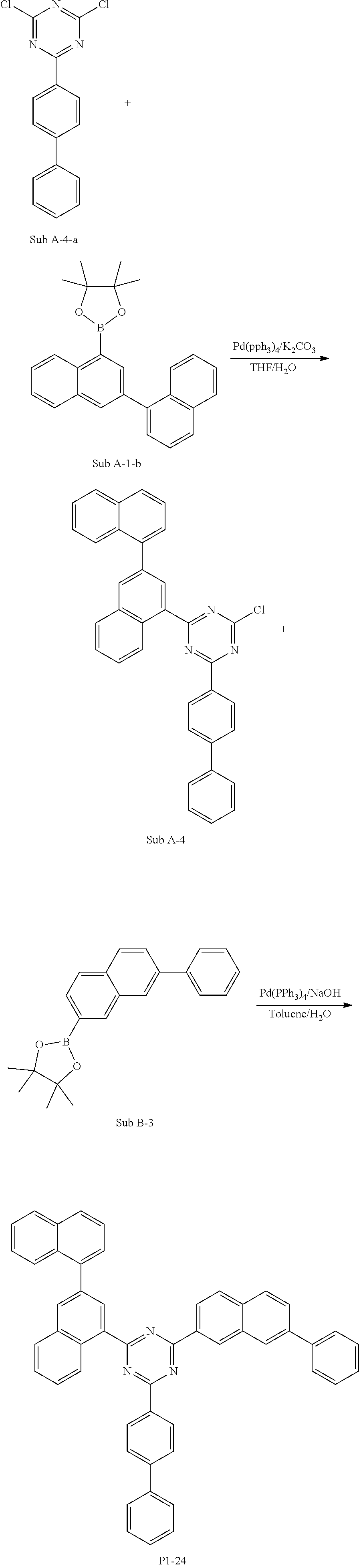 [SMILES: Clc1nc(Cl)nc(-c2ccc(-c3ccccc3)cc2)n1 ; CC1(C)OB(c2cc(-c3cccc4ccccc34)cc3ccccc23)OC1(C)C ; Clc1nc(-c2ccc(-c3ccccc3)cc2)nc(-c2cc(-c3cccc4ccccc34)cc3ccccc23)n1 ; CC1(C)OB(c2ccc3ccc(-c4ccccc4)cc3c2)OC1(C)C ; c1ccc(-c2ccc(-c3nc(-c4ccc5ccc(-c6ccccc6)cc5c4)nc(-c4cc(-c5cccc6ccccc56)cc5ccccc45)n3)cc2)cc1]

### (1) Synthesis of SubA-4

Sub A-4-a (30.2 g, 100.0 mmol), Sub A-1-b (19.0 g, 50.0 mmol), Pd(pph₃)₄ (1.7 g, 1.5 mmol), K₂CO₃ (13.8 g, 100.0 mmol) were added, and 19.5 g of the product (yield 75%) was obtained using the synthesis method of Sub A-1.

### (2) Synthesis of P1-24

Sub A-4 (15.0 g, 28.8 mmol), Sub B-3 (9.5 g, 28.8 mmol), Pd(PPh₃)₄ (1.0 g, 0.9 mmol), NaOH (2.3 g, 57.6 mmol) were added, and 12.8 g of the product (yield 65%) was obtained using the synthesis method of P1-2.

### 7. Synthesis Example of P1-88

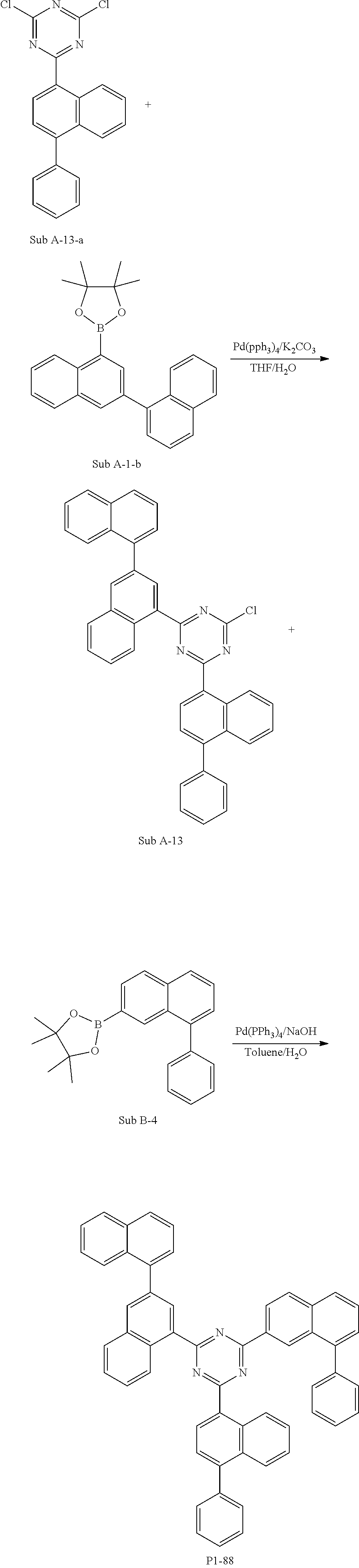 [SMILES: Clc1nc(Cl)nc(-c2ccc(-c3ccccc3)c3ccccc23)n1 ; CC1(C)OB(c2cc(-c3cccc4ccccc34)cc3ccccc23)OC1(C)C ; Clc1nc(-c2cc(-c3cccc4ccccc34)cc3ccccc23)nc(-c2ccc(-c3ccccc3)c3ccccc23)n1 ; CC1(C)OB(c2ccc3cccc(-c4ccccc4)c3c2)OC1(C)C ; c1ccc(-c2cccc3ccc(-c4nc(-c5cc(-c6cccc7ccccc67)cc6ccccc56)nc(-c5ccc(-c6ccccc6)c6ccccc56)n4)cc23)cc1]

### (1) Synthesis of SubA-13

Sub A-13-a (30.0 g, 85.2 mmol), Sub A-1-b (16.2 g, 42.6 mmol), Pd(pph₃)₄ (1.5 g, 1.3 mmol), K₂CO₃ (13.8 g, 85.2 mmol) were added, and 19.9 g of the product (yield 82%) was obtained using the synthesis method of Sub A-1.

### (2) Synthesis of P1-88

Sub A-13 (10.0 g, 17.5 mmol), Sub B-4 (5.8 g, 17.5 mmol), Pd(PPh₃)₄ (0.6 g, 0.5 mmol), NaOH (1.4 g, 35.0 mmol) were added, and 10.0 g of the product (yield 77%) was obtained using the synthesis method of P1-2.

### 8. Synthesis Example of P1-146

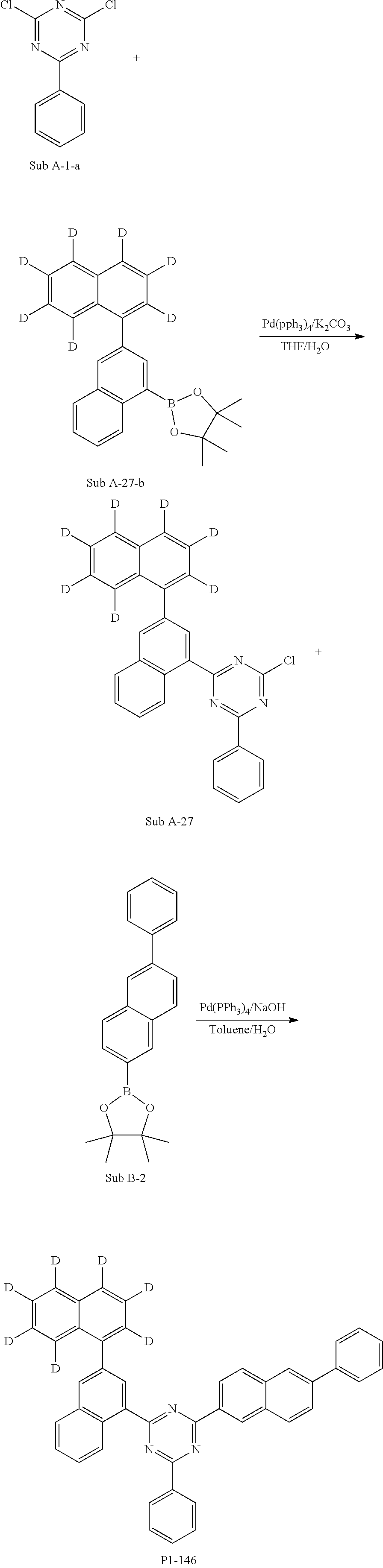 [SMILES: Clc1nc(Cl)nc(-c2ccccc2)n1 ; [2H]c1c([2H])c([2H])c2c(-c3cc(B4OC(C)(C)C(C)(C)O4)c4ccccc4c3)c([2H])c([2H])c([2H])c2c1[2H] ; [2H]c1c([2H])c([2H])c2c(-c3cc(-c4nc(Cl)nc(-c5ccccc5)n4)c4ccccc4c3)c([2H])c([2H])c([2H])c2c1[2H] ; CC1(C)OB(c2ccc3cc(-c4ccccc4)ccc3c2)OC1(C)C ; [2H]c1c([2H])c([2H])c2c(-c3cc(-c4nc(-c5ccccc5)nc(-c5ccc6cc(-c7ccccc7)ccc6c5)n4)c4ccccc4c3)c([2H])c([2H])c([2H])c2c1[2H]]

### (1) Synthesis of SubA-27

Sub A-1-a (35.0 g, 110.5 mmol), Sub A-27-b (21.4 g, 55.3 mmol), Pd(pph₃)₄ (1.9 g, 1.6 mmol), K₂CO₃ (15.2 g, 110.5 mmol) were added, and 20.2 g of the product (yield 81%) was obtained using the synthesis method of Sub A-1.

### (2) Synthesis of P1-146

Sub A-27 (20.0 g, 44.3 mmol), Sub B-2 (14.6 g, 44.3 mmol), Pd(PPh₃)₄ (1.5 g, 1.3 mmol), NaOH (3.5 g, 88.6 mmol) were added, and 19.7 g of the product (yield 72%) was obtained using the synthesis method of P1-2.

### 9. Synthesis Example of P1-166

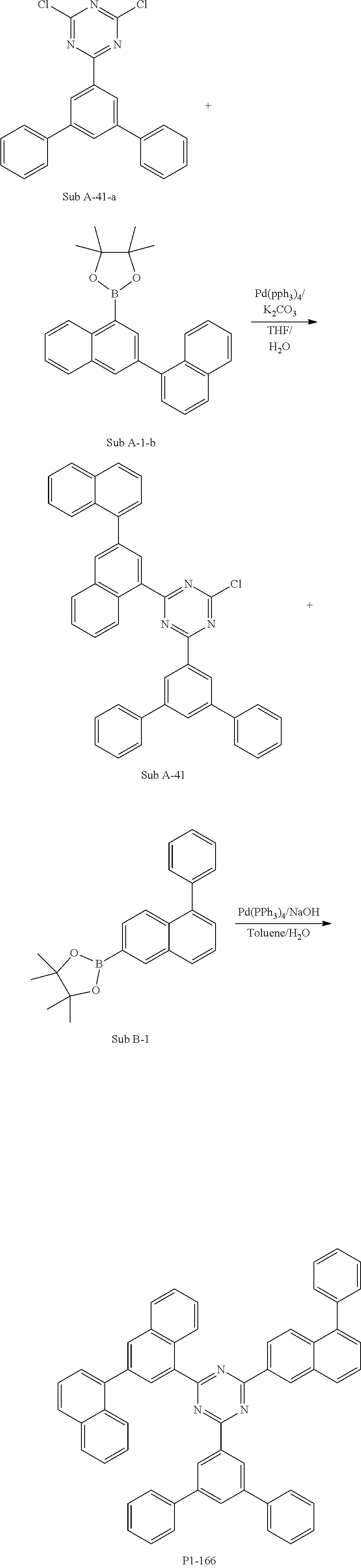 [SMILES: Clc1nc(Cl)nc(-c2cc(-c3ccccc3)cc(-c3ccccc3)c2)n1 ; CC1(C)OB(c2cc(-c3cccc4ccccc34)cc3ccccc23)OC1(C)C ; Clc1nc(-c2cc(-c3ccccc3)cc(-c3ccccc3)c2)nc(-c2cc(-c3cccc4ccccc34)cc3ccccc23)n1 ; CC1(C)OB(c2ccc3c(-c4ccccc4)cccc3c2)OC1(C)C ; c1ccc(-c2cc(-c3ccccc3)cc(-c3nc(-c4ccc5c(-c6ccccc6)cccc5c4)nc(-c4cc(-c5cccc6ccccc56)cc5ccccc45)n3)c2)cc1]

### (1) Synthesis of SubA-41

Sub A-41-a (30.0 g, 79.3 mmol), Sub A-1-b (15.0 g, 39.6 mmol), Pd(pph₃)₄ (1.3 g, 1.2 mmol), K₂CO₃ (10.9 g, 79.3 mmol) were added, and 17.9 g of the product (yield 76%) was obtained using the synthesis method of Sub A-1.

### (2) Synthesis of P1-166

Sub A-41 (10.0 g, 16.8 mmol), Sub B-1 (5.5 g, 16.8 mmol), Pd(PPh₃)₄ (0.6 g, 0.5 mmol), NaOH (1.3 g, 33.6 mmol) were added, and 8.8 g of the product (yield 69%) was obtained using the synthesis method of P1-2.

### 10. Synthesis Example of P2-1

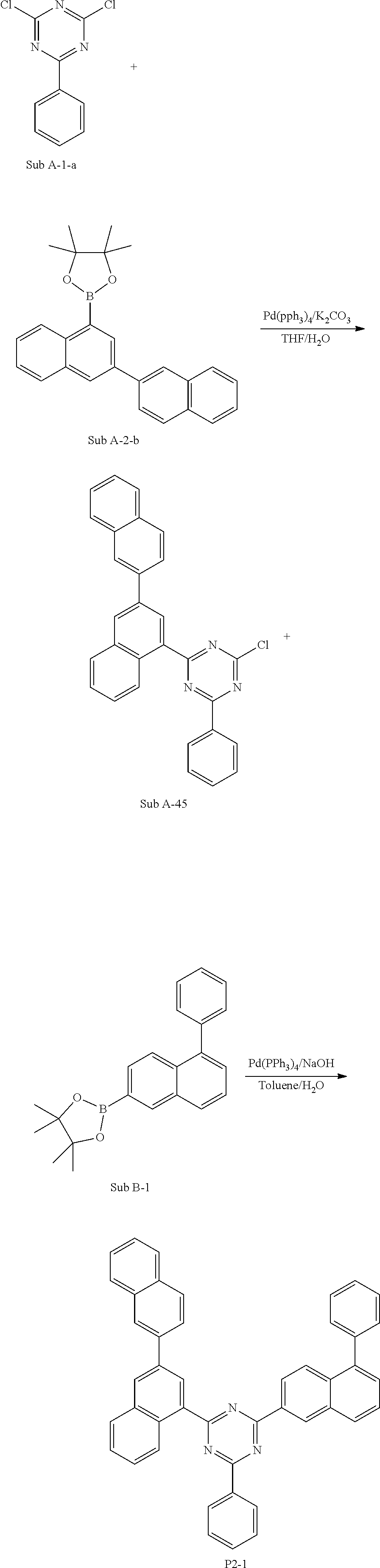

### (1) Synthesis of SubA-45

Sub A-41-a (30.0 g, 79.3 mmol), Sub A-1-b (15.0 g, 39.6 mmol), Pd(pph₃)₄ (1.3 g, 1.2 mmol), K₂CO₃ (10.9 g, 79.3 mmol) were added, and 15.5 g of the product (yield 79%) was obtained using the synthesis method of Sub A-1.

### (2) Synthesis of P2-1

Sub A-45 (15.0 g, 33.8 mmol), Sub B-1 (11.1 g, 33.8 mmol), Pd(PPh₃)₄ (1.1 g, 1.0 mmol), NaOH (2.7 g, 67.6 mmol) were added, and 14.6 g of the product (yield 71%) was obtained using the synthesis method of P1-2.

### 11. Synthesis Example of P2-12

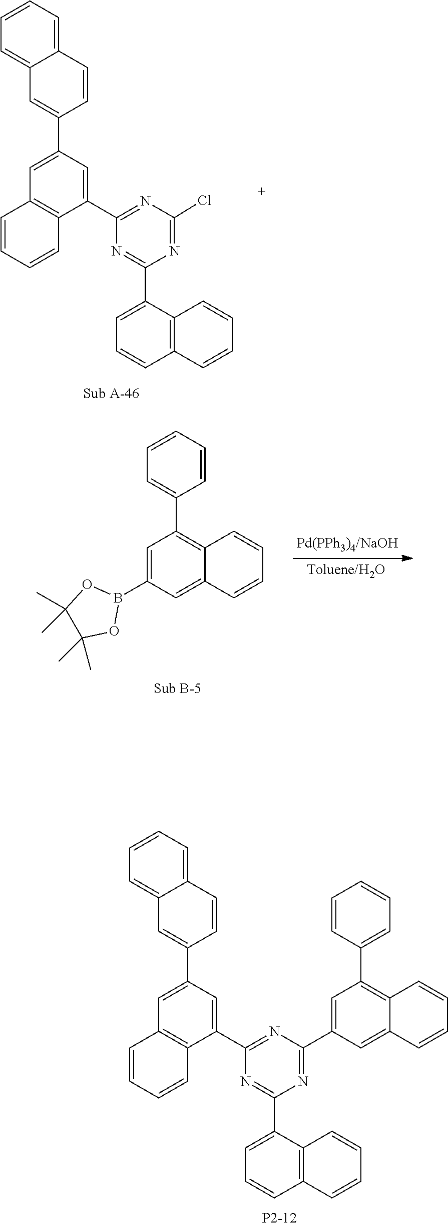 [SMILES: Clc1nc(-c2cccc3ccccc23)nc(-c2cc(-c3ccc4ccccc4c3)cc3ccccc23)n1 ; CC1(C)OB(c2cc(-c3ccccc3)c3ccccc3c2)OC1(C)C ; c1ccc(-c2cc(-c3nc(-c4cccc5ccccc45)nc(-c4cc(-c5ccc6ccccc6c5)cc5ccccc45)n3)cc3ccccc23)cc1]

Sub A-46 (15.0 g, 30.3 mmol), Sub B-5 (10.0 g, 30.3 mmol), Pd(PPh₃)₄ (1.0 g, 0.9 mmol), NaOH (2.4 g, 60.6 mmol) were added, and 15.6 g of the product (yield 78%) was obtained using the synthesis method of P1-2.

### 12. Synthesis Example of P2-29

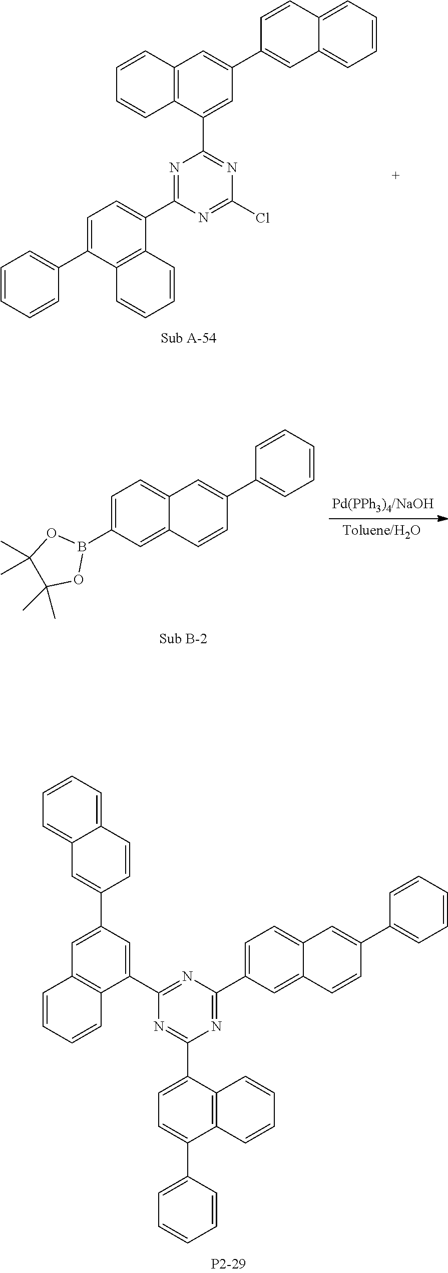 [SMILES: Clc1nc(-c2cc(-c3ccc4ccccc4c3)cc3ccccc23)nc(-c2ccc(-c3ccccc3)c3ccccc23)n1 ; CC1(C)OB(c2ccc3cc(-c4ccccc4)ccc3c2)OC1(C)C ; c1ccc(-c2ccc3cc(-c4nc(-c5cc(-c6ccc7ccccc7c6)cc6ccccc56)nc(-c5ccc(-c6ccccc6)c6ccccc56)n4)ccc3c2)cc1]

Sub A-54 (10.0 g, 17.5 mmol), Sub B-2 (5.8 g, 17.5 mmol), Pd(PPh₃)₄ (0.6 g, 0.5 mmol), NaOH (1.4 g, 35.0 mmol) were added, and 10.0 g of the product (yield 78%) was obtained using the synthesis method of P1-2.

TABLE 3

| Compound | FD-MS | Compound | FD-MS |
|---|---|---|---|
| P1-1 | m/z = 611.24(C₄₅H₂₉N₃ = 611.75) | P1-2 | m/z = 611.24(C₄₅H₂₉N₃ = 611.75) |
| P1-3 | m/z = 611.24(C₄₅H₂₉N₃ = 611.75) | P1-4 | m/z = 611.24(C₄₅H₂₉N₃ = 611.75) |
| P1-5 | m/z = 661.25(C₄₉H₃₁N₃ = 661.81) | P1-6 | m/z = 661.25(C₄₉H₃₁N₃ = 661.81) |
| P1-7 | m/z = 661.25(C₄₉H₃₁N₃ = 661.81) | P1-8 | m/z = 661.25(C₄₉H₃₁N₃ = 661.81) |
| P1-9 | m/z = 661.25(C₄₉H₃₁N₃ = 661.81) | P1-10 | m/z = 661.25(C₄₉H₃₁N₃ = 661.81) |
| P1-11 | m/z = 661.25(C₄₉H₃₁N₃ = 661.81) | P1-12 | m/z = 661.25(C₄₉H₃₁N₃ = 661.81) |
| P1-13 | m/z = 711.27(C₅₃H₃₃N₃ = 711.87) | P1-14 | m/z = 711.27(C₅₃H₃₃N₃ = 711.87) |
| P1-15 | m/z = 661.25(C₄₉H₃₁N₃ = 661.81) | P1-16 | m/z = 661.25(C₄₉H₃₁N₃ = 661.81) |
| P1-17 | m/z = 661.25(C₄₉H₃₁N₃ = 661.81) | P1-18 | m/z = 661.25(C₄₉H₃₁N₃ = 661.81) |
| P1-19 | m/z = 661.25(C₄₉H₃₁N₃ = 661.81) | P1-20 | m/z = 661.25(C₄₉H₃₁N₃ = 661.81) |
| P1-21 | m/z = 711.27(C₅₃H₃₃N₃ = 711.87) | P1-22 | m/z = 687.27(C₅₁H₃₃N₃ = 687.85) |
| P1-23 | m/z = 687.27(C₅₁H₃₃N₃ = 687.85) | P1-24 | m/z = 687.27(C₅₁H₃₃N₃ = 687.85) |
| P1-25 | m/z = 687.27(C₅₁H₃₃N₃ = 687.85) | P1-26 | m/z = 687.27(C₅₁H₃₃N₃ = 687.85) |
| P1-27 | m/z = 687.27(C₅₁H₃₃N₃ = 687.85) | P1-28 | m/z = 687.27(C₅₁H₃₃N₃ = 687.85) |
| P1-29 | m/z = 687.27(C₅₁H₃₃N₃ = 687.85) | P1-30 | m/z = 687.27(C₅₁H₃₃N₃ = 687.85) |
| P1-31 | m/z = 687.27(C₅₁H₃₃N₃ = 687.85) | P1-32 | m/z = 687.27(C₅₁H₃₃N₃ = 687.85) |
| P1-33 | m/z = 737.28(C₅₅H₃₅N₃ = 737.91) | P1-34 | m/z = 737.28(C₅₅H₃₅N₃ = 737.91) |
| P1-35 | m/z = 737.28(C₅₅H₃₅N₃ = 737.91) | P1-36 | m/z = 687.27(C₅₁H₃₃N₃ = 687.85) |
| P1-37 | m/z = 687.27(C₅₁H₃₃N₃ = 687.85) | P1-38 | m/z = 687.27(C₅₁H₃₃N₃ = 687.85) |
| P1-39 | m/z = 687.27(C₅₁H₃₃N₃ = 687.85) | P1-40 | m/z = 687.27(C₅₁H₃₃N₃ = 687.85) |
| P1-41 | m/z = 737.28(C₅₅H₃₅N₃ = 737.91) | P1-42 | m/z = 687.27(C₅₁H₃₃N₃ = 687.85) |
| P1-43 | m/z = 737.28(C₅₅H₃₅N₃ = 737.91) | P1-44 | m/z = 737.28(C₅₅H₃₅N₃ = 737.91) |
| P1-45 | m/z = 737.28(C₅₅H₃₅N₃ = 737.91) | P1-46 | m/z = 737.28(C₅₅H₃₅N₃ = 737.91) |
| P1-47 | m/z = 737.28(C₅₅H₃₅N₃ = 737.91) | P1-48 | m/z = 737.28(C₅₅H₃₅N₃ = 737.91) |
| P1-49 | m/z = 787.3(C₅₉H₃₇N₃ = 787.97) | P1-50 | m/z = 737.28(C₅₅H₃₅N₃ = 737.91) |
| P1-51 | m/z = 737.28(C₅₅H₃₅N₃ = 737.91) | P1-52 | m/z = 737.28(C₅₅H₃₅N₃ = 737.91) |
| P1-53 | m/z = 737.28(C₅₅H₃₅N₃ = 737.91) | P1-54 | m/z = 737.28(C₅₅H₃₅N₃ = 737.91) |
| P1-55 | m/z = 737.28(C₅₅H₃₅N₃ = 737.91) | P1-56 | m/z = 737.28(C₅₅H₃₅N₃ = 737.91) |
| P1-57 | m/z = 737.28(C₅₅H₃₅N₃ = 737.91) | P1-58 | m/z = 737.28(C₅₅H₃₅N₃ = 737.91) |
| P1-59 | m/z = 737.28(C₅₅H₃₅N₃ = 737.91) | P1-60 | m/z = 737.28(C₅₅H₃₅N₃ = 737.91) |
| P1-61 | m/z = 737.28(C₅₅H₃₅N₃ = 737.91) | P1-62 | m/z = 737.28(C₅₅H₃₅N₃ = 737.91) |
| P1-63 | m/z = 737.28(C₅₅H₃₅N₃ = 737.91) | P1-64 | m/z = 737.28(C₅₅H₃₅N₃ = 737.91) |
| P1-65 | m/z = 737.28(C₅₅H₃₅N₃ = 737.91) | P1-66 | m/z = 737.28(C₅₅H₃₅N₃ = 737.91) |
| P1-67 | m/z = 737.28(C₅₅H₃₅N₃ = 737.91) | P1-68 | m/z = 737.28(C₅₅H₃₅N₃ = 737.91) |
| P1-69 | m/z = 737.28(C₅₅H₃₅N₃ = 737.91) | P1-70 | m/z = 737.28(C₅₅H₃₅N₃ = 737.91) |
| P1-71 | m/z = 737.28(C₅₅H₃₅N₃ = 737.91) | P1-72 | m/z = 737.28(C₅₅H₃₅N₃ = 737.91) |
| P1-73 | m/z = 737.28(C₅₅H₃₅N₃ = 737.91) | P1-74 | m/z = 737.28(C₅₅H₃₅N₃ = 737.91) |
| P1-75 | m/z = 737.28(C₅₅H₃₅N₃ = 737.91) | P1-76 | m/z = 737.28(C₅₅H₃₅N₃ = 737.91) |
| P1-77 | m/z = 787.3(C₅₉H₃₇N₃ = 787.97) | P1-78 | m/z = 737.28(C₅₅H₃₅N₃ = 737.91) |
| P1-79 | m/z = 737.28(C₅₅H₃₅N₃ = 737.91) | P1-80 | m/z = 737.28(C₅₅H₃₅N₃ = 737.91) |
| P1-81 | m/z = 737.28(C₅₅H₃₅N₃ = 737.91) | P1-82 | m/z = 737.28(C₅₅H₃₅N₃ = 737.91) |
| P1-83 | m/z = 737.28(C₅₅H₃₅N₃ = 737.91) | P1-84 | m/z = 737.28(C₅₅H₃₅N₃ = 737.91) |
| P1-85 | m/z = 737.28(C₅₅H₃₅N₃ = 737.91) | P1-86 | m/z = 737.28(C₅₅H₃₅N₃ = 737.91) |
| P1-87 | m/z = 737.28(C₅₅H₃₅N₃ = 737.91) | P1-88 | m/z = 737.28(C₅₅H₃₅N₃ = 737.91) |
| P1-89 | m/z = 737.28(C₅₅H₃₅N₃ = 737.91) | P1-90 | m/z = 737.28(C₅₅H₃₅N₃ = 737.91) |
| P1-91 | m/z = 737.28(C₅₅H₃₅N₃ = 737.91) | P1-92 | m/z = 737.28(C₅₅H₃₅N₃ = 737.91) |
| P1-93 | m/z = 737.28(C₅₅H₃₅N₃ = 737.91) | P1-94 | m/z = 737.28(C₅₅H₃₅N₃ = 737.91) |
| P1-95 | m/z = 737.28(C₅₅H₃₅N₃ = 737.91) | P1-96 | m/z = 737.28(C₅₅H₃₅N₃ = 737.91) |
| P1-97 | m/z = 737.28(C₅₅H₃₅N₃ = 737.91) | P1-98 | m/z = 737.28(C₅₅H₃₅N₃ = 737.91) |
| P1-99 | m/z = 837.31(C₆₃H₃₉N₃ = 838.03) | P1-100 | m/z = 837.31(C₆₃H₃₉N₃ = 838.03) |
| P1-101 | m/z = 737.28(C₅₅H₃₅N₃ = 737.91) | P1-102 | m/z = 737.28(C₅₅H₃₅N₃ = 737.91) |
| P1-103 | m/z = 737.28(C₅₅H₃₅N₃ = 737.91) | P1-104 | m/z = 737.28(C₅₅H₃₅N₃ = 737.91) |
| P1-105 | m/z = 737.28(C₅₅H₃₅N₃ = 737.91) | P1-106 | m/z = 737.28(C₅₅H₃₅N₃ = 737.91) |
| P1-107 | m/z = 737.28(C₅₅H₃₅N₃ = 737.91) | P1-108 | m/z = 737.28(C₅₅H₃₅N₃ = 737.91) |
| P1-109 | m/z = 737.28(C₅₅H₃₅N₃ = 737.91) | P1-110 | m/z = 737.28(C₅₅H₃₅N₃ = 737.91) |
| P1-111 | m/z = 737.28(C₅₅H₃₅N₃ = 737.91) | P1-112 | m/z = 737.28(C₅₅H₃₅N₃ = 737.91) |
| P1-113 | m/z = 787.3(C₅₉H₃₇N₃ = 787.97) | P1-114 | m/z = 787.3(C₅₉H₃₇N₃ = 787.97) |
| P1-115 | m/z = 837.31(C₆₃H₃₉N₃ = 838.03) | P1-116 | m/z = 887.33(C₆₇H₄₁N₃ = 888.09) |
| P1-117 | m/z = 737.28(C₅₅H₃₅N₃ = 737.91) | P1-118 | m/z = 737.28(C₅₅H₃₅N₃ = 737.91) |
| P1-119 | m/z = 737.28(C₅₅H₃₅N₃ = 737.91) | P1-120 | m/z = 737.28(C₅₅H₃₅N₃ = 737.91) |
| P1-121 | m/z = 737.28(C₅₅H₃₅N₃ = 737.91) | P1-122 | m/z = 737.28(C₅₅H₃₅N₃ = 737.91) |
| P1-123 | m/z = 737.28(C₅₅H₃₅N₃ = 737.91) | P1-124 | m/z = 737.28(C₅₅H₃₅N₃ = 737.91) |
| P1-125 | m/z = 737.28(C₅₅H₃₅N₃ = 737.91) | P1-126 | m/z = 737.28(C₅₅H₃₅N₃ = 737.91) |
| P1-127 | m/z = 737.28(C₅₅H₃₅N₃ = 737.91) | P1-128 | m/z = 737.28(C₅₅H₃₅N₃ = 737.91) |
| P1-129 | m/z = 737.28(C₅₅H₃₅N₃ = 737.91) | P1-130 | m/z = 737.28(C₅₅H₃₅N₃ = 737.91) |
| P1-131 | m/z = 737.28(C₅₅H₃₅N₃ = 737.91) | P1-132 | m/z = 737.28(C₅₅H₃₅N₃ = 737.91) |
| P1-133 | m/z = 737.28(C₅₅H₃₅N₃ = 737.91) | P1-134 | m/z = 737.28(C₅₅H₃₅N₃ = 737.91) |
| P1-135 | m/z = 737.28(C₅₅H₃₅N₃ = 737.91) | P1-136 | m/z = 737.28(C₅₅H₃₅N₃ = 737.91) |
| P1-137 | m/z = 737.28(C₅₅H₃₅N₃ = 737.91) | P1-138 | m/z = 737.28(C₅₅H₃₅N₃ = 737.91) |
| P1-139 | m/z = 737.28(C₅₅H₃₅N₃ = 737.91) | P1-140 | m/z = 737.28(C₅₅H₃₅N₃ = 737.91) |
| P1-141 | m/z = 617.27(C₄₅H₂₃D₆N₃ = 617.78) | P1-142 | m/z = 717.31(C₅₃H₂₇D₆N₃ = 717.9) |
| P1-143 | m/z = 717.31(C₅₃H₂₇D₆N₃ = 717.9) | P1-144 | m/z = 693.31(C₅₁H₂₇D₆N₃ = 693.88) |
| P1-145 | m/z = 618.28(C₄₅H₂₂D₇N₃ = 618.79) | P1-146 | m/z = 618.28(C₄₅H₂₂D₇N₃ = 618.79) |
| P1-147 | m/z = 618.28(C₄₅H₂₂D₇N₃ = 618.79) | P1-148 | m/z = 668.3(C₄₉H₂₄D₇N₃ = 668.85) |
| P1-149 | m/z = 617.27(C₄₅H₂₃D₆N₃ = 617.78) | P1-150 | m/z = 667.29(C₄₉H₂₅D₆N₃ = 667.84) |
| P1-151 | m/z = 667.29(C₄₉H₂₅D₆N₃ = 667.84) | P1-152 | m/z = 743.32(C₅₅H₂₉D₆N₃ = 743.94) |
| P1-153 | m/z = 616.27(C₄₅H₂₄D₅N₃ = 616.78) | P1-154 | m/z = 666.28(C₄₉H₂₆D₅N₃ = 666.84) |
| P1-155 | m/z = 692.3(C₅₁H₂₈D₅N₃ = 692.88) | P1-156 | m/z = 742.31(C₅₅H₃₀D₅N₃ = 742.94) |
| P1-157 | m/z = 640.42(C₄₅D₂₉N₃ = 640.92) | P1-158 | m/z = 640.42(C₄₅D₂₉N₃ = 640.92) |
| P1-159 | m/z = 692.45(C₄₉D₃₁N₃ = 693) | P1-160 | m/z = 640.42(C₄₅D₂₉N₃ = 640.92) |
| P1-161 | m/z = 692.45(C₄₉D₃₁N₃ = 693) | P1-162 | m/z = 720.47(C₅₁D₃₃N₃ = 721.05) |
| P1-163 | m/z = 772.5(C₅₅D₃₅N₃ = 773.12) | P1-164 | m/z = 772.5(C₅₅D₃₅N₃ = 773.12) |
| P1-165 | m/z = 824.53(C₅₉D₃₇N₃ = 825.19) | P1-166 | m/z = 763.3(C₅₇H₃₇N₃ = 763.94) |
| P1-167 | m/z = 763.3(C₅₇H₃₇N₃ = 763.94) | P1-168 | m/z = 763.3(C₅₇H₃₇N₃ = 763.94) |
| P1-169 | m/z = 763.3(C₅₇H₃₇N₃ = 763.94) | P1-170 | m/z = 763.3(C₅₇H₃₇N₃ = 763.94) |
| P1-171 | m/z = 763.3(C₅₇H₃₇N₃ = 763.94) | P1-172 | m/z = 813.31(C₆₁H₃₉N₃ = 814) |
| P1-173 | m/z = 813.31(C₆₁H₃₉N₃ = 814) | P1-174 | m/z = 813.31(C₆₁H₃₉N₃ = 814) |
| P1-175 | m/z = 813.31(C₆₁H₃₉N₃ = 814) | P1-176 | m/z = 813.31(C₆₁H₃₉N₃ = 814) |
| P1-177 | m/z = 813.31(C₆₁H₃₉N₃ = 814) | P1-178 | m/z = 839.33(C₆₃H₄₁N₃ = 840.04) |
| P1-179 | m/z = 839.33(C₆₃H₄₁N₃ = 840.04) | P1-180 | m/z = 839.33(C₆₃H₄₁N₃ = 840.04) |
| P1-181 | m/z = 800.53(C₅₇D₃₇N₃ = 801.17) | P1-182 | m/z = 800.53(C₅₇D₃₇N₃ = 801.17) |
| P1-183 | m/z = 800.53(C₅₇D₃₇N₃ = 801.17) | P1-184 | m/z = 800.53(C₅₇D₃₇N₃ = 801.17) |
| P1-185 | m/z = 800.53(C₅₇D₃₇N₃ = 801.17) | P1-186 | m/z = 800.53(C₅₇D₃₇N₃ = 801.17) |
| P1-187 | m/z = 800.53(C₅₇D₃₇N₃ = 801.17) | P1-188 | m/z = 800.53(C₅₇D₃₇N₃ = 801.17) |
| P1-189 | m/z = 800.53(C₅₇D₃₇N₃ = 801.17) | | |
| P2-1 | m/z = 611.24(C₄₅H₂₉N₃ = 611.75) | P2-2 | m/z = 611.24(C₄₅H₂₉N₃ = 611.75) |
| P2-3 | m/z = 611.24(C₄₅H₂₉N₃ = 611.75) | P2-4 | m/z = 611.24(C₄₅H₂₉N₃ = 611.75) |
| P2-5 | m/z = 611.24(C₄₅H₂₉N₃ = 611.75) | P2-6 | m/z = 661.25(C₄₉H₃₁N₃ = 661.81) |
| P2-7 | m/z = 711.27(C₅₃H₃₃N₃ = 711.87) | P2-8 | m/z = 661.25(C₄₉H₃₁N₃ = 661.81) |
| P2-9 | m/z = 661.25(C₄₉H₃₁N₃ = 661.81) | P2-10 | m/z = 661.25(C₄₉H₃₁N₃ = 661.81) |
| P2-11 | m/z = 661.25(C₄₉H₃₁N₃ = 661.81) | P2-12 | m/z = 661.25(C₄₉H₃₁N₃ = 661.81) |
| P2-13 | m/z = 661.25(C₄₉H₃₁N₃ = 661.81) | P2-14 | m/z = 661.25(C₄₉H₃₁N₃ = 661.81) |
| P2-15 | m/z = 661.25(C₄₉H₃₁N₃ = 661.81) | P2-16 | m/z = 661.25(C₄₉H₃₁N₃ = 661.81) |
| P2-17 | m/z = 661.25(C₄₉H₃₁N₃ = 661.81) | P2-18 | m/z = 661.25(C₄₉H₃₁N₃ = 661.81) |
| P2-19 | m/z = 661.25(C₄₉H₃₁N₃ = 661.81) | P2-20 | m/z = 661.25(C₄₉H₃₁N₃ = 661.81) |
| P2-21 | m/z = 661.25(C₄₉H₃₁N₃ = 661.81) | P2-22 | m/z = 687.27(C₅₁H₃₃N₃ = 687.85) |
| P2-23 | m/z = 687.27(C₅₁H₃₃N₃ = 687.85) | P2-24 | m/z = 687.27(C₅₁H₃₃N₃ = 687.85) |
| P2-25 | m/z = 737.28(C₅₅H₃₅N₃ = 737.91) | P2-26 | m/z = 737.28(C₅₅H₃₅N₃ = 737.91) |
| P2-27 | m/z = 737.28(C₅₅H₃₅N₃ = 737.91) | P2-28 | m/z = 737.28(C₅₅H₃₅N₃ = 737.91) |
| P2-29 | m/z = 737.28(C₅₅H₃₅N₃ = 737.91) | P2-30 | m/z = 737.28(C₅₅H₃₅N₃ = 737.91) |
| P2-31 | m/z = 737.28(C₅₅H₃₅N₃ = 737.91) | P2-32 | m/z = 737.28(C₅₅H₃₅N₃ = 737.91) |
| P2-33 | m/z = 640.42(C₄₅D₂₉N₃ = 640.92) | P2-34 | m/z = 640.42(C₄₅D₂₉N₃ = 640.92) |
| P2-35 | m/z = 640.42(C₄₅D₂₉N₃ = 640.92) | P2-36 | m/z = 640.42(C₄₅D₂₉N₃ = 640.92) |
| P2-37 | m/z = 692.45(C₄₉D₃₁N₃ = 693) | P2-38 | m/z = 720.47(C₅₁D₃₃N₃ = 721.05) |
| P2-39 | m/z = 763.3(C₅₇H₃₇N₃ = 763.94) | P2-40 | m/z = 763.3(C₅₇H₃₇N₃ = 763.94) |
| P2-41 | m/z = 763.3(C₅₇H₃₇N₃ = 763.94) | P2-42 | m/z = 813.31(C₆₁H₃₉N₃ = 814) |
| P2-43 | m/z = 863.33(C₆₅H₄₁N₃ = 864.06) | P2-44 | m/z = 913.35(C₆₉H₄₃N₃ = 914.12) |

### Synthesis Example 2

The compound represented by Formula 4 or Formula 5 can be manufactured by a named reaction or by referring to published patent publications, such as Korean Patent Registration No. 10-2395819 and U.S. Patent Publication No. 2023-0129535, but is not limited thereto.

Meanwhile, the FD-MS values of the compounds H-1 to H-129 and S-1 to S-116 of the present invention are as shown in Tables 4 and 5.

TABLE 4

| Compound | FD-MS | Compound | FD-MS |
|---|---|---|---|
| H-1 | m/z = 487.19(C₃₆H₂₅NO = 487.60) | H-2 | m/z = 553.19(C₄₀H₂₇NS = 553.72) |
| H-3 | m/z = 563.26(C₄₃H₃₃N = 563.74) | H-4 | m/z = 602.27(C₄₅H₃₄N₂ = 602.78) |
| H-5 | m/z = 517.15(C₃₆H₂₃NOS = 517.65) | H-6 | m/z = 603.20(C₄₄H₂₉NS = 603.78) |
| H-7 | m/z = 735.29(C₅₇H₃₇N = 735.93) | H-8 | m/z = 562.24(C₄₂H₃₀N₂ = 562.72) |
| H-9 | m/z = 565.17(C₄₀H₂₃NO₃ = 565.63) | H-10 | m/z = 581.14(C₄₀H₂₃NO₂S = 581.69) |
| H-11 | m/z = 823.24(C₅₉H₃₇NS₂ = 824.07) | H-12 | m/z = 727.30(C₅₄H₃₇N₃ = 727.91) |
| H-13 | m/z = 627.22(C₄₆H₂₉NO₂ = 627.74) | H-14 | m/z = 633.16(C₄₄H₂₇NS₂ = 633.83) |
| H-15 | m/z = 675.29(C₅₂H₃₇N = 675.88) | H-16 | m/z = 678.30(C₅₁H₃₈N₂ = 678.88) |
| H-17 | m/z = 669.21(C₄₈H₃₁NOS = 669.84) | H-18 | m/z = 785.22(C₅₆H₃₅NS₂ = 786.02) |
| H-19 | m/z = 617.18(C₄₄H₂₇NOS = 617.77) | H-20 | m/z = 601.20(C₄₄H₂₇NO₂ = 601.71) |
| H-21 | m/z = 779.32(C₅₉H₄₁NO = 779.98) | H-22 | m/z = 583.23(C₄₂H₃₃NS = 583.79) |
| H-23 | m/z = 679.32(C₅₂H₄₁N = 679.91) | H-24 | m/z = 726.27(C₅₄H₃₄N₂O = 726.88) |
| H-25 | m/z = 593.18(C₄₂H₂₇NOS = 593.74) | H-26 | m/z = 774.22(C₅₄H₃₄N₂S₂ = 775.00) |
| H-27 | m/z = 557.24(C₄₀H₃₁NO₂ = 557.69) | H-28 | m/z = 652.25(C₄₈H₃₂N₂O = 652.80) |
| H-29 | m/z = 619.29(C₄₆H₃₇NO = 619.81) | H-30 | m/z = 603.20(C₄₄H₂₉NS = 603.78) |
| H-31 | m/z = 813.30(C₆₂H₃₉NO = 814.00) | H-32 | m/z = 784.29(C₅₇H₄₀N₂S = 785.02) |
| H-33 | m/z = 577.20(C₄₂H₂₇NO₂ = 577.68) | H-34 | m/z = 607.14(C₄₂H₂₅NS₂ = 607.79) |
| H-35 | m/z = 801.34(C₆₂H₄₃N = 802.03) | H-36 | m/z = 575.24(C₄₂H₂₉N₃ = 575.72) |
| H-37 | m/z = 577.20(C₄₂H₂₇NO₂ = 577.68) | H-38 | m/z = 607.14(C₄₂H₂₅NS₂ = 607.79) |
| H-39 | m/z = 801.34(C₆₂H₄₃N = 802.03) | H-40 | m/z = 575.24(C₄₂H₂₉N₃ = 575.72) |
| H-41 | m/z = 601.20(C₄₄H₂₇NO₂ = 601.71) | H-42 | m/z = 471.11(C₃₁H₂₁NS₂ = 471.64) |
| H-43 | m/z = 675.29(C₅₂H₃₇N = 675.88) | H-44 | m/z = 727.30(C₅₄H₃₇N₃ = 727.91) |
| H-45 | m/z = 603.20(C₄₄H₂₉NS = 603.78) | H-46 | m/z = 561.16(C₃₈H₂₇NS₂ = 561.76) |
| H-47 | m/z = 799.32(C₆₂H₄₁N = 800.02) | H-48 | m/z = 702.27(C₅₂H₃₄N₂O = 702.86) |
| H-49 | m/z = 729.27(C₅₄H₃₅NO₂ = 729.88) | H-50 | m/z = 785.22(C₅₆H₃₅NS₂ = 786.02) |
| H-51 | m/z = 812.32(C₆₂H₄₀N₂ = 813.02) | H-52 | m/z = 681.22(C₄₈H₃₁N₃S = 681.86) |
| H-53 | m/z = 615.18(C₄₄H₂₅NO₃ = 615.69) | H-54 | m/z = 763.15(C₅₂H₂₉NS₃ = 763.99) |
| H-55 | m/z = 593.31(C₄₅H₃₉N = 593.81) | H-56 | m/z = 840.33(C₆₂H₄₀N₄ = 841.03) |
| H-57 | m/z = 657.18(C₄₆H₂₇NO₂S = 657.79) | H-58 | m/z = 824.23(C₅₈H₃₆N₂S₂ = 825.06) |
| H-59 | m/z = 1195.42(C₉₁H₅₇NS = 1196.52) | H-60 | m/z = 656.19(C₄₆H₂₈N₂OS = 656.80) |
| H-61 | m/z = 607.16(C₄₂H₂₅NO₂S = 607.73) | H-62 | m/z = 773.20(C₅₄H₃₁NO₃S = 773.91) |
| H-63 | m/z = 1013.4(C₇₉H₅₁N = 1014.28) | H-64 | m/z = 758.24(C₅₄H₃₄N₂OS = 758.94) |
| H-65 | m/z = 623.14(C₄₂H₂₅NOS₂ = 623.79) | H-66 | m/z = 763.16(C₅₂H₂₉NO₂S₂ = 763.93) |
| H-67 | m/z = 799.20(C₅₆H₃₃NOS₂ = 800.01) | H-68 | m/z = 743.23(C₅₄H₃₃NOS = 743.92) |
| H-69 | m/z = 872.25(C₆₂H₃₆N₂O₂S = 873.04) | H-70 | m/z = 772.22(C₅₄H₃₂N₂O₂S = 772.92) |
| H-71 | m/z = 830.28(C₆₁H₃₈N₂S = 831.05) | H-72 | m/z = 808.25(C₅₈H₃₃FN₂O₂ = 808.91) |
| H-73 | m/z = 929.21(C₆₄H₃₅NO₃S₂ = 930.11) | H-74 | m/z = 963.27(C₆₈H₄₁N₃S₂ = 964.22) |
| H-75 | m/z = 809.24(C₅₈H₃₅NO₂S = 809.98) | H-76 | m/z = 893.29(C₆₆H₃₉NO₃ = 894.04) |
| H-77 | m/z = 794.28(C₅₈H₃₈N₂S = 795.02) | H-78 | m/z = 900.26(C₆₄H₄₀N₂S₂ = 901.16) |
| H-79 | m/z = 758.28(C₅₅H₃₈N₂S = 758.98) | H-80 | m/z = 1082.37(C₈₁H₅₀N₂S = 1083.37) |
| H-81 | m/z = 573.25(C₄₄H₃₁N = 573.74) | H-82 | m/z = 649.28(C₅₀H₃₅N = 649.84) |
| H-83 | m/z = 699.29(C₅₄H₃₇N = 699.90) | H-84 | m/z = 699.29(C₅₄H₃₇N = 699.90) |
| H-85 | m/z = 673.28(C₅₂H₃₅N = 673.86) | H-86 | m/z = 649.28(C₅₀H₃₅N = 649.84) |
| H-87 | m/z = 625.28(C₄₈H₃₅N = 625.82) | H-88 | m/z = 673.28(C₅₂H₃₅N = 673.86) |
| H-89 | m/z = 773.31(C₆₀H₃₉N = 773.98) | H-90 | m/z = 749.31(C₅₈H₃₉N = 749.96) |
| H-91 | m/z = 699.29(C₅₄H₃₇N = 699.90) | H-92 | m/z = 599.26(C₄₆H₃₃N = 599.78) |
| H-93 | m/z = 639.26(C₄₈H₃₃NO = 639.8) | H-94 | m/z = 765.25(C₅₇H₃₅NS = 765.97) |
| H-95 | m/z = 677.31(C₅₂H₃₉N = 677.89) | H-96 | m/z = 727.30(C₅₄H₃₇N₃ = 727.91) |
| H-97 | m/z = 552.18(C₃₉H₂₄N₂O₂ = 552.63) | H-98 | m/z = 628.22(C₄₅H₂₈N₂O₂ = 628.73) |
| H-99 | m/z = 614.24(C₄₅H₃₀N₂O = 614.75) | H-100 | m/z = 614.24(C₄₅H₃₀N₂O = 614.75) |
| H-101 | m/z = 691.21(C₅₀H₂₉NO₃ = 691.79) | H-102 | m/z = 739.29(C₅₆H₃₇NO = 739.92) |
| H-103 | m/z = 673.15(C₄₆H₂₇NOS₂ = 673.85) | H-104 | m/z = 726.27(C₅₄H₃₄N₂O = 726.88) |
| H-105 | m/z = 617.18(C₄₄H₂₇NOS = 617.77) | H-106 | m/z = 611.22(C₄₆H₂₉NO = 611.74) |
| H-107 | m/z = 769.24(C₅₆H₃₅NOS = 769.96) | H-108 | m/z = 701.28(C₅₂H₃₅N₃ = 701.87) |
| H-109 | m/z = 527.22(C₃₉H₂₉NO = 527.67) | H-110 | m/z = 643.20(C₄₆H₂₉NOS = 643.80) |
| H-111 | m/z = 593.18(C₄₂H₂₇NOS = 593.74) | H-112 | m/z = 726.27(C₅₄H₃₄N₂O = 726.88) |
| H-113 | m/z = 726.27(C₅₄H₃₄N₂O = 726.88) | H-114 | m/z = 558.14(C₃₇H₂₂N₂O₂S = 558.65) |
| H-115 | m/z = 620.19(C₄₃H₂₈N₂OS = 620.77) | H-116 | m/z = 686.27(C₅₂H₃₄N₂ = 686.86) |
| H-117 | m/z = 718.24(C₅₂H₃₄N₂S = 718.92) | H-118 | m/z = 728.28(C₅₄H₃₆N₂O = 728.89) |
| H-119 | m/z = 592.20(C₄₂H₂₈N₂S = 592.76) | H-120 | m/z = 756.22(C₅₄H₃₂N₂OS = 756.92) |
| H-121 | m/z = 547.70(C₄₂H₂₉N = 547.70) | H-122 | m/z = 672.28(C₄₉H₂₄D₇NO₂ = 672.83) |
| H-123 | m/z = 626.28(C₄₈H₂₆D₅N = 558.75) | H-124 | m/z = 558.22(C₄₀H₂₂D₅NS = 558.75) |
| H-125 | m/z = 638.27(C₄₈H₃₄N₂ = 638.81) | H-126 | m/z = 579.23(C₄₁H₂₉N₃O = 579.70) |
| H-127 | m/z = 624.17(C₄₂H₂₈N₂S₂ = 624.82) | H-128 | m/z = 552.18(C₃₉H₂₄N₂O₂ = 552.63) |
| H-129 | m/z = 641.18(C₄₆H₂₇NOS = 641.79) | | |

TABLE 5

| compound | FD-MS | compound | FD-MS |
|---|---|---|---|
| S-1 | m/z = 408.16(C₃₀H₂₀N₂ = 408.50) | S-2 | m/z = 534.21(C₄₀H₂₆N₂ = 534.66) |
| S-3 | m/z = 560.23(C₄₂H₂₈N₂ = 560.70) | S-4 | m/z = 584.23(C₄₄H₂₈N₂ = 584.72) |
| S-5 | m/z = 560.23(C₄₂H₂₈N₂ = 560.70) | S-6 | m/z = 634.24(C₄₈H₃₀N₂ = 634.78) |
| S-7 | m/z = 610.24(C₄₆H₃₀N₂ = 610.76) | S-8 | m/z = 498.17(C₃₆H₂₂N₂O = 498.59) |
| S-9 | m/z = 574.20(C₄₂H₂₆N₂O = 574.68) | S-10 | m/z = 660.26(C₅₀H₃₂N₂ = 660.82) |
| S-11 | m/z = 686.27(C₅₂H₃₄N₂ = 686.86) | S-12 | m/z = 620.14(C₄₂H₂₄N₂S₂ = 620.79) |
| S-13 | m/z = 640.20(C₄₆H₂₈N₂S = 640.80) | S-14 | m/z = 560.23(C₄₂H₂₈N₂ = 560.70) |
| S-15 | m/z = 558.21(C₄₂H₂₆N₂ = 558.68) | S-16 | m/z = 548.19(C₄₀H₂₄N₂O = 548.65) |
| S-17 | m/z = 573.22(C₄₂H₂₇N₃ = 573.70) | S-18 | m/z = 564.17(C₄₀H₂₄N₂S = 564.71) |
| S-19 | m/z = 574.20(C₄₂H₂₆N₂O = 574.68) | S-20 | m/z = 564.17(C₄₀H₂₄N₂S = 564.71) |
| S-21 | m/z = 564.17(C₄₀H₂₄N₂S = 564.71) | S-22 | m/z = 813.31(C₆₁H₃₉N₃ = 814.00) |
| S-23 | m/z = 696.26(C₅₃H₃₂N₂ = 696.85) | S-24 | m/z = 691.23(C₄₉H₂₉N₃O₂ = 691.79) |
| S-25 | m/z = 710.27(C₅₄H₃₄N₂ = 710.88) | S-26 | m/z = 610.24(C₄₆H₃₀N₂ = 610.76) |
| S-27 | m/z = 670.15(C₄₆H₂₆N₂S₂ = 670.85) | S-28 | m/z = 640.29(C₄₈H₃₆N₂ = 640.83) |
| S-29 | m/z = 598.20(C₄₄H₂₆N₂O = 598.71) | S-30 | m/z = 623.24(C₄₆H₂₉N₃ = 623.76) |
| S-31 | m/z = 458.18(C₃₄H₂₂N₂ = 458.56) | S-32 | m/z = 548.19(C₄₀H₂₄N₂O = 548.65) |
| S-33 | m/z = 508.19(C₃₈H₂₄N₂ = 508.62) | S-34 | m/z = 508.19(C₃₈H₂₄N₂ = 508.62) |
| S-35 | m/z = 623.24(C₄₆H₂₉N₃ = 623.76) | S-36 | m/z = 564.17(C₄₀H₂₄N₂S = 564.71) |
| S-37 | m/z = 627.20(C₄₆H₂₉NS = 627.81) | S-38 | m/z = 505.10(C₃₄H₁₉NS₂ = 505.65) |
| S-39 | m/z = 514.15(C₃₆H₂₂N₂S = 514.65) | S-40 | m/z = 575.17(C₄₂H₂₅NS = 575.73) |
| S-41 | m/z = 642.21(C₄₆H₃₀N₂S = 642.82) | S-42 | m/z = 575.17(C₄₂H₂₅NS = 575.73) |
| S-43 | m/z = 606.18(C₄₂H₂₆N₂OS = 606.74) | S-44 | m/z = 575.17(C₄₂H₂₅NS = 575.73) |
| S-45 | m/z = 551.17(C₄₀H₂₅NS = 551.71) | S-46 | m/z = 607.14(C₄₂H₂₅NS₂ = 607.79) |
| S-47 | m/z = 525.16(C₃₈H₂₃NS = 525.67) | S-48 | m/z = 642.21(C₄₆H₃₀N₂S = 642.82) |
| S-49 | m/z = 548.19(C₄₀H₂₄N₂O = 548.65) | S-50 | m/z = 473.14(C₃₄H₁₉NO₂ = 473.53) |
| S-51 | m/z = 566.15(C₃₉H₂₂N₂OS = 566.68) | S-52 | m/z = 459.16(C₃₄H₂₁NO = 459.55) |
| S-53 | m/z = 473.14(C₃₄H₁₉NO₂ = 473.53) | S-54 | m/z = 523.16(C₃₈H₂₁NO₂ = 523.59) |
| S-55 | m/z = 539.13(C₃₈H₂₁NOS = 539.65) | S-56 | m/z = 548.19(C₄₀H₂₄N₂O = 548.65) |
| S-57 | m/z = 489.12(C₃₄H₁₉NOS = 489.59) | S-58 | m/z = 545.09(C₃₆H₁₉NOS₂ = 545.67) |
| S-59 | m/z = 549.17(C₄₀H₂₃NO₂ = 549.63) | S-60 | m/z = 565.15(C₄₀H₂₃NOS = 565.69) |
| S-61 | m/z = 523.16(C₃₈H₂₁NO₂ = 523.59) | S-62 | m/z = 598.2(C₄₄H₂₆N₂O = 598.71) |
| S-63 | m/z = 539.13(C₃₈H₂₁NOS = 539.65) | S-64 | m/z = 589.15(C₄₂H₂₃NOS = 589.71) |
| S-65 | m/z = 498.17(C₃₆H₂₂N₂O = 498.59) | S-66 | m/z = 509.18(C₃₈H₂₃NO = 509.61) |
| S-67 | m/z = 548.19(C₄₀H₂₄N₂O = 548.65) | S-68 | m/z = 549.17(C₄₀H₂₃NO₂ = 549.63) |
| S-69 | m/z = 449.12(C₃₂H₁₉NS = 449.57) | S-70 | m/z = 439.1(C₃₀H₁₇NOS = 439.53) |
| S-71 | m/z = 647.22(C₄₉H₂₉NO = 647.78) | S-72 | m/z = 550.20(C₄₀H₂₆N₂O = 550.66) |
| S-73 | m/z = 459.16(C₃₄H₂₁NO = 459.55) | S-74 | m/z = 533.18(C₄₀H₂₃NO = 533.63) |
| S-75 | m/z = 525.16(C₃₈H₂₃NS = 525.67) | S-76 | m/z = 564.17(C₄₀H₂₄N₂S = 564.71) |
| S-77 | m/z = 575.19(C₄₂H₂₅NO₂ = 575.67) | S-78 | m/z = 663.22(C₄₉H₂₉NO₂ = 663.78) |
| S-79 | m/z = 647.22(C₄₉H₂₉NO = 647.78) | S-80 | m/z = 496.16(C₃₆H₂₀N₂O = 496.57) |
| S-81 | m/z = 565.15(C₄₀H₂₃NOS = 565.69) | S-82 | m/z = 505.1(C₃₄H₁₉NS₂ = 505.65) |
| S-83 | m/z = 765.25(C₅₆H₃₅NOSi = 765.99) | S-84 | m/z = 615.17(C₄₄H₂₅NOS = 615.75) |
| S-85 | m/z = 603.17(C₄₃H₂₅NOS = 603.74) | S-86 | m/z = 772.29(C₅₉H₃₆N₂ = 772.95) |
| S-87 | m/z = 802.33(C₆₁H₄₂N₂ = 803.02) | S-88 | m/z = 607.23(C₄₇H₂₉N = 607.76) |
| S-89 | m/z = 524.23(C₃₉H₂₈N₂ = 524.67) | S-90 | m/z = 665.22(C₄₉H₃₁NS = 665.85) |
| S-91 | m/z = 633.25(C₄₉H₃₁N = 633.79) | S-92 | m/z = 775.29(C₅₉H₃₇NO = 775.95) |
| S-93 | m/z = 535.23(C₄₁H₂₉N = 535.69) | S-94 | m/z = 623.22(C₄₇H₂₉NO = 623.76) |
| S-95 | m/z = 687.20(C₅₁H₂₉NS = 687.86) | S-96 | m/z = 735.29(C₅₇H₃₇N = 735.93) |
| S-97 | m/z = 611.26(C₄₇H₃₃N = 611.79) | S-98 | m/z = 679.23(C₅₀H₃₃NS = 679.88) |
| S-99 | m/z = 787.32(C₆₁H₄₁N = 788.01) | S-100 | m/z = 743.33(C₅₅H₄₁N₃ = 743.95) |
| S-101 | m/z = 485.21(C₃₇H₂₇N = 485.63) | S-102 | m/z = 471.20(C₃₆H₂₅N = 471.60) |
| S-103 | m/z = 571.19(C₄₃H₂₅NO = 571.68) | S-104 | m/z = 584.23(C₄₄H₂₈N₂ = 584.72) |
| S-105 | m/z = 539.24(C₄₀H₂₁D₅N₂ = 539.69) | S-106 | m/z = 453.15(C₃₂H₁₅NS = 471.6) |
| S-107 | m/z = 563.26(C₄₃H₂₆D₄NO = 563.74) | S-108 | m/z = 589.26(C₄₄H₂₃D₅N₂ = 584.72) |
| S-109 | m/z = 589.26(C₄₄H₂₃D₅N₂ = 589.75) | S-110 | m/z = 562.23(C₄₂H₂₂D₄N₂ = 562.71) |
| S-111 | m/z = 660.26(C₅₀H₃₂N₂ = 660.82) | S-112 | m/z = 553.22(C₄₀H₁₉D₅N₂O = 553.68) |
| S-113 | m/z = 634.24(C₄₈H₃₀N₂ = 634.78) | S-114 | m/z = 589.26(C₄₄H₂₃D₅N₂ = 589.75) |
| S-115 | m/z = 588.25(C₄₄H₂₄D₄N₂ = 588.75) | S-116 | m/z = 513.23(C₃₈H₁₉D₅N₂ = 513.65) |

Meanwhile, exemplary synthesis examples of the present invention represented by Formula 1, Formula 4 and Formula 5 have been described, but these are all based on the Buchwald-Hartwig cross coupling reaction, Miyaura boration reaction, Suzuki cross-coupling reaction, Intramolecular acid-induced cyclization reaction (J. mater. Chem. 1999, 9, 2095), Pd(II)-catalyzed oxidative cyclization reaction (Org. Lett. 2011, 13, 5504), and PPh₃-mediated reductive cyclization reaction (J. Org. Chem. 2005, 70, 5014.), and it will be easily understood by those skilled in the art that the reaction proceeds even when other substituents defined in Formula 1, Formula 4 and Formula 5 are bonded in addition to the substituents specified in the specific synthesis examples.

### [Example 1] to [Example 23] Red Organic Electroluminescent Device (Phosphorescent Host)

Compound A and Compound B were used on an ITO layer (anode) formed on a glass substrate, and Compound B was doped at a weight ratio of 98:2 to form a hole injection layer with a thickness of 10 nm. Then, Compound A was vacuum-deposited on the hole injection layer with a thickness of 110 nm to form a hole transport layer. In the next step, compound C-R was vacuum-deposited on the hole transport layer to a thickness of 20 nm to form an emitting auxiliary layer. Thereafter, the host material of the emitting layer was formed using a mixture described in Table 6 as a compound of the present invention, and a dopant material bis-(1-phenylisoquinolyl)iridium(III)acetylacetonate (hereinafter ‘(piq)₂Ir(acac)’) was used, but the dopant was doped so that the weight ratio of the host and the dopant was 95:5 to form an emitting layer with a thickness of 30 nm.

Subsequently, compound E was vacuum-deposited on the emitting layer to form a hole-blocking layer with a thickness of 10 nm, and a mixture of compound F and compound G at a weight ratio of 5:5 was used on the hole-blocking layer to form an electron transport layer with a thickness of 30 nm. Thereafter, compound G was deposited on the electron transport layer to form an electron injection layer with a thickness of 0.2 nm, and then Al was deposited to form a cathode with a thickness of 150 nm.
compound A: N-([1,1′-biphenyl]-4-yl)-9,9-dimethyl-N-(4-(9-phenyl-9H-carbazol-3-yl)phenyl)-9H-fluoren-2-amine
compound B: 4,4′,4″-((1E,1′E,1″E)-cyclopropane-1,2,3-triylidenetris(cyanomethaneylylidene))tris(2,3,5,6-tetrafluorobenzonitrile)
compound C-R: N7-(dibenzo[b,d]thiophen-2-yl)-N²,N²,N⁷-triphenyldibenzo[b,d]thiophene-2,7-diamine
compound E: 2-(4′-(9,9-dimethyl-9H-fluoren-2-yl)-[1,1′-biphenyl]-3-yl)-4,6-diphenyl-1,3,5-triazine
compound F: 2,7-bis(4-(4,6-diphenyl-1,3,5-triazin-2-yl)phenyl)naphthalene
compound G: (8-quinolinolato)lithium

### [Comparative Example 1] to [Comparative Example 3]

An organic light-emitting device was manufactured in the same manner as in Example 1, except that Comparative Compounds 1 to 3 were used as the first host material of the emitting layer.

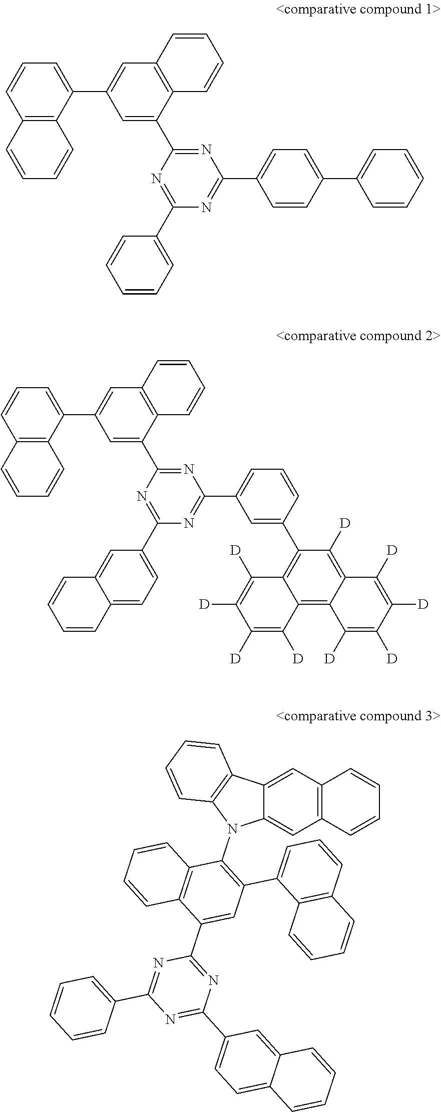 [SMILES: c1ccc(-c2ccc(-c3nc(-c4ccccc4)nc(-c4cc(-c5cccc6ccccc56)cc5ccccc45)n3)cc2)cc1 ; [2H]c1c([2H])c([2H])c2c(c1[2H])c([2H])c(-c1cccc(-c3nc(-c4ccc5ccccc5c4)nc(-c4cc(-c5cccc6ccccc56)cc5ccccc45)n3)c1)c1c([2H])c([2H])c([2H])c([2H])c12 ; c1ccc(-c2nc(-c3ccc4ccccc4c3)nc(-c3cc(-c4cccc5ccccc45)c(-n4c5ccccc5c5cc6ccccc6cc54)c4ccccc34)n2)cc1]

The electroluminescence (EL) characteristics were measured using a PR-650 from Photoresearch by applying a forward bias DC voltage to the organic electroluminescent devices manufactured in this manner, and the T95 lifespan was measured using a lifespan measuring device manufactured by Maxscience at a standard luminance of 2500 cd/m². Table 6 shows the results of the device fabrication and evaluation.

The measuring apparatus can evaluate the performance of new materials compared to comparative compounds under identical conditions, without being affected by possible daily fluctuations in deposition rate, vacuum quality or other parameters.

During the evaluation, one batch contains 4 identically prepared OLEDs including a comparative compound, and the performance of a total of 12 OLEDs is evaluated in 3 batches, so the value of the experimental results obtained in this way indicates statistical significance.

TABLE 6

| | | | Driving | Current | | |
|---|---|---|---|---|---|---|
| | First | Second | Voltage | Density | Efficiency | |
| | compound | compound | (V) | (mA/cm²) | (cd/A) | T(95) |
| comparative | comparative | H-5 | 5.6 | 12.4 | 20.1 | 91.4 |
| example(1) | compound1 | | | | | |
| comparative | comparative | H-5 | 5.4 | 11.6 | 21.6 | 93.2 |
| example(2) | compound2 | | | | | |
| comparative | comparative | H-5 | 5.5 | 11.7 | 21.4 | 92.7 |
| example(3) | compound3 | | | | | |
| example(1) | P1-2 | H-5 | 4.5 | 6.5 | 38.2 | 136.2 |
| example(2) | P1-5 | H-5 | 4.3 | 7.1 | 35.4 | 127.2 |
| example(3) | P1-14 | H-5 | 4.3 | 6.9 | 36.5 | 129.5 |
| example(4) | P1-17 | H-5 | 4.4 | 7.1 | 35.4 | 125.5 |
| example(5) | P1-22 | H-5 | 4.2 | 6.5 | 38.7 | 137.3 |
| example(6) | P1-39 | H-5 | 4.2 | 6.9 | 36.2 | 130.2 |
| example(7) | P1-48 | H-5 | 4.3 | 6.3 | 39.6 | 126.4 |
| example(8) | P1-68 | H-5 | 4.3 | 6.4 | 39.2 | 135.5 |
| example(9) | P1-102 | H-5 | 4.3 | 6.6 | 38.1 | 132.0 |
| example(10) | P1-158 | H-5 | 4.3 | 7.1 | 35.0 | 137.2 |
| example(11) | P1-172 | H-5 | 4.4 | 6.1 | 41.1 | 130.3 |
| example(12) | P2-6 | H-5 | 4.4 | 6.9 | 36.1 | 129.5 |
| example(13) | P2-23 | H-5 | 4.3 | 6.8 | 37.0 | 133.1 |
| example(14) | P2-33 | H-100 | 4.4 | 6.2 | 40.3 | 131.3 |
| example(15) | P1-2 | H-100 | 4.5 | 6.9 | 36.4 | 136.5 |
| example(16) | P1-2 | H-126 | 4.4 | 6.2 | 40.5 | 134.9 |
| example(17) | P1-2 | S-21 | 4.5 | 5.7 | 43.8 | 136.1 |
| example(18) | P1-2 | S-50 | 4.5 | 5.9 | 42.2 | 131.7 |
| example(19) | P1-2 | S-109 | 4.5 | 5.8 | 43.4 | 131.0 |
| example(20) | P1-5 | H-100 | 4.5 | 6.7 | 37.6 | 125.7 |
| example(21) | P1-5 | S-109 | 4.5 | 6.9 | 36.4 | 130.9 |
| example(22) | P2-23 | H-100 | 4.3 | 7.0 | 35.9 | 131.2 |
| example(23) | P2-23 | S-109 | 4.5 | 7.0 | 35.7 | 129.2 |

As can be seen in Table 6, when a red organic electroluminescent device is manufactured using the material for an organic electroluminescent device of the present invention as a phosphorescent host material, the compound of the present invention exhibits remarkable characteristics in device performance compared to the case where comparative compounds 1 to 3 are used, and particularly, exhibits excellent characteristics in terms of efficiency and lifespan.

As can be seen above, when a host for an emitting layer is formed by mixing multiple compounds, it can be confirmed that significant differences in characteristics are observed depending on the type of the first and second hosts. Similarly, differences in driving voltage, efficiency, and lifespan are observed depending on the type of the second host.

Comparative compounds 1 to 3 are similar to the compounds of the present invention in that one of the substituents of triazine is bonded to a [naphthylene-naphthyl group] having a specific bonding position, but some of the substituents are different.

More specifically, the compound of the present invention comprises a naphthylene group in which one of the remaining substituents of the triazine is necessarily further substituted with an aryl moiety, whereas the comparative compound 1 comprises only a monocyclic aryl moiety. Comparative compound 2 has one more naphthyl group attached as a substituent of triazine, but does not contain an additional substituent on that naphthyl group

Additionally, while the compound of the present invention has a [naphthylene-naphthyl group] aryl moiety having a specific bonding position that can be substituted only with hydrogen and deuterium, Comparative Compound 3 not only has a benzocarbazole substituent further bonded to the naphthylene bonded to the triazine, but also does not include an additional substituent on the naphthyl group bonded to the triazine, similar to Comparative Compound 2.

As a result, it can be confirmed that the device results using the compound of the present invention, in which one of the substituents of the triazine is bonded to a [naphthylene-naphthyl group]aryl moiety having a specific bonding position where only hydrogen or deuterium can be substituted, and one of the remaining substituents of the triazine necessarily includes a naphthylene group at a specific bonding position where aryl is substituted, are significantly improved compared to the device results using comparative compounds 1 to 3.

That is, these structural differences may result in differences in the physical properties of the compounds, which are believed to have a significant impact on device performance.

Additionally, as can be seen in Table 7, compound P1-2 of the present invention exhibits a lower LUMO value than comparative compound 1 and comparative compound 2. Accordingly, when the compound of the present invention is used as a host, electron injection from the electron transport layer to the emitting layer is smoother than in the comparative compounds, so excitons are better generated in the emitting layer, and the device characteristics appear to be improved.

TABLE 7

| | Comparative | Comparative | |
|---|---|---|---|
| | compound 1 | compound 2 | P1-2 |
| G. LUMO (eV) | −1.924 | −1.926 | −1.958 |

Accordingly, even if the molecular components are similar, the properties of the compound, such as hole characteristics, light efficiency characteristics, energy level, hole injection and mobility characteristics, charge balance of holes and electrons, volume density, and intermolecular distance, can be significantly different to an extent that it is difficult to predict depending on the presence or absence of substituted substituents and the type of substituents. Additionally, it suggests that the performance of the device may vary due to complex factors rather than a single component influencing the results of the entire device.

Further, although not described as a comparative example in the specification of the present invention, it can be seen that a device using the compound of the present invention will exhibit significantly superior device characteristics compared to a device using a compound having a similar structure to the compound of the present invention or a simple structural isomer compound.

Although exemplary embodiments of the present invention have been described for illustrative purposes, those skilled in the art will appreciate that various modifications, additions and substitutions are possible, without departing from the scope and spirit of the invention as disclosed in the accompanying claims. Therefore, the embodiment disclosed in the present invention is intended to illustrate the scope of the technical idea of the present invention, and the scope of the present invention is not limited by the embodiment. The scope of the present invention shall be construed on the basis of the accompanying claims, and it shall be construed that all of the technical ideas included within the scope equivalent to the claims belong to the present invention.

What is claimed is:

1. A compound represented by Formula 1: 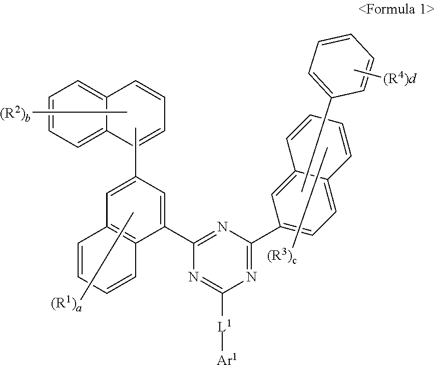
wherein:
R¹, R² and R³ are each deuterium,
R⁴ is selected from the group consisting of deuterium; a C₆-C₆₀ aryl group; a fluorenyl group; a C₃-C₆₀ aliphatic ring; a C₁-C₅₀ alkyl group; a C₂-C₂₀ alkenyl group; a C₂-C₂₀ alkynyl group; a C₁-C₃₀ alkoxy group; and a C₆-C₃₀ an aryloxy group, and where R⁴ is in plural, a plurality of R⁴ may be the same or different from each other, and the adjacent groups thereof may be bonded to each other to form an aromatic ring,
L¹ is a single bond; or a C₆-C₁₂ arylene group,
Ar¹ is a C₆-C₁₂ aryl group,
a and c are independently an integer of 0 to 6, b is an integer of 0 to 7, and d is an integer of 0 to 5,
wherein the aryl group, arylene group, fluorenyl group, aliphatic ring group, alkyl group, alkenyl group, alkynyl group, alkoxyl group and aryloxy group may each be substituted with one or more substituents selected from the group consisting of deuterium; a C₁-C₂₀ alkyl group; a C₁-C₂₀ alkyl group substituted with deuterium; a C₆-C₁₂ aryl group; and a C₆-C₁₂ aryl group substituted with deuterium.

2. The compound according to claim 1, wherein the compound represented by Formula 1 is represented by any of Formulas 1-3 to 1-6: 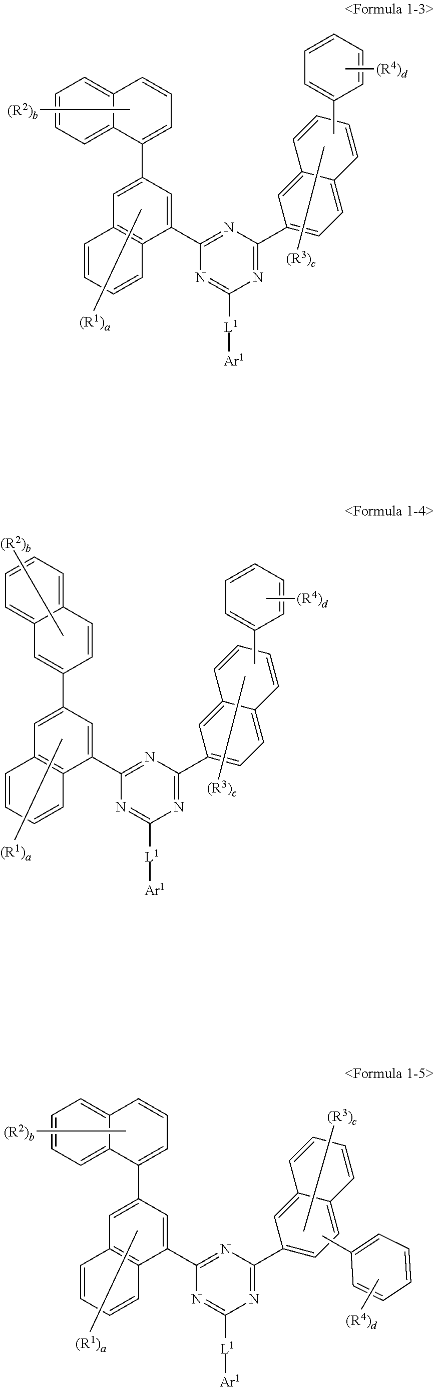 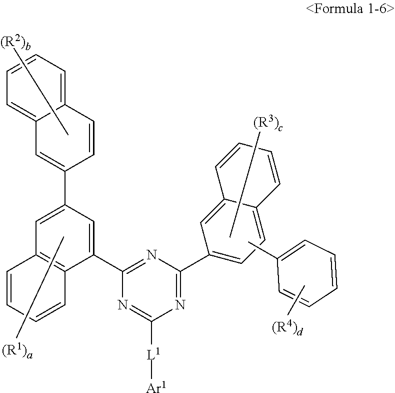
wherein: R¹, R², R³, R⁴, L¹, Ar¹, a, b, c and d are the same as defined in claim 1.

3. The compound according to claim 1, wherein the compound represented by Formula 1 is represented by Formula 2-1 or Formula 2-2: 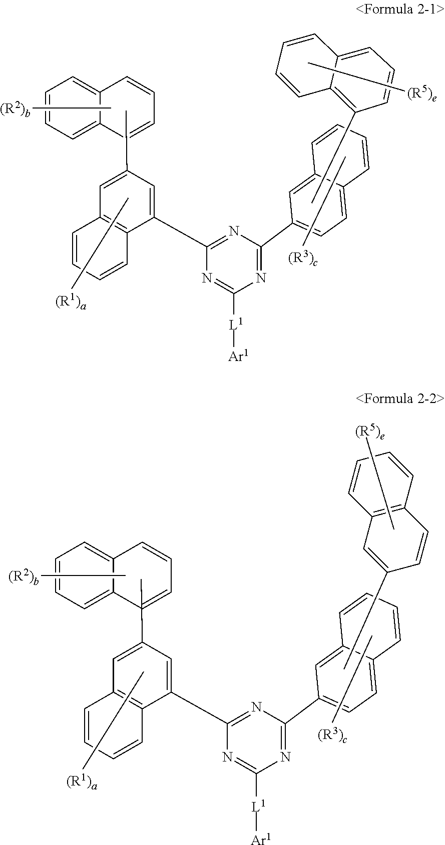
wherein: R¹, R², R³, R⁴, L¹, Ar¹, a, b, c and d are the same as defined in claim 1.

4. The compound according to claim 1, wherein Ar¹ of Formula 1 is represented by any of Formulas Ar-1 to Ar-3: 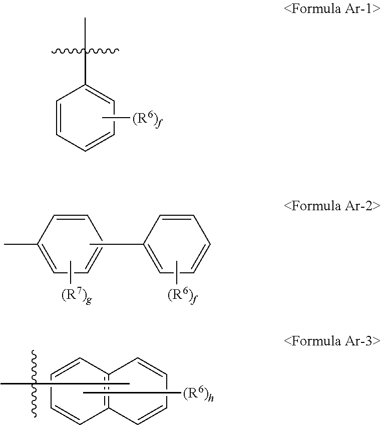
wherein:
R⁶ and R⁷ are independently the same or different from each other and are independently selected from the group consisting of deuterium; a C₁-C₂₀ alkyl group; a C₁-C₂₀ alkyl group substituted with deuterium; a C₆-C₁₂ aryl group; and a C₆-C₁₂ aryl group substituted with deuterium, and a plurality of adjacent groups thereof may be bonded to each other to form a ring,
f is an integer from 0 to 5; g is an integer from 0 to 4; h is an integer from 0 to 7, and [symbol]
indicates the position to be bonded.

5. The compound according to claim 1, wherein L¹ of Formula 1 is represented by any one of Formulas L-1 to L-4: 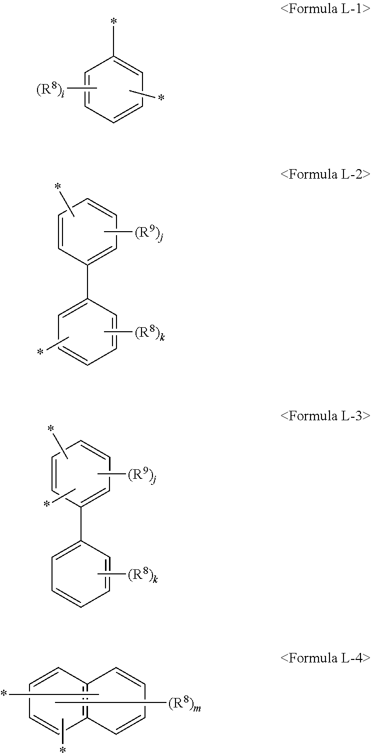
wherein:
R⁸ and R⁹ are independently the same or different from each other and are independently selected from the group consisting of deuterium; a C₁-C₂₀ alkyl group;
a C₁-C₂₀ alkyl group substituted with deuterium; a C₆-C₁₂ aryl group; and a C₆-C₁₂ aryl group substituted with deuterium, and a plurality of adjacent groups thereof may be bonded to each other to form a ring,
i and j are independently an integer from 0 to 4; k is an integer from 0 to 5; l is an integer from 0 to 3; m is an integer from 0 to 6, and
* indicates the position to be bonded.

6. A composition for an organic electronic element comprising a first compound represented by Formula 1 of claim 1; and a second compound represented by Formula 4 or Formula 5: 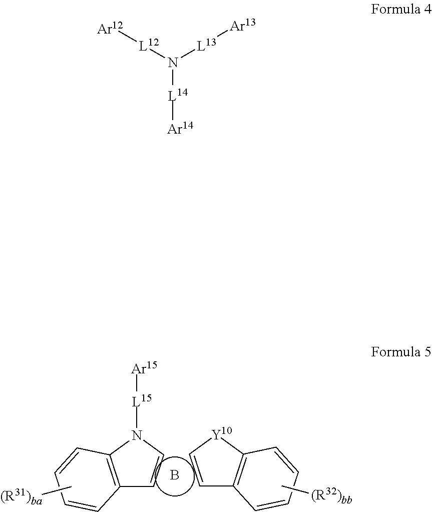
wherein:
L¹², L¹³, L¹⁴ and L¹⁵ are independently selected from the group consisting of a single bond; a C₆-C₆₀ arylene group; a fluorenylene group; a C₂-C₆₀ heterocyclic group including at least one heteroatom of O, N, S, Si or P; and a fused ring group of a C₃-C₆₀ aliphatic ring and a C₆-C₆₀ aromatic ring,
Ar¹², Ar¹³ and Ar¹⁴ are independently selected from the group consisting of a C₆-C₆₀ aryl group; a fluorenyl group; a C₂-C₆₀ heterocyclic group including at least one heteroatom of O, N, S, Si or P; a fused ring group of a C₃-C₆₀ aliphatic ring and a C₆-C₆₀ aromatic ring; a C₃-C₆₀ aliphatic ring; and -L′-N(R^{c})(R^{d}),
Ar¹⁵ is selected from the group consisting of a C₆-C₆₀ aryl group; a fluorenyl group; a C₂-C₆₀ heterocyclic group including at least one heteroatom of O, N, S, Si or P; a fused ring group of a C₃-C₆₀ aliphatic ring and a C₆-C₆₀ aromatic ring; and a C₃-C₆₀ aliphatic ring,
Y¹⁰ is O, S, C(R⁵¹)(R⁵²) or NR⁵³,
Ring B is an C₆-C₂₀ aryl,
L′ is selected from the group consisting of a single bond; a C₆-C₆₀ arylene group; a fluorenylene group; a C₂-C₆₀ heterocyclic group including at least one heteroatom of O, N, S, Si or P; and a C₃-C₆₀ aliphatic ring,
R³¹ and R³² are independently the same or different from each other and are independently selected from the group consisting of deuterium; a C₆-C₆₀ aryl group; a fluorenyl group; a C₂-C₆₀ heterocyclic group including at least one heteroatom of O, N, S, Si or P; a fused ring group of a C₃-C₆₀ aliphatic ring and a C₆-C₆₀ aromatic ring; a C₃-C₆₀ aliphatic ring; a C₁-C₅₀ alkyl group; a C₂-C₂₀ alkenyl group; a C₂-C₂₀ alkynyl group; a C₁-C₃₀ alkoxy group; and a C₆-C₃₀ aryloxy group, and a plurality of adjacent groups thereof may be bonded to each other to form a ring,
R⁵¹, R⁵², R⁵³, R^{c} and R^{d} are independently the same or different from each other and are independently selected from the group consisting of a C₆-C₆₀ aryl group; a fluorenyl group; a C₂-C₆₀ heterocyclic group including at least one heteroatom of O, N, S, Si or P; a fused ring group of a C₃-C₆₀ aliphatic ring and a C₆-C₆₀ aromatic ring; a C₃-C₆₀ aliphatic ring; a C₁-C₅₀ alkyl group; a C₂-C₂₀ alkenyl group; a C₂-C₂₀ alkynyl group; a C₁-C₃₀ alkoxy group; and a C₆-C₃₀ aryloxy group, and a plurality of adjacent groups thereof may be bonded to each other to form a spiro,
ba and bb are independently an integer from 0 to 4,
wherein the aryl group, arylene group, heterocyclic group, fluorenyl group, fluorenylene group, aliphatic ring group, fused ring group, alkyl group, alkenyl group, alkynyl group, alkoxy group and aryloxy group may be substituted with one or more substituents selected from the group consisting of deuterium; halogen; silane group; silane group substituted with an C₆-C₂₀ aryl group; siloxane group; boron group; germanium group; a cyano group; a nitro group; a C₁-C₂₀ alkylthio group; a C₁-C₂₀ alkoxyl group; a C₁-C₂₀ alkyl group; a C₂-C₂₀ alkenyl group; a C₂-C₂₀ alkynyl group; a C₆-C₂₀ aryl group; a C₆-C₂₀ aryl group substituted with deuterium; a fluorenyl group; a C₂-C₂₀ heterocyclic group; a C₃-C₂₀ aliphatic ring; a C₇-C₂₀ arylalkyl group; a C₈-C₂₀ arylalkenyl group; and a C₇-C₂₀ alkylaryl group; and -L′-N(R^{c})(R^{d}), and the hydrogen of these substituents may be substituted with one or more deuterium, and the substituents may be bonded to each other to form a saturated or unsaturated ring, wherein the term ‘ring’ means a C₃-C₆₀ aliphatic ring or a C₆-C₆₀ aromatic ring or a C₂-C₆₀ heterocyclic group or a fused ring formed by the combination thereof.

7. An organic electronic element comprising a first electrode; a second electrode; and an organic material layer formed between the first electrode and the second electrode, wherein the organic material layer comprises a compound according to claim 1.

8. The organic electronic element according to claim 7, wherein the organic material layer comprises a hole transport area, an emitting layer and an electron transport area.

9. The organic electronic element according to claim 7, wherein the organic material layer is an emitting layer.

10. The organic electronic element according to claim 7, wherein the organic material layer comprises 2 or more stacks comprising a hole transport layer, an emitting layer and an electron transport layer sequentially formed on a first electrode.

11. The organic electronic element according to claim 10, wherein the organic material layer further comprises a charge generation layer formed between the 2 or more stacks.

12. The organic electronic element according to claim 7, further comprising a light efficiency enhancing layer formed on at least one surface of the first electrode and the second electrode, the surface being opposite to the organic material layer.

13. An electronic device comprising a display device comprising the organic electronic element of claim 7; and a control unit for driving the display device.

14. The electronic device according to claim 13, wherein the organic electronic element is at least one of an OLED, an organic solar cell, an organic photo conductor (OPC), organic transistor (organic TFT) and an element for monochromic or white illumination.

15. The electronic device according to claim 13, wherein the electronic device is one of a wired communication terminal, wireless mobile communication terminal, PDAs, electronic dictionaries, PMPs, remote controls, navigation systems, game consoles, TVs, computers, watches, tablets, and virtual reality (VR) devices.

16. An organic electronic element comprising a first electrode; a second electrode; and an organic material layer formed between the first electrode and the second electrode, wherein the organic material layer comprises a composition for an organic electronic element of claim 6.

17. An electronic device comprising a display device comprising the organic electronic element of claim 16; and a control unit for driving the display device.

18. A method for reusing the compound of Formula 1 of claim 1 comprising:
recovering a crude organic light emitting material comprising a compound of Formula 1 from a deposition apparatus used in a process for depositing the organic emitting material to prepare an organic light emitting device;
removing impurities from the crude organic light emitting material;
recovering the organic light emitting material after the impurities are removed; and
purifying the recovered organic light emitting material to have a purity of 99.9% or higher.
